SEQUENCE LISTING (48 sequences)

SEQ ID NO: 1 - DNA, 6107 bp, Artificial Sequence
  pETDuet-1 HcI DU67866
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgtttaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 2 - DNA, 6125 bp, Artificial Sequence
  pETDuet-1 HcI 6His
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 3 - DNA, 6167 bp, Artificial Sequence
  pETDuet-1 C7 linker TEV IL13 70076
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaactaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 4 - DNA, 6305 bp, Artificial Sequence
  pETDuet-1 C7 linker TEV IL33 70051
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccagcattcagggcaccagcctgctgacccagagcccggcgagcctgagcacctataacgatcagagcgtgagctttgtgctggaaaacggctgctatgtgattaacgtggataaaagcggcaaagatcaggaacaggatcaggtgctgctgcgctattataaaagcccgtgcccggcgagccagagcggcgatggcgtggatggcaagaaactgatggtgaacatgagcccgattaaagataccgatatttggctgcatgcgaacgataaagattatagcgtggaactgcagcgcggcgatgtgagcccgccagaacaggcgttctttgtgctgcataagaaaagcagcgattttgtgagctttgaatgcaaaaacctgccgggcacctatattggcgtgaaagataaccagctggcgctggtggaagagaaagatgaaagctgcaacaacattatgtttaaactgagcaaaatttaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 5 - DNA, 6872 bp, Artificial Sequence
  pETDuet-1 HcI F L 6His - C7 linker TEV IL13 70080
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaactaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 6 - DNA, 7010 bp, Artificial Sequence
  pETDuet-1 HcI F L 6His - C7 linker TEV IL33 70059
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccagcattcagggcaccagcctgctgacccagagcccggcgagcctgagcacctataacgatcagagcgtgagctttgtgctggaaaacggctgctatgtgattaacgtggataaaagcggcaaagatcaggaacaggatcaggtgctgctgcgctattataaaagcccgtgcccggcgagccagagcggcgatggcgtggatggcaagaaactgatggtgaacatgagcccgattaaagataccgatatttggctgcatgcgaacgataaagattatagcgtggaactgcagcgcggcgatgtgagcccgccagaacaggcgttctttgtgctgcataagaaaagcagcgattttgtgagctttgaatgcaaaaacctgccgggcacctatattggcgtgaaagataaccagctggcgctggtggaagagaaagatgaaagctgcaacaacattatgtttaaactgagcaaaatttaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 7 - DNA, 7010 bp, Artificial Sequence
  pETDuet-1 HcI F L 6His - C7 linker TEV IL33 with mutated T7 70126
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcataatgcttaagtcgaacagaaagtaatcgtattgtacacggccgcataatcgaaattaatacgcctcactataggggaattgtgagcggataacaattccccatcttagtatattagttaagtataagaaggagatatacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccagcattcagggcaccagcctgctgacccagagcccggcgagcctgagcacctataacgatcagagcgtgagctttgtgctggaaaacggctgctatgtgattaacgtggataaaagcggcaaagatcaggaacaggatcaggtgctgctgcgctattataaaagcccgtgcccggcgagccagagcggcgatggcgtggatggcaagaaactgatggtgaacatgagcccgattaaagataccgatatttggctgcatgcgaacgataaagattatagcgtggaactgcagcgcggcgatgtgagcccgccagaacaggcgttctttgtgctgcataagaaaagcagcgattttgtgagctttgaatgcaaaaacctgccgggcacctatattggcgtgaaagataaccagctggcgctggtggaagagaaagatgaaagctgcaacaacattatgtttaaactgagcaaaatttaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 8 - DNA, 7139 bp, Artificial Sequence
  65750 pETDuet1 HcI F L 6His + Protein G Linker TEV C7
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgacctataaactgattctgaacggcaaaacgctgaaaggtgaaaccaccaccgaagcagtggatgccgcaaccgccgaaaaagttttcaaacagtacgccaacgataatggcgttgatggtgaatggacctacgatgatgcaacgaaaaccttcacggtgaccgaaaaaccggaagttattgatgccagcgaactgacgccggcagtgacgacctacaaactggttatcaatggcaaaaccctgaaaggtgaaacgaccaccgaagccgtggatgcagccaccgcagaaaaagtgtttaaacagtacgcgaacgataatggcgtggacggcgaatggacgtatgatgatgccaccaaaacgtttaccgttacggaaaaaccggaagtgatcgatgcatctgaactgaccccggcggtgaccacgtataaactggttatcaacggtaaaacgctgaaaggcgaaaccacgaccaaagccgtggatgcagaaaccgcggaaaaagcctttaaacagtatgcaaacgataatggcgtggatggtgtttggacctacgatgatgccacgaaaaccttcacggttaccgaaggcggtgggagtagcggaagtgggggtggtggatccagtggttctggggaaaatctgtacttccagggtgagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagctgaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 9 - DNA, 6317 bp, Artificial Sequence
  DU67808_pcDNA3D_FRT_SignalPeptide_ColE7_Spike_RBD_hexahis
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatctgccaccatgtggtggcgcctgtggtggctgctgctgctgctgctgctgctgtggcccatggtgtgggccgcagcagagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttccatcatcatcaccaccactgagcggccgcttacccatacgatgtgccagattacgcctaagtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 7031 bp, Artificial Sequence
  DU67793_pcDNA5D FRT TO SP C7 S-RBD His GFP 41574
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatctgccaccatgtggtggcgcctgtggtggctgctgctgctgctgctgctgctgtggcccatggtgtgggccgcagcagagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggaggtcatcatcatcaccaccacggaggtggctcatcaggatcaggtgcggccgctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactctaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 6752 bp, Artificial Sequence
  DU67817 pcDNA5D FRT TO SP C7 S-RBD N-ntd His
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatctgccaccatgtggtggcgcctgtggtggctgctgctgctgctgctgctgctgtggcccatggtgtgggccgcagcagagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggaggtggctcatcaggatcaggtggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagacctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcatcatcatcaccaccactgagcggccgcttacccatacgatgtgccagattacgcctaagtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - protein, 253 aa, Artificial Sequence
  HBcIm7
MDIDPYKEFGASVELLSFLPSDFFPSIRDLLDTASALYREALESPEHCSPHHTALRQAILCWGELMNLATWVGSNLGGGGSGGGGSELKNSISDYTEAEFVQLLKEIEKENVAATDDVLDVLLEHFVKITEHPDGTDLIYYPSDNRDDSPEGIVKEIKEWRAANGKPGFKQGGGGGSGGGGSPASRELVVSYVNVNMGLKIRQLLWFHISCLTFGRETVLEYLVSFGVWIRTPPAYRPPNAPILSTLPETTVV

SEQ ID NO: 13 - protein, 259 aa, Artificial Sequence
  HBcIm7hh
MDIDPYKEFGASVELLSFLPSDFFPSIRDLLDTASALYREALESPEHCSPHHTALRQAILCWGELMNLATWVGSNLGGGGSGGGGSELKNSISDYTEAEFVQLLKEIEKENVAATDDVLDVLLEHFVKITEHPDGTDLIYYPSDNRDDSPEGIVKEIKEWRAANGKPGFKQGGGGGSGGGGSPASRELVVSYVNVNMGLKIRQLLWFHISCLTFGRETVLEYLVSFGVWIRTPPAYRPPNAPILSTLPETTVVHHHHHH

SEQ ID NO: 14 - protein, 259 aa, Artificial Sequence
  HBcIm7-F/L
MDIDPYKEFGASVELLSFLPSDFFPSIRDLLDTASALYREALESPEHCSPHHTALRQAILCWGELMNLATWVGSNLGGGGSGGGGSELKNSISDYTEAEFVQLLKEIEKENVAATDDVLDVLLEHLVKITEHPDGTDLIYYPSDNRDDSPEGIVKEIKEWRAANGKPGFKQGGGGGSGGGGSPASRELVVSYVNVNMGLKIRQLLWFHISCLTFGRETVLEYLVSFGVWIRTPPAYRPPNAPILSTLPETTVVHHHHHH

SEQ ID NO: 15 - protein, 263 aa, Artificial Sequence
  HBcBsAAE
MDIDPYKEFGASVELLSFLPSDFFPSIRDLLDTASALYREALESPEHCSPHHTALRQAILCWGELMNLATWVGSNLGGGGSGGGGSKKAVINGEQIRSISDLHQTLKKELALPEYYGENLDALWDALTGWVEYPLVLEWRQFEQSKQLTENGAESVLQVFREAKAEGADITIELSGGGGGSGGGGSPASRELVVSYVNVNMGLKIRQLLWFHISCLTFGRETVLEYLVSFGVWIRTPPAYRPPNAPILSTLPETTVVHHHHHH

SEQ ID NO: 16 - protein, 254 aa, Artificial Sequence
  C7-IL13
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKSGGGSSGSGGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 17 - protein, 261 aa, Artificial Sequence
  C7-IL13-TEV
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKSGGGSSGSGENLYFQGGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 18 - protein, 301 aa, Artificial Sequence
  C7-IL33
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKSGGGSSGSGSSIQGTSLLTQSPASLSTYNDQSVSFVLENGCYVINVDKSGKDQEQDQVLLRYYKSPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 19 - protein, 308 aa, Artificial Sequence
  C7-IL33-TEV
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKSGGGSSGSGENLYFQGSSIQGTSLLTQSPASLSTYNDQSVSFVLENGCYVINVDKSGKDQEQDQVLLRYYKSPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 20 - protein, 352 aa, Artificial Sequence
  C7-PG
TYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGGGSSGSGGGGSSGSGENLYFQGESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKS

SEQ ID NO: 21 - protein, 237 aa, Artificial Sequence
  Bn-IL13
AQVINTFDGVADYLQTYHKLPDNYITKSEAQALGWVASKGNLADVAPGKSIGGDIFSNREGKLPGKSGRTWRWADINYTSGFRNSDRILYSSDWLIYKTTDHYQTFTKIRGGGSSGSGENLYFQGGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 22 - DNA, 759 bp, Artificial Sequence
  HBcIm7
atggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgtt

SEQ ID NO: 23 - DNA, 777 bp, Artificial Sequence
  HBcIm7hh
atggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccac

SEQ ID NO: 24 - DNA, 777 bp, Artificial Sequence
  HBcIm7-F/L
atggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccac

SEQ ID NO: 25 - DNA, 789 bp, Artificial Sequence
  HBcBsAAE
atggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctaaaaaagcagtcattaacggggaacaaatcagaagtatcagcgacctccaccagacattgaaaaaggagcttgcccttccggaatactacggtgaaaacctggacgctttatgggatgcgctgaccggatgggtggagtacccgctcgttttggaatggaggcagtttgaacaaagcaagcagctgactgaaaatggcgccgagagtgtgcttcaggttttccgtgaagcgaaagcggaaggcgcggacatcaccatcgaactttctggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccac

SEQ ID NO: 26 - DNA, 764 bp, Artificial Sequence
  C7-IL13
tggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaac

SEQ ID NO: 27 - DNA, 789 bp, Artificial Sequence
  C7-IL13-TEV
atggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaac

SEQ ID NO: 28 - DNA, 788 bp, Artificial Sequence
  C7-IL33
tggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaac

SEQ ID NO: 29 - DNA, 927 bp, Artificial Sequence
  C7-IL33-TEV
atggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccagcattcagggcaccagcctgctgacccagagcccggcgagcctgagcacctataacgatcagagcgtgagctttgtgctggaaaacggctgctatgtgattaacgtggataaaagcggcaaagatcaggaacaggatcaggtgctgctgcgctattataaaagcccgtgcccggcgagccagagcggcgatggcgtggatggcaagaaactgatggtgaacatgagcccgattaaagataccgatatttggctgcatgcgaacgataaagattatagcgtggaactgcagcgcggcgatgtgagcccgccagaacaggcgttctttgtgctgcataagaaaagcagcgattttgtgagctttgaatgcaaaaacctgccgggcacctatattggcgtgaaagataaccagctggcgctggtggaagagaaagatgaaagctgcaacaacattatgtttaaactgagcaaaatt

SEQ ID NO: 30 - DNA, 1059 bp, Artificial Sequence
  C7-PG
atgacctataaactgattctgaacggcaaaacgctgaaaggtgaaaccaccaccgaagcagtggatgccgcaaccgccgaaaaagttttcaaacagtacgccaacgataatggcgttgatggtgaatggacctacgatgatgcaacgaaaaccttcacggtgaccgaaaaaccggaagttattgatgccagcgaactgacgccggcagtgacgacctacaaactggttatcaatggcaaaaccctgaaaggtgaaacgaccaccgaagccgtggatgcagccaccgcagaaaaagtgtttaaacagtacgcgaacgataatggcgtggacggcgaatggacgtatgatgatgccaccaaaacgtttaccgttacggaaaaaccggaagtgatcgatgcatctgaactgaccccggcggtgaccacgtataaactggttatcaacggtaaaacgctgaaaggcgaaaccacgaccaaagccgtggatgcagaaaccgcggaaaaagcctttaaacagtatgcaaacgataatggcgtggatggtgtttggacctacgatgatgccacgaaaaccttcacggttaccgaaggcggtgggagtagcggaagtgggggtggtggatccagtggttctggggaaaatctgtacttccagggtgagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagc

SEQ ID NO: 31 - DNA, 19 bp, Artificial Sequence
  T7-mutant
taatacgactcactatagg

SEQ ID NO: 32 - DNA, 786 bp, Artificial Sequence
  Bn-IL13
atggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgttgggcggatattaactatacatcaggcttcagaaattcagaccggattctttactcaagcgactggctgatttacaaaacaacggaccattatcagacctttacaaaaatcagaggaggtggctcatcaggatctggtgaaaacctgtattttcagggatccggcccggtgcctccaagcactgccctgcgtgagttaatcgaagaattagtcaacattacccaaaaccagaaggcaccgctgtgcaacggttcaatggtctggagcatcaatctgacagccggtatgtattgcgcggcgcttgaaagcctgattaacgtgagtggttgttctgccatcgaaaaaactcaacgtatgttgtccggcttttgcccacacaaggtatctgccggtcaattcagttcattgcacgttcgtgatacaaaaattgaggtagcccagttcgtgaaggacttactgttacatttgaaaaagttattccgtgaagggcaatttaactaataaggtaccctcgagtctggtaaagaaaccgctgctgcgaaatttgaacgccagcacatggactcg

SEQ ID NO: 33 - protein, 10 aa, Artificial Sequence
  linker
GGGGSGGGGS

SEQ ID NO: 34 - protein, 11 aa, Artificial Sequence
  linker
GGGGGSGGGGS

SEQ ID NO: 35 - protein, 9 aa, Artificial Sequence
  linker
SGGGSSGSG

SEQ ID NO: 36 - protein, 89 aa, Artificial Sequence
  Barstar
KKAVINGEQIRSISDLHQTLKKELALPEYYGENLDALWDALTGWVEYPLVLEWRQFEQSKQLTENGAESVLQVFREAKAEGADITIELS

SEQ ID NO: 37 - protein, 85 aa, Artificial Sequence
  Im7
ELKNSISDYTEAEFVQLLKEIEKENVAATDDVLDVLLEHFVKITEHPDGTDLIYYPSDNRDDSPEGIVKEIKEWRAANGKPGFKQ

SEQ ID NO: 38 - protein, 110 aa, Artificial Sequence
  Barnase
AQVINTFDGVADYLQTYHKLPDNYITKSEAQALGWVASKGNLADVAPGKSIGGDIFSNREGKLPGKSGRTWRWADINYTSGFRNSDRILYSSDWLIYKTTDHYQTFTKIR

SEQ ID NO: 39 - protein, 134 aa, Artificial Sequence
  ColE7
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKS

SEQ ID NO: 40 - protein, 148 aa, Artificial Sequence
  HBc
  MISC_FEATURE (77)..(77): X indicates the position of an insertion, suitably of an amino acid insertion, suitably of an amino acid sequence encoding the first binding protein.
MDIDPYKEFGASVELLSFLPSDFFPSIRDLLDTASALYREALESPEHCSPHHTALRQAILCWGELMNLATWVGSNLXPASRELVVSYVNVNMGLKIRQLLWFHISCLTFGRETVLEYLVSFGVWIRTPPAYRPPNAPILSTLPETTVV

SEQ ID NO: 41 - DNA, 1094 bp, Artificial Sequence
  C7-RBD
gagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaattt

SEQ ID NO: 42 - DNA, 1530 bp, Artificial Sequence
  C7-RBD-nucleocapsid
gagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggaggtggctcatcaggatcaggtggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagacctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagt

SEQ ID NO: 43 - DNA, 1869 bp, Artificial Sequence
  C7-RBD-GFP
gagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatccggtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcggaggtcatcatcatcaccaccacggaggtggctcatcaggatcaggtgcggccgctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 44 - DNA, 7 bp, Artificial Sequence
  T7  promoter parent sequence
gcataat

SEQ ID NO: 45 - DNA, 867 bp, Artificial Sequence
  C7-IL17
atggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggaggtggctcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaataa

SEQ ID NO: 46 - protein, 281 aa, Artificial Sequence
  ColE7-IL17
MESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKATSFALHHEKPISQNGGVYDMDNISVVTPKRAIDIHRGKSGGGSSGSGENLYFQGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 47 - DNA, 7634 bp, Artificial Sequence
  pETDuet-1 HBc-Im7 F->L 6His - tet-inducible_ColE7-linker- TEV_site_hIL17A
gggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcttaacctaggctgctgccaccgctgagcaataactagcataaccccttggggcctctaaacgggtcttgaggggttttttgttgtacacggccgcataatcggttgacactctatcattgatagagttattttaccactccctatcagtgatagagaaaagtgaaatgaatagttcgacaaaaatctagataacgagggcaacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccggaggtggctcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctttaggacccactttcacatttaagttgtttttctaatccgcagatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactatag

SEQ ID NO: 48 - DNA, 7700 bp, Artificial Sequence
  DU70612. pETDuet-1 HBc-Im7 F->L 6His - tet-inducible_ColE7-linker- TEV_site_mIL33
ggggaattgtgagcggataacaattcccctctagaaataattttgtttaactttaagaaggagatataccatggacattgacccgtataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatttggccacctgggtgggaagtaatttgggaggaggtggaagcggaggaggtggatctgaactgaaaaatagtattagtgattacacagaggctgagtttgttcaacttcttaaggaaattgaaaaagagaatgttgctgcaactgatgatgtgttagatgtgttactcgaacacctagtaaaaattactgagcatccagatggaacggatctgatttattatcctagtgataatagagacgatagccccgaagggattgtcaaggaaattaaagaatggcgagctgctaacggtaagccaggatttaaacagggcggaggaggtggaagcggaggaggtggatctccagcatccagggaattagtagtcagctatgttaatgttaatatgggcctaaaaatcagacaactactgtggtttcacatttcctgtcttacttttggaagagaaactgttcttgaatatttggtgtcttttggagtgtggattcgcactcctcctgcttacagaccaccaaatgcccctatcttatcaacacttccggaaactactgttgttcatcaccatcatcaccactaataagcggccgcttaacctaggctgctgccaccgctgagcaataactagcataaccccttggggcctctaaacgggtcttgaggggttttttgttgtacacggccgcataatcggttgacactctatcattgatagagttattttaccactccctatcagtgatagagaaaagtgaaatgaatagttcgacaaaaatctagataacgagggcaacatatggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtcgaaacaataatgatcgaatgaaggttggaaaagcgcccaagactagaacccaggatgtttcagggaaggcaacttcattcgcacttcatcatgagaagccgatcagccaaaatggtggtgtctatgatatggataacatcagcgtggtaacacctaaacgtgctattgatattcaccgaggtaaaagcggaggtggctcatcaggatctggtgaaaacctgtattttcagggatccagcattcagggcaccagcctgctgacccagagcccggcgagcctgagcacctataacgatcagagcgtgagctttgtgctggaaaacggctgctatgtgattaacgtggataaaagcggcaaagatcaggaacaggatcaggtgctgctgcgctattataaaagcccgtgcccggcgagccagagcggcgatggcgtggatggcaagaaactgatggtgaacatgagcccgattaaagataccgatatttggctgcatgcgaacgataaagattatagcgtggaactgcagcgcggcgatgtgagcccgccagaacaggcgttctttgtgctgcataagaaaagcagcgattttgtgagctttgaatgcaaaaacctgccgggcacctatattggcgtgaaagataaccagctggcgctggtggaagagaaagatgaaagctgcaacaacattatgtttaaactgagcaaaatttaataaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctttaggacccactttcacatttaagttgtttttctaatccgcagatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

(19) United States
(12) Patent Application Publication
(10) Pub. No.: US 2024/0093159 A1
(43) Pub. Date: Mar. 21, 2024

(54) VIRUS-LIKE PARTICLES AND METHODS OF PRODUCTION THEREOF

(71) Applicant: University of Dundee, Dundee (GB)

(72) Inventor: John FOERSTER, Dundee (GB)

(21) Appl. No.: 18/038,585

(22) PCT Filed: Nov. 26, 2021
(86) PCT No.: PCT/GB2021/053093

§ 371 Date: May 24, 2023

(30) Foreign Application Priority Data
Nov. 30, 2020 (GB) 2018816.5

(51) Int. Cl. C12N 7/00; A61K 39/12; C07K 14/005; C07K 14/32; C07K 14/54; C12N 9/16; C12N 9/88

(52) U.S. Cl. CPC C12N 7/00; A61K 39/12; C07K 14/005; C07K 14/32; C07K 14/54; C07K 14/5437; C12N 9/16; C12N 9/88; C12Y 301/21001; A61K 2039/5258; C07K 2319/55; C12N 2730/10122; C12N 2730/10123; C12N 2730/10151

(57) ABSTRACT

The present invention relates to virus-like particles (VLPs) having a high affinity protein attachment system which allows interchangeable decoration with any functional molecule of choice. The present invention further relates to processes of producing the VLPs, including a rapid single cell process, and uses of the VLPs in research, diagnosis and as vaccines for use in prevention/treatment of diseases.

The present invention relates to virus-like particles (VLPs) having a high affinity protein attachment system which allows interchangeable decoration with any functional molecule of choice. The present invention further relates to processes of producing the VLPs, including a rapid single cell process, and uses of the VLPs in research, diagnosis and as vaccines for use in prevention/treatment of diseases.

### INTRODUCTION

Virus-like particles (VLPs) are molecules that closely resemble viruses, but contain no viral genetic material. They are formed from viral structural proteins, such as viral capsid proteins that, when individually expressed, self-assemble into a particle. Most Virus like particles look like hollow ‘nano-footballs’ where the entire surface of the football is made up by many copies of a single self-assembled protein. For production purposes this means that production of one single protein is sufficient to generate a big nano-football type VLP structure.

This has been exploited in medicine. The most common use of VLPs is as vaccines. The basic mechanism behind this is that mammals have evolved immune sensing mechanisms to recognise highly repetitive patterns seen on viral capsids as intruders. These patterns are still present in VLPs, which contain repetitive, high density displays of viral surface proteins, but the harmful viral genome is removed. This is the form of the VLP used as the vaccine against human papillomavirus (HPV) which causes cervical cancer. There are currently a selection of commercially available HPV vaccines of this type such as Cervarix by GlaxoSmithKline along with Gardasil and Gardasil-9, produced by Merck & Co.

Further developments of VLPs for use as vaccines involve tethering of other agents to the VLP shell. In this case, the VLP shell serves to present an additional agent as an ‘epitope’ to the immune system. In some cases, the viral capsid proteins forming the VLP shell can be modified to directly incorporate the epitope for display through genetic fusion. However, this approach commonly leads to impaired VLP assembly and large proteins routinely cause VLP instability. Further, this approach cannot be used if the agent is not protein-based. Current COVID19 vaccines that are under development use this form of VLP, where the spike protein from the coronavirus is directly fused to a viral capsid protein forming a VLP shell from an unrelated virus.

A further alternative is to assemble the VLP and then use attachment means to secure the agent to the VLP shell. Such VLPs with additional attachment means may be termed ‘compound VLPs’. Compound VLPs may be manufactured by methods such as chemical crosslinking, reactive unnatural amino acids, or the use of binding proteins such as the SpyTag/SpyCatcher system, to covalently attach the desired agent to the viral capsid proteins forming the shell.

The latter method allows the attachment of other non-protein epitopes to the VLP, but requires a complicated production process and cannot yet be used for any agent. Some desired proteins are simply too large to attach to the VLP shell using current attachment means, and some complex epitopes include multimers with numerous components that must be separately linked together, which must be achieved by additional chemical crosslinking.

The current binding proteins which are used as attachment means, such as in the SpyTag/SpyCatcher system, have further issues in that the binding between the proteins whilst being strong does not occur instantly but requires time for the reactants to fuse, and can result in VLP-shell aggregation depending on which agent is attached to the shell.

For VLPs used in clinical human or veterinary applications, regulators classify VLPs as “biological” active drug intermediates (ADI's). “Biologic” drugs are produced in living cells, followed by purification according to a regulator—approved process. Each cell line (regardless whether bacterial/plant/yeast/insect/mammalian) used for the production is minutely characterized so as to guarantee long-term stability of the ADI and stored under highly specified conditions as a so-called “Master Cell Bank” (MCB). If a VLP requires two (or even more) proteins to assembled, for example where proteins are used to attach an epitope to the VLP shell, then currently one MCB is required for each protein component of the drug and both require a separate purification process, each requiring separate characterisation procedures, as both are classed as “critical drug intermediates”. Also, a separate quality-control release of required for each critical drug intermediate, as well as the final ADI, multiplying manufacturing cost.

In addition to these complexities, the production of the epitope must be established from scratch for each epitope. Financially, the most efficient type of production cell is bacteria (specifically: E. coli). However, many proteins do not assume their native shape when produced in E. coli but must be re-folded into their proper form from a denatured state as part of the purification process, which results in huge drop of overall yield and significantly adds to process complexity.

As a result of all these features, the production process of compound VLP-type drugs which attempt to attach agents such as epitopes to the viral capsid proteins is complex and expensive. This has limited wide-spread exploration of VLP applications to fields where inexpensive mass production could make them more competitive.

It would be desirable to provide compound VLPs with the modular ability to display almost any desired agent, including non-proteins, those proteins that experience difficulty in folding within E. coli, and those proteins that are complex and large such as multimers. It would further be desirable to produce such compound VLPs by a much simpler process requiring the use of only one cell-line, and in which the VLP shell and the agent to be displayed can auto-assemble within the cell line.

One or more aspects of the present invention are aimed at solving one or more of the above-mentioned problems.

### STATEMENTS OF INVENTION

According to a first aspect of the present invention, there is provided a virus-like particle (VLP) comprising:
One or more hepatitis B capsid proteins
One or more pairs of binding proteins, each pair of binding proteins comprising a first binding protein and a second binding protein, wherein the pair of binding proteins comprises a bacterial toxin and its inhibitor
One or more functional molecules
   wherein each hepatitis B capsid protein is attached to a first binding protein, wherein each functional molecule is attached to a second binding protein, and wherein the first and second binding proteins are bound to each other.

In one embodiment, the one or more first binding proteins may comprise a chemical modification.

In one embodiment, the chemical modification may be attached to a second functional molecule.

In one embodiment, the first and second functional molecules may be different. In one embodiment, the first functional molecule may be selected from an antigen or an antigen binding protein such as an antibody. In one embodiment, the second functional molecule may be a fluorescent label.

In one embodiment, if one of the functional molecule(s) is an antigen binding protein, then the VLP may further comprise a third binding protein, wherein the antigen binding protein is attached to the third binding protein, and the third binding protein in turn is attached to the second binding protein. In one embodiment, the third binding protein is protein G. Therefore, suitably the functional molecule may be directly or indirectly attached to the second binding protein. Suitably the functional molecule may be indirectly attached to the second binding protein via a third binding protein.

According to a second aspect of the present invention, there is provided a virus-like particle (VLP) comprising:
One or more viral capsid proteins
One or more binding proteins comprising a chemical modification, wherein each binding protein is a bacterial toxin inhibitor;
One or more functional molecules
   wherein each viral capsid protein is attached to a binding protein, and wherein each chemical modification is attached to a functional molecule.
In one embodiment, the functional molecule a non-protein molecule, suitably a non-protein antigen or a flourescent molecule.
In one embodiment, the VLP may further comprise a second binding protein, wherein the second binding protein is bound to the first binding protein. In one embodiment, the second binding protein may be attached to a second functional molecule.
In one embodiment, the second binding protein may be a bacterial toxin. Suitably a bacterial toxin which binds to the bacterial toxin inhibitor.
In one embodiment, the first and second functional molecules may be different. In one embodiment, the first functional molecule may be a fluorescent label. In one embodiment, second functional molecule may be selected from an antigen or an antigen binding protein such as an antibody.
In one embodiment, if one of the functional molecule(s) is an antigen binding protein, then the VLP further comprises a third binding protein, wherein the antigen binding protein is attached to the third binding protein, and the third binding protein is in turn attached to the second binding protein. In one embodiment, the third binding protein is protein G. Therefore, suitably the second functional molecule may be directly or indirectly attached to the second binding protein. Suitably the second functional molecule may be indirectly attached to the second binding protein via a third binding protein.

According to a third aspect of the present invention, there is provided a capsid fusion protein comprising a viral capsid protein fused to a binding protein, wherein the binding protein is a bacterial toxin inhibitor.
In one embodiment, the viral capsid protein is a hepatitis B viral capsid protein.
In one embodiment, the capsid fusion protein comprises SEQ ID NO: 12, 13, 14, or 15.
In one embodiment, the binding protein comprises a chemical modification.

According to a fourth aspect of the present invention, there is provided a functional fusion protein comprising a functional molecule fused to a binding protein wherein the binding protein is a bacterial toxin.
In one embodiment, the functional fusion protein comprises SEQ ID NO:16, 17, 18, 19, 21, or 46.

According to an alternative fourth aspect of the present invention, there is provided a functional fusion protein comprising a first binding protein fused to a further binding protein, wherein the first binding protein is a bacterial toxin, and the further binding protein is able to capture functional molecules that are antigen binding proteins.
In one embodiment, the further binding protein is capable of binding to a functional molecule. Suitably, in such an embodiment, the functional molecule is an antigen binding protein such as an antibody. Sutiably the further binding protein is the third binding protein as described elsewhere herein. In one embodiment, the further binding protein is protein G.
In one embodiment, the functional fusion protein comprises SEQ ID NO: 20.

According to a fifth aspect of the present invention, there is provided one or more nucleic acids encoding the capsid fusion protein of the third aspect or the functional fusion protein of the fourth aspects.
In one embodiment, the nucleic acid encodes the capsid fusion protein and comprises a sequence selected from SEQ ID NO: 22, 23, 24, or 25.
In one embodiment, the nucleic acid encodes the functional fusion protein and comprises a sequence selected from SEQ ID NO: 26, 27, 28, 29, 30 32, 41, or 45.

According to a sixth aspect of the present invention, there is provided one or more vectors comprising the one or more nucleic acid(s) of the fifth aspect.
In one embodiment, the vector may comprise a sequence selected from SEQ ID NO: 1-11, 47 or 48.

According to a seventh aspect of the present invention, there is provided a host cell comprising the one or more nucleic acid(s) of the fifth aspect, or a vector of the sixth aspect.

According to an eighth aspect of the present invention, there is provided a host cell comprising one or more vectors, the one or more vectors comprising a first nucleic acid encoding a hepatitis B capsid protein attached to a first binding protein; and a second nucleic acid encoding a functional molecule attached to a second binding protein; wherein the first and second binding proteins are capable of binding to each other.

According to a ninth aspect of the present invention, there is provided a host cell comprising one or more vectors, the one or more vectors comprising a first nucleic acid encoding a viral capsid protein attached to a first binding protein; and a second nucleic acid encoding a functional molecule; wherein the functional molecule is capable of binding to the first binding protein via a chemical modification of the first binding protein.

According to a tenth aspect of the present invention, there is provided a process of producing a virus-like particle (VLP) in a single host cell comprising:
(a) Providing a host cell comprising one or more vectors, wherein the one or more vectors comprise:
   (i) a first nucleic acid encoding a viral capsid protein attached to a first binding protein; and
   (ii) (a) a second nucleic acid encoding a functional molecule optionally attached to a second binding protein; and/or
(b) a third nucleic acid encoding a second binding protein attached to a third binding protein;
(b) Culturing the host cell under conditions to express the proteins from the first, second and/or third nucleic acids respectively;
(c) Forming virus-like particles from the proteins.
In one embodiment, the viral capsid protein is a hepatitis B capsid protein. In one embodiment, the first binding protein is a bacterial toxin inhibitor. In one embodiment, the second binding protein is a bacterial toxin.
In one embodiment, step (b) further comprises culturing under conditions such that the proteins bind to each other.
In some embodiments, during the culturing step the first binding protein may be chemically modified. Suitably therefore the conditions are such that the first binding protein is chemically modified.
In other embodiments, the method further comprises a step of recovering the proteins, and subsequently chemically modifying the first binding protein. Suitably these steps take place after step (b) but prior to step (c).
In some embodiments, the one or more vectors may further comprise a further nucleic acid encoding a second functional molecule. In some embodiments, this may occur when the second nucleic acid encodes a functional molecule attached to a second binding protein. Suitably in such embodiments, the first binding protein is chemically modified.
In some embodiments, the one or more vectors may comprise option (ii)(a) a second nucleic acid encoding a functional molecule attached to a second binding protein. Suitably in such an embodiment, the first binding protein may or may not be chemically modified.
In some embodiments, the one or more vectors may comprise option (ii)(b) a nucleic acid encoding a second binding protein attached to a third binding protein. Suitably in such an embodiment, a functional molecule for attachment to the third binding protein may be added to the VLPs once formed, suitably after step (d). Alternatively, a functional molecule for attachment to the third binding protein may be encoded on the second nucleic acid of option (ii)(a), suitably wherein the second nucleic acid may encode only a functional molecule.
In some embodiments therefore, the one or more vectors may comprise both option (ii)(a) a second nucleic acid encoding a functional molecule, and option (ii)(b) a nucleic acid encoding a second binding protein attached to a third binding protein.
Suitably in such embodiments, the host cell is cultured under conditions to express the proteins from each of the nucleic acids.
In one embodiment, the first binding protein is chemically modified. Suitably, in such an embodiment, the one or more vectors may comprise option (ii)(a) and the second nucleic acid may encode only a functional molecule.
In one embodiment, step (c) comprises each first binding protein binding to each second binding protein. In an alternative embodiment, step (c) comprises each first binding protein binding to a functional molecule, suitably via a chemical modification. In one embodiment, step (c) comprises both of these steps.
Suitably the method may further comprise step (d) adding a functional molecule to the virus-like particles, suitably by attachment to the third binding protein if present.

According to an eleventh aspect of the present invention, there is provided a process of producing a virus-like particle (VLP), comprising;
(a) Providing a first host cell comprising one or more vectors, wherein the one or more vectors comprise:
   (i) a first nucleic acid encoding a hepatitis B capsid protein attached to a first binding protein, wherein the first binding protein is a bacterial toxin inhibitor;
(b) Providing one or more further host cell(s) comprising one or more vectors, wherein the one or more vectors comprise:
   (i) a second nucleic acid encoding a functional molecule optionally attached to a second binding protein, wherein the second binding protein is a bacterial toxin; and/or
   (ii) a third nucleic acid encoding a second binding protein attached to a third binding protein, wherein the second binding protein is a bacterial toxin;
(c) Culturing the host cells under conditions to express the proteins from the first, second and/or third nucleic acids respectively;
(d) Recovering the proteins;
(e) Mixing the proteins to form virus-like particles.
In some embodiments, during the culturing step the first binding protein may be chemically modified. Suitably therefore the conditions for culturing the first host cell are such that the first binding protein is chemically modified.
In other embodiments, the method may comprise a step of chemically modifying the first binding protein. Suitably this step takes place after step (d) but prior to step (e).
In one embodiment, the first binding protein is chemically modified. Suitably, in such an embodiment, the second nucleic acid may encode only a functional molecule.
In one embodiment, the second nucleic acid encodes a functional molecule attached to a second binding protein. Suitably in such an embodiment, the first binding protein may or may not be chemically modified.
In some embodiments, the one or more vectors may further comprise a nucleic acid encoding a second functional molecule. In some embodiments, this may occur when the second nucleic acid encodes a functional molecule attached to a second binding protein. Suitably the further nucleic acid may be comprised on a vector in the first or further host cells. Suitably in such embodiments, the first binding protein is chemically modified.
In some embodiments, the one or more vectors may comprise option (b)(i) a second nucleic acid encoding a functional molecule attached to a second binding protein, wherein the second binding protein is a bacterial toxin. Suitably in such an embodiment, the first binding protein may or may not be chemically modified.
In some embodiments, the one or more vectors may comprise option (b)(ii) a third nucleic acid encoding a second binding protein attached to a third binding protein. Suitably in such an embodiment, a functional molecule for attachment to the third binding protein may be added to the VLPs once formed, suitably after step (e). Alternatively, a functional molecule for attachment to the third binding protein may be encoded on the second nucleic acid. Suitably in such an embodiment, the second nucleic acid may encode only a functional molecule.
In some embodiments therefore, the one or more vectors in any host cell may comprise both (b)(i) a second nucleic acid encoding a functional molecule, and (b)(ii) a third nucleic acid encoding a second binding protein attached to a third binding protein.
Suitably a second host cell may comprise one or more vectors encoding the nucleic acid(s) of (b)(i) alone, or of (b)(i) and (b)(ii).
Suitably a third host cell may comprise one or more vectors encoding the nucleic acid(s) of (b)(ii).
Suitably in such embodiments, the host cells are cultured under conditions to express the proteins from each of the nucleic acids.
In one embodiment, step (e) comprises each first binding protein binding to each second binding protein. In an alternative embodiment, step (e) comprises each first binding protein binding to each functional molecule, suitably via a chemical modification. In one embodiment, step (e) comprises both of these steps.
In one embodiment, step (e) further comprises mixing under conditions such that the proteins bind to each other.
Suitably the method may comprise step (f) of adding a functional molecule to the virus-like particles, suitably by attachment to the third binding protein if present.

According to a twelfth aspect of the present invention, there is provided an immunogenic composition comprising the virus-like particle of the first or second aspects.

According to a thirteenth aspect of the present invention, there is provided a virus-like particle (VLP) of the first or second aspects or an immunogenic composition of the eleventh aspect for use as a medicament.

According to a fourteenth aspect of the present invention, there is provided a virus-like particle (VLP) of the first or second aspects or an immunogenic composition of the eleventh aspect for use in the prevention and/or treatment of infectious diseases, cardiovascular diseases, cancer, inflammatory diseases, autoimmune diseases, neurological disease, metabolic disease, rheumatological degenerative disease, or addiction.

According to a fifteenth aspect of the present invention, there is provided use of the virus-like particle (VLP) of the first or second aspects in research, or in the diagnosis of a disease.

According to a sixteenth aspect of the present invention, there is provided a method of diagnosing a disease in a subject comprising:
(a) Providing a virus like particle according to the first or second aspects, wherein the functional molecule is an antibody directed towards an antigen derived from a disease causing agent;
(b) Mixing the virus like particle with a suitable sample from the subject;
(c) Detecting whether the virus like particle precipitates;
(d) Diagnosing the presence of a disease if the VLP precipitates.

Advantageously the present invention provides a novel VLP structure which makes use of the desirable characteristics of bacterial toxin and inhibitor protein binding pairs to attach a functional molecule to the VLP shell. The use of this type of protein binding pair in a VLP setting has never been tested prior to the present invention. The inventors found that in combination with the Hepatitis B capsid protein, the use of bacterial toxin and inhibitor protein binding pairs provides a very stable VLP to which almost any functional molecule can be attached. The high affinity binding can be applied to any functional molecule without having to determine conditions for a given molecule to connect to the VLP shell. Moreover, this system allows even large and entire proteins to be “pasted” onto the VLP-shell which could not be done as direct fusion to the VLP-shell protein. The inventors designed the Hepatitis B capsid protein and toxin/inhibitor combination to be placed in a particular orientation such that when the VLP is formed, the binding proteins face away from one another and do not interfere with each other, thus avoiding capsid instability. Furthermore, a hepatitis B viral capsid was also found to tolerate such a fusion without loss of structure. Still further, the hepatitis B capsid protein core unit is dimeric, which allows two hepatitis B capsid monomers to each be attached to a monomer of a dimeric functional molecule. When the two hepatitis B capsid monomers come together during assembly, then the VLP can display dimeric functional molecules such as certain key cytokines in their natural form. The inventors have successfully managed to display dimeric functional molecules such as IL17, which have previously been difficult to genetically fuse into a VLP structure.

The invention further provides a novel method of producing VLPs which takes place within one host cell. This method is significantly less complex and therefore less costly than current methods of VLP production. This process has the potential to hugely lower drug treatment costs and increase patient access to treatment with VLPs. The inventors surprisingly found that the novel VLP structure described above also provides advantages in production which enable such a single cell method. The inventors found that by fusing the VLP capsid protein to the toxin protein, and by fusing the functional molecule to the partner inhibitor protein, then once both fusion proteins are produced in a cell, the VLP will auto-assemble within the single cell by virtue of the high affinity binding of the bacterial toxin and inhibitor pair.

Furthermore, the use of a bacterial toxin and inhibitor pair also provided the advantage there is a differential electrical charge distribution create a homogenous negative surface charge on the VLP shells. This prevents formation of undesired aggregates and clumps between VLPs during production. Further still, the fusion of the positively charged binding-protein (generally the toxin) to the functional molecule simultaneously serves as a “chaperone” for the functional molecule during production. This means that functional molecules such as certain proteins which could, when produced on their own, either be non-soluble or even be toxic to a host cell, in many cases lose their toxicity and/or become soluble due to fusion with the bacterial toxin. This further adds to the general utility of the system by facilitating the production of many difficult-to-produce proteins.

Surprisingly, in addition, the inventors have discovered that chemical modification of the toxin inhibitor can occur using chemicals such as DEAE or octylamine which allows further functional molecules to be attached to the protein binding pair in addition to, and independently of the toxin inhibitor binding to the toxin partner. This chemistry allows a second way to decorate VLPs, for example with small chemicals, such as fluorescent dyes, linked to octylamine, in parallel, or instead of, decoration with the toxin-fused protein. This, in turn allows huge flexibility and versatility to the VLP structure with the option of attaching multiple functional molecules to one protein binding pair, which may have different functions.

In summary, the inventors have created a novel VLP system and method of production thereof which allows fast manufacturing of compound VLPs presenting any type of functional molecule as a single-release drug for biomedical applications; allows the production VLP-linkage of functional molecules which, in the absence of a ‘chaperone’ would not be producible in cells such as bacteria; allows the presentation of complex functional molecules such as dimers without having to use additional chemical crosslinking and without disrupting the VLP stability; and allows stable VLPs that are functionally flexible.

Features and embodiments of the above aspects are described further under headed sections below. Any feature or embodiment may be combined with any aspect in any workable combination.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

The present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

The terms “identity” and “identical” and the like refer to the sequence similarity between two polymeric molecules, e.g., between two nucleic acid molecules, such as between two DNA molecules, or between two protein molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated.

The term “operably linked”, “operably connected” or equivalent expressions as used herein refer to the arrangement of various nucleic acid elements relative to each other such that the elements are functionally connected and are able to interact with each other in the manner intended.

The terms “therapy” “therapeutic” “treatment” or “treating” refer to reducing, ameliorating or eliminating one or more signs, symptoms, or effects of a disease or condition. “Treatment,” or “therapy” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The “administration” of an agent to a subject includes any route of introducing or delivering to a subject the agent to perform its intended function. Administration can be carried out by any suitable route, including orally, intranasally, intraocularly, ophthalmically, parenterally (intravascularly, intramuscularly, intraperitoneally, or subcutaneously), or topically. Administration includes self-administration and the administration by another.

The terms “individual,” “subject,” and “patient” are used interchangeably, and refer to any individual subject with a disease or condition in need of therapy, suitably in need of therapy by treatment with the present invention. For the purposes of the present disclosure, the subject may be a human or animal, for example primate, preferably a human, or another mammal, such as a dog, cat, horse, pig, goat, or bovine, and the like.

### FIGURES

The invention may be described with reference to the following figures in which:

FIG. 1 shows: (A) chromatography fractions with pure VLP enriched and (B) an electron microscope image of VLPs demonstrating that HBc VLP shells can be fused with the Im7 domain and can then be purified and are stable VLPs of the expected size;

FIG. 2 shows: (A) exemplary plasm id diagram for prokaryotic cells; (B) exemplary plasmid diagram for eukaryotic cells; (C) exemplary plasm id diagram for independent induction of VLP-backbone versus functional molecule (IL-33 cytokine), via T7-promoter and tetR-promoters respectively.

FIG. 3 shows: SDS-PAGE of candidate HBc-Im7 and colE7-IL13 proteins expressed in E. coli from 3 different clones; showing that significant amounts of both proteins are soluble, indicative of native protein folding.

FIG. 4 shows: SDS-PAGE of proteins isolated and purified from (A) DEAE-anion exchange chromatography of parent HBc VLPs; (B) DEAE-anion exchange chromatography of HBc-Im7 fused VLPs, showing that the elution profile of HBc-Im7 is significantly altered, resembling that typically encountered in affinity chromatography.

FIG. 5 shows: SDS-PAGE of proteins isolated and purified from multimodal hydrophobic/weak anion exchange chromatography of Hbc-Im7 fused VLPs, showing that HBc-Im7 irreversibly binds to the stationary phase.

FIG. 6 shows: SDS-PAGE of flow-through fractions of DEAE-partially purified HBc-Im7 VLPs subjected to mixed mode size exclusion chromatography (cut-off 700 kD), suggesting that proteins elute in the form of intact VLPs and showing that two-step purification yields highly pure VLP fractions.

FIG. 7A shows: transmission electron microscope images of parent HBc capsid VLPs compared to HBc-Im7 VLPs (scale bar: 30 nm), showing a significantly increased size of the electron-dense rim in HBc-Im7 particles compared to the parent capsid.

FIG. 7B shows: quantitative comparison of the rim width and diameter of the parent HBc capsid VLPs compared to HBc-Im7 VLPs, indicating that HBc-Im7 VLPs have a slightly increased overall diameter but a significantly increased size of the rim, consistent with the added layer of Im7 proteins on top of the HBc capsid protein.

FIG. 7C shows: histograms of the size distribution of HBc VLPs and HBC-Im7 VLP's, respectively, indicating uniform size distribution with the size expected of HBc VLPs and slightly, but statistically significantly increased size of the HBcIm7 VLPs (28.3±3.3 vs. 27.3±2.5 nm, p=0.009).

FIG. 8 shows: ELISA analysis for the presence of serum anti-Hbc-Im7 antibodies in old-age mice (15 months at begin of study) at different timepoints after vaccination with HBc-Im7 VLPs;

FIG. 9 shows: SDS-PAGE of cytosolic fractions of HBc-Im7 VLP shells (lane 1 and ColE7-IL33 fusions (lane 2) expressed in E. coli; cytosolic fractions containing both protein fusions (lane 3), VLP fractions purified as flow-through after mixed-mode size-exclusion chromatography on CaptoCore700 resin (lanes 5-7).

FIG. 10 shows: SDS-PAGE of cell supernatants of ColE7-RBD-GFP fusions expressed as secreted proteins in HEK293T cells (lane 1) and of HBc-Im7 VLP shells and expressed in E. coli and partially purified (lane 2); and VLP's coupled to ColE7-RBD-GFP by mixing of fractions, followed by mixed mode size exclusion chromatography (CaptoCore700) to remove non-VLP proteins (lane 3).

FIG. 11 shows: fluorescence of HBc-Im7 VLPs (dark shaded) compared with parent HBc VLPs (light shaded) before (left) and after (right) incubation with octylamine-derivatized rhodamine.

FIG. 12 shows: cartoon diagrams of some embodiments of a VLP of the invention (A) a VLP of the first aspect where the functional molecule is an antigen (B) a VLP of an embodiment of the first aspect including the third binding protein where the functional molecule is an antibody, and (C) a VLP of the second aspect where the functional molecule is a non-protein antigen. Note that embodiment (C) can also be combined with embodiments (A) or (B), not shown here, but described elsewhere.

FIG. 13 shows: SDS-PAGE analysis following sucrose density gradient ultracentrifugation showing the biophysical sedimentation properties of VLPs harbouring Im7 integrated in the HBc Major-Immunodominant Region compared to wild type VLPs.

FIG. 14 shows: Native Agarose Gel Electrophoresis (NAGE) following sucrose density gradient ultracentrifugation showing similar distribution of T3 and T4 configured VLPs and similar RNA content in HBc-Im7 VLPs and wild type HBc VLPs.

FIG. 15 shows: An immuno-dot blot confirming the abolishment of cross-reactivity to wild type Hepatitis B virus core antigen by integration of Im7 into the Major Immunodominant Region (MIR).

FIG. 16 shows: TEM and DLS analysis of Wild type and HBc-Im7 VLPs (purified by sucrose gradient centrifugation), to determine shape and size of the VLPs.

FIG. 17 shows: TEM analysis of Wild type and HBc-Im7 VLPs (purified by sucrose gradient centrifugation), to determine the rim thickness of the VLPs.

FIG. 18 shows: SDS-PAGE analysis following sucrose density gradient ultracentrifugation of VLPs consisting of HBc-Im7 decorated with ColE7-IL33.

FIG. 19 shows: Native Agarose Gel Electrophoresis (NAGE) of sucrose density gradient ultracentrifugation fractions of IL-33 decorated HBc-Im7 VLPs and wild type HBc VLPs.

FIG. 20 shows: Panel A shows TEM images of HBc, HBc-Im7, and HBc-Im7-IL33 VLPs illustrating the thickened rim in VLPs carrying the Im7 insert. Panel B shows a bar graph of the diameter of HBc-Im7-IL33 VLPs determined by TEM, and a DLS graph demonstrating the size increase of IL-33 decorated VLPs.

FIG. 21 shows: An immuno-dot blot confirming native protein folding of IL33 attached to the surface of HBC-Im7 VLPs through detection of an antibody raised against a conformational epitope of IL33 (as well as absence of cross-reactivity to HBc-wild type VLPs).

FIG. 22 shows: Graphs A to C showing the immunogenicity of IL33-decorated HBC-Im7 VLPs in mice vaccinated with IL33-decorated HBc-Im7 (black) compared to CuMV derived VLPs (grey).

FIG. 23 shows: Im7 harbours novel chemistry allowing single-step affinity purification of Im7 decorated VLPs. Panel A top and bottom show SDS PAGE analyses of eluted fractions on DEAE CimMultus monolith columns, Panel B shows SDS PAGE analyses of eluted fractions on Qa CimMultus monolith column, Panel C shows TEM images of the 1 M NaCl fraction from DEAE column purification of HBc-Im7, and cartoons of the mechanism of DEAE or Q binding.

FIG. 24 shows: SDS-PAGE analysis gels showing the ColE7-Im7 interaction allows disassociation—reassembly purification of decorated VLPs. (A) SDS PAGE after affinity purification via IMAC following urea-mediated VLP-capsid disassembly or without prior capsid disassembly (marked ‘con’), showing that treatment with urea preserves the binding of HBc-Im7 (grey arrow) and ColE7-IL33 (white arrow), while non-dissassembled VLPs cannot bind to the Ni-resin as the histidine-tag on the C-terminus of HBc-Im7 is not accessible. (B) SDS PAGE after modified size exclusion chromatography on CaptoCore700 (Cc700) resin shows that the disassembled VLPs reassemble to large particles, allowing unhindered flow through the Cc700 matrix. The amounts of HBc-Im7 and ColE7-IL33 are stoichiometrically equal, indicating full decoration of the VLP with the cytokine.

FIG. 25 shows: the use of Colicin E7 to fuse proteins to the surface of VLPs simultaneously provides a chaperone function allowing native protein folding of proteins not soluble in E. coli on their own, Panel A shows SDS PAGE of E. coli lysates (left) and fractionation into cytosol vs. insoluble pellet, showing that significant amount of IL17 in the cytosol when expressed fused to ColE7. Panel B shows purification via interaction with Im7-agarose of both ColE7-IL17 and ColE7-IL33, followed by cleavage of the fused ColicinE7 with TEV protease and below a cartoon showing the arrangement of the fusion protein. Panel (C) shows the results of a receptor binding ELISA using Human IL17RA protein to either IL-17 or IL-33, confirming native folding of IL17 which selectively binds to its receptor with high affinity while IL33 does not.

FIG. 26 shows: Independent and sequential induction of VLP backbone and epitope proteins within single cells allows initial VLP formation followed by epitope assembly. Panel A shows a cartoon diagram illustrating the induction system; a plasmid harbours the tetR protein, constitutively expressed driven by a ribosomal binding site downstream of the AmpR gene. This allows selective induction of epitope proteins (here shown for C7-IL17) by addition of anhydro-tetracycline (aTc), whereas the VLP backbone (HBc-Im7) can be separately induced by IPTG. Panels B and C show SDS-PAGE gels of three independent clones for each cytokine (IL33 and IL17) documenting exceptionally tight regulation without any leakiness, Panel D shows an SDS-PAGE gel of a time course of aTc induction of IL17 in E. coli already induced with IPTG.

FIG. 27 shows: Barstar can be incorporated into HBc to form VLPs. Panel A shows an SDS-PAGE gel following sucrose density gradient centrifugation of HBc-Barstar VLPs. Panel B shows DLS analysis highlighting the particles show a peak at 34 nm size. Panel C demonstrates that the particles exhibit a thickened rim, when analysed by TEM.

FIG. 28 shows: An SDS-PAGE gel showing the Barnase—Barstar interaction allows disassociation—reassembly purification of decorated VLPs. SDS PAGE after affinity purification via IMAC and after modified size exclusion chromatography on CaptoCore700 (Cc700) resin are shown on the same gel.

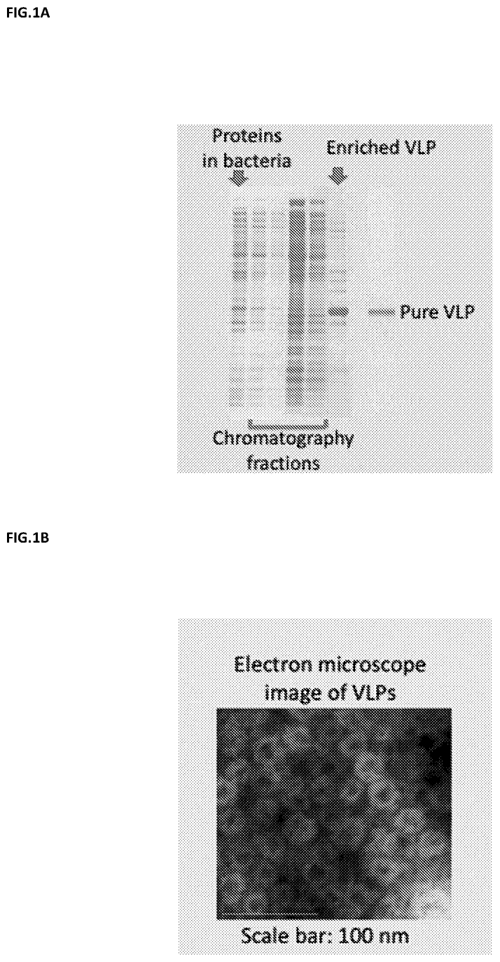

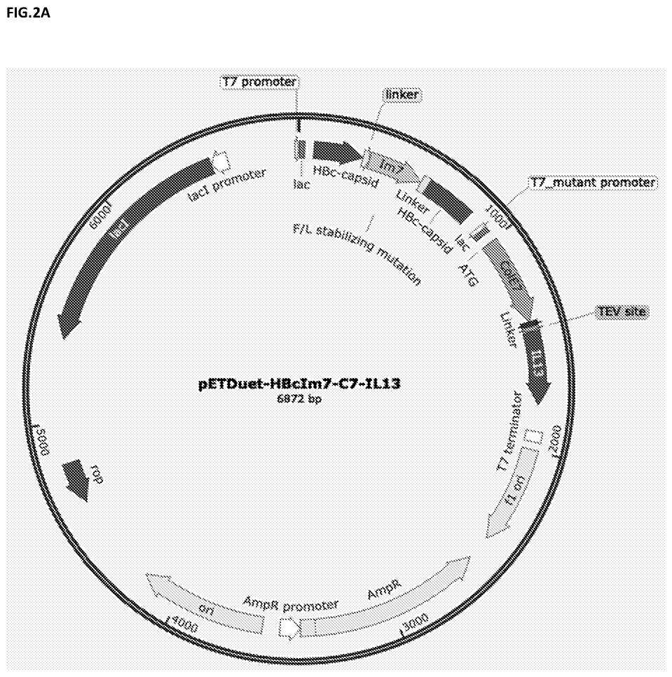

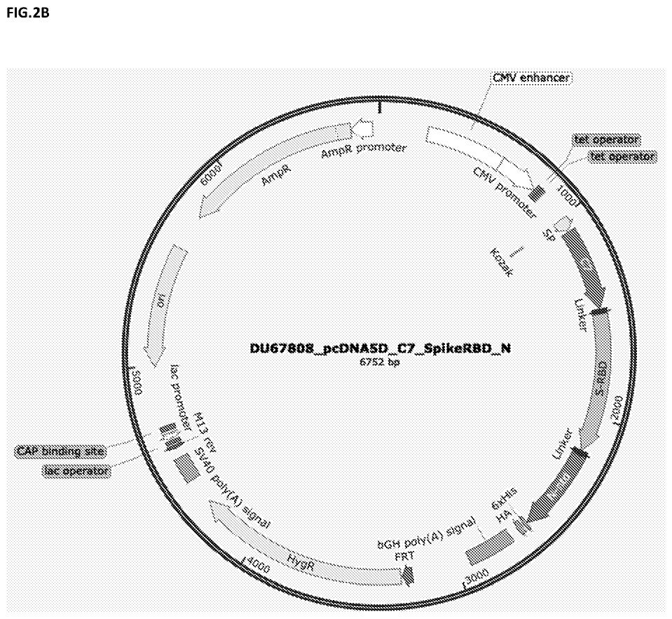

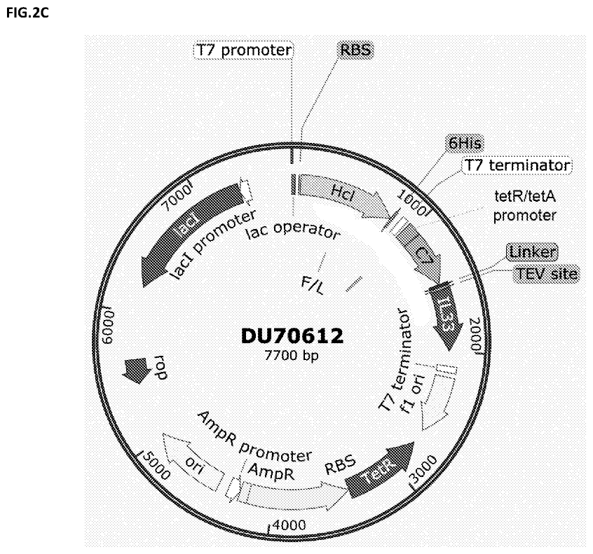

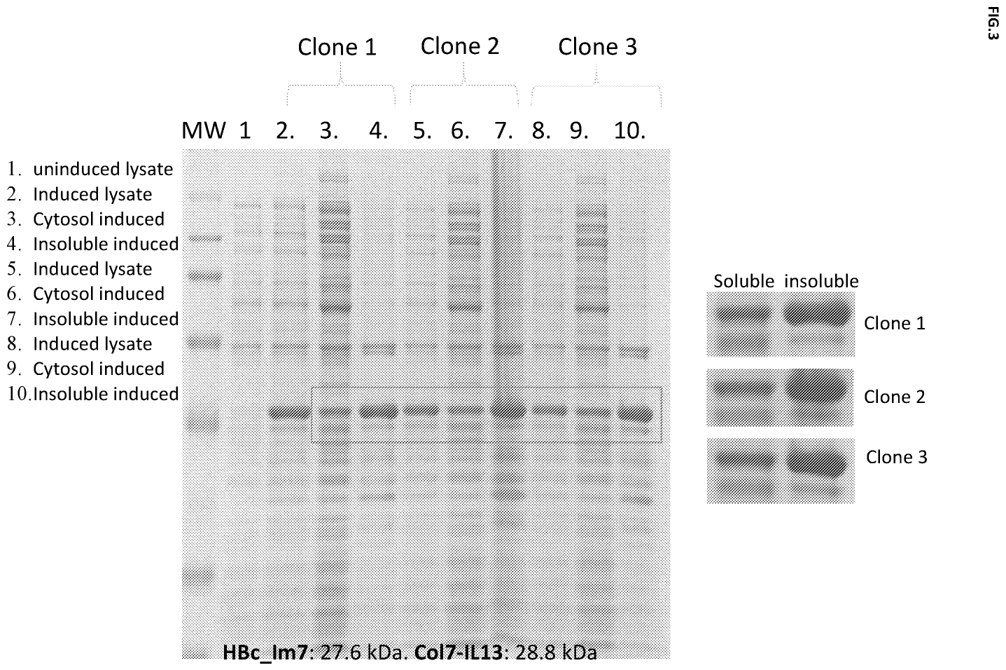

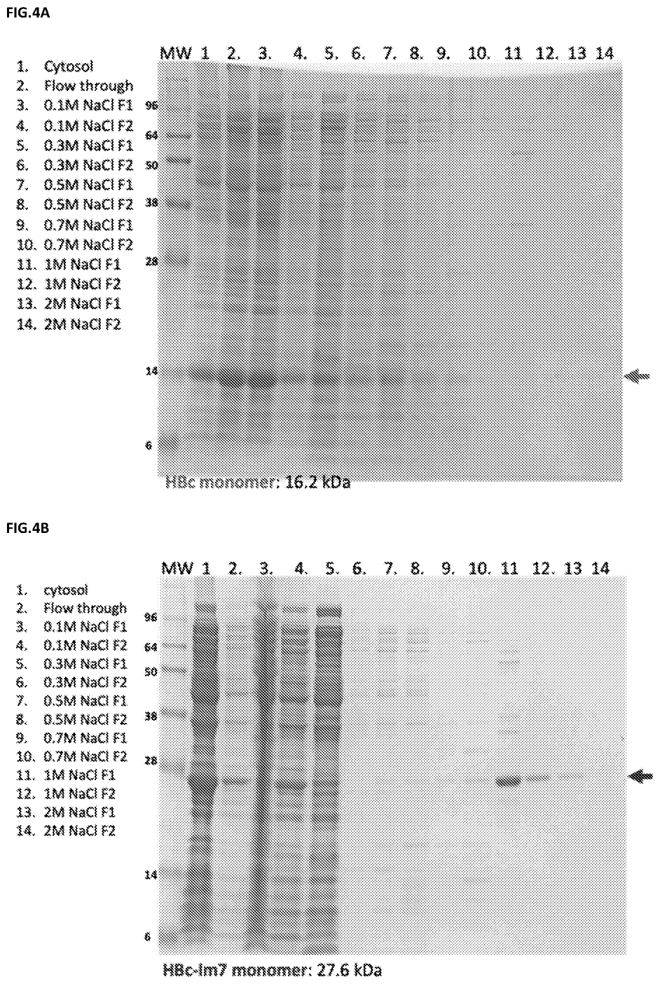

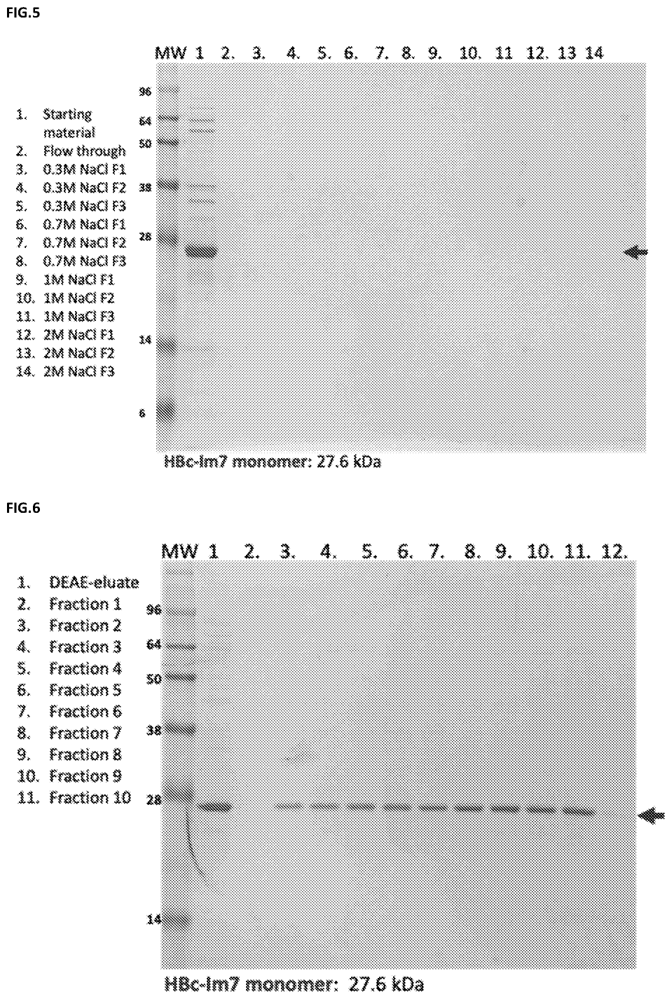

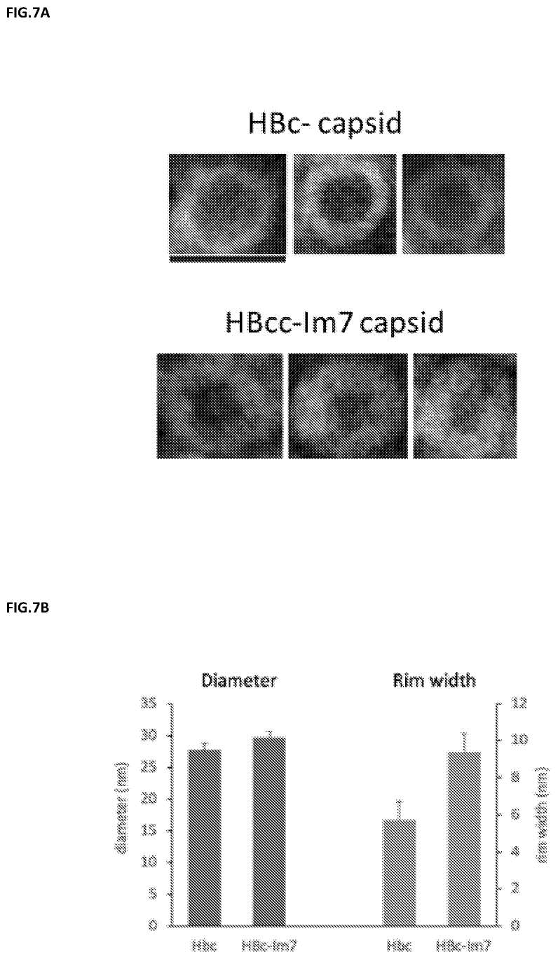

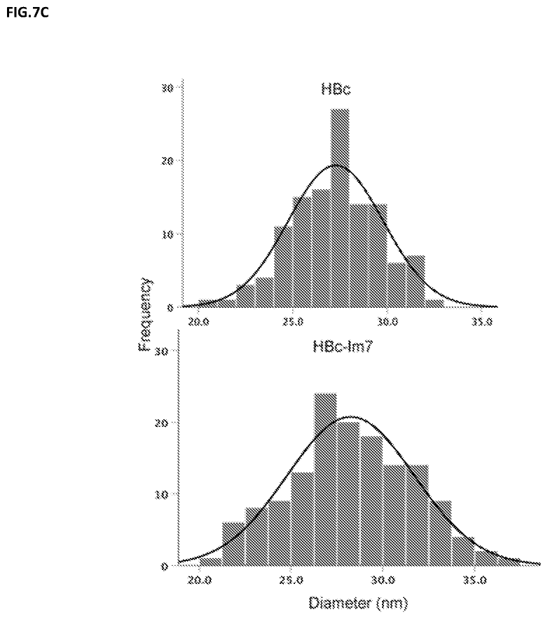

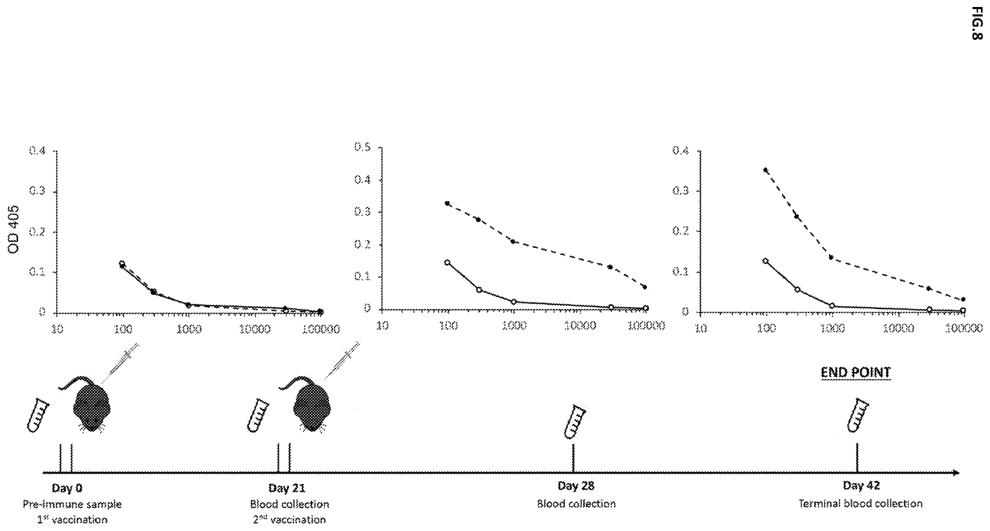

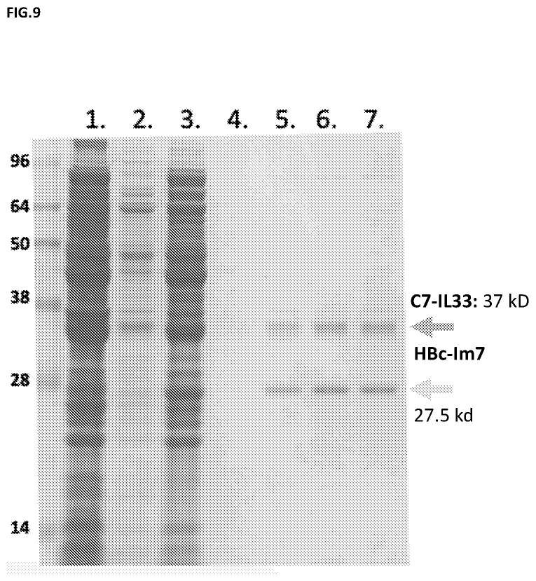

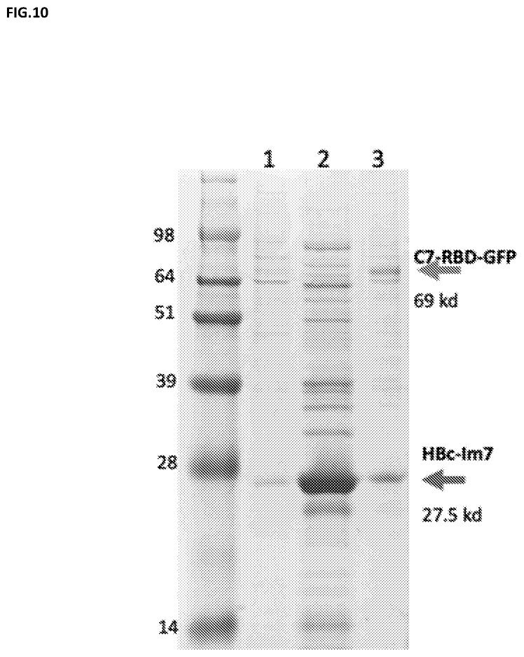

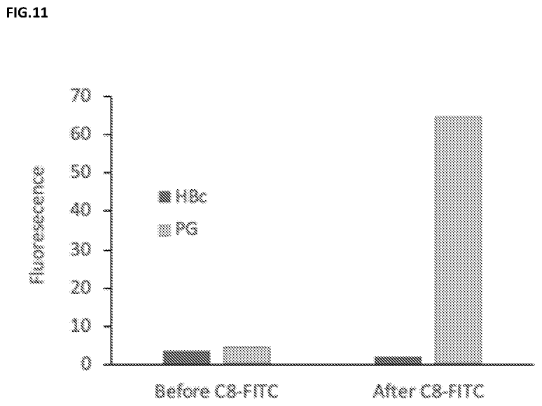

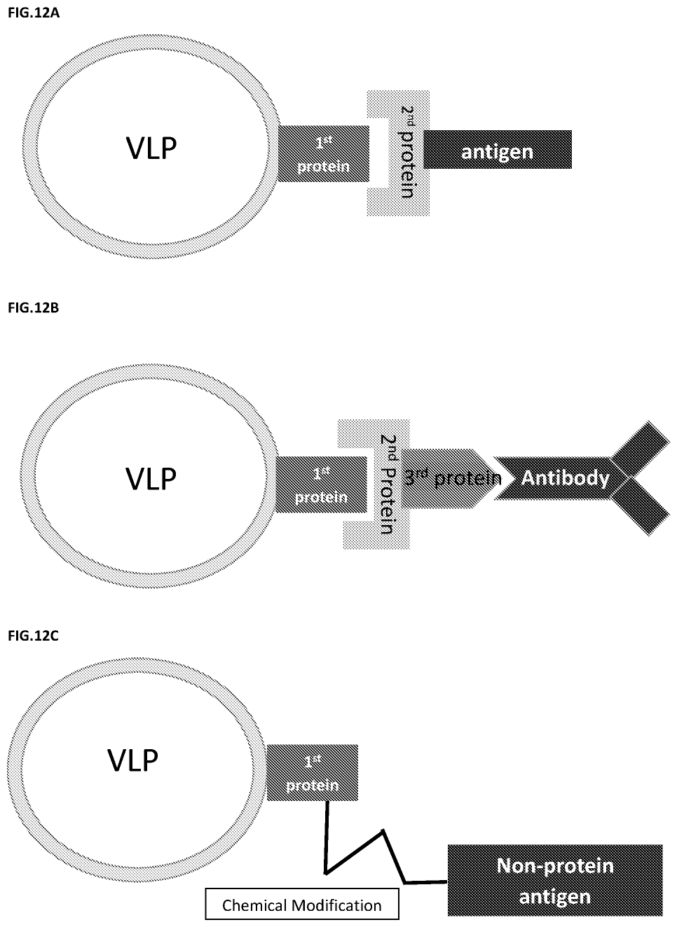

### DESCRIPTION

Pair of Binding Proteins

The present invention relates to VLPs which make use of a pair of binding proteins to form a bridge which can attach an agent of interest, typically an antigen, to the viral capsid proteins forming the VLP shell.

The pair of binding proteins may be covalently bound or non-covalently bound.

Suitably the pair of binding proteins are non-covalently bound. Suitably the pair of binding proteins are bound quasi-covalently. Suitably the pair of binding proteins are bound by any non-covalent type of bonding such as; electrostatic interactions, hydrogen bonds, van der waals interactions or hydrophobic interactions. However, in some embodiments, the pair of binding proteins are not bound by hydrophobic bonding.

Alternatively, the pair of binding proteins may be covalently bound. Suitably the pair of binding proteins may be bound by any covalent type of bonding.

Suitably the pair of binding proteins comprises one net positively charged protein and one net negatively charged protein. Suitably the first binding protein comprises a net negative charge. Suitably the second binding protein comprises a net positive charge.

Advantageously, the first binding protein having a net negative charge increases stability of the VLP and reduced aggregation or clumping.

In one embodiment, therefore, the pair of binding proteins are bound non-covalently by electrostatic interactions.

Suitably, in any case, the pair of binding proteins are bound with high affinity. Suitably the pair of binding proteins are bound with a Kd in the femtomolar to picomolar range. Suitably with a Kd of between: 10 fM to 10 pM, 10 fM to 1 pM, 10 fM to 0.1 pM, 10 fM to 0.01 pM, 1 fM to 1 pM, 1 fM to 0.1 pM, 1 fM to 0.01 pM.

Advantageously, high affinity binding between the proteins means that the VLP is more stable.

Suitably the pair of binding proteins have low homology to proteins of the subjects which may be treated with the VLP. Suitably the pair of binding proteins have low homology to human proteins. Suitably the pair of binding proteins have low homology with the tertiary structure of any human proteins.

Advantageously, low homology with human proteins means that the binding proteins themselves are is less likely to stimulate an off-target immune reaction.

Suitably the pair of binding proteins do not contain any disulphide bonds.

Suitably the pair of binding proteins are not glycosylated.

Suitably each of the proteins in the pair of binding proteins is relatively small in size. Suitably each of the proteins in the pair of binding proteins comprises a relatively short sequence length. Suitably each of the proteins in the pair of binding proteins comprises a length of between 84-134 amino acids. Suitably each of the proteins in the pair of binding proteins comprises a length of less than 135 amino acids.

Advantageously, the lack of disulphide bonds, lack of glycosylation, and small size means that the binding proteins are easier to produce in bacterial cells such as E. coli.

Suitably the pair of binding proteins comprises a bacterial toxin and its corresponding inhibitor or antitoxin. Suitably the first binding protein of the VLP is a bacterial toxin inhibitor. Suitably the second binding protein of the VLP (if present) is a bacterial toxin.

Suitable bacterial toxin and inhibitor pairs are: a colicin and colicin immunity protein. Suitably ColE7 and Im7, ColE8 and Im8, ColE9 and Im9, ColE2 and Im2, or Barnase and Barstar. Suitably the bacterial toxin and inhibitor pair comprises a bacterial nuclease and its inhibitor. Suitably the first binding protein is the inhibitor and the second binding protein is the bacterial nuclease. Suitable bacterial nuclease and inhibitor pairs are: ColE7/Im7 and Barnase/Barstar.

In one embodiment, the pair of binding proteins is ColE7 and Im7, wherein the first binding protein is Im7 and the second binding protein is ColE7.

In one embodiment, the pair of binding proteins is Barnase and Barstar, wherein the first binding protein is Barstar and the second binding protein is Barnase.

Suitably the first or second binding protein may be the wild-type proteins, or they may be modified. Suitably the first or second binding proteins may be modified to improve their function as a binding protein in the context of the VLP of the invention. Suitable modifications may include: insertions, deletions, substituents, truncations, reversals, repeats, or the like in the amino acid sequence encoding the protein.

Suitably, any property of the toxin (second binding protein) detrimental to either the host cell and/or the recipient organism intended for VLP administration is neutralized by targeted modifications.

Suitably the first or second binding proteins may comprise one or more amino acid substitutions. Suitably the amino acid substitutions may increase the binding affinity between the first and second binding proteins. Suitably the amino acid substitutions may remove undesirable disulphide bonds from the first and/or second binding proteins.

Suitably the first binding protein may comprise one or more amino acid substitutions.

In an embodiment where the first binding protein is Barstar, suitably the amino acid sequence of Barstar comprises one or more of the following substitutions: C40A, C82A, and I87E. Suitably the amino acid sequence of Barstar may comprise all of the following substitutions: C40A, C82A, and I87E. Suitably the amino acid sequence of Barstar comprises:

(SEQ ID NO: 36)
KKAVINGEQIRSISDLHQTLKKELALPEYYGENLDALWDALTGWVEYPL
VLEWRQFEQSKQLTENGAESVLQVFREAKAEGADITIELS

In an embodiment where the first binding protein is Im7, suitably the amino acid sequence of Im7 comprises the following substitution: F41L. Suitably the amino acid sequence of Im7 comprises:

(SEQ ID NO: 37)
ELKNSISDYTEAEFVQLLKEIEKENVAATDDVLDVLLEHFVKITEHPDG
TDLIYYPSDNRDDSPEGIVKEIKEWRAANGKPGFKQ

Suitably the second binding protein may comprise one or more amino acid substitutions. Suitably the amino acid substitutions in the amino acid sequence of the second binding protein may increase the negative charge of the second binding protein.

In an embodiment where the second binding protein is Barnase, suitably the amino acid sequence of Barnase comprises the following substitution: E73W. Suitably the amino acid sequence of Barnase comprises

(SEQ ID NO: 38)
AQVINTFDGVADYLQTYHKLPDNYITKSEAQALGWVASKGNLADVAPGK
SIGGDIFSNREGKLPGKSGRTWRWADINYTSGFRNSDRILYSSDWLIYK
TTDHYQTFTKIR

In an embodiment where the second binding protein is ColE7, suitably the amino acid sequence of ColE7 comprises one or more of the following substitutions: Arg538Ala, Glu542Ala, and His569Ala. Suitably the amino acid sequence of ColE7 may comprise all of the following substitutions: Arg538Ala, Glu542Ala, and His569Ala. Suitably the amino acid sequence of ColE7 comprises

(SEQ ID NO: 39)
ESKRNKPGKATGKGKPVNNKWLNNAGKDLGSPVPDRIANKLRDKEFKSF
DDFRKKFWEEVSKDPELSKQFSRNNNDRMKVGKAPKTRTQDVSGKATSF
ALHHEKPISQNGGVYDMDNISVVTPKRAIDIHRGKS

Suitably the first or second binding proteins may be truncated.

Suitably the second binding protein is truncated.

In an embodiment where the second binding protein is ColE7, suitably the whole or a part of the ColE7 protein may be used as the second binding protein. Suitably only a part of the ColE7 protein is used as the second binding protein. Suitably the ColE7 protein is truncated, suitably so that it only comprises the catalytic domain of ColE7. Suitably the second binding protein comprises the catalytic domain of ColE7.

In an embodiment where the second binding protein is Barnase, suitably the whole or a part of the Barnase protein may be used as the second binding protein. Suitably only a part of the Barnase protein is used as the second binding protein. Suitably the Barnase protein is truncated, suitably so that it only comprises the catalytic domain of Barnase. Suitably the second binding protein comprises the catalytic domain of Barnase.

In accordance with the third aspect of the invention there is provided a capsid fusion protein comprising a viral capsid protein fused to a binding protein, wherein the binding protein is a bacterial toxin inhibitor.

Suitably, the viral capsid protein may be a hepatitis B viral capsid protein (HBc).

Suitably the binding protein is fused into the immunodominant region of the viral capsid protein, as explained elsewhere herein for HBc.

Suitably, the capsid fusion protein may comprise a sequence according to SEQ ID NO: 12, 13, 14, or 15. Suitably the capsid fusion protein may comprise a sequence having at least 60%, at least 65%, at least 70%, at least 75%, at least 80%, at least 85%, at least 90%, at least 95% identity with SEQ ID NO: 12, 13, 14, or 15. Suitably the capsid fusion protein may consist of a sequence according to SEQ ID NO: 12, 13, 14, or 15.

In one embodiment, the capsid fusion protein comprises a hepatitis B viral capsid protein fused to binding protein Im7. In such an embodiment, the capsid fusion protein may comprise a sequence according to SEQ ID NO: 12, 13, or 14. Suitably the capsid fusion protein may comprise a sequence having at least 60%, at least 65%, at least 70%, at least 75%, at least 80%, at least 85%, at least 90%, at least 95% identity with SEQ ID NO: 12, 13, or 14. Suitably the capsid fusion protein may consist of a sequence according to SEQ ID NO: 12, 13, or 14.

In one embodiment, the capsid fusion protein comprises a hepatitis B viral capsid protein fused to binding protein Barstar. In such an embodiment, the capsid fusion protein may comprise a sequence according to SEQ ID NO: 15. Suitably the capsid fusion protein may comprise a sequence having at least 60%, at least 65%, at least 70%, at least 75%, at least 80%, at least 85%, at least 90%, at least 95% identity with SEQ ID NO: 15. Suitably the capsid fusion protein may consist of a sequence according to SEQ ID NO: 15.

Suitably, the binding protein may comprise a chemical modification.

In accordance with a fourth aspect of the present invention, there is provided a functional fusion protein comprising a functional molecule fused to a binding protein wherein the binding protein is a bacterial toxin.

Suitably, the functional fusion protein may comprise a sequence according to SEQ ID NO: 16, 17, 18, 19, 21, or 46. Suitably the functional fusion protein may comprise a sequence having at least 60%, at least 65%, at least 70%, at least 75%, at least 80%, at least 85%, at least 90%, at least 95% identity with SEQ ID NO: 16, 17, 18, 19, 21, or 46. Suitably the functional fusion protein may consist of a sequence according to SEQ ID NO: 16, 17, 18, 19,21, or 46.

In one embodiment, the functional fusion protein may comprise IL13 fused to binding protein ColE7. In such an embodiment, the functional fusion protein may comprise a sequence according to SEQ ID NO: 16, or 17. Suitably the functional fusion protein may comprise a sequence having at least 60%, at least 65%, at least 70%, at least 75%, at least 80%, at least 85%, at least 90%, at least 95% identity with SEQ ID NO: 16 or 17. Suitably the functional fusion protein may consist of a sequence according to SEQ ID NO: 16 or 17.

In one embodiment, the functional fusion protein may comprise IL33 fused to binding protein ColE7. In such an embodiment, the functional fusion protein may comprise a sequence according to SEQ ID NO: 18 or 19. Suitably the functional fusion protein may comprise a sequence having at least 60%, at least 65%, at least 70%, at least 75%, at least 80%, at least 85%, at least 90%, at least 95% identity with SEQ ID NO: 18 or 19. Suitably the functional fusion protein may consist of a sequence according to SEQ ID NO: 18 or 19.

In one embodiment, the functional fusion protein may comprise IL13 fused to binding protein Barnase. In such an embodiment, the functional fusion protein may comprise a sequence according to SEQ ID NO: 21. Suitably the functional fusion protein may comprise a sequence having at least 60%, at least 65%, at least 70%, at least 75%, at least 80%, at least 85%, at least 90%, at least 95% identity with SEQ ID NO: 21. Suitably the functional fusion protein may consist of a sequence according to SEQ ID NO: 21.

In one embodiment, the functional fusion protein may comprise IL17 fused to binding protein ColE7. In such an embodiment, the functional fusion protein may comprise a sequence according to SEQ ID NO: 46. Suitably the functional fusion protein may comprise a sequence having at least 60%, at least 65%, at least 70%, at least 75%, at least 80%, at least 85%, at least 90%, at least 95% identity with SEQ ID NO: 46.

Suitably the functional fusion protein may consist of a sequence according to SEQ ID NO: 46.

Alternatively, there is provided a functional fusion protein comprising a first binding protein fused to a further binding protein, wherein the first binding protein is a bacterial toxin, and the further binding protein is able to capture functional molecules that are antigen binding proteins.

Sutiably, the further binding protein is capable of binding to a functional molecule. Suitably, in such an embodiment, the functional molecule is an antigen binding protein such as an antibody. Suitably the further binding protein is the third binding protein as described elsewhere herein. Sutiably the further binding protein is protein G.

In one embodiment, the functional fusion protein comprises a sequence according to SEQ ID NO: 20. Suitably the functional fusion protein may comprise a sequence having at least 60%, at least 65%, at least 70%, at least 75%, at least 80%, at least 85%, at least 90%, at least 95% identity with SEQ ID NO: 20. Suitably the functional fusion protein may consist of a sequence according to SEQ ID NO:20.

Suitably, each of the fusion proteins comprises one or more linkers. Suitably the linkers are located between the protein coding sequences. Suitably a linker is located at the N and C terminus of the binding protein, suitably to link to the viral capsid protein. Suitably a linker is located between the functional molecule and the binding protein. Suitably a linker is located between the binding protein and the further binding protein. Suitably each linker is between 5 to 50 amino acids in length. Suitably each linker is 5, 10, 15, 20, 21, 25, 30, 35, 40 amino acids in length. Suitably each linker is 9, 10 or 11 amino acids in length. Suitably each linker comprises the sequence: GGGGSGGGGS (SEQ ID NO:33) or GGGGGSGGGGS (SEQ ID NO:34) or SGGGSSGSG (SEQ ID NO: 35).

Suitably the first binding protein may comprise additional modifications. Suitably the first binding protein may comprise chemical modification. Suitably Im7 may comprise chemical modification.

Suitably the chemical modification is capable of binding to a functional molecule. Suitably the chemical modification is capable of covalently binding to a functional molecule. In one example, the functional molecule bound to the chemical modification may be a fluorescent molecule. Other suitable functional molecules are described elsewhere herein.

Suitably the chemical is attached to the first binding protein by non-covalent binding. Suitably the chemical is attached to the first binding protein by electrostatic and/or hydrophobic bonding.

Suitable chemical modifications include alkanes having an amine group. Suitably the alkane may have any chain length. Suitably the alkane is a lower alkane. Suitably the alkane may have a chain length of between 1 and 10 carbons. Sutiably the alkane may have a chain length of between 4 and 8 carbons. Suitably the alkane may be branched.

Suitably, the length of the carbon chain and the length of branched substitutions on the amine group are chosen such as to allow either irreversible attachment to the protein or reversible attachment, dependent on the desired application. In one embodiment, the chemical is attached irreversibly to the first binding protein. Suitably, in such an embodiment, conferring irreversible binding, the alkane has eight carbon atoms and a terminal nitrogen (octylamine). In another embodiment, the chemical is attached reversibly to the first binding protein. Suitably, in such an embodiment, allowing reversible binding the alkane has 4 carbon atoms in a branched structure (diethylethanolamine).

Suitably the first binding protein may be chemically modified at one or more sites, suitably at one or more amino acids. Suitably the first binding protein is chemically modified at one amino acid.

In one embodiment, the first binding protein is chemically modified with DEAE.

In one embodiment, the first binding protein is chemically modified with octylamine.

Suitably, in such embodiments, the first binding protein may be Im7.

Suitably, modification with DEAE allows the first binding protein to be purified. Suitably purification by chromatography.

Suitably modification with octylamine allows the first binding protein to directly bind to a functional molecule.

In one embodiment, the chemical modification of the binding protein occurs within the host cell. Suitably by post-translational modification. In another embodiment, the chemical modification of the binding protein occurs outside of the host cell. Suitably by means of a chemical reaction. Suitably by means of a non-enzymatically catalyzed non-covalent attachment.

Viral Capsid Protein

The present invention relates to VLPs which comprise one or more viral capsid proteins, the viral capsid proteins self-assemble into the VLP shell, to which functional molecules can then be attached using the protein binding pair and/or chemical modification as discussed above.

In accordance with the first aspect, the viral capsid protein is a Hepatitis B viral capsid protein (HBc).

In accordance with the second aspect, the viral capsid protein may be selected from any suitable viral capsid protein, for example: Hepatitis B viral capsid protein, Hepatitis C capsid protein, HPV capsid protein, AAV capsid protein, HIV capsid protein, influenza capsid protein, Newcastle diseases virus capsid protein, Nipah virus capsid protein.

In one embodiment of any of the aspects, the viral capsid protein is a dimeric viral capsid protein.

In one embodiment of any of the aspects, the viral capsid protein is a Hepatitis B viral capsid protein.

Suitably each viral capsid protein is attached to a first binding protein. Suitably therefore each viral capsid protein displays a first binding protein.

Suitably each viral capsid protein is modified to display a first binding protein. Suitably each viral capsid protein is fused to a first binding protein. Suitably each viral capsid protein is modified to display a first binding protein by fusing the first binding protein to the viral capsid protein. Suitably each viral capsid protein is modified to display a first binding protein by inserting the first binding protein into the viral capsid protein. Suitably the first binding protein is inserted into the major immunodominant region of the viral capsid protein. Suitably the first binding protein is fused to the major immunodominant region of the viral capsid protein. Suitably the first binding protein is inserted between amino acid residues 76 and 80 of the major immunodominant region of the viral capsid protein. Suitably the first binding protein is inserted between amino acid residues 77 and 79 of the major immunodominant region of the viral capsid protein. Suitably the first binding protein is inserted between amino acid residues 77 and 78 of the major immunodominant region of the viral capsid protein.

Suitably the viral capsid protein may comprise further modifications. Suitable modifications may include: insertions, deletions, substituents, truncations, reversals, repeats, or the like in the amino acid sequence encoding the protein. Suitably the viral capsid protein may comprise further modifications in the major immunodominant region. Suitably such modifications aid the insertion of the first binding protein into the viral capsid protein. Suitably the viral capsid protein may comprise amino acid deletions. Suitably the viral capsid protein may comprise amino acid deletions in the major immunodominant region. Suitably the viral capsid protein may comprise amino acid deletions in the major immunodominant region which remove negatively charged amino acids.

In one embodiment, the viral capsid protein is a hepatitis B capsid protein and comprises the following amino acid deletions: E77 and D78. Suitably the amino acid sequence of HBc comprises:

MDIDPYKEFGASVELLSFLPSDFFPSIRDLLDTASALYREALESPEHCSPHHTALRQ AILCWGELMNLATWVGSNL[X]PASRELVVSYVNVNMGLKIRQLLWFH ISCLTFGRE TVLEYLVSFGVWIRTPPAYRPPNAPILSTLPETTVV (SEQ ID NO: 40) wherein [X] indicates the position of an insertion, suitably of an amino acid insertion, suitably of an amino acid sequence encoding the first binding protein.

In one embodiment, the viral capsid protein is a hepatitis B capsid protein, comprising a first binding protein inserted within the major immunodominant region thereof, between residues 76 and 80, and further comprising the following amino acid deletions: E77 and D78.

Functional Molecule

The present invention relates to VLPs which are able to display various functional molecules on their surface by virtue of the protein binding pair or by virtue of chemical modifications to the first binding protein.

Suitably each pair of binding proteins is attached to at least one functional molecule. Suitably each pair of binding proteins may be attached to more than one functional molecule. Suitably the functional molecules may be of the same type or different types. For example, each pair of binding proteins may be attached to any combination of one or more antigens, antigen binding proteins, or flourescent molecules.

Suitably each pair of binding proteins is attached to one functional molecule. Suitably, in such an embodiment, the functional molecule may be attached to the second binding protein in accordance with the first aspect, or may be attached to the third binding protein if present.

Suitably each chemical modification is attached to one functional molecule. Suitably, in such an embodiment, the functional molecule is attached to the first binding protein via the chemical modification in accordance with the second aspect. Suitably in such embodiments, the functional molecule is a non-protein antigen or epitope thereof, or a flourescent molecule.

Suitably, in some embodiments, there may be more than one functional molecule per pair of binding proteins. Suitably a first functional molecule may be attached to the first binding protein via a chemical modification, and suitably a second functional molecule may be attached to the second binding protein, or the third binding protein if present.

Alternatively, a first and second functional molecule may be attached to the second binding protein. Optionally, in addition, a third functional molecule maybe attached to the first binding protein via a chemical modification.

Suitable functional molecules may include:
protein or non-protein antigens;
antigen binding proteins such as antibodies or binding fragments thereof, antibody mimetics, and aptamers;
flourescent molecules.

Suitable antigens may include the whole or part of an antigen. Suitably the antigen may be a subunit or monomer of an antigen. Suitably the functional molecule may be an epitope of an antigen. Suitably the use of an antigen as a functional molecule produces a VLP which is capable of stimulating an immune response to the antigen. Suitably this is useful as a vaccine.

Suitably the antigen may be a protein or non-protein antigen. Suitable non-protein antigens may include sugars, lipids or carbohydrates, or small molecule chemicals to which an immune response is desired, or who need to be detected, such as nicotine, cocaine, or other exogenous toxins.

Suitably the antigen may be a self or non-self antigen relative to the subject intended to be treated with the VLP. Suitably the antigen may be a human or non-human antigen.

Suitably the antigen may be derived from the causative agent in a disease or disorder. Suitably the causative agent may be self or non-self.

Suitably a non-self causative agent may be an infectious agent. Suitably therefore the antigen may be derived from an infectious agent such as a virus, bacterium, fungus, protozoan, archaeon.

Suitably the antigen may be derived from a virus selected from: Adeno-associated virus, Chikungunya virus, Crimean-Congo hemorrhagic fever virus, Dengue virus, Ebolavirus, Echovirus, Encephalomyocarditis virus, Epstein-Barr virus, Hantaan virus, Hepatitis A virus, Hepatitis B virus, Hepatitis C virus, Hepatitis D virus, Hepatitis E virus, Human adenovirus, Human astrovirus, Human coronavirus, Human cytomegalovirus, Human enterovirus, Human herpesvirus, Human immunodeficiency virus, Human papillomavirus, Human parainfluenza, Human respiratory syncytial virus, Human rhinovirus, Human torovirus, Influenza A virus, Influenza B virus, Influenza C virus, Japanese encephalitis virus, Polyomavirus, Kunjin virus, Lassa virus, Measles virus, Molluscum contagiosum virus, Mumps virus, Nipah virus, Poliovirus, Rabies virus, Rotavirus A, Rotavirus B, Rotavirus C, Rubella virus, Sagiyama virus, Salivirus A, Sapporo virus, Sindbis virus, Toscana virus, Uukuniemi virus, Varicella-zoster virus, Venezuelan equine encephalitis virus, Vesicular stomatitis virus, Western equine encephalitis virus, West Nile virus, Yellow fever virus, Zika virus.

Suitably the antigen may be derived from a bacterium selected from: Actinomyces israelii, Bacillus anthracis, Bacillus cereus, Bartonella henselae, Bartonella quintana, Bacteroides fragilis, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Klebsiella pneumoniae, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Nocardia asteroides, Pseudomonas aeruginosa, Rickettsia rickettsia, Salmonella enterica subsp. enterica, Salmonella typhi, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Yersinia pestis, Yersinia enterocolitica, Yersinia pseudotuberculosis.

In one embodiment the antigen is derived from a coronavirus, suitably from SARS-CoV-2. Suitably the antigen is the whole or part of a spike protein derived from SARS-CoV-2, or the whole or part of a nucleocapsid protein derived from SARS-CoV-2.

In one embodiment, therefore, the functional molecule is part of a spike protein derived from SARS-CoV-2. Suitably the receptor binding domain.

In another embodiment, therefore, the functional molecule is part of a nucleocapsid protein derived from SARS-CoV-2. Suitably the C-terminus.

Suitably a self-causative agent may be a non-infectious agent. Suitably therefore the antigen may be derived from a non-infectious agent such as an inflammatory molecule, or a molecule causing degenerative changes in nervous (such as beta-amyloid), cartilage or bone tissue, or a molecule causing worsening of a neoplastic disease.

Suitably the antigen may be an inflammatory molecule or a molecule causing degenerative changes or a molecule conducive to a neoplastic disease which is a causative agent in a disease or disorder. Suitably the molecule may operate in humans or in non-human mammals. Suitably the molecule may cause a disease or disorder in a specific species.

Suitable inflammatory molecules may include chemokines or cytokines, or proteases. Suitable chemokines or cytokines may include: interleukins, tumour necrosis factors, interferons, and colony stimulating factors. Suitable chemokines or cytokines may include: IL1, IL2, Il3, Il4, IL5, Il6, Il7, IL8, IL9, IL10, IL11, IL12, IL13, IL17, IL33, TNFα, TNFβ, IFNα, IFNβ, IFNγ, G-CSF, GM-CSF, M-CSF, erythropoietin, and TGFβ. Suitable proteases may include ADAMTS4, ADAMTS5. Suitably the antigen is an interleukin or a protease. Suitably the antigen is IL13, IL17 or IL33 or a fragment thereof.

In one embodiment, therefore, the functional molecule is IL13, IL17 or IL33.

Suitable molecules which case degenerative changes in nervous tissue or worsening of neoplastic diseases may include: ADAMTS4/5, angiogenesis factors, or factors allowing escape of tumours such as galectin proteins.

References to any antigens herein may equally refer to an epitope of said antigen.

Suitable antigen binding proteins such as antibodies for use as a functional molecule are capable of binding an antigen of interest. Suitably the use of an antigen binding protein such as an antibody as a functional molecule produces a VLP which is capable of binding to an antigen. Suitably this is useful for detecting an antigen, or for targeting the VLP to an antigen.

An antigen of interest may be any of those listed above. For example, an antigen of interest may be from a disease causing agent such as a virus, bacterium, fungus, protozoan, or archaeon. Alternatively, an antigen of interest may be from a non-infectious agent, for example, a cell surface receptor.

Suitably the antibody may be capable of binding to an antigen from a virus, bacterium, fungus, protozoan, archaeon as listed above. Suitable viruses may be selected from, for example: Adeno-associated virus, Chikungunya virus, Crimean-Congo hemorrhagic fever virus, Dengue virus, Ebolavirus, Echovirus, Encephalomyocarditis virus, Epstein-Barr virus, Hantaan virus, Hepatitis A virus, Hepatitis B virus, Hepatitis C virus, Hepatitis D virus, Hepatitis E virus, Human adenovirus, Human astrovirus, Human coronavirus, Human cytomegalovirus, Human enterovirus, Human herpesvirus, Human immunodeficiency virus, Human papillomavirus, Human parainfluenza, Human respiratory syncytial virus, Human rhinovirus, Human torovirus, Influenza A virus, Influenza B virus, Influenza C virus, Japanese encephalitis virus, Polyomavirus, Kunjin virus, Lassa virus, Measles virus, Molluscum contagiosum virus, Mumps virus, Nipah virus, Poliovirus, Rabies virus, Rotavirus A, Rotavirus B, Rotavirus C, Rubella virus, Sagiyama virus, Salivirus A, Sapporo virus, Sindbis virus, Toscana virus, Uukuniemi virus, Varicella-zoster virus, Venezuelan equine encephalitis virus, Vesicular stomatitis virus, Western equine encephalitis virus, West Nile virus, Yellow fever virus, Zika virus.

In one embodiment, the functional molecule is an antibody capable of binding to an antigen from a coronavirus. In one embodiment, the antibody is capable of binding to an antigen from SARS-CoV-2.

Suitable bacteria may be selected from: Actinomyces israelii, Bacillus anthracis, Bacillus cereus, Bartonella henselae, Bartonella quintana, Bacteroides fragilis, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Klebsiella pneumoniae, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Nocardia asteroides, Pseudomonas aeruginosa, Rickettsia rickettsia, Salmonella enterica subsp. enterica, Salmonella typhi, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Yersinia pestis, Yersinia enterocolitica, Yersinia pseudotuberculosis.

Suitably in such an embodiment, the VLP may be targeted to a particular virus. Suitably targeted to bind to a particular virus. Suitably the VLP may therefore be used for detecting the presence of a virus. Further details on this use are provided elsewhere.

Suitably the antigen binding protein such as an antibody may be capable of binding to an antigen from a cell surface receptor. Suitably the cell surface receptor may be an ion-channel linked receptor, a G-protein coupled receptor, or an enzyme-linked receptor. Suitably the cell surface receptor is selected from: 5-HT receptor, nAch-receptor, Zinc-activated ion channel, GABA_{A} receptor, Wnt-family member receptors, co-receptors contained in lipid rafts, T-cell and T-cell co-receptors, B-cell receptors and B-cell costimulatory molecules, Glycine receptor, AMPA receptor, Kainate receptor, NMDA receptor, Glutamate receptor, ATP-gated channel, PIP₂ gated channel, Erb receptor, GDNF receptor, NP receptor, trk receptor, toll-like receptor, GABA_{B} receptor, GBPCR class A, B, C, D, E, or F.

Suitably in such an embodiment, the VLP may be targeted to a particular cell. Suitably targeted to bind to a particular cell. Suitably the VLP may be used to deliver cargo to a cell. Further details on this use are provided elsewhere.

Suitable antibodies may include IgG, IgM, IgE, IgA, IgD antibodies. Suitably, the antibody is an IgG antibody. Suitably IgG subclasses include IgG1, IgG2, IgG3 and IgG4.

Suitable further antigen binding proteins may include antibody binding fragments or antibody mimetics which perform the same function as an antibody. Suitably they are also capable of binding an antigen of interest. Suitably the use of an antibody binding fragment or mimetic as a functional molecule also produces a VLP which is capable of binding to an antigen. Suitably this is useful for detecting an antigen, or for targeting the VLP to an antigen as described above.

Suitable antibody binding fragments may include: Fab, monospecific or bispecific F(ab)2, F(ab′)2, monospecific or bispecific diabody, nanobody, ScFv, ScFv-Fc, F(ab)3.

Suitable antibody mimetics may include affibodies, affilins, affimers, affitins, alphabodies, anticalins, avimers, DARPins, fynomers, Kunitz domain peptides, monobodies, nanCLAMPs.

Suitably the use of a fluorescent molecule as a functional molecule produces a VLP which is visible. Suitably this is useful for labelling, especially when combined with a second functional molecule which can bind to an antigen, for example antibodies or binding fragments thereof, antibody mimetics, or aptamers.

Suitable flourescent molecules may include: GFP, EBFP, EBFP2, Azurite, GFPuv, T-saphhire, Cerulean, CFP, mCFP, mTurquoise2, CyPet, mKeima-red, tagCFP, AmCyan1, mTFP1, midoriishi cyan, turboGFP, tagGFP, emerald, azami green, ZsGreen1, YFP, tagYFP, EYFP, topaz, venus, mCtrine, YPet, turboYFP, ZsYellow1, Kusabira Orange, mOrange, allophycocyanin, mkO, RFP, turboRFP, tdTomato, tagRFP, dsRed, mStrawberry, turboFP602, asRed2, J-red, R-phycoerythrin, B-phycoerythrin, mCherry, HcRed, Katusha, P3, peridin chlorophyll, mKate, turboFP635, mPlum, mRaspberry.

Suitably the flourescent molecule is GFP or any modified form of GFP.

In one embodiment of the first aspect, the or each functional molecule is IL13, IL17, IL33, the receptor binding domain of SARS Cov-2 spike protein, or the C-terminus of the SARS Cov-2 nucleocapsid protein.

In another embodiment of the first aspect, the or each functional molecule is an IgG antibody or binding fragment thereof. In one embodiment, the antibody or binding fragment thereof is an antibody or binding fragment thereof directed towards SARS-CoV-2.

In one embodiment of the second aspect, the or each functional molecule is GFP.

In a further embodiment, where the VLP comprises a first functional molecule attached to the first binding protein via a chemical modification, and a second functional molecule attached to the second binding protein, suitably the first functional molecule is GFP and the second functional molecule is an IgG antibody or binding fragment thereof.

Advantageously, in such an embodiment, the VLP can be used to detect an antigen or target the VLP to an antigen and at the same time visibly label the antigen.

In a further embodiment of the first aspect, each second binding protein may be attached to two functional molecules, wherein the first functional molecule is an antigen and the second functional molecule is a different antigen or a flourescent molecule. In one embodiment of the first aspect, each second binding protein is attached to a receptor binding domain of SARS Cov-2 spike protein and a C-terminus of the SARS Cov-2 nucleocapsid protein. In one embodiment of the first aspect, each second binding protein is attached to a receptor binding domain of SARS Cov-2 spike protein and GFP.

Virus-Like Particle (VLP)

The present invention relates to VLPs, their uses and methods of manufacture thereof.

Suitably the VLP comprises one or more viral capsid proteins which suitably form a VLP shell. Suitably the one or more viral capsid proteins self-assemble into the VLP shell.

Suitably the VLP comprises one or more binding proteins which are attached to the viral capsid proteins.

Suitably the VLP shell comprises one or more functional molecules which are suitably attached to the binding proteins, and/or chemical modifications present on the binding proteins.

Suitably, in such a way, the VLP of the invention stably displays the functional molecules on its surface.

Suitably the VLP may comprise a plurality of subunits. Suitably each subunit comprises a complete viral capsid protein, one or more binding proteins and one or more functional molecules. Suitably the subunits self-assemble into a VLP. Suitably therefore the VLP comprises a plurality of viral capsid proteins, a plurality of binding proteins and a plurality of functional molecules. Suitably therefore, in the case of the first aspect, the VLP comprises a plurality of hepatitis B capsid proteins, a plurality of pairs of binding proteins and a plurality of functional molecules.

Suitably each VLP subunit comprises a viral capsid protein dimer, at least two binding proteins, and at least two functional molecules. Suitably each viral capsid monomer is attached to at least one binding protein, and at least one functional molecule.

Optionally each VLP subunit may comprise more than one functional molecule. Suitably each viral capsid monomer may be attached to more than one functional molecule, suitably two functional molecules. Suitably the or each functional molecule may be the same or different. For example, one VLP subunit may comprise functional molecules attached to the second binding proteins, and further functional molecules to the first binding proteins via chemical modification thereof.

In one embodiment of the first aspect, suitably each VLP subunit comprises a hepatitis B capsid protein dimer, two pairs of binding proteins and two functional molecules. Suitably each pair of binding proteins comprises a first binding protein attached to a hepatitis B capsid protein monomer, and a second binding protein attached to a functional molecule.

Suitably in such an embodiment, the two functional molecules may each comprise a monomer of a dimeric protein. Suitably therefore when the hepatitis B capsid protein dimer is formed, the two functional molecules may also come together to form a dimer. One example of a functional molecule where this is the cases is IL17. Suitably when the VLP is intended to display IL17, each functional molecule comprises a monomer of IL17.

In one embodiment of the second aspect, suitably each VLP subunit comprises a hepatitis B capsid protein dimer, two binding proteins and two functional molecules. Suitably each binding protein is attached to a hepatitis B capsid protein monomer and a functional molecule. Suitably the binding proteins are directly or indirectly attached to the viral capsid protein and to the functional molecule. Suitably the binding proteins are directly attached to the viral capsid protein and in some cases directly attached to the functional molecule.

Suitably in the first aspect, the first binding protein is fused to the hepatitis B capsid protein. Suitably in the second aspect, the binding protein is fused to the viral capsid protein.

Suitably in the first aspect, the second binding protein may be fused to the functional molecule. Alternatively, the second binding protein may be indirectly attached to the functional molecule.

In some embodiments, the second binding protein may be indirectly attached to the functional molecule via a third binding protein. Suitably this is used in such embodiments where the functional molecule is an antigen binding molecule such as an antibody. Suitably the third binding protein is a generic antibody binding protein. Suitably the antibody binding protein is selected from protein G, protein A, protein AG, and streptavidin. Suitably the second binding protein may be fused to the third binding protein which may be attached to the functional molecule. In one embodiment, the second binding protein may be fused to the third binding protein which is capable of binding to the functional molecule.

Suitably in the second aspect, the binding protein is indirectly attached to the functional molecule. Sutiably via chemical modification.

Suitably the VLP comprises a negative surface charge, suitably a homogenous negative surface charge.

Nucleic Acids and Vectors

The present invention relates to nucleic acids encoding component protein parts which form the VLP, and vectors comprising said nucleic acids which may be used in host cells to produce VLPs.

Suitably the invention relates to, and makes use of, a first nucleic acid encoding a viral capsid protein attached to a first binding protein. Suitably the first nucleic acid may encode a fusion protein comprising the viral capsid protein fused to a first binding protein. Suitably the viral capsid protein may be a hepatitis B capsid protein. Suitably this may be known as the ‘capsid fusion protein’.

Suitably the first nucleic acid may comprise a sequence according to SEQ ID NO:22, 23, 24, or 25. Suitably the first nucleic acid may comprise a sequence having at least 60%, at least 65%, at least 70%, at least 75%, at least 80%, at least 85%, at least 90%, at least 95% identity with SEQ ID NO: 22, 23, 24, or 25. Suitably the first nucleic acid may consist of a sequence according to SEQ ID NO: 22, 23, 24, or 25.

Suitably the invention relates to, and makes use of, a second nucleic acid encoding a functional molecule optionally attached to a second binding protein. Suitably the second nucleic acid may encode a fusion protein comprising the functional molecule optionally fused to a second binding protein. Suitably this may be known as the ‘functional fusion protein’.

In one embodiment, the second nucleic acid encodes only a functional molecule.

In one embodiment, the second nucleic acid encodes a functional molecule attached to a second binding protein. In one embodiment, the second nucleic acid encodes a functional molecule fused to a second binding protein.

Suitably the second nucleic acid may comprise a sequence according to SEQ ID NO:26, 27, 28, 29, 41, 32, or 45. Suitably the second nucleic acid may comprise a sequence having at least 60%, at least 65%, at least 70%, at least 75%, at least 80%, at least 85%, at least 90%, at least 95% identity with SEQ ID NO: 26, 27, 28, 29, 41, 32, or 45. Suitably the second nucleic acid may consist of a sequence according to SEQ ID NO: 26, 27, 28, 29, 41, 32 or 45

Suitably the functional molecule is an epitope. Suitably an epitope selected from IL-13, IL-33, IL-17, or SARS-Cov2 spike protein receptor binding domain.

In embodiments where the second nucleic acid encodes an IL-13 epitope fused to a second binding protein, suitably the second nucleic acid may comprise a sequence according to SEQ ID NO: 26 or 27, or a sequence having at least 60%, at least 65%, at least 70%, at least 75%, at least 80%, at least 85%, at least 90%, at least 95% identity with SEQ ID NO:26 or 27.

In embodiments where the second nucleic acid encodes an IL-33 epitope fused to a second binding protein, suitably the second nucleic acid may comprise a sequence according to SEQ ID NO: 28 or 29, or a sequence having at least 60%, at least 65%, at least 70%, at least 75%, at least 80%, at least 85%, at least 90%, at least 95% identity with SEQ ID NO:28 or 29.

In embodiments where the second nucleic acid encodes a SARS-CoV2 spike protein epitope fused to a second binding protein, suitably the second nucleic acid may comprise a sequence according to SEQ ID NO:41, or a sequence having at least 60%, at least 65%, at least 70%, at least 75%, at least 80%, at least 85%, at least 90%, at least 95% identity with SEQ ID NO:41.

In embodiments where the second nucleic acid encodes an IL-17 epitope fused to a second binding protein, suitably the second nucleic acid may comprise a sequence according to SEQ ID NO: 45, or a sequence having at least 60%, at least 65%, at least 70%, at least 75%, at least 80%, at least 85%, at least 90%, at least 95% identity with SEQ ID NO:45.

In embodiments where the second nucleic acid encodes two functional molecules fused to a second binding protein, suitably the second nucleic acid may comprise a sequence according to SEQ ID NO: 42 or 43, or a sequence having at least 60%, at least 65%, at least 70%, at least 75%, at least 80%, at least 85%, at least 90%, at least 95% identity with SEQ ID NO: 42 or 43. Suitably the functional molecules may comprise one or more epitopes and/or a flourescent molecule. Suitably the functional molecules may comprise two epitopes. Suitably the functional molecules may comprise an epitope and a flourescent molecule.

In embodiments where the second nucleic acid encodes two epitopes, they may be any two epitopes fused to a second binding protein. In one embodiment, the second nucleic acid encodes a SARS-Cov2 spike protein receptor binding domain and a C-terminal fragment of the nucleocapsid protein. Suitably the second nucleic acid may comprise a sequence according to SEQ ID NO:42, or a sequence having at least 60%, at least 65%, at least 70%, at least 75%, at least 80%, at least 85%, at least 90%, at least 95% identity with SEQ ID NO:42.

In embodiments where the second nucleic acid encodes an epitope and a flourescent molecule fused to a second binding protein, they may be any epitope and any flourescent molecule. In one embodiment, the second nucleic acid encodes a SARS-Cov2 spike protein receptor binding domain and eGFP. Suitably the second nucleic acid may comprise a sequence according to SEQ ID NO:43, or a sequence having at least 60%, at least 65%, at least 70%, at least 75%, at least 80%, at least 85%, at least 90%, at least 95% identity with SEQ ID NO: 43.

In some embodiments, the invention relates to, and makes use of a further nucleic acid encoding a second functional molecule. Suitably this further nucleic acid may be known as the fourth nucleic acid. In some embodiments, this may occur when the second nucleic acid already encodes a first functional molecule attached to a second binding protein. Suitably in such embodiments, the first binding protein is chemically modified.

In some embodiments, the invention relates to, and makes use of, a further nucleic acid encoding a second binding protein attached to a third binding protein. Suitably this further nucleic acid may be known as the third nucleic acid. Suitably the third binding protein is a protein capable of binding to an antigen binding protein such as an antibody. Suitably the third binding protein may be protein G, for example.

Suitably the third nucleic acid may comprise a sequence according to SEQ ID NO: 30. Suitably the third nucleic acid may comprise a sequence having at least 60%, at least 65%, at least 70%, at least 75%, at least 80%, at least 85%, at least 90%, at least 95% identity with SEQ ID NO: 30. Suitably the third nucleic acid may consist of a sequence according to SEQ ID NO: 30.

Suitably the first, second, and third binding proteins are defined elsewhere herein. However, suitably the first binding protein may be a bacterial toxin inhibitor and the second binding protein may be a bacterial toxin. Suitably the third binding protein may be an antibody binding protein.

In some embodiments, the invention may make use of the first and second nucleic acids.

In some embodiments, the invention may make use of the first, second and third nucleic acids.

In some embodiments, the invention may make use of the first, second and fourth nucleic acids.

In some embodiments, the invention may make use of the first and third nucleic acids.

In some embodiments, the invention may make use of the first, second, third, and fourth nucleic acids.

Suitably the first, second, third and fourth nucleic acids described herein may be provided as one contiguous nucleic acid sequence, or may be provided as a plurality of separate nucleic acid sequences. References to the first, second, third, and fourth nucleic acids include embodiments where plurality of nucleic acid sequences may be used to encode the same proteins as the first, second, third, and fourth nucleic acids.

Suitably the nucleic acids may comprise one or more expression elements to aid in expression of the proteins encoded thereon.

Suitable expression elements include promoters, operators, enhancers, activators, repressors, 5′UTRs, 3′UTRs, introns, IRES, etc.

Suitably each of the nucleic acids comprises one or more expression elements which ensure equal expression of the proteins encoded thereon. Suitably each of the nucleic acids comprises a promoter which ensures equal expression of the proteins encoded therein. Suitably the promoter may comprise one or more modifications which adapt the level of expression therefrom. Suitably the promoter may comprise one or more mutations. Suitably the or each nucleic acid described herein is operably linked to a promoter.

Suitable promoters may be selected from: CMV-IE, EF1a, SV40, PGK1, CAG, human beta actin, T7, TetR/TetA, T7lac, SP6, LP1, TTR, CK8, Synapsin, Glial fibrillary acidic protein (GFAP), CaMKII, TBG, and albumin promoter.

Suitably each nucleic acid may be linked to the same promoter or a different promoter.

Suitably each nucleic acid may be linked to the same promoter. Suitably therefore each nucleic acid may be expressed at the same time. Suitably each nucleic acid may be linked to a T7 promoter, optionally with one or more modifications to ensure equal expression levels of the proteins encoded by the nucleic acids.

Suitably each nucleic acid may be linked to a different promoter. Suitably therefore each nucleic acid may be expressed at different times. Suitably the or each nucleic acid may be independently expressed. Suitably expression of each nucleic acid may be induced at different times. Suitably therefore the or each promoter may be an inducible promoter. Suitably which may be induced by contacting the promoter with a suitable inducer, at a concentration effective to induce expression therefrom. In one embodiment, the first nucleic acid sequence may be linked to a first promoter and the second nucleic acid may be linked to a second promoter. Suitably the first promoter may be a T7 promoter to modified T7 promoter as described herein. Suitably the second promoter may be a TetR/TetA promoter.

In one embodiment, the T7 promoter operably linked to the second nucleic acid is modified. Suitably the T7 promoter operably linked to the second nucleic acid is modified to reduce the expression level of the functional molecule attached to a second binding protein encoded thereon. Suitably the T7 promoter is modified by a point mutation. Suitably the T7 promoter may comprise any of the following modifications in the nomenclature according to Konczal et al, PLoS One 2019: 1C, 1T, 2T, 5A, 8G, 4C, or any combination thereof, wherein the parent sequence is: agcataat (SEQ ID NO:44). Suitably the modified T7 promoter comprises a sequence according to SEQ ID NO:31. In such an embodiment, suitably the T7 promoter operably linked to the first nucleic acid is not modified.

Suitably therefore the first nucleic acid expresses the viral capsid protein attached to a first binding protein at the same level as the second nucleic acid expresses the functional molecule attached to a second binding protein, or at the same level as the third nucleic acid.

Suitably therefore the capsid fusion protein is expressed at a 1:1 level compared to the functional fusion protein, or the functional molecule.

Suitably the nucleic acids may be comprised on one or more vectors. Suitably the first, second, third, and/or fourth nucleic acids may be comprised on one vector. Alternatively, first, second, third, and/or fourth nucleic acids may be comprised on multiple vectors. In one embodiment, the first nucleic acid may be comprised on one vector and the second nucleic acid may be comprised on another vector.

In one embodiment, the first nucleic acid is comprised on a first vector, suitably the vector of SEQ ID NO:1 or 2.

In one embodiment, the second nucleic acid is comprised on a second vector, suitably the vector of SEQ ID NO: 3, 4, 9, 10 or 11.

Alternatively, in one embodiment, the first and second nucleic acids may be comprised on the same vector, suitably the vector of SEQ ID NO: 5, 6, 7, 47 or 48.

In one embodiment, the first nucleic acid and the third nucleic acid are comprised on the same vector, suitably the vector of SEQ ID NO:8.

Suitably the one or more vectors may be comprised in one or more host cells. Suitably the one or more vectors may be comprised in a single host cell. Suitably, for example in the tenth aspect. Alternatively the one or more vectors may be comprised in a plurality of host cells in any combination. Suitably for example in the eleventh aspect.

Suitably in the process of the tenth aspect, the first, second and/or third nucleic acids may be comprised on one vector or on a first and second vector, or on a first, second and third vector respectively. In one embodiment of the process of the tenth aspect, the first and second nucleic acids are comprised on one vector. Suitably the or each vector is present in the single host cell. In one embodiment of the process of the tenth aspect, the first and second nucleic acids are comprised on a single vector of SEQ ID NO:5, 6,7, 47 or 48. In one embodiment of the process of the tenth aspect, the first and third nucleic acids are comprised on a single vector of SEQ ID NO:8. Suitably the single host cell comprises a single vector of SEQ ID NO:5, 6, 7, 8, 47 or 48. In one embodiment of the process of the tenth aspect, the first, second and/or third nucleic acids are comprised on two different vectors. Suitably the first nucleic acid may be comprised on a first vector selected from SEQ ID NO:1 or 2. Suitably the second nucleic acid may be comprised on a second vector selected from SEQ ID NO: 3, 4, 9, 10, or 11. Suitably any workable combination of first and second vectors may be used in the single host cell. For example, the first vector may comprise SEQ ID NO:1 and may be combined with any of the second vectors of SEQ ID NO: 3, 4, 9, 10, or 11. For example, the first vector may comprise SEQ ID NO:2 and may be combined with any of the second vectors of SEQ ID NO: 3, 4, 9, 10, or 11.

Suitably in the process of the eleventh aspect, the first and second nucleic acids are comprised on a first and second vector respectively. Suitably the third nucleic acid may be comprised on a second vector together with the second nucleic acid or alone. Alternatively the third nucleic acid may be comprised on a third vector. Suitably the first vector is present in the first host cell and the second and/or third vector is present in a second host cell. Alternatively, the third vector may be present in a third host cell. In one embodiment of the process of the eleventh aspect, suitably the first vector is of SEQ ID NO:1 or 2, and the second vector is of SEQ ID NO: 3, 4, 9, 10 or 11. Suitably any workable combination of first and second vectors may be used in the host cells. For example, the first host cell may comprise a first vector of SEQ ID NO:1 and may be combined with a second host cell comprising a second vector of any of SEQ ID NO: 3, 4, 9, 10, or 11. For example, the first host cell may comprise a first vector of SEQ ID NO:2 and may be combined with a second host cell comprising a second vector of any of SEQ ID NO: 3, 4, 9, 10, or 11.

Suitably, the one or more vectors may further comprise the third and/or fourth nucleic acids. In one embodiment, the one or more vectors may further comprise both a third nucleic acid encoding a second binding protein attached to a third binding protein, and a fourth nucleic acid encoding a second functional molecule.

Suitably the further third and/or fourth nucleic acids may be comprised on a vector in the first or second host cells. Suitably the further third and/or fourth nucleic acids may be comprised on the same vector as the first and/or second nucleic acids, or on different vectors. Suitably the third and/or fourth nucleic acids may both be comprised on a third vector. Alternatively, the third and/or fourth nucleic acids may be comprised on a third and a fourth vector respectively. Suitably the third and/or fourth vector may be present in the first or second host cells. Alternatively, the third and/or fourth vector may be present in a third host cell. Alternatively, the third vector may be present in a third host cell and the fourth vector may be present in a fourth host cell.

Any suitable vector may be used for the chosen host cell/s. Suitable host cells are discussed below. Suitably the vector is selected from: a plasmid, a cosmid, a phage, a virus, an artificial chromosome. Suitably the or each vector is a plasmid.

Suitable plasm id vectors for a host E. coli cell may include, for example: pALTER-Ex1, pALTER-Ex2, pBAD/His, pBAD/Myc-His, pBAD/gIII, pCal-n, pCal-n-EK, Cal-c, pCal-Kc, pcDNA 2.1, pDUAL, pET-3a-c, pET-9a-d, pET-11a-d, pET-12a-c, pET-14b, pET-15b, pET-16b, pET-17b, pET-19b, pET-20b(+), pET-21a-d(+), pET-22b(+), pET-23a-d(+), pET-24a-d(+), pET-25b(+), pET-26b(+), pET-27b(+), pET-28a-c(+), pET-29a-c(+), pET-30a-c(+), pET-31b(+), pET-32a-c(+), pET-33b(+), pET-34b(+), pET-35b(+), pET-36b(+), pET-37b(+), pET-38b(+), pET-39b(+), pET-40b(+), pET-41a-c(+), pET-42a-c(+), pET-43a-c(+), pETBlue-1, pETBlue-2, pETBlue-3, pGEMEX-1, pGEMEX-2, pGEX-1IT, pGEX-2T, pGEX-2TK, pGEX-3X, pGEX-4T, pGEX-5X, pGEX-6P, pHAT10/11/12, pHAT20, pHAT-GFPuv, pKK223-3, pLEX, pMAL-c2X, pMAL-c2E, pMAL-c2G, pMAL-p2X, pMAL-p2E, pMAL-p2G, pProEX HT, pPROLar.A, pPROTet.E, pQE-9, pQE-16, pQE-30/31/32, pQE-40, pQE-60, pQE-70, pQE-80/81/82L, pQE-100, pRSET, pSE280, pSE380, pSE420, pThioHis, pTrc99A, pTrcHis, pTrcHis2, pTriEx-1, pTriEx-2, pTrxFus.

In one embodiment, the vector used is pET-Duet.

Suitable plasmid vectors for a host mammalian cell may include: the pSV and the pCMV series of vectors.

In one embodiment, the vector used is pcDNA5D. In one embodiment, host mammalian cells are HEK293 cells or CHO cells or derivatives thereof.

Suitably if more than one vector is used, it is the same vector.

Suitably the vector may comprise a variety of other functional nucleic acid sequences, such as one or more selectable markers, one or more origins of replication, multiple cloning sites and the like.

Process of Producing a VLP

The present invention further relates to processes for the production of VLPs. Two different processes are described herein, one is a single cell process, the other is a process which takes place in at least two cells and requires mixing of component parts to form the VLP.

In accordance with the tenth aspect of the invention, there is provided a single cell process of producing a VLP.

In accordance with the eleventh aspect of the invention, there is provided a multiple cell process of producing a VLP.

Suitably the processes may further comprise transfecting the one or more vectors comprising the nucleic acids into the or each host cell. Suitably prior to culturing the or each host cell. Suitably transfection may take place by any suitable method such as electroporation, microinjection, particle delivery, chemical mediated endocytosis, calcium phosphate co-precipitation, or liposome mediated delivery.

Suitably culturing the host cells under conditions to express the proteins comprises culturing the host cells under optimum growth conditions. Suitably the optimum growth conditions will vary depending on the host cell being used.

Suitably the host cell may be selected from any bacterium, yeast, insect cell or human cell. Suitably the host cell is a bacterial host cell. Suitably the host cell is selected from E. coli, B. subtilis, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus brevis, Bacillus megaterium, Bacillus licheniformis, Bacillus amyloliquefacien, Lactococcus lactis, Lactobacillus plantarum, Lactobacillus casei, Lactobacillus reuteri, Lactobacillus gasseri.

In one embodiment, the host cell is E. coli. Suitably the E. coli strain is selected from BL21, lemo21, NiCo21, NEB Express, SHuffle, T7 Express, BLR, HMS174, Tuner, Origami2, Rosetta2, m15.

In one embodiment, the E. coli strain is BL21(DE3) where the additional genes regulating disulfide formation, dsbC and erv1P, are integrated genomically. Suitably, the genomic integration is within the recAX locus.

In an alternative embodiment, the host cell is a human cell, such as a HEK293T cell.

Suitably optimum growth conditions comprise culturing at a temperature of 15-25° C. Suitably optimum growth conditions comprise culturing in a medium compatible with bioprocess applications for medicines intended for use in humans, such as chemically defined medium. Suitably optimum growth conditions comprise culturing in an aerated culture medium.

Suitably the host cells are cultured to a high density. Suitably to a density OD₆₀₀ of 4-20.

Suitably culturing the host cells under conditions to express the proteins may also comprise inducing the host cells to express the proteins. Suitably inducing the host cells may comprise addition of an inducer into the culture medium, or the creation of certain inducive conditions within the culture medium such as acid/alkali pH, heat shock, hypoxia or the like. Suitably the inducer or inducive condition stimulates transcription of the nucleic acids. Suitably an inducer or inducive condition does so by stimulating an inducible expression control sequence within the nucleic acids. Suitably the inducible expression control sequence may be an inducible promoter. Suitable inducers include isopropyl-β-d-thiogalactoside (IPTG) for lactose driven promoters or tetracycline for tetracycline—regulated promoters.

Suitably the host cells are induced to express the proteins once the culture has reached the optimal density described above. Suitably the host cells are induced to express the proteins during logarithmic growth.

Suitably the concentration of proteins may be varied by adjusting the concentration of an inducer or altering the inducive conditions to which the host cells are exposed.

Suitably the culturing step takes between 4-24 hours.

Sutiably the host cells are induced to express the proteins after 2-6 h of culturing or when an OD of 6-8 has been achieved.

In a further aspect of the invention, there is provided a cell culture comprising one or more host cells of the seventh, eighth or ninth aspects and a culture medium. Suitably a plurality of said cells.

Alternatively, the process may not be conducted within one or more cells, and may be conducted in a cell-free system. Suitably in the process of the eleventh aspect, step (a) is conducted within a host cell, to ensure proper production of the VLP shell. However, suitably step (b) may occur outside of a host cell, in a cell free system.

Suitably the processes may further comprise a step of recovering the VLPs. Suitably recovering the VLPs from the host cells. Suitably after the VLPs have been formed.

Suitably recovering the VLPs may comprise disrupting the host cells. Alternatively, the host cells may secrete the VLPs into the culture solution. Suitably disrupting the host cells may be carried out by any suitable method such as homogenisation, sonication, or freeze-thaw.

Recovery of the VLPs may take place by any suitable method such as filtration, pull-down, centrifugation, or chromatography.

Suitably, in an embodiment where the binding protein comprises a chemical modification, suitably the recovery and purification of VLPs takes place by chromatography. Suitably involving a sequence of steps including mixed mode (hydrophobic interaction and size exclusion) chromatography, anion exchange chromatography, and ultrafiltration. Suitably by anion exchange chromatography. Suitably when anion exchange chromatography is used to recover the VLPs, the VLP may comprise chemical modification, suitably in such an embodiment the first binding protein of the VLP is modified with DEAE. Sutiably the DEAE molecules can bind to the chromatography column.

Suitably, in the process of the eleventh aspect, step (d) comprises recovering the proteins. Suitably recovering the proteins from the host cells. Suitably recovering the proteins may be performed by similar techniques. Suitably recovering the proteins may comprise disrupting the host cells as above. Alternatively, the host cells may secrete the proteins into the culture solution.

Suitably the VLPs form by self-assembly, suitably automatic self-assembly. Suitably once the component proteins are mixed, either within a single host cell as per the tenth aspect or outside of a cell as per the eleventh aspect, they will assemble to form VLPs.

In respect of the single cell process of the tenth aspect, suitably the step of culturing the host cell further comprises culturing under conditions such that the proteins expressed from the first and second nucleic acids, or from any further nucleic acids, bind to each other.

In some embodiments, after the culturing step the first binding protein may be chemically modified. Suitably therefore the method may comprise a step of recovering the proteins, and subsequently chemically modifying the first binding protein. Suitably these steps take place after step (b) but prior to step (c).

In some embodiments, the one or more vectors may further comprise a further (fourth) nucleic acid encoding a second functional molecule. Suitably in such embodiments, the first binding protein is chemically modified. Suitably the second functional molecule binds to the chemical modification. Suitably in such embodiments, the host cell is cultured under conditions to express the proteins from the first, second and fourth nucleic acids.

In some embodiments, the one or more vectors may further comprise a third nucleic acid encoding a second binding protein attached to a third binding protein. Suitably in such embodiments, the host cell is cultured under conditions to express the proteins from the first and third nucleic acids. Suitably in such an embodiment, the second nucleic acid may be present, and may encode only a functional molecule. Suitably in such an embodiment, the functional molecule is an antigen binding protein.

In some embodiments, the host cell may be cultured under conditions so as to express proteins from the first, second, third and fourth nucleic acids.

In one embodiment, the second nucleic acid encodes only a functional molecule. Suitably, in such an embodiment, the first binding protein is chemically modified, or the third nucleic acid is present.

In one embodiment, the second nucleic acid encodes a functional molecule attached to a second binding protein. Suitably in such an embodiment, the first binding protein may or may not be chemically modified.

In one embodiment, step (c) of the tenth aspect comprises each first binding protein binding to each second binding protein. In an alternative embodiment, step (c) comprises each first binding protein binding to a functional molecule, suitably via a chemical modification. In one embodiment, step (c) comprises both of these steps.

In respect of the multiple cell process of the eleventh aspect, suitably during the culturing step the first binding protein may be chemically modified. Suitably therefore the conditions for culturing the first host cell are such that the first binding protein is chemically modified. Suitably such chemical modification of the first binding protein may take place post-translationally. Alternatively, the method may comprise a step of chemically modifying the first binding protein. Suitably this step takes place after step (d) but prior to step (e).

In some embodiments, the one or more vectors may further comprise a further (fourth) nucleic acid encoding a second functional molecule. Suitably the fourth nucleic acid may be comprised on a vector in the first or second host cells, or may be comprised on a vector in a third host cell. Suitably in such embodiments, the first binding protein is chemically modified. Suitably in such embodiments, the host cells are cultured under conditions to express the proteins from the first, second and fourth nucleic acids.

In some embodiments, the one or more vectors may comprise a third nucleic acid encoding a second binding protein attached to a third binding protein. Suitably in such embodiments, the host cells are cultured under conditions to express the proteins from the first, and third nucleic acids.

Suitably in such an embodiment, the second nucleic acid if present encodes only a functional molecule. Suitably in such an embodiment, the functional molecule is an antigen binding protein.

In some embodiments, the host cells may be cultured under conditions so as to express proteins from the first, second, third and fourth nucleic acids.

In one embodiment, step (e) comprises each first binding protein binding to each second binding protein. In an alternative embodiment, step (e) comprises each first binding protein binding to each functional molecule, suitably via a chemical modification. In one embodiment, step (e) comprises both of these steps.

In one embodiment, step (e) further comprises mixing under conditions such that the proteins bind to each other. Suitably step (e) comprises mixing host cell supernatants or host cell lysates. Suitably mixing the first host cell supernatant or lysate with the further host cell(s) supernatant or lysate. Suitably the mixing is such that the ratio of the binding proteins confers an even stoichiometric concentration. Suitably the mixing is such that the ratio of first host cell supernatant or lysate to further host cell(s) supernatant or lysate is about 1:1. Suitably the mixing step takes place at room temperature, suitably around 18-22° C. Suitably mixing takes place for between 15 minutes to 2 hours, suitably between 20 minutes and 1 hour, suitably between 25 minutes and 45 minutes, suitably for about 30 minutes.

In one embodiment of the tenth aspect of the present invention, there is provided a process of producing a virus-like particle (VLP) in a single host cell comprising:
(a) Providing a host cell comprising one or more vectors, wherein the one or more vectors comprise:
   (i) a first nucleic acid encoding a viral capsid protein attached to a first binding protein; and
   (ii) a second nucleic acid encoding a functional molecule;
(b) Culturing the host cell under conditions to express the proteins from the first and second nucleic acids respectively;
(c) Forming virus-like particles from the proteins.

Suitably wherein the first binding protein is chemically modified, and the functional molecule is capable of binding to the chemical.

In one embodiment of the tenth aspect of the present invention, there is provided a process of producing a virus-like particle (VLP) in a single host cell comprising:
(a) Providing a host cell comprising one or more vectors, wherein the one or more vectors comprise:
   (i) a first nucleic acid encoding a viral capsid protein attached to a first binding protein; and
   (ii) a second nucleic acid encoding a functional molecule attached to a second binding protein;
(b) Culturing the host cell under conditions to express the proteins from the first, and second nucleic acids respectively;
(c) Forming virus-like particles from the proteins.

In one embodiment of the tenth aspect of the present invention, there is provided a process of producing a virus-like particle (VLP) in a single host cell comprising:
(a) Providing a host cell comprising one or more vectors, wherein the one or more vectors comprise:
   (i) a first nucleic acid encoding a viral capsid protein attached to a first binding protein; and
   (ii) a third nucleic acid encoding a second binding protein attached to a third binding protein;
(b) Culturing the host cell under conditions to express the proteins from the first, and third nucleic acids respectively;
(c) Forming virus-like particles from the proteins.

Sutiably a functional molecule may be mixed with the VLPs once formed, suitably the functional molecule is capable of binding to the third binding protein.

In one embodiment of the tenth aspect of the present invention, there is provided a process of producing a virus-like particle (VLP) in a single host cell comprising:
(a) Providing a host cell comprising one or more vectors, wherein the one or more vectors comprise:
   (i) a first nucleic acid encoding a viral capsid protein attached to a first binding protein; and
   (ii) (a) a second nucleic acid encoding a functional molecule; and
      (b) a third nucleic acid encoding a second binding protein attached to a third binding protein;
(b) Culturing the host cell under conditions to express the proteins from the first, second and third nucleic acids respectively;
(c) Forming virus-like particles from the proteins.

Suitably wherein the functional molecule is capable of binding to the third binding protein.

In one embodiment of the eleventh aspect of the present invention, there is provided a process of producing a virus-like particle (VLP), comprising;
(a) Providing a first host cell comprising one or more vectors, wherein the one or more vectors comprise:
   (j) a first nucleic acid encoding a hepatitis B capsid protein attached to a first binding protein, wherein the first binding protein is a bacterial toxin inhibitor;
(b) Providing one or more further host cell(s) comprising one or more vectors, wherein the one or more vectors comprise:
   (iii) a second nucleic acid encoding a functional molecule;
(c) Culturing the host cells under conditions to express the proteins from the first, and second nucleic acids respectively;
(d) Recovering the proteins;
(e) Mixing the proteins to form virus-like particles.

Suitably wherein the first binding protein is chemically modified, and the functional molecule is capable of binding to the chemical.

Suitably step (b) comprises providing a second host cell.

In one embodiment of the eleventh aspect of the present invention, there is provided a process of producing a virus-like particle (VLP), comprising;
(a) Providing a first host cell comprising one or more vectors, wherein the one or more vectors comprise:
   (i) a first nucleic acid encoding a hepatitis B capsid protein attached to a first binding protein, wherein the first binding protein is a bacterial toxin inhibitor;
(b) Providing one or more further host cell(s) comprising one or more vectors, wherein the one or more vectors comprise:
   (i) a second nucleic acid encoding a functional molecule attached to a second binding protein wherein the second binding protein is a bacterial toxin;
(c) Culturing the host cells under conditions to express the proteins from the first, and second nucleic acids respectively;
(d) Recovering the proteins;
(e) Mixing the proteins to form virus-like particles.

Suitably step (b) comprises providing a second host cell.

In one embodiment of the eleventh aspect of the present invention, there is provided a process of producing a virus-like particle (VLP), comprising;
(a) Providing a first host cell comprising one or more vectors, wherein the one or more vectors comprise:
   (i) a first nucleic acid encoding a hepatitis B capsid protein attached to a first binding protein, wherein the first binding protein is a bacterial toxin inhibitor;
(b) Providing one or more further host cell(s) comprising one or more vectors, wherein the one or more vectors comprise:
   (i) a third nucleic acid encoding a second binding protein attached to a third binding protein, wherein the second binding protein is a bacterial toxin;
(c) Culturing the host cells under conditions to express the proteins from the first, and third nucleic acids respectively;
(d) Recovering the proteins;
(e) Mixing the proteins to form virus-like particles.

Suitably a functional molecule may be mixed with the VLPs once formed, suitably the functional molecule is capable of binding to the third binding protein.

Sutiably step (b) comprises providing a second host cell.

In one embodiment of the eleventh aspect of the present invention, there is provided a process of producing a virus-like particle (VLP), comprising;
(a) Providing a first host cell comprising one or more vectors, wherein the one or more vectors comprise:
   (i) a first nucleic acid encoding a hepatitis B capsid protein attached to a first binding protein, wherein the first binding protein is a bacterial toxin inhibitor;
(b) Providing one or more further host cell(s) comprising one or more vectors, wherein the one or more vectors comprise:
   (i) a second nucleic acid encoding a functional molecule; and
   (ii) a third nucleic acid encoding a second binding protein attached to a third binding protein, wherein the second binding protein is a bacterial toxin;
(c) Culturing the host cells under conditions to express the proteins from the first, second and third nucleic acids respectively;
(d) Recovering the proteins;
(e) Mixing the proteins to form virus-like particles.

Suitably wherein the functional molecule is capable of binding to the third binding protein.

Suitably step (b) may comprise providing a second host cell comprising (i) and (ii). Alternatively step (b) may comprise a providing a second host cell comprising (i) and providing a third host cell comprising (ii).

Immunogenic Composition

The present invention further relates to an immunogenic composition comprising the VLP of the invention.

Suitably the immunogenic composition may be a vaccine.

Suitably the immunogenic composition may further comprise one or more adjuvants. Suitable adjuvants include: mineral salts, emulsions, microorganism derived adjuvants, carbohydrates, cytokines, particulates or tensoactive compounds.

Suitable mineral salts include: adjumer, alhydrogel, aluminium hydroxide, aluminum phosphate, aluminium potassium sulphate, amorphous aluminium hydroxyphosphate sulfate (AAHSA), aluminium salts in general, calcium phosphate, Rehydragel HPA, or Rehydragel LV.

Suitable emulsions include: Freund's complete, Freund's incomplete, montanide ISA720, montanide ISA 51, montanide incomplete, Ribi, TiterMax, AF03, AS03, MF59, specol, SPT, or squalene.

Suitable microorganism derived include: cholera toxin or mutants thereof, cholera toxin subunit B, CpG DNA, LTR 192G, MPL, Bordella pertussis components, E. coli heat labile toxin, CTA1-DD gene fusion protein, Etx B subunit, lipopolysaccharides, flagellin, Corynebacterium derived P40, LTK72, MPL-SE, or Ty particles.

Suitably the immunogenic composition may further comprise one or more pharmaceutically acceptable excipients. Pharmaceutically acceptable excipients may include stabilizers, fillers, preservatives, diluents, nutrients, antioxidants, antimicrobial agents, buffers, solvents, inactivating agents, purifiers, emulsifiers, surfactants and the like.

Suitable excipients may be selected from, for example: monosodium glutamate, sucrose, D-mannose, D-fructose, dextrose, human serum albumin, potassium phosphate, plasdone C, anhydrous lactose, microcrystalline cellulose, polacrilin potassium, magnesium stearate, cellulose acetate phthalate, alcohol, acetone, castor oil, sodium chloride, benzethonium chloride, formaldehyde, ascorbic acid, hydrolyzed casein, sodium bicarbonate, sodium carbonate, glutaraldehyde, 2-phenoxyethanol, polysorbate 80 (Tween 80), neomycin, polymyxin B sulfate, bovine serum albumin, neomycin sulfate, polymyxin B, yeast protein, streptomycin sulfate, ammonium thiocyanate, rice protein, lactose, formalin, amino acid supplement, phosphate-buffered saline solution, disodium phosphate dihydrate, sodium dihydrogen phosphate dihydrate, yeast DNA, deoxycholate, phosphorothioate linked oligodeoxynucleotide, dibasic dodecahydrate, monobasic dehydrate, L-histidine, sodium borate, sodium taurodeoxycholate, ovalbumin, sorbitan trioleate, sodium citrate dehydrate, citric acid monohydrate, kanamycin, barium, hydrocortisone, egg proteins, cetyltrimethylammonium bromide (CTAB), octoxynol-10 (TRITON X-100), α-tocopheryl hydrogen succinate, gentamicin sulfate, monobasic sodium phosphate, dibasic sodium phosphate, cetyltrimethlyammonium bromide, and β-propiolactone, Thimerosal, α-tocopheryl hydrogen succinate, hydrolyzed porcine gelatin, arginine, dibasic potassium phosphate, monobasic potassium phosphate, protamine sulfate, sodium metabisulphite, Vero cell protein, CRM197 protein, vitamins, bovine calf serum, urea, succinate buffer, isotonic saline solution, phenol, M-199 medium, chicken protein, polygeline, chlortetracycline, dextran, Dulbecco's Modified Eagle Medium, magnesium sulfate, ferric (III) nitrate, L-cystine, L-tyrosine, sorbitol, xanthan, water, EDTA, dioleoyl phosphatidylcholine (DOPC), 3-O-desacl4′monophosphoryl lipid A (MPL), QS-21, and cholesterol.

In one embodiment, the excipients may be arginine, glutamine and trehalose.

Suitably the immunogenic composition is formulated as a fluid, suitably as a liquid. Suitably the excipients and additives are selected such that the formulation is a liquid. Suitably an injectable liquid.

Immunogenicity

The term “Immunogenic” means that a VLP or an immunogenic composition comprising the VLP of the invention is capable of eliciting an immune response in a subject. Suitably a potent and preferably a protective immune response in a subject.

Thus, the VLP or an immunogenic composition comprising the VLP of the invention may be capable of generating an antibody response in a subject and/or a non-antibody based immune response in a subject. Suitably this may be referred to as its immunogenic activity.

As set out in the Examples, the inventors have demonstrated that an immunogenic composition comprising the VLPs of the invention exhibit immunogenic activity that is comparable, if not improved, compared with a control vaccine. However, surprisingly, the inventors have found that a vaccine comprising the VLPs of the invention elicited an immunogenic response that was quicker and then more sustained and consistent as compared to a control vaccine. Therefore, the VLPs of the invention show immunogenic activity that is well suited to therapeutic use as a medicament.

Suitably the immunogenic activity of the VLP or an immunogenic composition comprising the VLP of the invention may be determined by the amount of antibodies present in a subject after administration of the VLP or an immunogenic composition comprising the VLP of the invention i.e. antibody production. Suitably the amount of antibodies which bind to the antigen of the VLP. Suitably the amount of antibodies present in a subject after administration of the VLP or an immunogenic composition comprising the VLP of the invention, i.e. antibody production, is sustained and consistent over a period of time. Suitably the immunogenic activity of the VLP or an immunogenic composition comprising the VLP of the invention may be determined by the amount of antibodies present in a subject after administration of the VLP or an immunogenic composition comprising the VLP of the invention over a given period of time, i.e. antibody production over a given period of time. Suitable periods of time are outlined below. By amount of antibodies it is meant the titre or concentration thereof. Suitably the concentration of antibodies in sera.

Suitably a VLP or an immunogenic composition comprising the VLP of the invention may sustain immunogenic activity for at least 5 days, at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, or at least 100 days or more in a subject. Suitably a VLP or an immunogenic composition comprising the VLP of the invention may sustain immunogenic activity for at least 110 days, at least 120 days, at least 130 days, at least 140 days, at least 150 days, at least 160 days, at least 170 days, at least 180 days, at least 190 days, at least 200 days, at least 210 days, at least 220 days, at least 230 days, at least 240 days, at least 250 days, at least 260 days, at least 270 days, at least 280 days, at least 290 days, at least 300 days or more in subject.

Suitably a VLP or an immunogenic composition comprising the VLP of the invention may sustain immunogenic activity for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 12 weeks, at least 14 weeks, at least 16 weeks, at least 18 weeks, at least 20 weeks days or more in a subject. Suitably a VLP or an immunogenic composition comprising the VLP of the invention may sustain immunogenic activity for at least at least 30 weeks, at least 40 weeks, at least 50 weeks, at least 60 weeks, at least 70 weeks, at least 80 weeks, at least 90 weeks, at least 100 weeks or more in a subject.

Suitably a VLP or an immunogenic composition comprising the VLP of the invention may sustain immunogenic activity for at least for at least 1 year, at least 2 years at least 3 years, at least 4 years, at least 5 years, at least 6 years, at least 7 years, at least 8 years, at least 9 years or at least 10 years or more in a subject. Suitably, a VLP or an immunogenic composition comprising the VLP of the invention may sustain immunogenic activity for at least 10 years, for at least 15 years, for at least 20 years, for at least 25 years, for at least 30 years, for at least 35 years, for at least 40 years, for at least 45 years, for at least 50 years or more in a subject.

Suitably wherein immunogenic activity may refer to immunogenic antibody production. Suitably antibody production at a concentration which is immunogenic. Suitably antibody production at a concentration in sera which is immunogenic. Suitably at a concentration of between 1-20 μg/ml, 1-18 μg/ml, 1-16 μg/ml, 1-14 μg/ml, 1-12 μg/ml, 2-18 μg/ml, 2-16 μg/ml, 2-14 μg/ml, 2-12 μg/ml, or 2-10 μg/ml in sera for example.

The skilled reader, on considering the information set out in the Examples, will recognise that the VLPs or the immunogenic compositions of the invention exhibit immunogenic activity that makes them well suited to therapeutic use in the manner described in this specification.

Medical Uses

The present invention further relates to use of the VLP or the immunogenic composition comprising the VLP for use in therapy, or in the prevention and/or treatment of a disease.

In further aspect, the present invention further provides a method of treating a subject having a disease, comprising administering an effective amount of a VLP according to the first or second aspects or an immunogenic composition according to the eleventh aspect, to the subject.

In further aspect, the present invention further provides a method of manufacturing a medicament for the treatment of a disease, the medicament comprising an effective amount of a VLP according to the first or second aspects or an immunogenic composition according to the eleventh aspect.

Suitably the disease may be selected from: an infectious disease, cancer, an autoimmune disease, a cardiovascular disease, a metabolic disease, an inflammatory disease, a neurological disease, or rheumatological degenerative disease, or an addiction.

Suitable infectious diseases include: viral, bacterial, fungal, or protozoan infections.

Suitable viral infections include: COVID-19, SARS, MERS, influenza, common cold, respiratory syncytial virus infection, adenovirus infection, parainfluenza virus infection, norovirus infection, rotavirus infection, astrovirus infection, measles, mumps, rubella, chickenpox, shingles, roseola, smallpox, fifth disease, chikungunya virus infection, HPV infection, Hepatitis A, B, C, D or E, warts, herpes, molluscum contagiosum, ebola, lassa fever, dengue fever, yellow fever, Marburg hemorrhagic fever, Crimean-Congo hemorrhagic fever, polio, viral meningitis, viral encephalitis, rabies, zika virus infection, west nile virus infection, HIV/AIDS, Hantavirus infection, HPS.

Suitable bacterial infections include: urinary tract infections, cystitis, impetigo, bacterial food poisoning, campylobacteriosis, C. difficile infection, bacterial cellulitis, MRSA, CRPA, VRSA, sepsis, erysipelas, necrotising fasciitis, bacterial folliculitis, gonorrhoea, chlamydia, syphilis, Mycoplasma genitalium, bacterila vaginosis, pelvic inflammatory disease, tuberculosis, whooping cough, Haemophilus influenzae disease, pneumonia, bacterial meningitis, lyme disease, cholera, botulism, tetanus, anthrax, Cryptosporidiosis, Diphtheria, E. coli infection, Legionnaires Disease, Leptospirosis, Listeriosis, salmonella infections, Shigellosis gastroenteritis, Staphylococcal infections, Streptococcal infections, TSS, typhoid fever, Yersenia infection.

Suitable cancers include: breast cancer, liver cancer, lung cancer, pancreatic cancer, brain cancer, prostate cancer, bowel cancer, rectal cancer, bone cancer, leukemia, bladder cancer, cervical cancer, endometrial cancer, eye cancer, retinoblastoma, ewing sarcoma, gallbladder cancer, head and neck cancer, kaposi's sarcoma, kidney cancer, laryngeal cancer, mesothelioma, myeloma, lymphoma, ovarian cancer, oesophageal cancer, mouth cancer, nasopharyngeal cancer, nose and sinus cancer, skin cancer, sarcoma, stomach cancer, testicular cancer, thyroid cancer, uterine cancer, vaginal cancer, penile cancer, vulval cancer.

Suitable autoimmune diseases include: asthma, psoriasis, MS, rheumatoid arthritis, reactive arthritis, lupus, inflammatory bowel syndrome/disease, type 1 diabetes, Guillain-Barre syndrome, demyelinating polyneuropathy, Graves' disease, Hashimo's thyroiditis, Myasthenia gravis, vasculitis, pernicious anemia, ulcerative colitis, antiphospholipid syndrome, Kawasaki disease, alopecia, vitiligo, scleroderma, Sjogren's syndrome, crohn's disease, coeliac disease, Addison's disease, narcolepsy.

Suitable cardiovascular diseases include: angina, heart attack, heart failure, coronary heart disease, stroke, transient ischemic attack, peripheral arterial disease, aortic disease, atherosclerosis, hypertension, cerebrovascular disease, renal artery stenosis, aneurysm, cardiomyopathy, pulmonary heart disease, arrythmia, dysrhythmia, endocarditis, cardiomegaly, myocarditis, valvular heart disease, congenital heart disease, rheumatic heart disease.

Suitable metabolic diseases include: hypercholesterolemia, hypertriglyceridemia, diabetes, hyperlipidemia, hyperbilirubinemia, hypercalcemia.

Suitable inflammatory diseases may include any of the above infections or autoimmune diseases. Suitable inflammatory diseases may include include: arthritis, asthma, tuberculosis, periodontis, chronic ulcers, sinusitis, hepatitis, glomerulonephritis, inflammatory bowel syndrome/disease, preperfusion injury, transplant rejection, sickle cell disease, allergies, cardiovascular disease, psoriasis, cytokine-mediated pruritus, COPD, diabetes, bronchitis, Crohn's disease, atherosclerosis, dermatitis, arteritis, lupus.

Suitable neurological diseases include: Alzheimer's, ataxia, ALS, Bells palsy, brain tumours, aneurysms, epilepsy, Guillain-Barre syndrome, hydrocephalus, Meningitis, MS, muscular dystrophy, neurocutaneous syndromes, Parkinson's, migraines, encephalitis, myasthenia gravis, dementia, seizures, spinal muscular atrophy, motor neuron disease, scoliosis, neuropathy, chronic fatigue syndrome, cerebal palsy.

Suitable rheumatological degenerative diseases include: rheumatoid arthritis, psoriasis arthritis, spondylarthropathy, osteoarthritis, lupus, systemic sclerosis.

Suitable addictions include: alcohol, nicotine, caffeine, amphetamines, opioids, sedatives, hypnotics, anxiolytics, cocaine, cannabinoids, hallucinogenics, phenycylcidine.

In one embodiment, the VLP or the immunogenic composition are for use in the prevention or treatment of COVID-19. Suitably in such an embodiment, the functional molecule may be a SARS-CoV-2 antigen, suitably a SARS-CoV-2 spike protein. Alternatively in such an embodiment, the functional molecule may be an inflammatory cytokine, suitably IL-33.

In one embodiment, the VLP or the immunogenic composition are for use in the prevention or treatment of psoriasis or arthritis. Suitably in such an embodiment, the functional molecule may be an inflammatory cytokine, suitably IL17.

In one embodiment the VLP or the immunogenic composition are for use in the prevention or treatment of asthma or atopic dermatitis. Suitably in such an embodiment, the functional molecule may be an inflammatory cytokine, suitably IL13 or IL33.

Suitably, an effective amount for administration to the subject is an effective amount to prevent or treat the disease. Suitable effective amounts can be readily determined by the skilled medical practitioner.

Suitably a dose comprises an effective amount. A suitable dose of the VLP may comprise: 10-100 micrograms, suitably 10-80 micrograms, suitably 20-60 micrograms, suitably 20-40 micrograms.

Suitably the VLP or immunogenic composition may be administered by any route. Suitably the VLP or immunogenic composition may be administered enterally or parenterally. Suitably the VLP or immunogenic composition may be administered orally, rectally, vaginally, sublingually, by injection, transdermally, or by inhalation.

In one embodiment, the VLP or immunogenic composition may be administered by injection, suitably by subcutaneous injection.

In one embodiment, the VLP or immunogenic composition may be administered by inhalation, suitably by nasal inhalation.

Subject

The present invention relates to the prevention and/or treatment of a disease in a subject by using the VLP or immunogenic composition thereof.

Suitably the subject may be human or animal. Suitably therefore the prevention and/or treatment of disease may be in the veterinary field.

Suitably the subject may be adult or child. Suitably the subject may be male or female. In one embodiment, the subject is an adult human.

Suitably the subject may have been diagnosed with a disease.

Alternatively, the subject may be suspected of having a disease. Suitably the subject may display one or more symptoms of a disease.

Alternatively, the subject may be at risk of contracting a disease. Suitably the subject may have one or more risk factors associated with a disease. Suitable risk factors may include: weight, smoking, alcohol or substance addiction, age, sex, race, inheritance for example. Suitable risk factors may further include a genetic predisposition to a disease, for example by expression of particular gene, or by the presence of a particular mutation in a gene.

In one embodiment, subjects that have been diagnosed with a disease or who have one or more symptoms of a disease are provided with the VLP or immunogenic composition for treatment of the disease.

In one embodiment, subjects that are at risk of developing a disease are provided with the VLP or immunogenic composition for prevention of the disease.

Other Uses

The present invention further relates to use of the VLP in research and in the diagnosis of diseases.

Suitably the VLP of the first or second aspects may be used in research. Suitably the VLP may be used as a detection tool. Suitably the VLP may be used as a label. Suitably in such embodiments, the VLP comprises a functional molecule which is a flourescent molecule.

Sutiably the VLP may comprise a first functional molecule which is an antigen binding molecule such as an antibody, and a second functional molecule which is a flourescent molecule. Suitably the antigen binding molecule may specifically bind a cell surface receptor. Suitable cell surface receptors are discussed elsewhere herein, however suitably the cell surface receptor is specific to a cell type. Suitably therefore the VLP is capable of binding to, and labelling, specific cell types.

Suitably the VLP may be used as a carrier. Suitably in such embodiments, the VLP may comprise a cargo. Suitably the cargo may be contained within the VLP, suitably within the VLP shell. Suitably the cargo may be a therapeutic molecule. Suitably therefore the VLP may not in itself be a therapeutic, but may be a carrier of a therapeutic molecule. Suitable therapeutic molecules may include oligonucleotides, small molecules, peptides, for example. In one embodiment, the therapeutic molecule may comprise an antisense oligonucleotide which may act to repress expression of a particular nucleic acid. In another embodiment, the therapeutic molecule may comprise a cytotoxic chemical which may act to trigger cell death.

Suitably, in such embodiments, the VLP is targeted to a particular site, for example to a particular cell or cell type where the therapeutic molecule is required. Suitably this is achieved by the VLP comprising a functional molecule which is an antigen binding molecule such as an antibody. Suitably the antigen binding molecule may specifically bind to a cell surface receptor. Sutiably to a cell surface receptor specific to the target cell. Suitably binding to the cell surface receptor may stimulate uptake of the VLP into the cell. Suitably therefore, the VLP is capable of binding to specific cell types and delivering cargo thereto.

In a further aspect of the invention, there is provided a carrier VLP comprising the features of the first or second aspects, and in addition a cargo, wherein the cargo is contained within the VLP shell. Suitably the cargo is a therapeutic molecule.

Suitably the VLP of the first or second aspects may also be used in diagnosis.

Suitably the VLP comprises a first functional molecule which is an antigen binding molecule, such as an antibody. Suitably the antibody specifically binds an antigen derived from a disease causing agent as discussed hereinabove. Suitably from an infectious agent such as a virus, bacterium, fungus, protozoan, or archaeon.

Suitably, therefore, the VLP is capable of binding to a disease causing agent and allowing detection thereof.

Suitably therefore the VLP of the invention may be used in a method of diagnosing a disease in accordance with the sixteenth aspect of the present invention. Suitably there is provided a method of diagnosing a disease in a subject comprising:
(a) Providing a VLP according to the first or second aspects of the invention, wherein the functional molecule is an antigen binding molecule directed towards an antigen derived from a disease causing agent;
(b) Mixing the VLP with a suitable sample from the subject;
(c) Detecting whether the VLP precipitates;
(d) Diagnosing the presence of a disease if the VLP precipitates.

Suitably, given that the functional molecule is an antigen binding molecule, the VLP further comprises a third binding protein. The third binding protein is described elsewhere herein. Suitably the antigen binding protein is indirectly attached to the second binding protein via a third binding protein.

Suitably detection is via precipitation of the VLP bound to the disease causing agent. Suitably detecting precipitation may comprise visual confirmation, or testing with a spectrometer.

Suitably if no precipitation occurs, the disease is not present.

Suitably, the VLP may also comprise a second functional molecule which is a flourescent molecule. Suitably such a second functional molecule may be attached to a chemical modification of the first binding protein. In such embodiments, suitably the detection step may comprise detecting the presence of fluorescence in the sample. Suitably the detection step may comprise detecting the presence of fluorescent precipitation in the sample. Suitably diagnosing the presence of a disease if fluorescent precipitation occurs.

Advantageously, the use of fluorescence allows more sensitive detection of the precipitation in a sample.

In one embodiment, the VLP used in the method of diagnosis comprises:
One or more viral capsid proteins;
One or more first binding proteins comprising a chemical modification;
One or more second binding proteins attached to one or more third binding proteins;
One or more functional molecules;

wherein each viral capsid protein is attached to a first binding protein, wherein each first binding protein is attached to a second binding protein, and wherein each third binding protein is attached to a first functional molecule, and each chemical modification is attached to a second functional molecule.

Suitably wherein the first functional molecule attached to the third binding protein is an antigen binding molecule. Suitably wherein the second functional molecule attached to the chemical medication on the first binding protein is a flourescent molecule.

A suitable sample from a subject may be a blood sample, saliva sample, serum sample, sputum sample, sperm sample, mucus sample, CSF sample. Suitably the sample is a fluid sample.

Suitably the method of diagnosis may further comprise a step of incubating the sample with the VLP. Suitably for a period of time sufficient to allow the VLP to bind to any antigens in the sample and precipitate. Suitably for at least 1 minute, suitably up to 30 minutes, suitably up to 25 minutes, suitably up to 20 minutes, suitably up to 15 minutes.

Suitable diseases which may be detected by the method may be any of those listed herein above.

Suitably the method of diagnosis may further comprise a step of treatment of the subject if a disease is diagnosed. Suitably treatment of the subject may comprise administering an effective amount of any known treatment for the relevant disease to the subject.

Certain embodiments of the invention will now be described with reference to the following examples:

### Examples

Materials and Methods

All plasm ids were supplied by University of Dundee sequencing and cloning services (https://www.dnaseq.co.uk) who also performed cloning services as well as sequence verification. Plasmids used: Pet-28 a (+), pETDuet 1, pcDNA5D

Cloning of Modified VLP Shells

Cloning Strategy for HBc Fusion to Im7 or Barstar:

ORFs for Im7 (GenBank accession Genbank: KJ470776.1) and Barstar (GenBank ARW38026.1) were extended on either side with linker consisting of GGGGSGGGGS (SEQ ID NO:33) and extended on the N-terminal end with a sequence encoding M1-Leu76 of Hepatitic B core antigen (GenBank accession Genbank: KJ470776.1), and on the C-terminal end with a sequence encoding Pro79-V145 of Hepatitic B core antigen followed by a STOP codon, respectively. The resultant sequences (Table 3: nucleotide sequences) were purchased as commercial genes synthesis with restriction sites for Xba1 and Not1 and cloned into pET-Duet 1 (Novagen).

Cloning Strategy for ColE7 Fusion to Chosen Epitope Proteins:

The ORF for the catalytic domain of Colicin E7 (GenBank accession Genbank: KJ470776.1), starting with E444, was modified to harbour the mutations: R538A, E542A (Ku, NAR, 2002), His569A (Ko, Structure, 1999), ensuring complete catalytic inactivity with retained Im7-binding capacity. An N-terminal methionine was added, as well as a TEV protease cleavage site, followed by a glycine/serine linker on the C-terminus. This sequence was flanked by Ndel and BamH1 restriction sites, respectively. The sequence was purchased as commercial gene synthesis and cloned into pET-Duet 1 (Novagen) harbouring ORFs encoding the Hepatitis B capsid fused to Im7 (see above).

Cloning Strategy for Barnase Fusion to Chosen Epitope Proteins:

The ORF for the catalytic domain of Barnase (GenBank accession Genbank: AAA86441.1, nucleotides 403-732), starting at residue A40 (omitting the signal peptide), was extended with an N-terminal methionine, mutated to E73W to eliminate catalytic activity, and C-terminally extended with a TEV protease cleavage site, followed by a glycine/serine linker on the C-terminus. This sequence was flanked by Ndel and BamH1 restriction sites, respectively. The sequence was purchased as commercial gene synthesis and cloned into pET-Duet 1 (Novagen) harbouring ORFs encoding the Hepatitis B capsid fused to Barstar (see above).

Cloning Strategy for Chosen Epitopes or Further Binding Protein to be Displayed:

The ORFs for epitope proteins to be displayed (C-terminal fragment of murine IL33, human IL13, or the further binding protein; Protein-G, respectively), were optimized for codon-usage in E. coli using publicly available software and extended with BamH1 and Xhol restriction sites, respectively, to allow in-frame cloning downstream of either ColE7, or Barnase, or upstream of ColE7 (for Protein G), as detailed in the respective sequences (Table 2: amino acid sequences). Nucleotide sequences were purchased as commercial gene synthesis and cloned into the pET-Duet 1 vectors (Novage) previously cloned to harbour ColE7 or Barnase ORFs, respectively, as above. NB: Sars-Cov2 epitope proteins were expressed in mammalian cells therefore no optimisation of these sequences was required.

Expression of Modified VLP Shells and Epitope Fusion Proteins

Transfection:

Competent BL21 DE3 and P812 cells (genetically engineered cell line, derived from BL21-DE3 to harbour disulfide isomerase genes, facilitating expression of proteins with disulfide bonds, such strains are known in the art) were transformed with 100 ng of corresponding plasmid and incubated on ice for 30 mins prior to heat shock at 42° C. for 30 secs. 100 ul of LB media was added to bacterial vials for a further incubation at 37° C. for 1 hr followed by spreading on agar plates and overnight cultivation.

Induction:

3× 4 ml starter cultures were prepared in 2× YT media (Sigma) and grown shaking 200 rpm overnight at 37° C. 1 ml of each starter culture was expanded to a final volume of 20 ml in 2× YT and cultivated at 37° C. until OD 600 reached 0.6-0.8. An uninduced sample was collected, centrifuged at 14,000 rpm for 1 min, supernatant removed and 100 ul of 2× Laemmli buffer (Sigma) added to the pellet. Cultures were induced with 0.5 mM IPTG and 5 mM MgCl₂ for overnight VLP expression shaking 200 rpm at 18° C. An induced 100 ul lysate was centrifuged at 14,000 rpm for 1 min, supernatant discarded, and pellet resuspended in 100 ml of 2× Laemmli buffer.

In plasmids designed for separate induction of VLP backbone and epitope proteins, the latter where induced by addition of 40 ng/ml anhydrotetracycline for 4-16 h at 15 20° C.

Cell Disruption (Analytical):

The culture was harvested by centrifuging at 4,000 rpm at 4° C. for 10 mins, supernatant discarded, pellet weighed and ultrasonicated at period of 30 secs for 2 mins and 10 secs pause intervals in lysis buffer (3 ml of lysis buffer per 1 g of culture). Addition of 10% of Triton×100 to a final concentration of 0.5% followed ultrasonication. 50 ul of lysed material was centrifuged at 14,000 rpm for 1 min, the supernatant was added to 150 ul of 2× Laemmli buffer and pellet resuspended in 200 ml 2× Laemmli buffer. The remaining lysed cultures were centrifuged at 4,000 rpm for 10 mins, supernatant transferred to 50 ml polypropylene tubes and frozen. VLP expression was analysed using 12% Bis-Tris Nu-Page SDS-PAGE (Thermofisher) gels run for 2.5 hrs at 100 V, followed by staining with Instant Blue Coomassie stain for 1 hr.

Cell Disruption (Preparative):

Cells were pelleted as above, followed by addition of Hepes 50 mM, pH 7.0, 200 mM NaCl, protease inhibitor cocktail (Pierce), followed by high pressure homogenization in the absence of any detergent. The resultant lysate was clarified by centrifugation at 18000 g for 30 min at 4° C.

Purification of VLPs

Ion Exchange Chromatography-Diethylamine (DEAE, BIA Separations/Sartorius):

The DEAE column (IBS) was equilibrated with 10 column volumes of deionised water and 10 column volumes of 20 mM/50 mM 50 mM Tris-HCl pH 7 at room temperature. The filtered crude extract was loaded with a 5 ml syringe and flow through collected. The column bound material was washed and eluted with a stepwise gradient of NaCl (0.1 M, 0.3 M, 0.5 M, 0.7 M, 1 M and 2 M). 3×1 ml fractions were collected per condition and stored on ice. The DEAE column was washed with 10 column volumes of 2 M NaCl, 10 column volumes of a buffer containing 2 M NaCl and 1 M NaOH, 10 column volumes of deionised water followed by storage in 20% isopropanol at 4° C. until subsequent use. The obtained samples were quantified using protein absorbance 280 nm on Nanodrop and BCA quantification (Thermofisher). Purification was analysed using 12% Bis-Tris NuPage SDS-PAGE gels run for 2.5 hrs at 100 V, followed by staining with Instant Blue (Abcam) Coomassie stain for 1 hr.

Ion Exchange Chromatography—Quaternary Amine (QA, BIA Separations/Sartorius):

The QA column (IBS) was equilibrated with 10 column volumes of deionised water and 10 column volumes of 20 mM/50 mM Tris-HCl pH 7 at room temperature. The filtered crude extract was loaded with a 5 ml syringe and flow through collected. The column bound material was washed and eluted with a stepwise gradient of NaCl (0.1 M, 0.3 M, 0.5 M, 0.7 M, 1 M and 2 M) 3×1 ml fractions were collected per condition and stored on ice. The DEAE column was sanitised with 10 column volumes of 2 M NaCl, 10 column volumes of a buffer containing 2 M NaCl and 1 M NaOH, 10 column volumes of deionised water followed by storage in 20% isopropanol at 4° C. until subsequent use. The obtained samples were quantified using protein absorbance 280 nm on Nanodrop and BCA quantification (Thermofisher). Purification was analysed using 12% Bis-Tris NuPage SDS-PAGE gels run for 2.5 hrs at 100 V, followed by staining with Instant Blue (Abcam) Coomassie stain for 1 hr.

Ion Exchange Chromatography—Mixed Mode (PrimaS, BIA Separations/Sartorius):

The PrimaS column (IBS) was equilibrated with 10 column volumes of deionised water and 10 column volumes of 20 mM/50 mM Tris-HCl pH 7 at room temperature. The filtered crude extract was loaded with a 5 ml syringe and flow through collected. The column bound material was washed and eluted with a stepwise gradient of NaCl (0.1 M, 0.3 M, 0.5 M, 0.7 M, 1 M and 2 M) 3×1 ml fractions were collected per condition and stored on ice. The DEAE column was sanitised with 10 column volumes of 2 M NaCl, 10 column volumes of a buffer containing 2 M NaCl and 1 M NaOH, 10 column volumes of deionised water followed by storage in 20% isopropanol at 4° C. until subsequent use. The obtained samples were quantified using protein absorbance 280 nm on Nanodrop and BCA quantification (Thermofisher). Purification was analysed using 12% Bis-Tris NuPage SDS-PAGE gels run for 2.5 hrs at 100 V, followed by staining with Instant Blue (Abcam) Coomassie stain for 1 hr.

Mixed Mode Size Exclusion Chromatography:

A self-packed 0.5 ml diameter, 10 cam long glass column was filled with CaptoCore 700 (GE Healthcare) and packaged with 20% ETOH. The column was equilibrated with 10 column volumes of deionised water and 10 column volumes of 20 mM Tris-HCl pH7 at room temperature. Semi-purified sample from ion exchange chromatography was loaded and 1 ml fractions collected. The column was sanitised with 10 column volumes of 1 M NaOH in 30% isopropanol followed by 10 column volumes of 2 M NaCl and replacement of 20% ETOH storage solution. Size exclusion was analysed using 12% Bis-Tris NuPage SDS-PAGE gels run for 2.5 hrs at 100 V, followed by staining with Instant Blue (Abcam) Coomassie stain for 1 hr.

### Example 1: Identification of Suitable VLP Shell and Optimisation

Candidate VLP shells exhibiting a precedent of successful in-frame fusion, a dimeric VLP protein structure to allow fusion of dimeric epitope proteins, and a precedent of suitability for clinical use were sought. The Hepatitis B capsid (HBc) was identified as a suitable candidate for the VLP.

The amino acid sequence of the HBc protein was then optimized to account for the insertion of a binding protein as follows: The negatively charged amino acids E77 and D78 were deleted to reduce the net-negative charge in the Major Immunodominant Region, the mutation F97L was added in order to accelerate protein folding, the C-terminal sequence which binds RNA in native virus was removed following residue V145, and a positive-net charge sequence was added on the C-terminus to stabilize the VLPs via insertion six histidine residues downstream of V145, which are not exposed to the protein surface. Resultant clones: DU67866 and DU67867.

### Example 2: Optimisation of Protein Binding Pair

ColE7/Im7, and Barnase/Barstar were chosen as suitable protein binding pairs which to attach functional molecules to the HBc VLP shell.

Both pairs were further optimised as follows and as explained above:

ColE7:
Choose required fragment of catalytic domain necessary to bind Im7, based on review of x-ray structures in public repositories. The ORF for the catalytic domain of Colicin E7 (GenBank accession: KJ470776.1), starting with E444, was modified to harbour the mutations: R538A, E542A (Ku, NAR, 2002), His569A (Ko, Structure, 1999), ensuring complete catalytic inactivity with retained Im7-binding capacity.
An N-terminal methionine was added, as well as a TEV protease cleavage site, followed by a glycine/serine linker on the C-terminus. This sequence was flanked by Ndel and BamH1 restriction sites, respectively and cloned into pET-Duet 1 (Novagen) harbouring ORFs for variants of the HepB-capsid fused to Im7 (see below).
Im7: The ORF for Im7 (GenBank accession: KJ470776.1) was extended on either side with linker sequences GGGSGGGS and and extended on the N-terminal end with sequence encoding M1-Leu76 of Hepatitis B core antigen (GenBank accession: KJ470776.1), and on the C-terminal end with sequence encoding Pro79-V145 followed by a STOP codon, respectively. The resultant sequences were supplied with restriction sites for Xba1 and Not1 and cloned into pET-Duet 1.
Insert a mutation into the primary sequence of Im7 specified above to trigger twenty—fold increase in protein folding speed: F41L (Capaldi, Nat Struct Biol 2002).
Insert a further mutation into the Im7 sequence specified above which is known to enhance capsid formation: F97L (Alexander, PNAS 2013).

Barnase:
Select a mutation that will retain Barstar binding but abrogate catalytic activity: based on the data in (Want, Biophys J 2004), E73->Q was tested but found to retain residual toxicity. Hence, E73->W was chosen and inserted into the sequence.
The ORF for the catalytic domain of Barnase (GenBank accession: AAA86441.1, nucleotides 403-732), starting at residue A40 (omitting the signal peptide), was extended with an N-terminal methionine, mutated to E73W, and C-terminally extended with a TEV protease cleavage site, followed by a glycine/serine linker (GGGGSGGGGS SEQ ID NO:33) on the C-terminus. This sequence was flanked by Ndel and BamH1 restriction sites, respectively and cloned into pET-Duet 1 (Novagen) harbouring ORFs for Barstar (see below).

Barstar:
For design: Remove cysteines (C40->A, C82->A)
For design: Insert mutation to abrogate dimerization mutations where chosen to abolish cysteines and to rule out protein dimerization based on literature characterization (Korchuganov, 2004): I87->E
For cloning: ORF for Barstar (GenBank ARW38026.1) was extended on either side with linker sequences (GGGGSGGGGS SEQ ID NO:33) and and extended on the N-terminal end with sequence encoding M1-Leu76 of Hepatitic B core antigen (Genbank: KJ470776.1), and on the C-terminal end with sequence encoding Pro79-V145 followed by a STOP codon, respectively. The resultant sequence was extended with restriction sites for Xba1 and Not1 and cloned into pET-Duet 1 (Novagen).

### Example 3: Selection of Relevant Test Epitopes

Suitable test epitopes were selected to act as functional molecules when attached to the VLP shell via the protein binding pair.

IL-33, IL-13 and SARS-CoV-2 Spike Protein receptor binding domain and nucleocapsid were selected as epitopes. IL-33 was selected as a relevant vaccine target and target for hyper-immune responses in COVID, as well as in asthma. IL-13 was selected as a relevant vaccine target for allergies. IL-17 was selected as relevant vaccine for psoriasis. The SARS-CoV-2-Spike Receptor binding domain was selected to demonstrate that the system works for expression of epitopes in mammalian cells, and as a relevant target for Covid-19.

For murine IL-33 (RefSeq NP_001158196), the extracellular-signaling caspase-cleaved C-terminal fragment (starting S109) was selected and, and a GGGGSGGGGS (SEQ ID NO:33) linker was inserted N-terminally to attach this fragment to the upstream ColE7 fragment. Two mutations were inserted in order to abrogade binding to the cognate ST2 receptor and thereby eliminate any non-vaccine related receptor-mediated toxicity: the murine equivalent of mutations D149K and E165K in the human IL33 sequence, respectively. This sequence was codon-optimization for E. coli was performed using publically available software and the resulting sequence extended with BamH1 and Xhol restriction sites, respectively, to allow in-frame cloning downstream of either ColE7, or Barnase, into pET Duet 1 plasmids. Resultant clone: DU 70051

For human IL-13 (RefSeq NM_002188), a SGGGSSGSG linker (SEQ ID NO:35) was inserted to attach this sequence to the upstream ColE7 fragment, then codon-optimization for E. coli was performed. Resultant clones: DU 70076, DU70080.

For human IL-17 (RefSeq NM_NM_002190.3), a SGGGSSGSG linker (SEQ ID NO:35) was inserted to attach this sequence to the upstream ColE7 fragment, then codon-optimization for E. coli was performed. Resultant clones: DU 70721.

For SARS-CoV-2-Spike protein, the structure-guided receptor-binding-domain (RBD) fragment was selected and isolated. A suitable mammalian expression vector was chosen (pcDNA5D). The linker GGGGSGGGGS (SEQ ID NO:33) was inserted between this sequence and the N-terminally located ColE7, yielding the resultant clone: DU 67808. A second clone was created including an additional C-terminal fusion of the SARS2-nucleocapsid protein C-terminal fragment for broader target vaccination, resultant clone: DU 67817. A third clone including added C-terminally fused eGFP to allow monitoring of expression was then created, resultant clone: DU 67793

### Example 4: Design of Expression Plasmids

For Prokaryotic expression, the plasmid backbone pETDuet was used. Further features added to the plasmid include:
A VLP-construct including the HBc-capsid with a first binding protein (e.g. Im7) in-frame fusion driven by IPTG-inducible T7 promoter
A second binding protein-epitope protein fusion (e.g. ColE7-1L13) driven by IPTG-inducible T7 promoter or a tuned T7 promoter with lesser activity to allow for equal expression of the VLP and the epitope protein
Alternatively, independent inducibility of the epitope protein (e.g. ColE7-1L33) by anhydrotetracycline by incorporating an independent tetR/tetA promoter system into the expression plasmid.
Lacl to drive inducible expression of VLP-construct ColE7-epitope protein fusion
Optional TEV protease site inserted for isolated expression of only the epitope to facilitate purification of epitope protein as antigen for ELISA purposes
Plasmid resistance gene such as Amp or non-beta-lactam for bioprocess applications.

General plasmid design applies to clones: 67866, 67867, 70076, 70051, 70080, 70059, 70126, 65750, 70680, 70612.

For Eukaryotic expression, the plasmid backbone pcDNA5D was used. Further features added to the plasmid include:
A CMV promoter for strong expression in mammalian cells
A strong signal peptide for secretion of product into cell culture medium
A binding protein (e.g. ColE7) fused N-terminally to the epitope protein (e.g. SARS-CoV-2-Spike RBD) to act as chaperone for the epitope protein
Optional tet repressor system
Optional C-terminal hexa-His tag for purification
Plasmid resistance gene such as AmpR, or HygR or non-beta-lactam for bioprocess applications.

General plasmid design applies to clones: DU67808, DU67793, DU67808.

### Example 5: Expression of Candidate Proteins

Protocol (more detail above): Inoculate starter culture of E. coli BL21D3 harbouring clone DU70080, then inoculate 25 ml culture, grow to OD 0.7, then induce with 1 mM IPTG for 22 h at 18 C. Pellet cells, resuspend in 20 mM Tris/HCl, pH 7.0, 100 mM NaCl, protease inhibitor cocktail, disrupt with high-pressure homogenizer, take aliquot as ‘lysate’, spin for 30 min at 4 C at 18,000G, take supernatant as ‘cytosol’, resuspend pellet in buffer containing triton X100 and urea as ‘insoluble’

Expected size of candidate proteins: HBc-Im7: 27.6 kDa. Col7-1L13: 28.8 kDa.

SDS-PAGE shows bands at both expected sizes across three clones. The HBc-Im7 fused VLP backbone is highly soluble in E. coli, despite identical T7-promoter usage, the ColE7-1L13 fusion has higher overall expression. In soluble cytosol, VLP-backbone and cytokine expression is more evenly distributed.

### Example 6: Purification of Candidate Proteins

Protocol (more detail above): Prepare VLP-containing cytosol fractions as above; clarify by 15G centrifugation and 0.2 micron filtration, run on CIM-monolith column (Sartorius) at flow rate 1 ml/min in Tris-HCl, pH 7.0 with indicated NaCl step-wise gradient. Fractions were analyzed by SDS-PAGE on 12% gels.

SDS-PAGE after DEAE-anion exchange chromatography shows HBc-Im7 VLPs show affinity-chromatography like elution profile on weak DEAE anion exchanger, eluting only at 1 M NaCl.

Protocol (more detail above): the obtained DEAE-purified HBc-Im7 VLP fraction was subjected to mixed mode chromatography (PrimaS 1 ml column, Sartorius) at pH 7.0, with step-wise NaCl gradient applied, as shown at flow rate of 1 ml/min. Fractions were taken and analyzed by SDS-PAGE.

SDS-PAGE after mixed mode chromatography shows HBc-Im7 fused VLP bind irreversibly to hydrophobic-derivatized polymethacrylate.

Protocol (more detail above): the obtained DEAE-purified HBc-Im7 VLP fraction was alternatively subjected to multi-mode size exclusion chromatography (CaptoCore 700, loaded into 1 ml column, diameter 0.5 cm, flow rate 1.5 ml/min in Tris/HCl 20 mM pH 7.0, 300 mM NaCl. Resin excludes large particles with approximate cut-off at 700 kD. Smaller particles are captured by octylamine contained on the inside of cross-linked agarose beads. Fractions were analyzed by SDS-PAGE.

SDS-PAGE after multimode size exclusion chromatography shows HBc-Im7 purify to apparent homogeneity with multi-mode size exclusion chromatography.

### Example 7: Formation of VLPs

Protocol: HBc-Im7 VLPs were purified to apparent homogeneity as detailed above and, made to a concentration of 1.5 mg/ml protein content in 10 mM Tris/HCl, pH 7.0, 200 mM NaCl. VLP samples were deposited onto carbon-coated mesh grids. Thereafter, sample were loaded onto the grid and incubated at RT for 1 min. Excess sample was drained off the grid. Samples were negatively stained with 2% uranyl acetate at RT for 1 min. Excess stain was drained off and grids were dried at RT. TEM examinations were performed using a JEOL JEM-2200FS electron microscope For assessment of VLP diameter, measurements were obtained from n=150 VLPs for each preparation. Two diameter and rim measurements were obtained for each VLP.

For statistical testing, two sided independent t-tests were performed after ascertaining of equal variance and normal distribution of size measurements.

HBc-Im7 fused VLPs form uniform VLPs, showing increased diameter compared to parent HBc-capsid derived VLPs (38.3±2 vs. 29±2 nm, p<0.001).

HBc-Im7 fused VLPs show a significantly increased thickness of outer electron-dense rim. The increase in observable rim thickness (10.2±1.5 vs. 5.7±1.2 nm, p<0.0001) is consistent with an extra layer of Im7 domain proteins protruding from the HBc-major immunodominant region.

### Example 8: Immunogenicity of VLPs

Protocol: Aged female C566/6j mice (15 months at begin of study) were vaccinated intranasally with 5 ug of HBc-Im7 VLPs (n=5 per group), as well as given a single booster vaccination at day 21, and serum collection as shown. Data were analyzed by ELISA, with HBc-Im7 used as coated antigen.

The data show that HBc-Im7 VLP's elicit strong immune response even in aged mice after intra-nasal vaccination.

### Example 9: Linking an Epitope to VLPs

Protocol: Cytosolic fractions of VLP shells (lane 1) and ColE7-fused IL33 (lane 2) expressed in E. coli were coupled (lane 3), followed by purification by mixed-mode size-exclusion chromatography on CaptoCore700 resin as explained above, and collection of flow-through fractions (lanes 5-7). Data show SDS-PAGE analysis. Successful coupling of the HBc-Im7 VLP shell to the ColE7-1L33 epitope fusion is achieved.

Protocol: Col-E7-RBD-GFP was expressed as secreted protein in 293T cells in suspension culture. Cell supernatants (lane 1) were collected and incubated with DEAE-purified HBc-Im7 VLP shells (lane 2). Subsequently, media were purified by batch incubation with CaptoCore700 resin as described above to remove non-VLP proteins (size exclusion approx. 700 kD), and depleted cell supernatant was concentrated using ultrafiltration spin columns (Pierce, cut-off 100 kD, lane 3). Data show SDS-PAGE analysis. Successful coupling of the HBc-Im7 VLP shell to the ColE7-RBD-GFP epitope fusion is achieved.

### Example 10: Non-Covalent Chemical Modification of VLPs

Protocol: Rhodamine-azide was derivatized with octyne by Copper-catalyzed azide— alkyne cycloaddition at room temperature for 1 h using TBTA and TCEP as reducing agent and a final concentration of 250 uM Rhodamine in the reaction mix consisting of 90% DMSO and 10% PBS. 10 ul this reaction mix was co-incubated by vigorous vortexing for 1 min with 90 ul of VLP-cytosolic fractions, containing either HBc-Im7-VLPs (orange) or HBc-capsid VLPs (blue), dissolved in 50 mM Hepes, pH 7.0, 200 mM NaCl, 20% glycerol. Subsequently, the 100 ul VLP/rhodamine mix was incubated in batch with 100 ul packed resin of Captocore700, pre-equilibrated in 50 mM Hepes, 20% glycerol, for 1 h rotating at RT, followed by centrifugation (5000 g for 5 min). Fluorescence of 50 ul of supernatants (“after C8-FITC”) and 50 ul of the original VLP-fraction (“before C8-FITC”) was measured with excitation at 488 nm and emission at 520 nm.

The data show that HBc-Im7 fused VLPs, but not the parent HBc-capsid VLPs, can be fluorescently labelled by incubation with octylamine-derivatized rhodamine.

### Example 11: Biophysical Sedimentation Properties of Modified VLPs

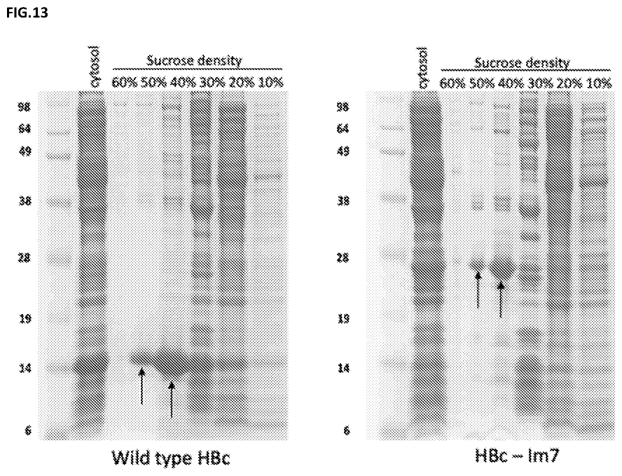

Protocol: Freshly obtained cytosolic fractions from E. coli expressing either HBc or HBcIm7 were manually overlaid on a 60-20% (w/v) continuous sucrose gradient using Beckman Coulter™ Ultra-clear centrifuge tubes. Separation was performed at 30,000 rpm for 16 hrs at 4° C. in a Sorvall Surespin 630 rotor. Sucrose fractions were harvested by puncture with a 21 G needle between the 50-60% interface. Individual fractions were subjected to reducing SDS-PAGE, as shown in the data (FIG. 13).

VLPs are large structures which routinely can be separated from smaller proteins by density centrifugation in sucrose. Large particles such as VLPs will equilibrate into higher density, typically 40-50% of sucrose, as opposed to monomeric proteins. The data confirms that exactly this is found for HBc-derived VLPs harbouring an integrated Im7 protein.

FIG. 13 shows SDS-PAGE analysis showing the biophysical sedimentation properties of VLPs harbouring Im7 integrated in the HBc Major-Immunodominant Region. VLP particles (black arrows) are expected in the 40-50% density fractions. Modified VLPs behave analogous to wild type VLPs.

### Example 12: Confirmation of the Integrity of VLPs in E. coli

Protocol: 1% Agarose gels were cast using 0.5× TBE buffer and run with 50 μl of sample per lane at RT for 1 hr at 60 V. Instant Blue Coomassie was stained overnight at 4° C. .SYBR™ safe (ThermoFisher) was used at 1:10,000 dilution and imaged on a ChemiDock™ Gel Imaging System (BioRad).

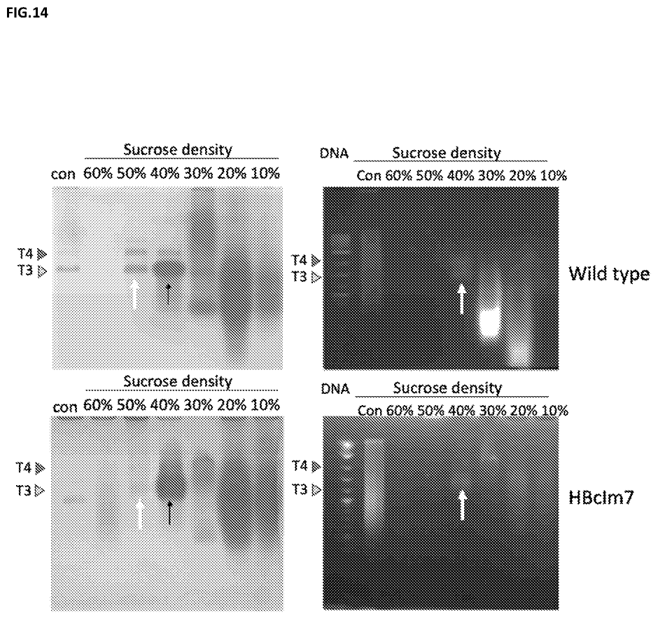

Sucrose density gradient fractions were subjected to native agarose gel electrophoresis (NAGE) (FIG. 14). This technique assesses the migration of intact VLPs, as opposed to SDS-PAGE which verifies the localization of monomeric VLP proteins. NAGE gels were run in parallel to be analyzed either by Coomassie blue staining for protein content (left) or by SYBR safe stain for nucleic acid content (right). A purified sample of HBc VLP was included as control (left-most lane).

HBc VLPs can exist in two configurations, either as slightly larger so-called T4 variant, composed of 240 protein subunits, or as smaller T3 variant, composed of 180 subunits. These can be distinguished in NAGE gels as distinct bands. In wild type VLPs (top) the T3 and T4 bands are clearly visible (marked by arrow heads) where the smaller T3 particles, as expected, are much more abundant in the less dense 40% fractions.

The Im7-containing modified VLPs (bottom) show a shift in migration within the gel relative to the HBc wild type control, consistent with their larger size. The relative T4/T3 densities are identical to those seen in the wild type VLPs: approximately equal distribution in the 50% sucrose fraction (grey arrow) and much higher abundance of the T3 band in the 40% cushion (black arrow).

During VLP assembly inside E. coli, E. coli-derived RNA is incorporated into the VLPs. Upon lysis, Benzonase is added to digest DNA. Therefore, after sucrose gradient centrifugation, nucleic acid colocalizing with VLPs in high density sucrose fractions represent RNA situated within the VLP. SYBR staining of a replicate gels shows a dense band colocalizing to the T3 VLP bands in the 40% sucrose fractions (marked by white arrow). This indicates that RNA localized inside the VLPs is protected from Benzonase digestion, which confirms the integrity of the VLPs.

### Example 13: Integration of the Fusion Proteins Abolishes any Cross-Reactivity to Hepatitis B Core Antigen

Protocol: VLPs purified by density sucrose gradient were dialyzed against PBS. The volumes shown in the figure were spotted onto nitrocellulose membrane. Membranes were blocked with BSA/PBST, followed by incubation with anti-HBcAg, (Invitrogen, MA1-7607, concentration 0.1 μg/ml) in PBST. The membrane was washed and exposed to secondary antibody (Donkey anti-goat HRP, diluted 1:10,000; Stratech, 705-035-147-JIR), followed by washing and development with Amersham ECL™ Western Blotting Detection Reagents.

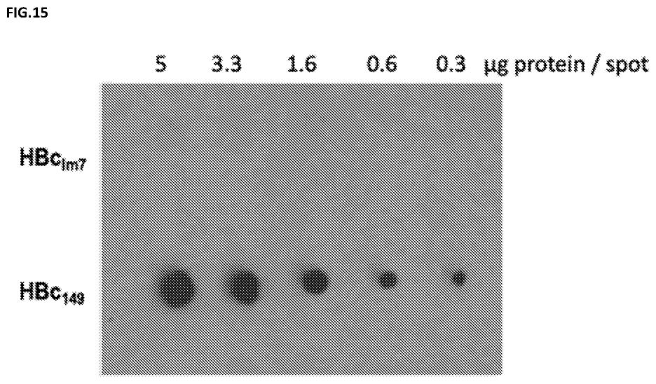

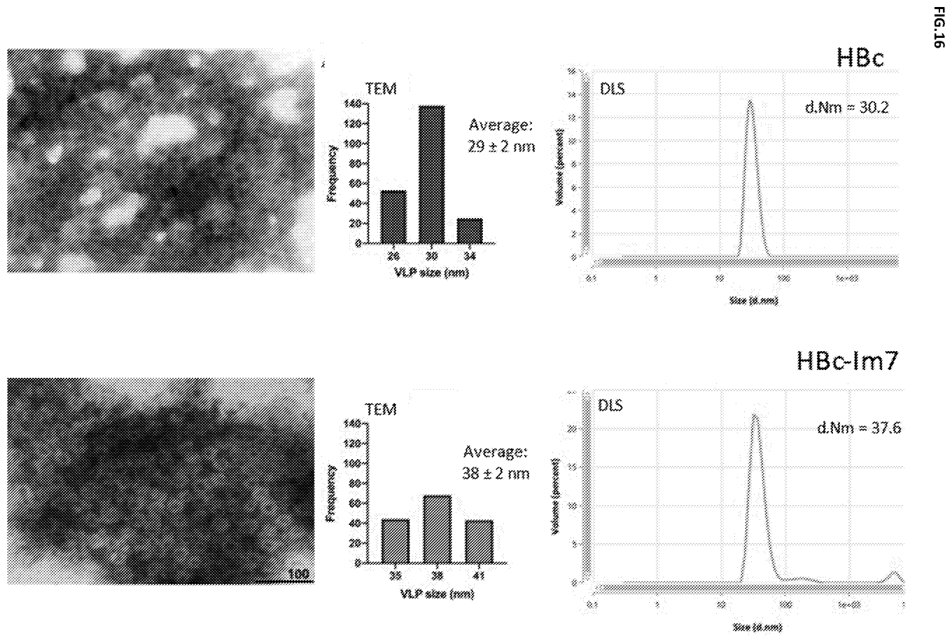

HBc-derived VLPs are intended to be used clinically as vaccines. Therefore, it is important that antibodies they generate do not cross-react with Hepatitis B antibodies in order for patients not to be falsely identified as Hepatitis B-positive. Past infection with Hepatitis B is routinely screened by serology to HBcAg (Hepatitis C Antigen). HBcAg recognizing antibodies react with the so-called “Major Immunodominant Region” (MIR) which is disrupted upon integration of either Im7 or Barstar. The data shows that a commercial antibody reacting strongly to wild-type Hepatitis C capsid to 300 ng of purified HBcAg (labelled ‘HBc149’) fails to react with all amounts tested (up to 5 μg) of purified HBc-Im7 VLP. This confirms that integration of the fusion proteins abolishes any cross-reactivity to Hepatitis B Core antigen (FIG. 15).

### Example 14: Size Comparison of HBc-Im7 VLPs Versus Wild-Type HBc VLP

Protocol: TEM: samples were dissolved in 25 mM Tris/HCl, pH 7.0 or 50 mM Hepes, pH 7.5 and adsorbed to glow discharged carbon-formvar-coated copper grids and negatively stained with a 1% aqueous uranyl acetate. The grids were examined at 80 kV. Size measurements (n=200 particles per VLP type) were performed using ImageJ. DLS: Samples contained in sucrose were dialyzed against Tris/HCl pH 7.0, 150 mM NaCl and analysed in 1 ml volume with a Zetasizer Ultra (Malvern Panalytical).

Wild type and HBc-Im7 VLPs purified by sucrose gradient centrifugation were subjected to electron microscopy (TEM) and Dynamic Light Scatter (DLS) analysis to determine shape and size.

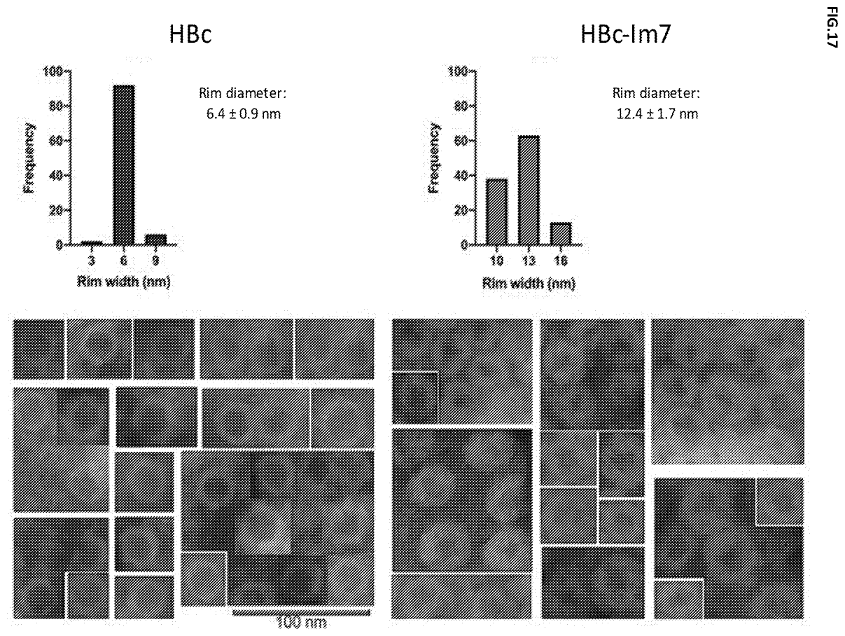

The resulting data (FIG. 16) show a size of around 30 nm both by TEM and by DLS measurement for wild type HBc (top), which is in good agreement for published results for a predominant T4 population of VLPs. By comparison, VLPs with integrated Im7 (bottom) show an increased size of around 38 nm both by TEM and DLS. The increase in size is expected, given the integrated Im7 protein on top of the VLP surface. The data show uniformity of the VLP population.

TEM was also used to determine the thickness of the VLP rim. As shown in the data (FIG. 17), the rims of Im7-bearing VLPs (right) were markedly thicker than those of wild type VLPs (left). This was quantified by ImageJ based measurement and yield a roughly doubled thickness of the modified rims (12.4 vs 6.5 nm). This result is in keeping with the placement of the Im7 protein uniformly on top of the HBc-surface layer.

### Example 15: IL33-Decorated VLPs Behave as Expected in Sucrose Density Ultracentrifugation

Protocol: Sucrose density gradient analysis by SDS-PAGE of combined HBcIm7 and ColE7_IL-33 cytosols. IL33 was coupled to HBc-Im7 VLPs by mixing cytosolic fractions containing HBc-Im7 and ColE7-1L33 at a 1:1 ratio for 1 h at 4° C. Coupled cytosol was overlaid on a continuous sucrose density gradient. Lanes: 1) cytosol containing ColE7_IL-33 and HBc-Im7 2) 60% sucrose, 3) 50% sucrose, 4) 40% sucrose, 5) 30% sucrose, 6) 20% sucrose, 7) 10% sucrose. The black arrow points to ColE7_IL-33 (34.2 kDa) and the red arrow points to HBc-Im7 (27.4 kDa).

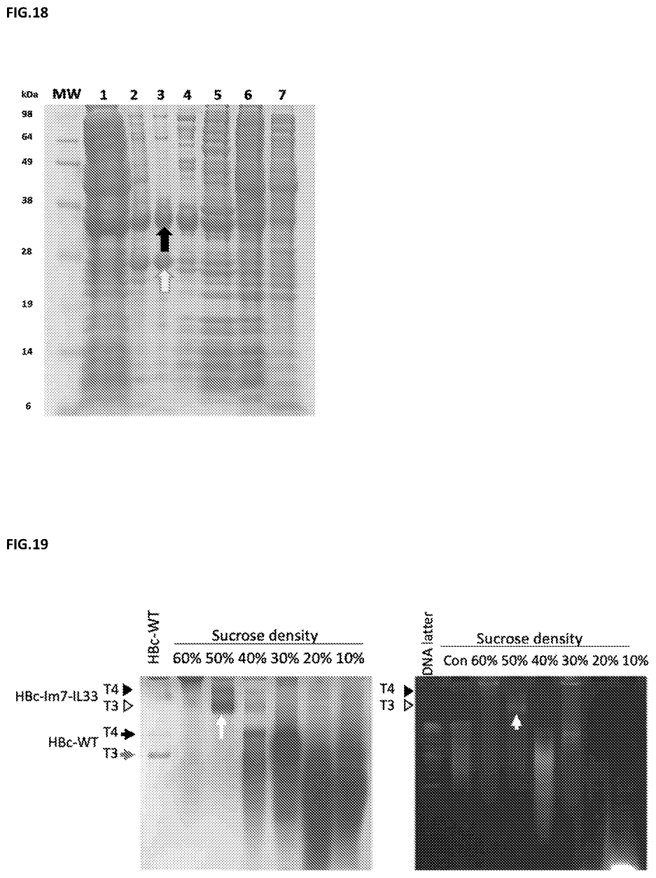

Sucrose density gradient centrifugation is sensitive to the size and density of particles. HBc and HBc-Im7 VLPs equilibrate into the 40% and 50% sucrose zones. Upon decoration of HBc-Im7 VLPs with an additional epitope protein, their size increases, which is expected to shift their equilibration in the sucrose gradient to higher densities. The data shows (FIG. 18), VLPs consisting of HBc-Im7 decorated with ColE7-1L33 equilibrate equimolar into the 50-60% zones, in keeping with their increased size. (The 60% sucrose fraction, situated at the bottom of the tube after equilibration, harbours a 50-60% interphase.)

### Example 16: IL33—Decorated VLPs Show Expected Ratio of T3 and T4 Sub-Populations and Retain RNA Content Also Seen in Wild Type VLPs

Protocol: 1% Agarose gels were cast using 0.5× TBE buffer and run with 50 μl of sample per lane at RT for 1 hr at 60 V. Instant Blue Coomassie was stained overnight at 4° C. SYBR™ safe (ThermoFisher) was used at 1:10,000 dilution and imaged on a ChemiDock™ Gel Imaging System (BioRad).

Consistent with the larger size of IL33-decorated VLPs detected by TEM and SDS-PAGE analysis of sucrose gradient density centrifugation (Examples 14 and 15) analysis of the sucrose density fractions by native agarose gel electrophoresis (NAGE) also showed that IL33-decorated VLPs localize to higher density, mostly in the 50% sucrose fraction with only a minor component in the 40% fraction (arrow in the Coomassie stained NAGE gel shown on the left of FIG. 19).

In addition, NAGE showed that the relative abundance of T4 and T3 VLP populations retain their relative ratio (T3>>T4) which had also been observed for HBc-Im7 and HBc-wild type VLPs.

SYBR SAFE staining of the NAGE gel (right of FIG. 19) shows the distinct RNA band migrating with the T3 and T4 bands, respectively, confirming integrity of the VLPs, protecting the E. coli-derived intra-VLP localised RNA from Benzonase digestion.

### Example 17: IL33—Decorated VLPs Exhibit Expected Size and Rim Structure

Protocol: TEM protocol and DLS protocol as described in Example 14.

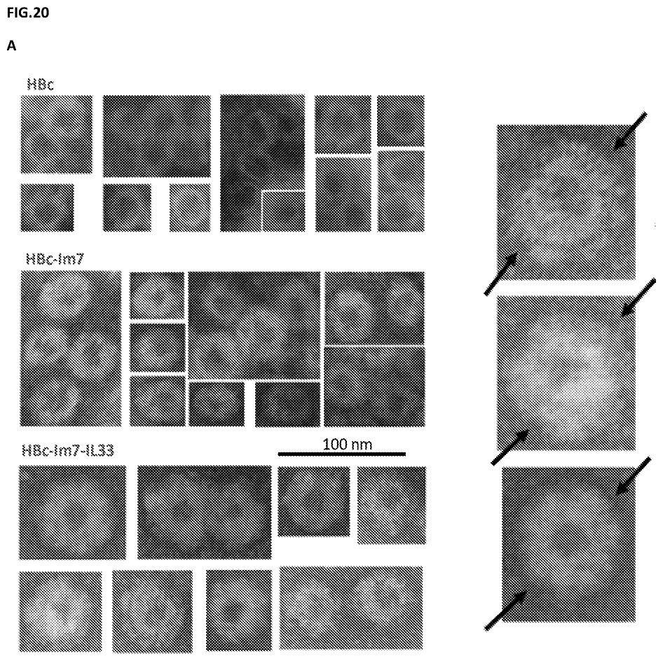

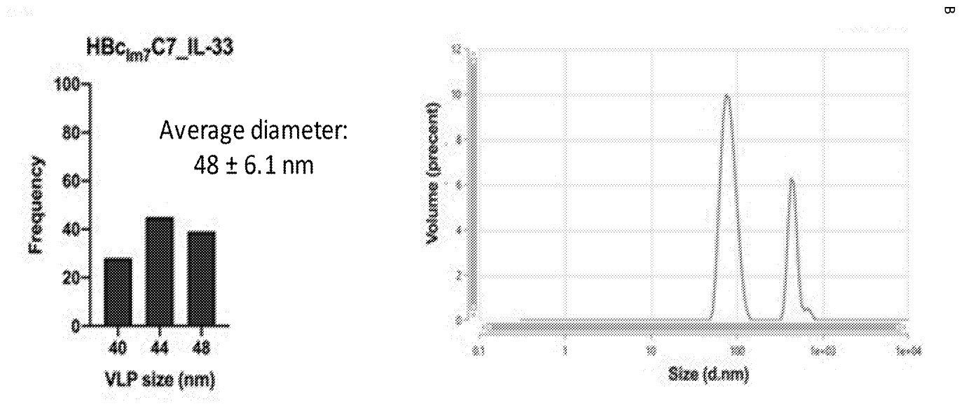

Transition Electron Microscopy (TEM) of wild type HBc, HBc-Im7, as well as HBc-Im7-IL33 VLPs illustrates the thickened rim in VLPs carrying the Im7 insert, as detailed in Example 5. Upon decoration of the HBc-Im7 VLP with ColicinE7-IL33 on the outside, a third dotted external protein layer becomes visible, especially at higher magnification (marked by black arrows in the close-up images on the right of FIG. 20 panel A), as would be expected from the added protein layer.

Size measurement using TEM images (left of FIG. 20 panel B) and Dynamic Light Scatter (right of FIG. 20 panel B) confirm a size increase in the IL33-decorated VLPs. The apparent discrepancy between average particle size observed by TEM (48 nm) and DLS (71 nm) is indicative of protein contraction artefacts of the flexibly linked outer IL33-layer upon attachment to the carbon grid during sample preparation for TEM.

### Example 18: Immuno-Dot Blot Confirms Native Protein Folding of IL33 Attached to the Surface of VLPs

Protocol: VLPs purified by density sucrose gradient were dialyzed against PBS. The volumes shown in the figure were spotted onto nitrocellulose membrane. Membranes were blocked with BSA/PBST, followed by incubation with anti-mIL33 (Invitrogen, PA5-4007, concentration 0.4 μg/ml) in PBST. The membrane was washed and exposed to secondary antibody (Donkey anti-goat HRP, diluted 1:10,000; Stratech, 705-035-147-JIR), followed by washing and development with Amersham ECL™ Western Blotting Detection Reagents.

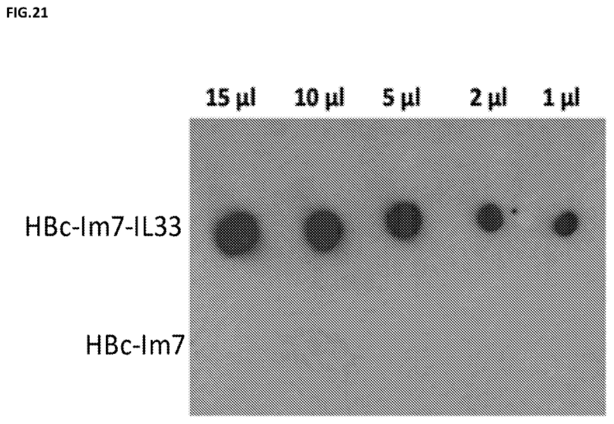

Native folding of IL33 bound to the surface of VLPs was verified by specific recognition of an antibody recognizing a conformational epitope. Decreasing amounts of either IL33-decorated or non-decorated HBc-Im7 VLPs were spotted onto nitrocellulose membrane. The data (FIG. 21) shows that even the lowest amount of IL33 present is clearly detected by the antibody whereas there is no signal detectable in VLPs in the absence of IL33, confirming specificity of the signal.

### Example 19: IL33-Decorated HBcIm7 VLPs are Immunogenic

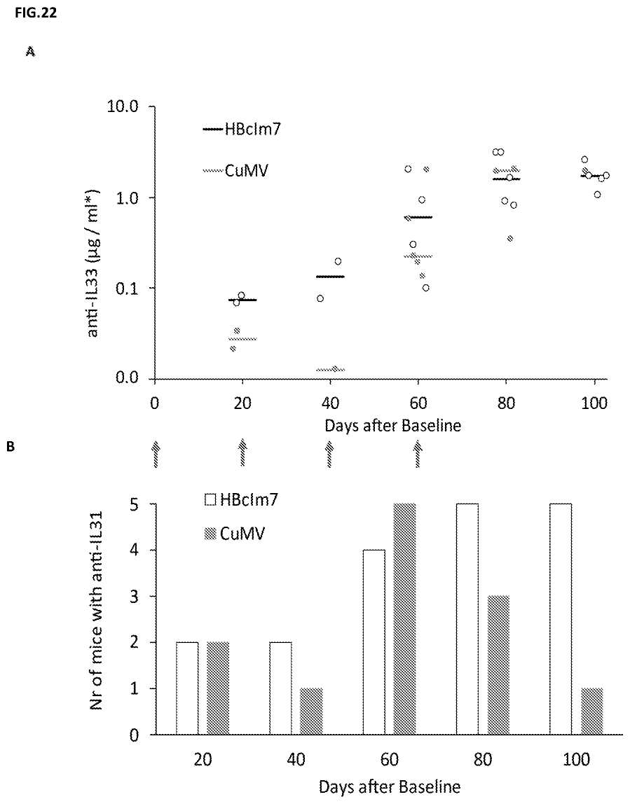

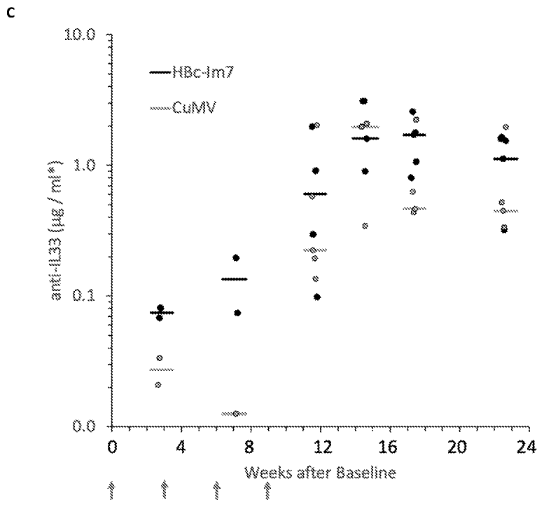

Protocol: Female C57Bl/6j mice, aged eight months were injected with 3 μg of either IL33-conjugated CuMV VLPs (provided by A. Zeltins, BMC Latvia, Riga) or IL33-decorated HBcIm7 VLPs (n=5 per group). Vaccines were sterile filtered and confirmed to have endotoxin<50 EU/ml by LAL assay prior to use. Both vaccines were formulated in PBS with 20% alum as adjuvant. Injections were placed subcutaneously in short term anaesthesia intrascapularly. Peripheral tail vein blood was sampled on the days indicated in the data (FIG. 22).

In order to test the immunogenic potential of VLPs presenting cytokines according to the method presented here, IL-33 decorated were used to vaccinate C57Bl/6j mice. This was done in direct head-to-head comparison with IL33-bearing VLPs made according to the previously used method, which is based on the chemical linkage of cytokines onto the surface of Cucumber Mosaic Virus-derived VLPs (CuMV, Zeltins, npj Vaccines 2, 30 (2017). https://doi.org/10.1038/s41541-017-0030-8).

As both vaccine types were expected to be immunogenic in principle, the experiment was carried out under limiting conditions, using minimal amount of vaccine per dose (3 μg) and using aged mice (8 months at the first dose).

Since the intended clinical use of the vaccine is the replacement of monoclonal antibodies, the observed accumulation of IL33— specific antibodies in vaccinated mice was not expressed as ‘titre’ of dilution, as is traditionally done. Rather, the samples sera were analysed in an ELISA calibrated by a commercially supplied high-affinity monoclonal antibody against IL33. Thus, any given measured optical density due to reaction of the vaccinated sera, which harbour polyclonal antibodies against IL33, can be assigned a value as being “equivalent to ×μg/ml of monoclonal anti-IL33”. The great advantage of this type of analysis, as opposed to reporting “titres”, is that the data give a direct indication of the potential efficacy of the prototype vaccines. For example, for many monoclonal antibodies in clinical use, the rough concentration in sera of patients is on the order of 2-10 μg/ml. This means that, if a vaccine triggers polyclonal antibodies achieving a response equivalent to this concentration, it has the potential to be clinically active.

As shown in the data (FIG. 22), under the limiting conditions studied, IL33-vaccine made using the invention showed a similar maximal antibody response (median concentration equivalent to 1.90 μg/ml in HBcIm7 vaccine versus 1.97 μg/ml in CuMV vaccine). However, the inventors have shown that mice vaccinated with the HBcIm7 vaccine responded earlier (see 3 and 7 weeks after baseline as shown in FIG. 22C). In addition, the median titre also persists at what would likely be a clinically effective level (1.12 μg/ml) to at least 23 weeks after baseline (as shown in FIGS. 22A and 22C).

Importantly, as shown in FIG. 22, HBcIm7 vaccinated mice showed a more sustained and consistent response between individual mice compared to CuMV vaccinated mice. Median titres were still in the therapeutic range at 23 weeks in HBcIm7 vaccinated mice but not in CuMV vaccinated mice.

Since the CuMV VLP platform is well established and shown to be effective with several vaccine models, these results suggest that the new HBcIm7 platform is suitably immunogenic for use as a vaccine.

### Example 20: Im7 Harbours Novel Chemistry Allowing Single-Step Affinity Purification of VLPs

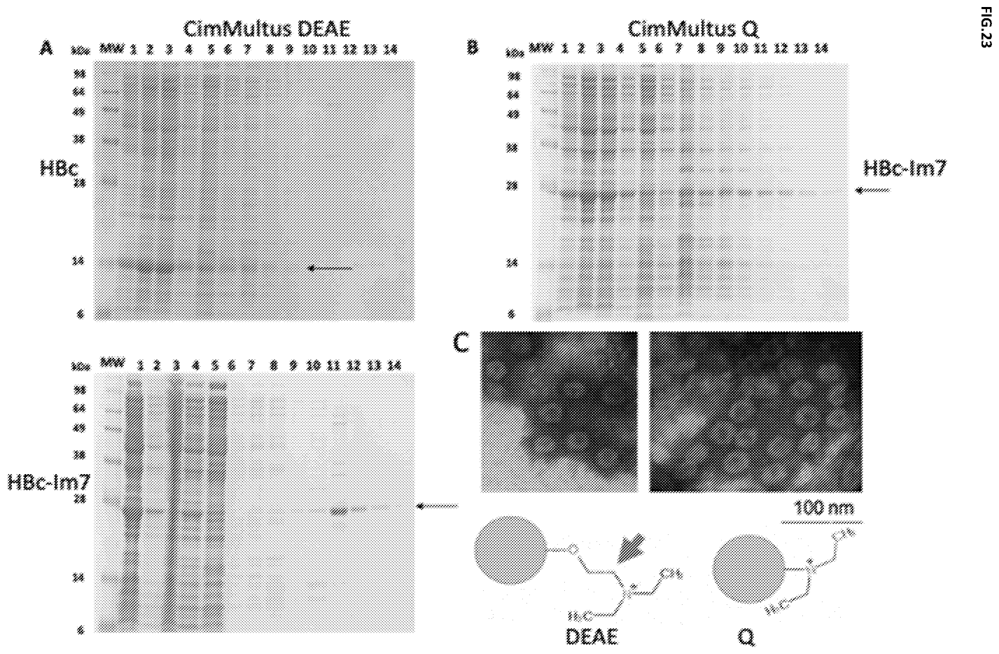

Protocol: Cytosolic fractions of E. coli expressing HBc or HBc-Im7 were subjected to anion exchange chromatography on 1 ml Qa or DEAE CimMultus monolith columns with a step-wise NaCl gradient. Lanes: 1—cytosols, 2—flowthrough, 3,4-0.1 M NaCl, 5,6-0.3 M NaCl, 7,8-0.5 M NaCl, 9,10-0.7 M NaCl, 11,12-1 M NaCl, 13,14-2 M NaCl. FIGS. 23A, B and D show SDS PAGE analyses of eluted fractions. Black arrows indicate the expected size of VLP monomer proteins. FIG. 23C shows representative TEM images of 1 M NaCl fraction from DEAE column purification of HBc-Im7.

During screening tests of VLP purification, it was noted that Im7-decorated VLPs bound extremely tight to the weak anion exchanger DEAE (FIG. 23A, bottom gel). This was in sharp contrast to wild type HBc VLPs, which only bound weakly to the same anion exchanger (FIG. 23A, top gel). The observed affinity of Im7-HBc VLPs was specific to DEAE as chemical group since binding of HBc-Im7 VLPs to the stronger anion exchanger Q was found to be weak (FIG. 23B, top).

The high affinity of the Im7-HBc interaction with DEAE allowed single-step purification of VLPs from crude cytosolic fractions. Purified VLPs appeared indistinguishable from sucrose-gradient purified VLPs under TEM (FIG. 23C).

In terms of mechanism, DEAE contains an added hydrophobic group compared to Q (marked by grey arrow, FIG. 23 C), which turns this moiety into a C4 carbon structure. Previous reports have documented high affinity interaction of C4 moieties for a number of specified enzymes (Hofstee, B. H. J., Biochem. Biophys. Res. Comm, 53, 1973, 1137-1144). By analogy, it is possible that a similar chemistry underlies the observed interaction of Im7 with DEAE.

This unexpected chemical property provides a straight forward and scalable means of VLP purification by affinity chromatography which has not been reported for any other type of VLP to date.

### Example 21: The ColE7-Im7 Interaction Allows Disassociation—Reassembly Purification of Decorated VLPs

Protocol: Decoration of PG to the VLP surface via the ColE7-Im7 interaction: Cytosolic fractions of each were mixed for 30 min at 4° C.

Disassembly of PG-decorated VLP capsids into VLP-dimers: cytosols were diluted 1:3 into 50 mM Tris, pH 8.0, final 67 mM NaCl, 30 mM imidazole and 3 M urea.

Ni-NTA purification: The disassembled dimers were purified using the now exposed c-terminal hexaHis tag on the VLP protein via IMAC (Ni-NTA) chromatography. After adsorption, the column was washed with 8 colume volumes (CV) of buffer containing 2 M urea and 30 mM imidazole, followed by 2 CV of buffer containing low urea (0.5 M). Capsid reassembly: The sample was eluted with 250 mM imi, 50 mM Arg/Glut, 10% glyc, 20 mM Tris pH 7.0 and 800 mM NaCl, followed by rotation at RT for 1 h.

VLP-isolation: the Ni-eluate was passed over a 1 ml column of CaptoCore700, which retains proteins<700 kD and 0.5 ml flow-through fractions collected.

Both ColicinE7 and Im7 preserve protein folding in the presence of 3 M urea. At this concentration of urea, HBc VLPs disassociate into protein dimers (Wingfield et al, Biochemistry 1995, 34, 4919-4932). The HBc-Im7 VLPs harbour a hexa-histidine tag at their C-terminal sequence, which is largely contained inside of the VLP. However, upon capsid disassembly with urea, the hexa-histidine tag becomes exposed, allowing affinity purification via IMAC.

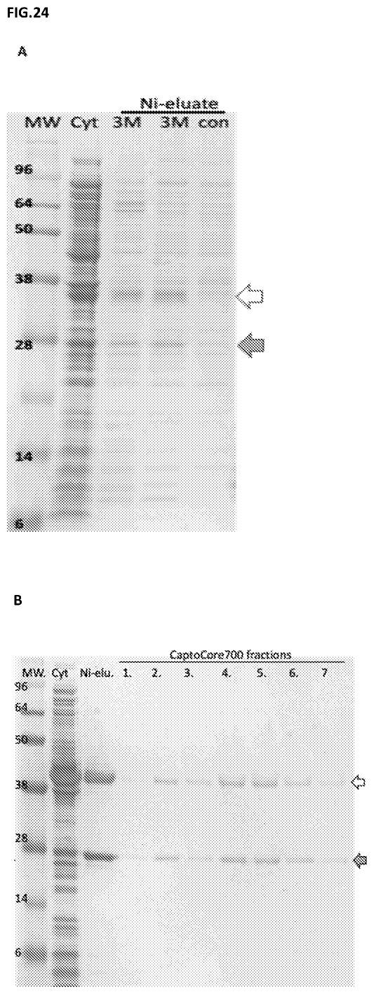

The SDS PAGE analysis shown in FIG. 24A confirms that intact VLPs do not bind to Ni-NTA unless the capsids are first disassembled using urea (lane labelled ‘con’). Shown are cytosolic fraction containing HBcIm7 (grey arrow) and ColE7-1L33 (white arrow), as well as eluate fractions of batch purifications performed in the absence or presence of 3 M urea, as indicated. ColicinE7-IL33, which is not histidine-tagged, co-elutes with HBcIm7 off the nickel column. This shows that the binding of ColE7 and Im7 is sustained despite 3 M urea.

Once eluted from the Nickel-column (after stepwise removal of urea from the buffer during washing), the dimeric proteins can be reassociated into capsids by incubation in high NaCl concentration (Ceres and Zlotnick, Biochemistry 2002, 41, 11525-11531). Therefore, dimeric proteins eluted off the Nickel column were incubated in the presence of 800 mM NaCl and additional stabilizing factors. Thereafter, they were subjected to modified size exclusion chromatography on CaptoCore700 (Cc700) resin, which retains all proteins smaller than 700 kD. The resulting SDS PAGE analysis shown in FIG. 24B, which shows that both HBc-Im7 and ColE7-1L33 co-elute in the flow-through of the Cc700 column, verifying re-assembly of capsids.

### Example 22: The Use of Colicin E7 to Fuse Proteins to the Surface of VLPs Simultaneously Provides a Chaperone Function Allowing Native Protein Folding of Proteins not Soluble in E. coli on their Own

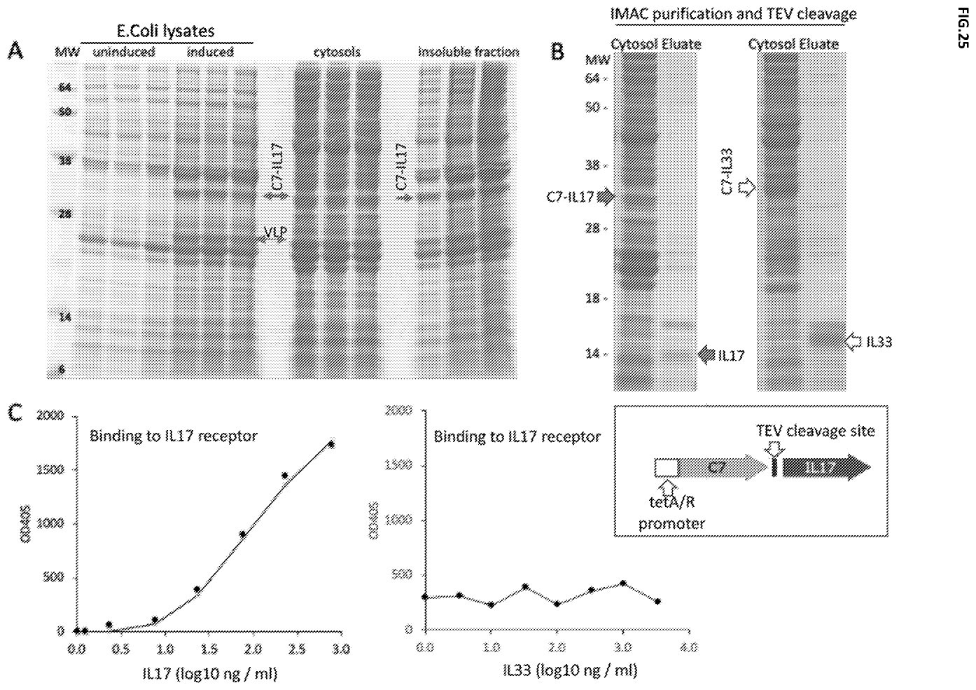

Protocol: Expression—A plasmid harbouring human IL17 with an N-terminal fusion of Colicin E7 (marked C7 in the cartoon diagram) was transfected in E. coli BL21/DE3 cells harbouring helper-enzymes for disulfide formation (Erv1P, DsbC). Induction was induced with anhydro-tetracycline for IL17) and IPTG (for the helper enzymes) for 16 h at 18° C. Cells were lysed. Cytosolic fractions were prepared by sonication, and addition of Benzonase, followed by incubation with 0.5% Triton X100 for 45 min at 4° C. and centrifugation. Supernatants were designated cytosol; insoluble pellets containing inclusion bodies were resuspended with 7 M urea. The SDS-PAGE analysis (FIG. 25 A) shows three independent clones, with IL17 marked in bold arrows and helper enzymes in a small arrow.

Purification—Human IL17 or IL33 were expressed as shown in (FIG. 25A). Cytosols were incubated with commercially sourced Im7 linked to Sepharose. After extensive washing, the Im7-Sepharose was incubated with TEV protease overnight at 4° C., filled into a column and eluted by gravity flow. The resulting SDS-PAGE analysis is shown in FIG. 25B, the cytokines before and after TEV digest, respectively, indicated as IL17 or IL33 with labelled arrows

Receptor binding ELISA—(FIG. 25C) Human IL17RA protein (SinoBiological) was coated to ELISA plates at 0.25 ug/well. 4 ul of Im7-Sepharose purified IL17 and 2.5 ul of IL33, respectively, was added in 200 ul PBST per well. 2nd antibody was 1:1000 anti-IL17/anti-IL33 (both mouse IgG). Anti-mouse IgG-AP was added at 1:10,000 for 30 min. For EC50 calculation, MW of 30 KD was used, equivalent to human IL17 dimer devoid of glycosylation (produced in E. coli).

Colicin E7 not only serves to achieve attachment of cytokines to VLPs via binding to Im7 but, simultaneously, also functions as chaperone allowing expression of difficult-to-produce proteins. Human IL17 on its own cannot be expressed in E. coli as soluble protein, even with the addition of helper enzymes (DsbC, Erv1P), assisting in folding of proteins containing disulfide bonds, such as IL17.

The FIG. 25 shows that, when adding Colicin E7 as N-terminally fused unit (shown in the cartoon), there is robust induction of IL17 using our system and approximately 50% of the induced protein is soluble in the cytosol (FIG. 25A). The Colicin-E7 moiety is properly folded, since the protein can be isolated via immobilized Im7-chromatography, followed by cleavage with TEV protease (FIG. 25B, also showing IL33). Finally, purified IL17 is functional, showing specific binding to its receptor IL17RA with a calculated kD of 5.3 nM (FIG. 25C), which is in good agreement with published data (2.3 nM, Wright, J Immunol 2015, doi 10.4049/jimmunol.181.4.2799). In confirmation of specificity, IL33 does not bind to the IL17RA receptor.

### Example 23: Independent and Sequential Induction of VLP Backbone and Epitope Proteins within Single Cells Allows Initial VLP Formation Followed by Epitope Assembly

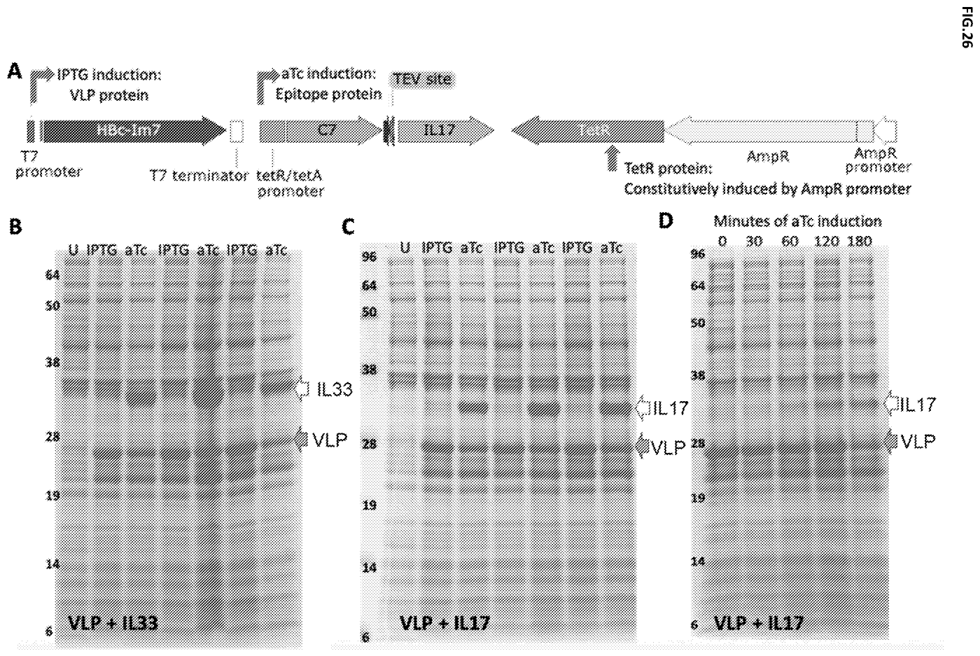

The expression system was refined to achieve independent and sequential inducibility of the VLP backbone and the epitope protein to be decorated at the surface. This reduces metabolic stress of the cells who are no longer required to produce all recombinant proteins at the same time. To this end, a plasmid was constructed where the tetR protein is constitutively expressed, driven by a ribosomal binding site downstream of the AmpR gene (cartoon diagram, FIG. 26A). Second, the epitope proteins were placed downstream of a tetR/tetA promoter also containing a dual tet operator. As a result, epitope proteins are only induced upon addition of anhydro-tetracycline (aTc) to the culture medium by causing dissociation of the tetR protein from the tetR/tetA promoter.

The system was tested with different epitopes. Shown here are data for IL33 and IL17. The plasmid was transfected into a BL21/DE3 E. coli strain which had been modified to also harbour the two enzymes DsbC and Erv1P as chromosomal integrated copies under the control of the T7 promoter. These enzymes are a disulfide transferase and disulfide isomerase enzymes, respectively, which assist in the folding of recombinant proteins harbouring disulfide bonds, such as IL17. Transfected E. coli were induced with IPTG for 20 h at 20° C. (marked ‘IPTG’). Compared to uninduced lysates (marked ‘U’) there is a strong induction of the HBc-Im7 VLP backbone, as well as the expected induction of DsbC and Erv1P visible. Subsequently, IPTG was removed by replacement of medium and aTc added, followed by incubation at 27° C. for 5 h. This led to a strong added induction of both IL33 and IL17 (marked ‘aTc’).

The SDS PAGE gels in FIG. 26, panels B and C show three independent clones for each cytokine, documenting exceptionally tight regulation without any leakiness. This allows prior formation of VLP and subsequent decoration by the surface epitopes within bacterial cells. The gel shown in FIG. 26 panel D shows a time course of aTc induction in E. coli already induced with IPTG. Induction of IL17 is notable after 60 min and complete within 3 h.

### Example 24: Barstar can be Incorporated into HBc to Form VLPs

Protocol: Sucrose gradient was carried out as described in Example 11. TEM: samples were dissolved in 50 mM Tris/HCl, pH 7.0, 150 mM NaCl buffer and adsorbed to glow discharged carbon-formvar-coated copper grids and negatively stained with a 1% aqueous uranyl acetate. The grids were examined at 80 kV. DLS: Samples contained in sucrose were dialyzed against Tris/HCl pH 7.0, 150 mM NaCl and analysed in 80 ul volume with a Zetasizer Ultra (Malvern Panalytical).

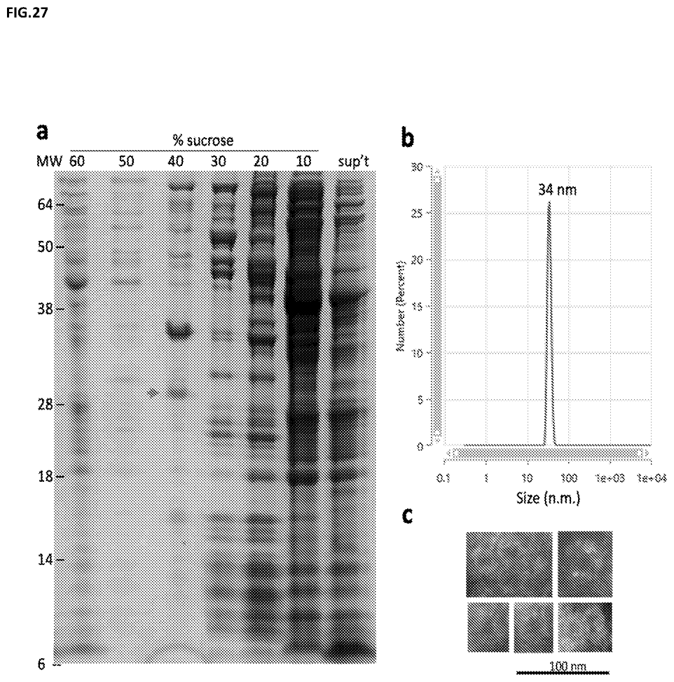

Analogous to the Im7 protein, the Barstar protein can be incorporated into HBc capsids to yield VLPs. When expressed as in-frame fusion in E. coli, large nanoparticles form which migrate in the 40% fraction when applied to sucrose density gradient centrifugation (marked by a grey arrow in panel “a” of FIG. 27). These particles show a peak at 34 nm size upon DLS measurement (panel b) and exhibit a thickened rim, when analysed by TEM (panel c) similar to those seen when Im7 is incorporated into HBc VLPs (see Example 14).

### Example 25: The Barnase—Barstar Interaction Allows Disassociation—Reassembly Purification of Decorated VLPs

Exactly as with ColicinE7 and Im7 (see Example 21), Barnase and Barstar preserve protein folding in the presence of 3 M urea. At this concentration of urea, HBc VLPs disassociate into protein dimers (Wingfield et al, Biochemistry 1995, 34, 4919-4932). The HBc-Barstar VLPs harbour a hexa-histidine tag at their C-terminal sequence, which is largely contained inside of the VLP. However, upon capsid disassembly with urea, the hexa-histidine tag becomes exposed, allowing affinity purification via IMAC.

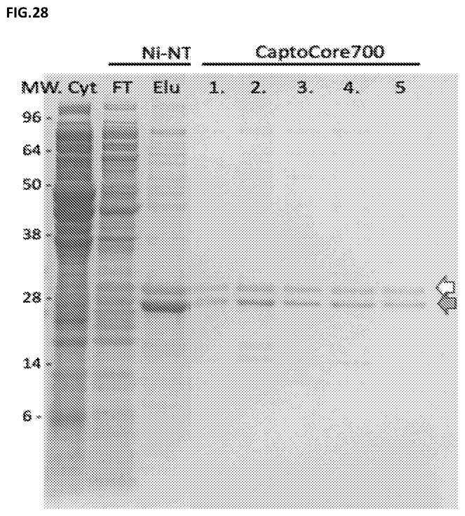

The SDS—PAGE analysis shown in FIG. 28 is analogous to that shown in Example 21, except that HBc-Barstar was used as VLP (grey arrow) and Barnase-IL13 as epitope (white arrow). The gel shows the mixed cytosolic fractions containing both HBc-Barstar and Barnase-IL13, followed by Ni-NTA eluate. The CaptoCore700 fractions represent the flow-through of the Cc700 column, containing only proteins>700 kD. The result indicates that capsid reassembly, as detailed in Example 21, was successful.

TABLE 1
Plasmid Clones used in examples

| | | | Clone- | | |
|---|---|---|---|---|---|
| | Short | | number | SEQ | |
| Name | name | Description | (DU) | ID NO. | Key features |
| pETDuet-1 Hcl | HBc- | Hepatitis-C Core-capsid, aa1- | 67866 | 1 | Im7-protein |
| | Im7 | 145, with in-frame fusion to | | | fused into HBc |
| | | Im7 protein | | | capsid protein |
| pETDuet-1 Hcl | HBc- | Hepatitis-C Core-capsid, aa1- | 67867 | 2 | Im7-protein |
| 6His | Im7-h | 145, with in-frame fusion to | | | fused into HBc |
| | | Im7 protein, with C-terminal | | | capsid protein, |
| | | histidine-extension | | | C-terminally |
| | | | | | stabilized |
| pETDuet-1 C7 | C7-IL13 | ColE7-N-terminally-fused-to- | 70076 | 3 | ColE7-IL13 alone |
| linker TEV IL13 | | murine-interleukin13 | | | |
| Duet-1 C7 linker | C7-IL33 | ColE7-N-terminally-fused-to- | 70051 | 4 | ColE7-IL33 alone |
| TEV IL33 | | murine-interleukin33 | | | |
| pETDuet-1 Hcl F L | Im7- | HBc-capsid, containing F−>L | 70080 | 5 | HBc-Im7 VLP |
| 6His - C7 linker | IL13 | mutation to accelerate | | | shell and ColE7- |
| TEV IL13 | | folding, in-frame fused to | | | IL13 |
| | | Im7, and ColE7-fused-IL13 | | | |
| pETDuet-1 Hcl F L | Im7- | HBc-capsid, containing F−>L | 70059 | 6 | HBc-Im7 VLP |
| 6His - C7 linker | IL33 | mutation to accelerate | | | shell and ColE7- |
| TEV IL33 | | folding, in-frame fused to | | | IL33 |
| | | Im7, and ColE7-fused-IL33 | | | |
| pETDuet-1 Hcl F L | Im7- | HBc-capsid, containing F−>L | 70126 | 7 | HBc-Im7 VLP |
| 6His - C7 linker | IL33- | mutation to accelerate | | | shell and ColE7- |
| TEV IL33 with | T7mut | folding, in-frame fused to | | | IL33 with tuned |
| mutated T7 | | Im7, and mutated-T7- | | | T7 expression |
| | | promoter-driven ColE7- | | | |
| | | fused-IL33 | | | |
| pETDuet1 Hcl F L | Im7-PG | HBc-capsid, containing F−>L | 65750 | 8 | HBc-Im7 VLP |
| 6His + Protein G | | mutation to accelerate | | | shell and ColE7- |
| Linker TEV C7 | | folding, in-frame fused to | | | Protein G |
| | | Im7, and ColE7-fused- | | | |
| | | Protein-G | | | |
| pcDNA5D C7 S- | RBD | ColE7 fused N-terminally to | 67808 | 9 | ColE7-Spike RBD |
| RBD | | SARS2-Spike RBD | | | fusion |
| pcDNA5D C7 Sp- | RBD- | ColE7-N-terminally fused to | 67793 | 10 | ColE7-Spike RBD- |
| RBD GFP | GFP | Spike-Receptor-binding- | | | eGFP fusion |
| | | domain with c-terminal eGFP | | | |
| | | for expression monitoring | | | |
| pcDNA5D C7 Sp- | RBD-N | ColE7-N-terminally fused to | 67817 | 11 | ColE7-Spike RBD- |
| RBD N-ntd | | Spike-Receptor-binding- | | | N fusion |
| | | domain in tandem with non- | | | |
| | | RNA-binding C-terminal | | | |
| | | fragment of SARS2- | | | |
| | | nucleocapsid protein | | | |
| pETDuet-1 HBc- | — | HBc-capsid, containing F−>L | 70680 | 47 | HBc-Im7 VLP |
| Im7 F−>L 6His - | | mutation to accelerate | | | shell and ColE7- |
| tet- | | folding, in-frame fused to | | | IL17 fusion with |
| inducible_ColE7- | | Im7, and ColE7-fused-IL17 | | | Independent |
| linker- | | under control of separate T7 | | | expression |
| TEV_site_hIL17A | | and TetR promoters | | | |
| pETDuet-1 HBc- | — | HBc-capsid, containing F−>L | 70612 | 48 | HBc-Im7 VLP |
| Im7 F−>L 6His - | | mutation to accelerate | | | shell and ColE7- |
| tet- | | folding, in-frame fused to | | | IL33 fusion with |
| inducible_ColE7- | | Im7, and ColE7-fused-IL33 | | | Independent |
| linker- | | under control of separate T7 | | | expression |
| TEV_site_mIL33 | | and TetR promoters | | | |

Sequences

SEQ ID NO: 1
>pETDuet-1 HcI DU67866 (6107 bp)
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacactttgtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgtttaataaGCGGCCGCATAATGCTT
AAGTCGAACAGAAAGTAATCGTATTGTACACGGCCGCATAATCGAAATTAATACGACTCACTATAGGGGAA
TTGTGAGCGGATAACAATTCCCCATCTTAGTATATTAGTTAAGTATAAGAAGGAGATATACATATGGCAGA
TCTCAATTGGATATCGGCCGGCCACGCGATCGCTGACGTCGGTACCCTCGAGTCTGGTAAAGAAACCGCTG
CTGCGAAATTTGAACGCCAGCACATGGACTCGTCTACTAGCGCAGCTTAATTAACCTAGGCTGCTGCCACC
GCTGAGCAATAACTAGCATAACCCCTTGGGGCCTCTAAACGGGTCTTGAGGGGTTTTTTGCTGAAAGGAGG
AACTATATCCGGATTGGCGAATGGGACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAGTGCTTTA
CGGCACCTCGACCCCAAAAAACTTGATTAGGGTGATGGTTCACGTAGTGGGCCATCGCCCTGATAGACGGT
TTTTCGCCCTTTGACGTTGGAGTCCACGTTCTTTAATAGTGGACTCTTGTTCCAAACTGGAACAACACTCA
ACCCTATCTCGGTCTATTCTTTTGATTTATAAGGGATTTTGCCGATTTCGGCCTATTGGTTAAAAAATGAG
CTGATTTAACAAAAATTTAACGCGAATTTTAACAAAATATTAACGTTTACAATTTCTGGCGGCACGATGGC
ATGAGATTATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCGATCTGTC
TATTTCGTTCATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTTGCC
GGGAAGCTAGAGTAAGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGGCGAGTTACATGATC
CCCCATGTTGTGCAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAACGTTCTTCGG
GGCGAAAACTCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGCCGCAAAAAA
GGGAATAAGGGCGACACGGAAATGTTGAATACTCATACTCTTCCTTTTTCAATCATGATTGAAGCATTTAT
CAGGGTTATTGTCTCATGAGCGGATACATATTTGAATGTATTTAGAAAAATAAACAAATAGGTCATGACCA
AAATCCCTTAACGTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATA
GTTACCGGATAAGGCGCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAAGAGCGCCTGATGCGGT
ATTTTCTCCTTACGCATCTGTGCGGTATTTCACACCGCATATATGGTGCACTCTCAGTACAATCTGCTCTG
ATGCCGCATAGTTAAGCCAGTATACACTCCGCTATCGCTACGTGACTGGGTCATGGCTGCGCCCCGACACC
CGCCAACACCCGCTGACGCGCCCTGACGGGCTTGTCTGCTCCCGGCATCCGCTTACAGACAAGCTGTGACC
GTCTCCGGGAGCTGCATGTGTCAGAGGTTTTCACCGTCATCACCGAAACGCGCGAGGCAGCTGCGGTAAAG
CTCATCAGCGTGGTCGTGAAGCGATTCACAGATGTCTGCCTGTTCATCCGCGTCCAGCTCGTTGAGTTTCT
CCAGAAGCGTTAATGTCTGGCTTCTGATAAAGCGGGCCATGTTAAGGGCGGTTTTTTCCTGTTTGGTCACT
GATGCCTCCGTGTAAGGGGGATTTCTGTTCATGGGGGTAATGATACCGATGAAACGAGAGAGGATGCTCAC
GATACGGGTTACTGATGATGAACATGCCCGGTTACTGGAACGTTGTGAGGGTAAACAACTGGCGGTATGGA
TGCGGCGGGACCAGAGAAAAATCACTCAGGGTCAATGCCAGCGCTTCGTTAATACAGATGTAGGTGTTCCA
CAGGGTAGCCAGCAGCATCCTGCGATGCAGATCCGGAACATAATGGTGCAGGGCGCTGACTTCCGCGTTTC
CAGACTTTACGAAACACGGAAACCGAAGACCATTCATGTTGTTGCTCAGGTCGCAGACGTTTTGCAGCAGC
AGTCGCTTCACGTTCGCTCGCGTATCGGTGATTCATTCTGCTAACCAGTAAGGCAACCCCGCCAGCCTAGC
CGGGTCCTCAACGACAGGAGCACGATCATGCTAGTCATGCCCCGCGCCCACCGGAAGGAGCTGACTGGGTT
GAAGGCTCTCAAGGGCATCGGTCGAGATCCCGGTGCCTAATGAGTGAGCTAACTTACATTAATTGCGTTGC
GCTCACTGCCCGCTTTCCAGTCGGGAAACCTGTCGTGCCAGCTGCATTAATGAATCGGCCAACGCGCGGGG
AGAGGCGGTTTGCGTATTGGGCGCCAGGGTGGTTTTTCTTTTCACCAGTGAGACGGGCAACAGCTGATTGC
CCTTCACCGCCTGGCCCTGAGAGAGTTGCAGCAAGCGGTCCACGCTGGTTTGCCCCAGCAGGCGAAAATCC
TGTTTGATGGTGGTTAACGGCGGGATATAACATGAGCTGTCTTCGGTATCGTCGTATCCCACTACCGAGAT
GTCCGCACCAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACATGGCACTC
CAGTCGCCTTCCCGTTCCGCTATCGGCTGAATTTGATTGCGAGTGAGATATTTATGCCAGCCAGCCAGACG
CAGACGCGCCGAGACAGAACTTAATGGGCCCGCTAACAGCGCGATTTGCTGGTGACCCAATGCGACCAGAT
GCTCCACGCCCAGTCGCGTACCGTCTTCATGGGAGAAAATAATACTGTTGATGGGTGTCTGGTCAGAGACA
TCAAGAAATAACGCCGGAACATTAGTGCAGGCAGCTTCCACAGCAATGGCATCCTGGTCATCCAGCGGATA
GTTAATGATCAGCCCACTGACGCGTTGCGCGAGAAGATTGTGCACCGCCGCTTTACAGGCTTCGACGCCGC
TTCGTTCTACCATCGACACCACCACGCTGGCACCCAGTTGATCGGCGCGAGATTTAATCGCCGCGACAATT
TGCGACGGCGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTTTCGCAG
AAACGTGGCTGGCCTGGTTCACCACGCGGGAAACGGTCTGATAAGAGACACCGGCATACTCTGCGACATCG
TATAACGTTACTGGTTTCACATTCACCACCCTGAATTGACTCTCTTCCGGGCGCTATCATGCCATACCGCG
AAAGGTTTTGCGCCATTCGATGGTGTCCGGGATCTCGACGCTCTCCCTTATGCGACTCCTGCATTAGGAAG
CAGCCCAGTAGTAGGTTGAGGCCGTTGAGCACCGCCGCCGCAAGGAATGGTGCATGCAAGGAGATGGCGCC
CAACAGTCCCCCGGCCACGGGGCCTGCCACCATACCCACGCCGAAACAAGCGCTCATGAGCCCGAAGTGGC
GAGCCCGATCTTCCCCATCGGTGATGTCGGCGATATAGGCGCCAGCAACCGCACCTGTGGCGCCGGTGATG
CCGGCCACGATGCGTCCGGCGTAGAGGATCGAGATCGATCTCGATCCCGCGAAATTAATACGACTCACTAT
A
SEQ ID NO: 2
>pETDuet-1 HcI 6His
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacactttgtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCATAATGCTTAAGTCGAACAGAAAGTAATCGTATTGTACACGGCCGCATAATCGAAATTAATA
CGACTCACTATAGGGGAATTGTGAGCGGATAACAATTCCCCATCTTAGTATATTAGTTAAGTATAAGAAGG
AGATATACATATGGCAGATCTCAATTGGATATCGGCCGGCCACGCGATCGCTGACGTCGGTACCCTCGAGT
CTGGTAAAGAAACCGCTGCTGCGAAATTTGAACGCCAGCACATGGACTCGTCTACTAGCGCAGCTTAATTA
ACCTAGGCTGCTGCCACCGCTGAGCAATAACTAGCATAACCCCTTGGGGCCTCTAAACGGGTCTTGAGGGG
TTTTTTGCTGAAAGGAGGAACTATATCCGGATTGGCGAATGGGACGCGCCCTGTAGCGGCGCATTAAGCGC
GGCGGGTGTGGTGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGGCTCCCTTTAGGG
TTCCGATTTAGTGCTTTACGGCACCTCGACCCCAAAAAACTTGATTAGGGTGATGGTTCACGTAGTGGGCC
ATCGCCCTGATAGACGGTTTTTCGCCCTTTGACGTTGGAGTCCACGTTCTTTAATAGTGGACTCTTGTTCC
AAACTGGAACAACACTCAACCCTATCTCGGTCTATTCTTTTGATTTATAAGGGATTTTGCCGATTTCGGCC
TATTGGTTAAAAAATGAGCTGATTTAACAAAAATTTAACGCGAATTTTAACAAAATATTAACGTTTACAAT
TTCTGGCGGCACGATGGCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCGTGTAGATAACTACGATA
CGGGAGGGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCTCCATCC
AGTCTATTAATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAGCAAAAACAGGAAG
GCAAAATGCCGCAAAAAAGGGAATAAGGGCGACACGGAAATGTTGAATACTCATACTCTTCCTTTTTCAAT
CATGATTGAAGCATTTATCAGGGTTATTGTCTCATGAGCGGATACATATTTGAATGTATTTAGAAAAATAA
ACAAATAGGTCATGACCAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAACTGGCTTCAGCAGAG
CGCAGATACCAAATACTGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACGCTT
CCCGAAGGGAGAAAGGCGGACAGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGATAACCGTATTACCGC
CTTTGAGTGAGCTGATACCGCTCGCCGCAGCCGAACGACCGAGCGCAGCGAGTCAGTGAGCGAGGAAGCGG
AAGAGCGCCTGATGCGGTATTTTCTCCTTACGCATCTGTGCGGTATTTCACACCGCATATATGGTGCACTC
TCAGTACAATCTGCTCTGATGCCGCATAGTTAAGCCAGTATACACTCCGCTATCGCTACGTGACTGGGTCA
TGGCTGCGCCCCGACACCCGCCAACACCCGCTGACGCGCCCTGACGGGCTTGTCTGCTCCCGGCATCCGCT
TACAGACAAGCTGTGACCGTCTCCGGGAGCTGCATGTGTCAGAGGTTTTCACCGTCATCACCGAAACGCGC
GAGGCAGCTGCGGTAAAGCTCATCAGCGTGGTCGTGAAGCGATTCACAGATGTCTGCCTGTTCATCCGCGT
CCAGCTCGTTGAGTTTCTCCAGAAGCGTTAATGTCTGGCTTCTGATAAAGCGGGCCATGTTAAGGGCGGTT
TTTTCCTGTTTGGTCACTGATGCCTCCGTGTAAGGGGGATTTCTGTTCATGGGGGTAATGATACCGATGAA
ACGAGAGAGGATGCTCACGATACGGGTTACTGATGATGAACATGCCCGGTTACTGGAACGTTGTGAGGGTA
AACAACTGGCGGTATGGATGCGGCGGGACCAGAGAAAAATCACTCAGGGTCAATGCCAGCGCTTCGTTAAT
ACAGATGTAGGTGTTCCACAGGGTAGCCAGCAGCATCCTGCGATGCAGATCCGGAACATAATGGTGCAGGG
CGCTGACTTCCGCGTTTCCAGACTTTACGAAACACGGAAACCGAAGACCATTCATGTTGTTGCTCAGGTCG
CAGACGTTTTGCAGCAGCAGTCGCTTCACGTTCGCTCGCGTATCGGTGATTCATTCTGCTAACCAGTAAGG
CAACCCCGCCAGCCTAGCCGGGTCCTCAACGACAGGAGCACGATCATGCTAGTCATGCCCCGCGCCCACCG
GAAGGAGCTGACTGGGTTGAAGGCTCTCAAGGGCATCGGTCGAGATCCCGGTGCCTAATGAGTGAGCTAAC
TTACATTAATTGCGTTGCGCTCACTGCCCGCTTTCCAGTCGGGAAACCTGTCGTGCCAGCTGCATTAATGA
ATCGGCCAACGCGCGGGGAGAGGCGGTTTGCGTATTGGGCGCCAGGGTGGTTTTTCTTTTCACCAGTGAGA
CGGGCAACAGCTGATTGCCCTTCACCGCCTGGCCCTGAGAGAGTTGCAGCAAGCGGTCCACGCTGGTTTGC
CCCAGCAGGCGAAAATCCTGTTTGATGGTGGTTAACGGCGGGATATAACATGAGCTGTCTTCGGTATCGTC
GTATCCCACTACCGAGATGTCCGCACCAACGCGCAGCCCGGACTCGGTAATGGCGCGCATTGCGCCCAGCG
CCATCTGATCGTTGGCAACCAGCATCGCAGTGGGAACGATGCCCTCATTCAGCATTTGCATGGTTTGTTGA
AAACCGGACATGGCACTCCAGTCGCCTTCCCGTTCCGCTATCGGCTGAATTTGATTGCGAGTGAGATATTT
ATGCCAGCCAGCCAGACGCAGACGCGCCGAGACAGAACTTAATGGGCCCGCTAACAGCGCGATTTGCTGGT
GACCCAATGCGACCAGATGCTCCACGCCCAGTCGCGTACCGTCTTCATGGGAGAAAATAATACTGTTGATG
GGTGTCTGGTCAGAGACATCAAGAAATAACGCCGGAACATTAGTGCAGGCAGCTTCCACAGCAATGGCATC
CTGGTCATCCAGCGGATAGTTAATGATCAGCCCACTGACGCGTTGCGCGAGAAGATTGTGCACCGCCGCTT
TACAGGCTTCGACGCCGCTTCGTTCTACCATCGACACCACCACGCTGGCACCCAGTTGATCGGCGCGAGAT
TTAATCGCCGCGACAATTTGCGACGGCGCGTGCAGGGCCAGACTGGAGGTGGCAACGCCAATCAGCAACGA
CTGTTTGCCCGCCAGTTGTTGTGCCACGCGGTTGGGAATGTAATTCAGCTCCGCCATCGCCGCTTCCACTT
TTTCCCGCGTTTTCGCAGAAACGTGGCTGGCCTGGTTCACCACGCGGGAAACGGTCTGATAAGAGACACCG
GCATACTCTGCGACATCGTATAACGTTACTGGTTTCACATTCACCACCCTGAATTGACTCTCTTCCGGGCG
CTATCATGCCATACCGCGAAAGGTTTTGCGCCATTCGATGGTGTCCGGGATCTCGACGCTCTCCCTTATGC
GACTCCTGCATTAGGAAGCAGCCCAGTAGTAGGTTGAGGCCGTTGAGCACCGCCGCCGCAAGGAATGGTGC
ATGCAAGGAGATGGCGCCCAACAGTCCCCCGGCCACGGGGCCTGCCACCATACCCACGCCGAAACAAGCGC
TCATGAGCCCGAAGTGGCGAGCCCGATCTTCCCCATCGGTGATGTCGGCGATATAGGCGCCAGCAACCGCA
CCTGTGGCGCCGGTGATGCCGGCCACGATGCGTCCGGCGTAGAGGATCGAGATCGATCTCGATCCCGCGAA
ATTAATACGACTCACTATA
SEQ ID NO: 3
>pETDuet-1 C7 linker TEV IL13 70076 (6167 bp)
GGGGAATTGTGAGCGGATAACAATTCCCCTCTAGAAATAATTTTGTTTAACTTTAAGAAGGAGATATACCA
TGGGCAGCAGCCATCACCATCATCACCACAGCCAGGATCCGAATTCGAGCTCGGCGCGCCTGCAGGTCGAC
AAGCTTGCGGCCGCATAATGCTTAAGTCGAACAGAAAGTAATCGTATTGTACACGGCCGCATAATCGAAAT
TAATACGACTCACTATAGGGGAATTGTGAGCGGATAACAATTCCCCATCTTAGTATATTAGTTAAGTATAA
GAAGGAGATATACATATGGAGAGTAAACGGAATAAGCCAGGGAAGGCAACAGGTAAAGGAAAACCTGTCAA
TAATAAGTGGTTAAATAATGCAGGTAAAGACTTAGGTTCTCCTGTTCCAGATCGTATAGCTAATAAACTAC
GTGATAAGGAGTTTAAAAGTTTCGATGATTTTCGTAAGAAATTCTGGGAAGAAGTGTCAAAAGATCCTGAG
TTAAGTAAACAATTTAGTCGAAACAATAATGATCGAATGAAGGTTGGAAAAGCGCCCAAGACTAGAACCCA
GGATGTTTCAGGGAAGGCAACTTCATTCGCACTTCATCATGAGAAGCCGATCAGCCAAAATGGTGGTGTCT
ATGATATGGATAACATCAGCGTGGTAACACCTAAACGTGCTATTGATATTCACCGAGGTAAAAGCGGAGGT
GGCTCATCAGGATCTGGTGAAAACCTGTATTTTCAGGGATCcGGCCCGGTGCCTCCAAGCACTGCCCTGCG
TGAGTTAATCGAAGAATTAGTCAACATTACCCAAAACCAGAAGGCACCGCTGTGCAACGGTTCAATGGTCT
GGAGCATCAATCTGACAGCCGGTATGTATTGCGCGGCGCTTGAAAGCCTGATTAACGTGAGTGGTTGTTCT
GCCATCGAAAAAACTCAACGTATGTTGTCCGGCTTTTGCCCACACAAGGTATCTGCCGGTCAATTCAGTTC
ATTGCACGTTCGTGATACAAAAATTGAGGTAGCCCAGTTCGTGAAGGACTTACTGTTACATTTGAAAAAGT
TATTCCGTGAAGGGCAATTTAACtaataaGGTACCCTCGAGTCTGGTAAAGAAACCGCTGCTGCGAAATTT
GAACGCCAGCACATGGACTCGTCTACTAGCGCAGCTTAATTAACCTAGGCTGCTGCCACCGCTGAGCAATA
ACTAGCATAACCCCTTGGGGCCTCTAAACGGGTCTTGAGGGGTTTTTTGCTGAAAGGAGGAACTATATCCG
GATTGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCAACCCTATCTCG
GTCTATTCTTTTGATTTATAAGGGATTTTGCCGATTTCGGCCTATTGGTTAAAAAATGAGCTGATTTAACA
AAAATTTAACGCGAATTTTAACAAAATATTAACGTTTACAATTTCTGGCGGCACGATGGCATGAGATTATC
AAAAAGGATCTTCACCTAGATCCTTTTAAATTAAAAATGAAGTTTTAAATCAATCTAAAGTATATATGAGT
AAACTTGGTCTGACAGTTACCAATGCTTAATCAGTGAGGCACCTATCTCAGCGATCTGTCTATTTCGTTCA
TCCATAGTTGCCTGACTCCCCGTCGTGTAGATAACTACGATACGGGAGGGCTTACCATCTGGCCCCAGTGC
TGCAATGATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGTGGTGTCACGCTC
GTCGTTTGGTATGGCTTCATTCAGCTCCGGTTCCCAACGATCAAGGCGAGTTACATGATCCCCCATGTTGT
GCAAAAAAGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGACTGGTGA
GTACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGAATAAGGG
CGACACGGAAATGTTGAATACTCATACTCTTCCTTTTTCAATCATGATTGAAGCATTTATCAGGGTTATTG
TCTCATGAGCGGATACATATTTGAATGTATTTAGAAAAATAAACAAATAGGTCATGACCAAAATCCCTTAA
CGTGAGTTTTCGTTCCACTGAGCGTCAGACCCCGTAGAAAAGATCAAAGGATCTTCTTGAGATCCTTTTTT
TCTGCGCGTAATCTGCTGCTTGCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATACCAAATACTGTCCTTCTAGTG
TAGCCGTAGTTAGGCCACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACGACCTACACCGAA
CTGAGATACCTACAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCTGATACCGCTCGCCGCA
GCCGAACGACCGAGCGCAGCGAGTCAGTGAGCGAGGAAGCGGAAGAGCGCCTGATGCGGTATTTTCTCCTT
ACGCATCTGTGCGGTATTTCACACCGCATATATGGTGCACTCTCAGTACAATCTGCTCTGATGCCGCATAG
TTAAGCCAGTATACACTCCGCTATCGCTACGTGACTGGGTCATGGCTGCGCCCCGACACCCGCCAACACCC
GCTGACGCGCCCTGACGGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGGCAGCTGCGGTAAAGCTCATCAGCGT
GGTCGTGAAGCGATTCACAGATGTCTGCCTGTTCATCCGCGTCCAGCTCGTTGAGTTTCTCCAGAAGCGTT
AATGTCTGGCTTCTGATAAAGCGGGCCATGTTAAGGGCGGTTTTTTCCTGTTTGGTCACTGATGCCTCCGT
GTAAGGGGGATTTCTGTTCATGGGGGTAATGATACCGATGAAACGAGAGAGGATGCTCACGATACGGGTTA
CTGATGATGAACATGCCCGGTTACTGGAACGTTGTGAGGGTAAACAACTGGCGGTATGGATGCGGCGGGAC
CAGAGAAAAATCACTCAGGGTCAATGCCAGCGCTTCGTTAATACAGATGTAGGTGTTCCACAGGGTAGCCA
GCAGCATCCTGCGATGCAGATCCGGAACATAATGGTGCAGGGCGCTGACTTCCGCGTTTCCAGACTTTACG
AAACACGGAAACCGAAGACCATTCATGTTGTTGCTCAGGTCGCAGACGTTTTGCAGCAGCAGTCGCTTCAC
GTTCGCTCGCGTATCGGTGATTCATTCTGCTAACCAGTAAGGCAACCCCGCCAGCCTAGCCGGGTCCTCAA
CGACAGGAGCACGATCATGCTAGTCATGCCCCGCGCCCACCGGAAGGAGCTGACTGGGTTGAAGGCTCTCA
AGGGCATCGGTCGAGATCCCGGTGCCTAATGAGTGAGCTAACTTACATTAATTGCGTTGCGCTCACTGCCC
GCTTTCCAGTCGGGAAACCTGTCGTGCCAGCTGCATTAATGAATCGGCCAACGCGCGGGGAGAGGCGGTTT
GCGTATTGGGCGCCAGGGTGGTTTTTCTTTTCACCAGTGAGACGGGCAACAGCTGATTGCCCTTCACCGCC
TGGCCCTGAGAGAGTTGCAGCAAGCGGTCCACGCTGGTTTGCCCCAGCAGGCGAAAATCCTGTTTGATGGT
GGTTAACGGCGGGATATAACATGAGCTGTCTTCGGTATCGTCGTATCCCACTACCGAGATGTCCGCACCAA
CGCGCAGCCCGGACTCGGTAATGGCGCGCATTGCGCCCAGCGCCATCTGATCGTTGGCAACCAGCATCGCA
GTGGGAACGATGCCCTCATTCAGCATTTGCATGGTTTGTTGAAAACCGGACATGGCACTCCAGTCGCCTTC
CCGTTCCGCTATCGGCTGAATTTGATTGCGAGTGAGATATTTATGCCAGCCAGCCAGACGCAGACGCGCCG
AGACAGAACTTAATGGGCCCGCTAACAGCGCGATTTGCTGGTGACCCAATGCGACCAGATGCTCCACGCCC
AGTCGCGTACCGTCTTCATGGGAGAAAATAATACTGTTGATGGGTGTCTGGTCAGAGACATCAAGAAATAA
CGCCGGAACATTAGTGCAGGCAGCTTCCACAGCAATGGCATCCTGGTCATCCAGCGGATAGTTAATGATCA
GCCCACTGACGCGTTGCGCGAGAAGATTGTGCACCGCCGCTTTACAGGCTTCGACGCCGCTTCGTTCTACC
ATCGACACCACCACGCTGGCACCCAGTTGATCGGCGCGAGATTTAATCGCCGCGACAATTTGCGACGGCGC
GTGCAGGGCCAGACTGGAGGTGGCAACGCCAATCAGCAACGACTGTTTGCCCGCCAGTTGTTGTGCCACGC
GGTTGGGAATGTAATTCAGCTCCGCCATCGCCGCTTCCACTTTTTCCCGCGTTTTCGCAGAAACGTGGCTG
GCCTGGTTCACCACGCGGGAAACGGTCTGATAAGAGACACCGGCATACTCTGCGACATCGTATAACGTTAC
TGGTTTCACATTCACCACCCTGAATTGACTCTCTTCCGGGCGCTATCATGCCATACCGCGAAAGGTTTTGC
GCCATTCGATGGTGTCCGGGATCTCGACGCTCTCCCTTATGCGACTCCTGCATTAGGAAGCAGCCCAGTAG
TAGGTTGAGGCCGTTGAGCACCGCCGCCGCAAGGAATGGTGCATGCAAGGAGATGGCGCCCAACAGTCCCC
CGGCCACGGGGCCTGCCACCATACCCACGCCGAAACAAGCGCTCATGAGCCCGAAGTGGCGAGCCCGATCT
TCCCCATCGGTGATGTCGGCGATATAGGCGCCAGCAACCGCACCTGTGGCGCCGGTGATGCCGGCCACGAT
GCGTCCGGCGTAGAGGATCGAGATCGATCTCGATCCCGCGAAATTAATACGACTCACTATA
SEQ ID NO: 4
>pETDuet-1 C7 linker TEV IL33 70051 (6305 bp)
GGGGAATTGTGAGCGGATAACAATTCCCCTCTAGAAATAATTTTGTTTAACTTTAAGAAGGAGATATACCA
TGGGCAGCAGCCATCACCATCATCACCACAGCCAGGATCCGAATTCGAGCTCGGCGCGCCTGCAGGTCGAC
AAGCTTGCGGCCGCATAATGCTTAAGTCGAACAGAAAGTAATCGTATTGTACACGGCCGCATAATCGAAAT
TAATACGACTCACTATAGGGGAATTGTGAGCGGATAACAATTCCCCATCTTAGTATATTAGTTAAGTATAA
GAAGGAGATATACATATGGAGAGTAAACGGAATAAGCCAGGGAAGGCAACAGGTAAAGGAAAACCTGTCAA
TAATAAGTGGTTAAATAATGCAGGTAAAGACTTAGGTTCTCCTGTTCCAGATCGTATAGCTAATAAACTAC
GTGATAAGGAGTTTAAAAGTTTCGATGATTTTCGTAAGAAATTCTGGGAAGAAGTGTCAAAAGATCCTGAG
TTAAGTAAACAATTTAGTCGAAACAATAATGATCGAATGAAGGTTGGAAAAGCGCCCAAGACTAGAACCCA
GGATGTTTCAGGGAAGGCAACTTCATTCGCACTTCATCATGAGAAGCCGATCAGCCAAAATGGTGGTGTCT
ATGATATGGATAACATCAGCGTGGTAACACCTAAACGTGCTATTGATATTCACCGAGGTAAAAGCGGAGGT
GGCTCATCAGGATCtGGTGAAAACCTGTATTTTCAGGGatccAgcattcagggcaccagcctgctgaccca
gagcccggcgagcctgagcacctataacgatcagagcgtgagctttgtgctggaaaacggctgctatgtga
ttaacgtggataaaagcggcaaagatcaggaacaggatcaggtgctgctgcgctattataaaagcccgtgc
ccggcgagccagagcggcgatggcgtggatggcaagaaactgatggtgaacatgagcccgattaaagatac
cgatatttggctgcatgcgaacgataaagattatagcgtggaactgcagcgcggcgatgtgagcccgccag
aacaggcgttctttgtgctgcataagaaaagcagcgattttgtgagctttgaatgcaaaaacctgccgggc
acctatattggcgtgaaagataaccagctggcgctggtggaagagaaagatgaaagctgcaacaacattat
gtttaaactgagcaaaatttaataaGGTACCCTCGAGTCTGGTAAAGAAACCGCTGCTGCGAAATTTGAAC
GCCAGCACATGGACTCGTCTACTAGCGCAGCTTAATTAACCTAGGCTGCTGCCACCGCTGAGCAATAACTA
GCATAACCCCTTGGGGCCTCTAAACGGGTCTTGAGGGGTTTTTTGCTGAAAGGAGGAACTATATCCGGATT
GGCGAATGGGACGCGCCCTGTAGCGGCGCATTAAGCGCGGCGGGTGTGGTGGTTACGCGCAGCGTGACCGC
TACACTTGCCAGCGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGGCACCTCGACCCC
AAAAAACTTGATTAGGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAACCCTATCTCGGTCT
ATTCTTTTGATTTATAAGGGATTTTGCCGATTTCGGCCTATTGGTTAAAAAATGAGCTGATTTAACAAAAA
TTTAACGCGAATTTTAACAAAATATTAACGTTTACAATTTCTGGCGGCACGATGGCATGAGATTATCAAAA
AGGATCTTCACCTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATTTCGTTCATCCA
TAGTTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTGGTGAGTAC
TCAACCAAGTCATTCTGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGGGCGAC
ACGGAAATGTTGAATACTCATACTCTTCCTTTTTCAATCATGATTGAAGCATTTATCAGGGTTATTGTCTC
ATGAGCGGATACATATTTGAATGTATTTAGAAAAATAAACAAATAGGTCATGACCAAAATCCCTTAACGTG
AGTTTTCGTTCCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGTCCTTCTAGTGTAGC
CGTAGTTAGGCCACCACTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCACATGTTCTTTCCT
GCGTTATCCCCTGATTCTGTGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCTGATGCGGTATTTTCTCCTTACGC
ATCTGTGCGGTATTTCACACCGCATATATGGTGCACTCTCAGTACAATCTGCTCTGATGCCGCATAGTTAA
GCCAGTATACACTCCGCTATCGCTACGTGACTGGGTCATGGCTGCGCCCCGACACCCGCCAACACCCGCTG
ACGCGCCCTGACGGGCTTGTCTGCTCCCGGCATCCGCTTACAGACAAGCTGTGACCGTCTCCGGGAGCTGC
ATGTGTCAGAGGTTTTCACCGTCATCACCGAAACGCGCGAGGCAGCTGCGGTAAAGCTCATCAGCGTGGTC
GTGAAGCGATTCACAGATGTCTGCCTGTTCATCCGCGTCCAGCTCGTTGAGTTTCTCCAGAAGCGTTAATG
TCTGGCTTCTGATAAAGCGGGCCATGTTAAGGGCGGTTTTTTCCTGTTTGGTCACTGATGCCTCCGTGTAA
GGGGGATTTCTGTTCATGGGGGTAATGATACCGATGAAACGAGAGAGGATGCTCACGATACGGGTTACTGA
TGATGAACATGCCCGGTTACTGGAACGTTGTGAGGGTAAACAACTGGCGGTATGGATGCGGCGGGACCAGA
GAAAAATCACTCAGGGTCAATGCCAGCGCTTCGTTAATACAGATGTAGGTGTTCCACAGGGTAGCCAGCAG
CATCCTGCGATGCAGATCCGGAACATAATGGTGCAGGGCGCTGACTTCCGCGTTTCCAGACTTTACGAAAC
ACGGAAACCGAAGACCATTCATGTTGTTGCTCAGGTCGCAGACGTTTTGCAGCAGCAGTCGCTTCACGTTC
GCTCGCGTATCGGTGATTCATTCTGCTAACCAGTAAGGCAACCCCGCCAGCCTAGCCGGGTCCTCAACGAC
AGGAGCACGATCATGCTAGTCATGCCCCGCGCCCACCGGAAGGAGCTGACTGGGTTGAAGGCTCTCAAGGG
CATCGGTCGAGATCCCGGTGCCTAATGAGTGAGCTAACTTACATTAATTGCGTTGCGCTCACTGCCCGCTT
TCCAGTCGGGAAACCTGTCGTGCCAGCTGCATTAATGAATCGGCCAACGCGCGGGGAGAGGCGGTTTGCGT
ATTGGGCGCCAGGGTGGTTTTTCTTTTCACCAGTGAGACGGGCAACAGCTGATTGCCCTTCACCGCCTGGC
CCTGAGAGAGTTGCAGCAAGCGGTCCACGCTGGTTTGCCCCAGCAGGCGAAAATCCTGTTTGATGGTGGTT
AACGGCGGGATATAACATGAGCTGTCTTCGGTATCGTCGTATCCCACTACCGAGATGTCCGCACCAACGCG
CAGCCCGGACTCGGTAATGGCGCGCATTGCGCCCAGCGCCATCTGATCGTTGGCAACCAGCATCGCAGTGG
GAACGATGCCCTCATTCAGCATTTGCATGGTTTGTTGAAAACCGGACATGGCACTCCAGTCGCCTTCCCGT
TCCGCTATCGGCTGAATTTGATTGCGAGTGAGATATTTATGCCAGCCAGCCAGACGCAGACGCGCCGAGAC
AGAACTTAATGGGCCCGCTAACAGCGCGATTTGCTGGTGACCCAATGCGACCAGATGCTCCACGCCCAGTC
GCGTACCGTCTTCATGGGAGAAAATAATACTGTTGATGGGTGTCTGGTCAGAGACATCAAGAAATAACGCC
GGAACATTAGTGCAGGCAGCTTCCACAGCAATGGCATCCTGGTCATCCAGCGGATAGTTAATGATCAGCCC
ACTGACGCGTTGCGCGAGAAGATTGTGCACCGCCGCTTTACAGGCTTCGACGCCGCTTCGTTCTACCATCG
ACACCACCACGCTGGCACCCAGTTGATCGGCGCGAGATTTAATCGCCGCGACAATTTGCGACGGCGCGTGC
AGGGCCAGACTGGAGGTGGCAACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTTTCGCAGAAACGTGGCTGGCCT
GGTTCACCACGCGGGAAACGGTCTGATAAGAGACACCGGCATACTCTGCGACATCGTATAACGTTACTGGT
TTCACATTCACCACCCTGAATTGACTCTCTTCCGGGCGCTATCATGCCATACCGCGAAAGGTTTTGCGCCA
TTCGATGGTGTCCGGGATCTCGACGCTCTCCCTTATGCGACTCCTGCATTAGGAAGCAGCCCAGTAGTAGG
TTGAGGCCGTTGAGCACCGCCGCCGCAAGGAATGGTGCATGCAAGGAGATGGCGCCCAACAGTCCCCCGGC
CACGGGGCCTGCCACCATACCCACGCCGAAACAAGCGCTCATGAGCCCGAAGTGGCGAGCCCGATCTTCCC
CATCGGTGATGTCGGCGATATAGGCGCCAGCAACCGCACCTGTGGCGCCGGTGATGCCGGCCACGATGCGT
CCGGCGTAGAGGATCGAGATCGATCTCGATCCCGCGAAATTAATACGACTCACTATA
SEQ ID NO: 5
>pETDuet-1 HCI F L 6His-C7 linker TEV IL13 70080 (6872 bp)
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacacctagtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCATAATGCTTAAGTCGAACAGAAAGTAATCGTATTGTACACGGCCGCATAATCGAAATTAATA
CGACTCACTATAGGGGAATTGTGAGCGGATAACAATTCCCCATCTTAGTATATTAGTTAAGTATAAGAAGG
AGATATACATATGGAGAGTAAACGGAATAAGCCAGGGAAGGCAACAGGTAAAGGAAAACCTGTCAATAATA
AGTGGTTAAATAATGCAGGTAAAGACTTAGGTTCTCCTGTTCCAGATCGTATAGCTAATAAACTACGTGAT
AAGGAGTTTAAAAGTTTCGATGATTTTCGTAAGAAATTCTGGGAAGAAGTGTCAAAAGATCCTGAGTTAAG
TAAACAATTTAGTCGAAACAATAATGATCGAATGAAGGTTGGAAAAGCGCCCAAGACTAGAACCCAGGATG
TTTCAGGGAAGGCAACTTCATTCGCACTTCATCATGAGAAGCCGATCAGCCAAAATGGTGGTGTCTATGAT
ATGGATAACATCAGCGTGGTAACACCTAAACGTGCTATTGATATTCACCGAGGTAAAAGCGGAGGTGGCTC
ATCAGGATCtGGTGAAAACCTGTATTTTCAGGGatccGGCCCGGTGCCTCCAAGCACTGCCCTGCGTGAGT
TAATCGAAGAATTAGTCAACATTACCCAAAACCAGAAGGCACCGCTGTGCAACGGTTCAATGGTCTGGAGC
ATCAATCTGACAGCCGGTATGTATTGCGCGGCGCTTGAAAGCCTGATTAACGTGAGTGGTTGTTCTGCCAT
CGAAAAAACTCAACGTATGTTGTCCGGCTTTTGCCCACACAAGGTATCTGCCGGTCAATTCAGTTCATTGC
ACGTTCGTGATACAAAAATTGAGGTAGCCCAGTTCGTGAAGGACTTACTGTTACATTTGAAAAAGTTATTC
CGTGAAGGGCAATTTAACtaataaGGTACCCTCGAGTCTGGTAAAGAAACCGCTGCTGCGAAATTTGAACG
CCAGCACATGGACTCGTCTACTAGCGCAGCTTAATTAACCTAGGCTGCTGCCACCGCTGAGCAATAACTAG
CATAACCCCTTGGGGCCTCTAAACGGGTCTTGAGGGGTTTTTTGCTGAAAGGAGGAACTATATCCGGATTG
GCGAATGGGACGCGCCCTGTAGCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAACCCTATCTCGGTCTA
TTCTTTTGATTTATAAGGGATTTTGCCGATTTCGGCCTATTGGTTAAAAAATGAGCTGATTTAACAAAAAT
TTAACGCGAATTTTAACAAAATATTAACGTTTACAATTTCTGGCGGCACGATGGCATGAGATTATCAAAAA
GGATCTTCACCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATCATGATTGAAGCATTTATCAGGGTTATTGTCTCA
TGAGCGGATACATATTTGAATGTATTTAGAAAAATAAACAAATAGGTCATGACCAAAATCCCTTAACGTGA
GTTTTCGTTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAGCCTATGGAA
AAACGCCAGCAACGCGGCCTTTTTACGGTTCCTGGCCTTTTGCTGGCCTTTTGCTCACATGTTCTTTCCTG
CGTTATCCCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCTGATGCGGTATTTTCTCCTTACGCA
TCTGTGCGGTATTTCACACCGCATATATGGTGCACTCTCAGTACAATCTGCTCTGATGCCGCATAGTTAAG
CCAGTATACACTCCGCTATCGCTACGTGACTGGGTCATGGCTGCGCCCCGACACCCGCCAACACCCGCTGA
CGCGCCCTGACGGGCTTGTCTGCTCCCGGCATCCGCTTACAGACAAGCTGTGACCGTCTCCGGGAGCTGCA
TGTGTCAGAGGTTTTCACCGTCATCACCGAAACGCGCGAGGCAGCTGCGGTAAAGCTCATCAGCGTGGTCG
TGAAGCGATTCACAGATGTCTGCCTGTTCATCCGCGTCCAGCTCGTTGAGTTTCTCCAGAAGCGTTAATGT
CTGGCTTCTGATAAAGCGGGCCATGTTAAGGGCGGTTTTTTCCTGTTTGGTCACTGATGCCTCCGTGTAAG
GGGGATTTCTGTTCATGGGGGTAATGATACCGATGAAACGAGAGAGGATGCTCACGATACGGGTTACTGAT
GATGAACATGCCCGGTTACTGGAACGTTGTGAGGGTAAACAACTGGCGGTATGGATGCGGCGGGACCAGAG
AAAAATCACTCAGGGTCAATGCCAGCGCTTCGTTAATACAGATGTAGGTGTTCCACAGGGTAGCCAGCAGC
ATCCTGCGATGCAGATCCGGAACATAATGGTGCAGGGCGCTGACTTCCGCGTTTCCAGACTTTACGAAACA
CGGAAACCGAAGACCATTCATGTTGTTGCTCAGGTCGCAGACGTTTTGCAGCAGCAGTCGCTTCACGTTCG
CTCGCGTATCGGTGATTCATTCTGCTAACCAGTAAGGCAACCCCGCCAGCCTAGCCGGGTCCTCAACGACA
GGAGCACGATCATGCTAGTCATGCCCCGCGCCCACCGGAAGGAGCTGACTGGGTTGAAGGCTCTCAAGGGC
ATCGGTCGAGATCCCGGTGCCTAATGAGTGAGCTAACTTACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGTA
TTGGGCGCCAGGGTGGTTTTTCTTTTCACCAGTGAGACGGGCAACAGCTGATTGCCCTTCACCGCCTGGCC
CTGAGAGAGTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATGTCCGCACCAACGCGC
AGCCCGGACTCGGTAATGGCGCGCATTGCGCCCAGCGCCATCTGATCGTTGGCAACCAGCATCGCAGTGGG
AACGATGCCCTCATTCAGCATTTGCATGGTTTGTTGAAAACCGGACATGGCACTCCAGTCGCCTTCCCGTT
CCGCTATCGGCTGAATTTGATTGCGAGTGAGATATTTATGCCAGCCAGCCAGACGCAGACGCGCCGAGACA
GAACTTAATGGGCCCGCTAACAGCGCGATTTGCTGGTGACCCAATGCGACCAGATGCTCCACGCCCAGTCG
CGTACCGTCTTCATGGGAGAAAATAATACTGTTGATGGGTGTCTGGTCAGAGACATCAAGAAATAACGCCG
GAACATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGGCGCGTGCA
GGGCCAGACTGGAGGTGGCAACGCCAATCAGCAACGACTGTTTGCCCGCCAGTTGTTGTGCCACGCGGTTG
GGAATGTAATTCAGCTCCGCCATCGCCGCTTCCACTTTTTCCCGCGTTTTCGCAGAAACGTGGCTGGCCTG
GTTCACCACGCGGGAAACGGTCTGATAAGAGACACCGGCATACTCTGCGACATCGTATAACGTTACTGGTT
TCACATTCACCACCCTGAATTGACTCTCTTCCGGGCGCTATCATGCCATACCGCGAAAGGTTTTGCGCCAT
TCGATGGTGTCCGGGATCTCGACGCTCTCCCTTATGCGACTCCTGCATTAGGAAGCAGCCCAGTAGTAGGT
TGAGGCCGTTGAGCACCGCCGCCGCAAGGAATGGTGCATGCAAGGAGATGGCGCCCAACAGTCCCCCGGCC
ACGGGGCCTGCCACCATACCCACGCCGAAACAAGCGCTCATGAGCCCGAAGTGGCGAGCCCGATCTTCCCC
ATCGGTGATGTCGGCGATATAGGCGCCAGCAACCGCACCTGTGGCGCCGGTGATGCCGGCCACGATGCGTC
CGGCGTAGAGGATCGAGATCGATCTCGATCCCGCGAAATTAATACGACTCACTATA
SEQ ID NO: 6
>pETDuet-1 HcI F L 6His-C7 linker TEV IL33 70059 (7010 bp)
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacacctagtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCATAATGCTTAAGTCGAACAGAAAGTAATCGTATTGTACACGGCCGCATAATCGAAATTAATA
CGACTCACTATAGGGGAATTGTGAGCGGATAACAATTCCCCATCTTAGTATATTAGTTAAGTATAAGAAGG
AGATATACATATGGAGAGTAAACGGAATAAGCCAGGGAAGGCAACAGGTAAAGGAAAACCTGTCAATAATA
AGTGGTTAAATAATGCAGGTAAAGACTTAGGTTCTCCTGTTCCAGATCGTATAGCTAATAAACTACGTGAT
AAGGAGTTTAAAAGTTTCGATGATTTTCGTAAGAAATTCTGGGAAGAAGTGTCAAAAGATCCTGAGTTAAG
TAAACAATTTAGTCGAAACAATAATGATCGAATGAAGGTTGGAAAAGCGCCCAAGACTAGAACCCAGGATG
TTTCAGGGAAGGCAACTTCATTCGCACTTCATCATGAGAAGCCGATCAGCCAAAATGGTGGTGTCTATGAT
ATGGATAACATCAGCGTGGTAACACCTAAACGTGCTATTGATATTCACCGAGGTAAAAGCGGAGGTGGCTC
ATCAGGATCtGGTGAAAACCTGTATTTTCAGGGatccAgcattcagggcaccagcctgctgacccagagcc
cggcgagcctgagcacctataacgatcagagcgtgagctttgtgctggaaaacggctgctatgtgattaac
gtggataaaagcggcaaagatcaggaacaggatcaggtgctgctgcgctattataaaagcccgtgcccggc
gagccagagcggcgatggcgtggatggcaagaaactgatggtgaacatgagcccgattaaagataccgata
tttggctgcatgcgaacgataaagattatagcgtggaactgcagcgcggcgatgtgagcccgccagaacag
gcgttctttgtgctgcataagaaaagcagcgattttgtgagctttgaatgcaaaaacctgccgggcaccta
tattggcgtgaaagataaccagctggcgctggtggaagagaaagatgaaagctgcaacaacattatgttta
aactgagcaaaatttaataaGGTACCCTCGAGTCTGGTAAAGAAACCGCTGCTGCGAAATTTGAACGCCAG
CACATGGACTCGTCTACTAGCGCAGCTTAATTAACCTAGGCTGCTGCCACCGCTGAGCAATAACTAGCATA
ACCCCTTGGGGCCTCTAAACGGGTCTTGAGGGGTTTTTTGCTGAAAGGAGGAACTATATCCGGATTGGCGA
ATGGGACGCGCCCTGTAGCGGCGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTCGCCACGTTCGCCGGCTTTCCC
CGTCAAGCTCTAAATCGGGGGCTCCCTTTAGGGTTCCGATTTAGTGCTTTACGGCACCTCGACCCCAAAAA
ACTTGATTAGGGTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAA
CGCGAATTTTAACAAAATATTAACGTTTACAATTTCTGGCGGCACGATGGCATGAGATTATCAAAAAGGAT
CTTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGGCGTCAATACGGGATAATACCG
CGCCACATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGAATAAGGGCGACACGGA
AATGTTGAATACTCATACTCTTCCTTTTTCAATCATGATTGAAGCATTTATCAGGGTTATTGTCTCATGAG
CGGATACATATTTGAATGTATTTAGAAAAATAAACAAATAGGTCATGACCAAAATCCCTTAACGTGAGTTT
TCGTTCCACTGAGCGTCAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCTAGTGTAGCCGTAG
TTAGGCCACCACTTCAAGAACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGCTCACATGTTCTTTCCTGCGTT
ATCCCCTGATTCTGTGGATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATTTTCTCCTTACGCATCTG
TGCGGTATTTCACACCGCATATATGGTGCACTCTCAGTACAATCTGCTCTGATGCCGCATAGTTAAGCCAG
TATACACTCCGCTATCGCTACGTGACTGGGTCATGGCTGCGCCCCGACACCCGCCAACACCCGCTGACGCG
CCCTGACGGGCTTGTCTGCTCCCGGCATCCGCTTACAGACAAGCTGTGACCGTCTCCGGGAGCTGCATGTG
TCAGAGGTTTTCACCGTCATCACCGAAACGCGCGAGGCAGCTGCGGTAAAGCTCATCAGCGTGGTCGTGAA
GCGATTCACAGATGTCTGCCTGTTCATCCGCGTCCAGCTCGTTGAGTTTCTCCAGAAGCGTTAATGTCTGG
CTTCTGATAAAGCGGGCCATGTTAAGGGCGGTTTTTTCCTGTTTGGTCACTGATGCCTCCGTGTAAGGGGG
ATTTCTGTTCATGGGGGTAATGATACCGATGAAACGAGAGAGGATGCTCACGATACGGGTTACTGATGATG
AACATGCCCGGTTACTGGAACGTTGTGAGGGTAAACAACTGGCGGTATGGATGCGGCGGGACCAGAGAAAA
ATCACTCAGGGTCAATGCCAGCGCTTCGTTAATACAGATGTAGGTGTTCCACAGGGTAGCCAGCAGCATCC
TGCGATGCAGATCCGGAACATAATGGTGCAGGGCGCTGACTTCCGCGTTTCCAGACTTTACGAAACACGGA
AACCGAAGACCATTCATGTTGTTGCTCAGGTCGCAGACGTTTTGCAGCAGCAGTCGCTTCACGTTCGCTCG
CGTATCGGTGATTCATTCTGCTAACCAGTAAGGCAACCCCGCCAGCCTAGCCGGGTCCTCAACGACAGGAG
CACGATCATGCTAGTCATGCCCCGCGCCCACCGGAAGGAGCTGACTGGGTTGAAGGCTCTCAAGGGCATCG
GTCGAGATCCCGGTGCCTAATGAGTGAGCTAACTTACATTAATTGCGTTGCGCTCACTGCCCGCTTTCCAG
TCGGGAAACCTGTCGTGCCAGCTGCATTAATGAATCGGCCAACGCGCGGGGAGAGGCGGTTTGCGTATTGG
GCGCCAGGGTGGTTTTTCTTTTCACCAGTGAGACGGGCAACAGCTGATTGCCCTTCACCGCCTGGCCCTGA
GAGAGTTGCAGCAAGCGGTCCACGCTGGTTTGCCCCAGCAGGCGAAAATCCTGTTTGATGGTGGTTAACGG
CGGGATATAACATGAGCTGTCTTCGGTATCGTCGTATCCCACTACCGAGATGTCCGCACCAACGCGCAGCC
CGGACTCGGTAATGGCGCGCATTGCGCCCAGCGCCATCTGATCGTTGGCAACCAGCATCGCAGTGGGAACG
ATGCCCTCATTCAGCATTTGCATGGTTTGTTGAAAACCGGACATGGCACTCCAGTCGCCTTCCCGTTCCGC
TATCGGCTGAATTTGATTGCGAGTGAGATATTTATGCCAGCCAGCCAGACGCAGACGCGCCGAGACAGAAC
TTAATGGGCCCGCTAACAGCGCGATTTGCTGGTGACCCAATGCGACCAGATGCTCCACGCCCAGTCGCGTA
CCGTCTTCATGGGAGAAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAATGATCAGCCCACTGA
CGCGTTGCGCGAGAAGATTGTGCACCGCCGCTTTACAGGCTTCGACGCCGCTTCGTTCTACCATCGACACC
ACCACGCTGGCACCCAGTTGATCGGCGCGAGATTTAATCGCCGCGACAATTTGCGACGGCGCGTGCAGGGC
CAGACTGGAGGTGGCAACGCCAATCAGCAACGACTGTTTGCCCGCCAGTTGTTGTGCCACGCGGTTGGGAA
TGTAATTCAGCTCCGCCATCGCCGCTTCCACTTTTTCCCGCGTTTTCGCAGAAACGTGGCTGGCCTGGTTC
ACCACGCGGGAAACGGTCTGATAAGAGACACCGGCATACTCTGCGACATCGTATAACGTTACTGGTTTCAC
ATTCACCACCCTGAATTGACTCTCTTCCGGGCGCTATCATGCCATACCGCGAAAGGTTTTGCGCCATTCGA
TGGTGTCCGGGATCTCGACGCTCTCCCTTATGCGACTCCTGCATTAGGAAGCAGCCCAGTAGTAGGTTGAG
GCCGTTGAGCACCGCCGCCGCAAGGAATGGTGCATGCAAGGAGATGGCGCCCAACAGTCCCCCGGCCACGG
GGCCTGCCACCATACCCACGCCGAAACAAGCGCTCATGAGCCCGAAGTGGCGAGCCCGATCTTCCCCATCG
GTGATGTCGGCGATATAGGCGCCAGCAACCGCACCTGTGGCGCCGGTGATGCCGGCCACGATGCGTCCGGC
GTAGAGGATCGAGATCGATCTCGATCCCGCGAAATTAATACGACTCACTATA
SEQ ID NO: 7
>pETDuet-1 HCI F L 6His-C7 linker TEV IL33 with mutated T7 70126
(7010 bp)
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacacctagtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCATAATGCTTAAGTCGAACAGAAAGTAATCGTATTGTACACGGCCGCATAATCGAAATTAATA
CGcCTCACTATAGGGGAATTGTGAGCGGATAACAATTCCCCATCTTAGTATATTAGTTAAGTATAAGAAGG
AGATATACATATGGAGAGTAAACGGAATAAGCCAGGGAAGGCAACAGGTAAAGGAAAACCTGTCAATAATA
AGTGGTTAAATAATGCAGGTAAAGACTTAGGTTCTCCTGTTCCAGATCGTATAGCTAATAAACTACGTGAT
AAGGAGTTTAAAAGTTTCGATGATTTTCGTAAGAAATTCTGGGAAGAAGTGTCAAAAGATCCTGAGTTAAG
TAAACAATTTAGTCGAAACAATAATGATCGAATGAAGGTTGGAAAAGCGCCCAAGACTAGAACCCAGGATG
TTTCAGGGAAGGCAACTTCATTCGCACTTCATCATGAGAAGCCGATCAGCCAAAATGGTGGTGTCTATGAT
ATGGATAACATCAGCGTGGTAACACCTAAACGTGCTATTGATATTCACCGAGGTAAAAGCGGAGGTGGCTC
ATCAGGATCtGGTGAAAACCTGTATTTTCAGGGatccAgcattcagggcaccagcctgctgacccagagcc
cggcgagcctgagcacctataacgatcagagcgtgagctttgtgctggaaaacggctgctatgtgattaac
gtggataaaagcggcaaagatcaggaacaggatcaggtgctgctgcgctattataaaagcccgtgcccggc
gagccagagcggcgatggcgtggatggcaagaaactgatggtgaacatgagcccgattaaagataccgata
tttggctgcatgcgaacgataaagattatagcgtggaactgcagcgcggcgatgtgagcccgccagaacag
gcgttctttgtgctgcataagaaaagcagcgattttgtgagctttgaatgcaaaaacctgccgggcaccta
tattggcgtgaaagataaccagctggcgctggtggaagagaaagatgaaagctgcaacaacattatgttta
aactgagcaaaatttaataaGGTACCCTCGAGTCTGGTAAAGAAACCGCTGCTGCGAAATTTGAACGCCAG
CACATGGACTCGTCTACTAGCGCAGCTTAATTAACCTAGGCTGCTGCCACCGCTGAGCAATAACTAGCATA
ACCCCTTGGGGCCTCTAAACGGGTCTTGAGGGGTTTTTTGCTGAAAGGAGGAACTATATCCGGATTGGCGA
ATGGGACGCGCCCTGTAGCGGCGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTCGCCACGTTCGCCGGCTTTCCC
CGTCAAGCTCTAAATCGGGGGCTCCCTTTAGGGTTCCGATTTAGTGCTTTACGGCACCTCGACCCCAAAAA
ACTTGATTAGGGTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAA
CGCGAATTTTAACAAAATATTAACGTTTACAATTTCTGGCGGCACGATGGCATGAGATTATCAAAAAGGAT
CTTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGGCGTCAATACGGGATAATACCG
CGCCACATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGAATAAGGGCGACACGGA
AATGTTGAATACTCATACTCTTCCTTTTTCAATCATGATTGAAGCATTTATCAGGGTTATTGTCTCATGAG
CGGATACATATTTGAATGTATTTAGAAAAATAAACAAATAGGTCATGACCAAAATCCCTTAACGTGAGTTT
TCGTTCCACTGAGCGTCAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCTAGTGTAGCCGTAG
TTAGGCCACCACTTCAAGAACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGCTCACATGTTCTTTCCTGCGTT
ATCCCCTGATTCTGTGGATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATTTTCTCCTTACGCATCTG
TGCGGTATTTCACACCGCATATATGGTGCACTCTCAGTACAATCTGCTCTGATGCCGCATAGTTAAGCCAG
TATACACTCCGCTATCGCTACGTGACTGGGTCATGGCTGCGCCCCGACACCCGCCAACACCCGCTGACGCG
CCCTGACGGGCTTGTCTGCTCCCGGCATCCGCTTACAGACAAGCTGTGACCGTCTCCGGGAGCTGCATGTG
TCAGAGGTTTTCACCGTCATCACCGAAACGCGCGAGGCAGCTGCGGTAAAGCTCATCAGCGTGGTCGTGAA
GCGATTCACAGATGTCTGCCTGTTCATCCGCGTCCAGCTCGTTGAGTTTCTCCAGAAGCGTTAATGTCTGG
CTTCTGATAAAGCGGGCCATGTTAAGGGCGGTTTTTTCCTGTTTGGTCACTGATGCCTCCGTGTAAGGGGG
ATTTCTGTTCATGGGGGTAATGATACCGATGAAACGAGAGAGGATGCTCACGATACGGGTTACTGATGATG
AACATGCCCGGTTACTGGAACGTTGTGAGGGTAAACAACTGGCGGTATGGATGCGGCGGGACCAGAGAAAA
ATCACTCAGGGTCAATGCCAGCGCTTCGTTAATACAGATGTAGGTGTTCCACAGGGTAGCCAGCAGCATCC
TGCGATGCAGATCCGGAACATAATGGTGCAGGGCGCTGACTTCCGCGTTTCCAGACTTTACGAAACACGGA
AACCGAAGACCATTCATGTTGTTGCTCAGGTCGCAGACGTTTTGCAGCAGCAGTCGCTTCACGTTCGCTCG
CGTATCGGTGATTCATTCTGCTAACCAGTAAGGCAACCCCGCCAGCCTAGCCGGGTCCTCAACGACAGGAG
CACGATCATGCTAGTCATGCCCCGCGCCCACCGGAAGGAGCTGACTGGGTTGAAGGCTCTCAAGGGCATCG
GTCGAGATCCCGGTGCCTAATGAGTGAGCTAACTTACATTAATTGCGTTGCGCTCACTGCCCGCTTTCCAG
TCGGGAAACCTGTCGTGCCAGCTGCATTAATGAATCGGCCAACGCGCGGGGAGAGGCGGTTTGCGTATTGG
GCGCCAGGGTGGTTTTTCTTTTCACCAGTGAGACGGGCAACAGCTGATTGCCCTTCACCGCCTGGCCCTGA
GAGAGTTGCAGCAAGCGGTCCACGCTGGTTTGCCCCAGCAGGCGAAAATCCTGTTTGATGGTGGTTAACGG
CGGGATATAACATGAGCTGTCTTCGGTATCGTCGTATCCCACTACCGAGATGTCCGCACCAACGCGCAGCC
CGGACTCGGTAATGGCGCGCATTGCGCCCAGCGCCATCTGATCGTTGGCAACCAGCATCGCAGTGGGAACG
ATGCCCTCATTCAGCATTTGCATGGTTTGTTGAAAACCGGACATGGCACTCCAGTCGCCTTCCCGTTCCGC
TATCGGCTGAATTTGATTGCGAGTGAGATATTTATGCCAGCCAGCCAGACGCAGACGCGCCGAGACAGAAC
TTAATGGGCCCGCTAACAGCGCGATTTGCTGGTGACCCAATGCGACCAGATGCTCCACGCCCAGTCGCGTA
CCGTCTTCATGGGAGAAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAATGATCAGCCCACTGA
CGCGTTGCGCGAGAAGATTGTGCACCGCCGCTTTACAGGCTTCGACGCCGCTTCGTTCTACCATCGACACC
ACCACGCTGGCACCCAGTTGATCGGCGCGAGATTTAATCGCCGCGACAATTTGCGACGGCGCGTGCAGGGC
CAGACTGGAGGTGGCAACGCCAATCAGCAACGACTGTTTGCCCGCCAGTTGTTGTGCCACGCGGTTGGGAA
TGTAATTCAGCTCCGCCATCGCCGCTTCCACTTTTTCCCGCGTTTTCGCAGAAACGTGGCTGGCCTGGTTC
ACCACGCGGGAAACGGTCTGATAAGAGACACCGGCATACTCTGCGACATCGTATAACGTTACTGGTTTCAC
ATTCACCACCCTGAATTGACTCTCTTCCGGGCGCTATCATGCCATACCGCGAAAGGTTTTGCGCCATTCGA
TGGTGTCCGGGATCTCGACGCTCTCCCTTATGCGACTCCTGCATTAGGAAGCAGCCCAGTAGTAGGTTGAG
GCCGTTGAGCACCGCCGCCGCAAGGAATGGTGCATGCAAGGAGATGGCGCCCAACAGTCCCCCGGCCACGG
GGCCTGCCACCATACCCACGCCGAAACAAGCGCTCATGAGCCCGAAGTGGCGAGCCCGATCTTCCCCATCG
GTGATGTCGGCGATATAGGCGCCAGCAACCGCACCTGTGGCGCCGGTGATGCCGGCCACGATGCGTCCGGC
GTAGAGGATCGAGATCGATCTCGATCCCGCGAAATTAATACGACTCACTATA
SEQ ID NO: 8
>65750 pETDuet1 HCI F L 6His + Protein G Linker TEV C7 (7139 bp)
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacacctagtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCATAATGCTTAAGTCGAACAGAAAGTAATCGTATTGTACACGGCCGCATAATCGAAATTAATA
CGACTCACTATAGGGGAATTGTGAGCGGATAACAATTCCCCATCTTAGTATATTAGTTAAGTATAAGAAGG
AGATATACAtatqACCTATAAACTGATTCTGAACGGCAAAACGCTGAAAGGTGAAACCACCACCGAAGCAG
TGGATGCCGCAACCGCCGAAAAAGTTTTCAAACAGTACGCCAACGATAATGGCGTTGATGGTGAATGGACC
TACGATGATGCAACGAAAACCTTCACGGTGACCGAAAAACCGGAAGTTATTGATGCCAGCGAACTGACGCC
GGCAGTGACGACCTACAAACTGGTTATCAATGGCAAAACCCTGAAAGGTGAAACGACCACCGAAGCCGTGG
ATGCAGCCACCGCAGAAAAAGTGTTTAAACAGTACGCGAACGATAATGGCGTGGACGGCGAATGGACGTAT
GATGATGCCACCAAAACGTTTACCGTTACGGAAAAACCGGAAGTGATCGATGCATCTGAACTGACCCCGGC
GGTGACCACGTATAAACTGGTTATCAACGGTAAAACGCTGAAAGGCGAAACCACGACCAAAGCCGTGGATG
CAGAAACCGCGGAAAAAGCCTTTAAACAGTATGCAAACGATAATGGCGTGGATGGTGTTTGGACCTACGAT
GATGCCACGAAAACCTTCACGGTTACCGAAggcggtgggagtagcggaagtgggggtggtggatccagtGG
tTCTGGgGAAAAtCTGTACTTCCAGGGTGAGAGTAAACGGAATAAGCCAGGGAAGGCAACAGGTAAAGGAA
AACCTGTCAATAATAAGTGGTTAAATAATGCAGGTAAAGACTTAGGTTCTCCTGTTCCAGATCGTATAGCT
AATAAACTACGTGATAAGGAGTTTAAAAGTTTCGATGATTTTCGTAAGAAATTCTGGGAAGAAGTGTCAAA
AGATCCTGAGTTAAGTAAACAATTTAGTCGAAACAATAATGATCGAATGAAGGTTGGAAAAGCGCCCAAGA
CTAGAACCCAGGATGTTTCAGGGAAGGCAACTTCATTCGCACTTCATCATGAGAAGCCGATCAGCCAAAAT
GGTGGTGTCTATGATATGGATAACATCAGCGTGGTAACACCTAAACGTGCTATTGATATTCACCGAGGTAA
AAGCtgaggtacCCTCGAGTCTGGTAAAGAAACCGCTGCTGCGAAATTTGAACGCCAGCACATGGACTCGT
CTACTAGCGCAGCTTAATTAACCTAGGCTGCTGCCACCGCTGAGCAATAACTAGCATAACCCCTTGGGGCC
TCTAAACGGGTCTTGAGGGGTTTTTTGCTGAAAGGAGGAACTATATCCGGATTGGCGAATGGGACGCGCCC
TGTAGCGGCGCATTAAGCGCGGCGGGTGTGGTGGTTACGCGCAGCGTGACCGCTACACTTGCCAGCGCCCT
AGCGCCCGCTCCTTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTGGAGTCCACGTTCTT
TAATAGTGGACTCTTGTTCCAAACTGGAACAACACTCAACCCTATCTCGGTCTATTCTTTTGATTTATAAG
GGATTTTGCCGATTTCGGCCTATTGGTTAAAAAATGAGCTGATTTAACAAAAATTTAACGCGAATTTTAAC
AAAATATTAACGTTTACAATTTCTGGCGGCACGATGGCATGAGATTATCAAAAAGGATCTTCACCTAGATC
CTTTTAAATTAAAAATGAAGTTTTAAATCAATCTAAAGTATATATGAGTAAACTTGGTCTGACAGTTACCA
ATGCTTAATCAGTGAGGCACCTATCTCAGCGATCTGTCTATTTCGTTCATCCATAGTTGCCTGACTCCCCG
TCGTGTAGATAACTACGATACGGGAGGGCTTACCATCTGGCCCCAGTGCTGCAATGATACCGCGAGACCCA
CGCTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATGGCAGCACTGCATA
ATTCTCTTACTGTCATGCCATCCGTAAGATGCTTTTCTGTGACTGGTGAGTACTCAACCAAGTCATTCTGA
GAATAGTGTATGCGGCGACCGAGTTGCTCTTGCCCGGCGTCAATACGGGATAATACCGCGCCACATAGCAG
AACTTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTTGAATACT
CATACTCTTCCTTTTTCAATCATGATTGAAGCATTTATCAGGGTTATTGTCTCATGAGCGGATACATATTT
GAATGTATTTAGAAAAATAAACAAATAGGTCATGACCAAAATCCCTTAACGTGAGTTTTCGTTCCACTGAG
CGTCAGACCCCGTAGAAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTACCAACTCTTTTTCCGAA
GGTAACTGGCTTCAGCAGAGCGCAGATACCAAATACTGTCCTTCTAGTGTAGCCGTAGTTAGGCCACCACT
TCAAGAACTCTGTAGCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTATGGAAAAACGCCAGCAACGCGG
CCTTTTTACGGTTCCTGGCCTTTTGCTGGCCTTTTGCTCACATGTTCTTTCCTGCGTTATCCCCTGATTCT
GTGGATAACCGTATTACCGCCTTTGAGTGAGCTGATACCGCTCGCCGCAGCCGAACGACCGAGCGCAGCGA
GTCAGTGAGCGAGGAAGCGGAAGAGCGCCTGATGCGGTATTTTCTCCTTACGCATCTGTGCGGTATTTCAC
ACCGCATATATGGTGCACTCTCAGTACAATCTGCTCTGATGCCGCATAGTTAAGCCAGTATACACTCCGCT
ATCGCTACGTGACTGGGTCATGGCTGCGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGTGTCAGAGGTTTTCA
CCGTCATCACCGAAACGCGCGAGGCAGCTGCGGTAAAGCTCATCAGCGTGGTCGTGAAGCGATTCACAGAT
GTCTGCCTGTTCATCCGCGTCCAGCTCGTTGAGTTTCTCCAGAAGCGTTAATGTCTGGCTTCTGATAAAGC
GGGCCATGTTAAGGGCGGTTTTTTCCTGTTTGGTCACTGATGCCTCCGTGTAAGGGGGATTTCTGTTCATG
GGGGTAATGATACCGATGAAACGAGAGAGGATGCTCACGATACGGGTTACTGATGATGAACATGCCCGGTT
ACTGGAACGTTGTGAGGGTAAACAACTGGCGGTATGGATGCGGCGGGACCAGAGAAAAATCACTCAGGGTC
AATGCCAGCGCTTCGTTAATACAGATGTAGGTGTTCCACAGGGTAGCCAGCAGCATCCTGCGATGCAGATC
CGGAACATAATGGTGCAGGGCGCTGACTTCCGCGTTTCCAGACTTTACGAAACACGGAAACCGAAGACCAT
TCATGTTGTTGCTCAGGTCGCAGACGTTTTGCAGCAGCAGTCGCTTCACGTTCGCTCGCGTATCGGTGATT
CATTCTGCTAACCAGTAAGGCAACCCCGCCAGCCTAGCCGGGTCCTCAACGACAGGAGCACGATCATGCTA
GTCATGCCCCGCGCCCACCGGAAGGAGCTGACTGGGTTGAAGGCTCTCAAGGGCATCGGTCGAGATCCCGG
TGCCTAATGAGTGAGCTAACTTACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGCCAGGGTGGT
TTTTCTTTTCACCAGTGAGACGGGCAACAGCTGATTGCCCTTCACCGCCTGGCCCTGAGAGAGTTGCAGCA
AGCGGTCCACGCTGGTTTGCCCCAGCAGGCGAAAATCCTGTTTGATGGTGGTTAACGGCGGGATATAACAT
GAGCTGTCTTCGGTATCGTCGTATCCCACTACCGAGATGTCCGCACCAACGCGCAGCCCGGACTCGGTAAT
GGCGCGCATTGCGCCCAGCGCCATCTGATCGTTGGCAACCAGCATCGCAGTGGGAACGATGCCCTCATTCA
GCATTTGCATGGTTTGTTGAAAACCGGACATGGCACTCCAGTCGCCTTCCCGTTCCGCTATCGGCTGAATT
TGATTGCGAGTGAGATATTTATGCCAGCCAGCCAGACGCAGACGCGCCGAGACAGAACTTAATGGGCCCGC
TAACAGCGCGATTTGCTGGTGACCCAATGCGACCAGATGCTCCACGCCCAGTCGCGTACCGTCTTCATGGG
AGAAAATAATACTGTTGATGGGTGTCTGGTCAGAGACATCAAGAAATAACGCCGGAACATTAGTGCAGGCA
GCTTCCACAGCAATGGCATCCTGGTCATCCAGCGGATAGTTAATGATCAGCCCACTGACGCGTTGCGCGAG
AAGATTGTGCACCGCCGCTTTACAGGCTTCGACGCCGCTTCGTTCTACCATCGACACCACCACGCTGGCAC
CCAGTTGATCGGCGCGAGATTTAATCGCCGCGACAATTTGCGACGGCGCGTGCAGGGCCAGACTGGAGGTG
GCAACGCCAATCAGCAACGACTGTTTGCCCGCCAGTTGTTGTGCCACGCGGTTGGGAATGTAATTCAGCTC
CGCCATCGCCGCTTCCACTTTTTCCCGCGTTTTCGCAGAAACGTGGCTGGCCTGGTTCACCACGCGGGAAA
CGGTCTGATAAGAGACACCGGCATACTCTGCGACATCGTATAACGTTACTGGTTTCACATTCACCACCCTG
AATTGACTCTCTTCCGGGCGCTATCATGCCATACCGCGAAAGGTTTTGCGCCATTCGATGGTGTCCGGGAT
CTCGACGCTCTCCCTTATGCGACTCCTGCATTAGGAAGCAGCCCAGTAGTAGGTTGAGGCCGTTGAGCACC
GCCGCCGCAAGGAATGGTGCATGCAAGGAGATGGCGCCCAACAGTCCCCCGGCCACGGGGCCTGCCACCAT
ACCCACGCCGAAACAAGCGCTCATGAGCCCGAAGTGGCGAGCCCGATCTTCCCCATCGGTGATGTCGGCGA
TATAGGCGCCAGCAACCGCACCTGTGGCGCCGGTGATGCCGGCCACGATGCGTCCGGCGTAGAGGATCGAG
ATCGATCTCGATCCCGCGAAATTAATACGACTCACTATA
SEQ ID NO: 9
DU67808_pcDNA3D_FRT_SignalPeptide_ColE7_Spike_RBD_hexahis
gacggatcgggagatctcccgatcccctatggtgcactctcagtacaatctgctctgatgccgcatagttaagccagta
tctgctccctgcttgtgtgttggaggtcgctgagtagtgcgcgagcaaaatttaagctacaacaaggcaaggcttgacc
gacaattgcatgaagaatctgcttagggttaggcgttttgcgctgcttcgcgatgtacgggccagatatacgcgttgaca
ttgattattgactagttattaatagtaatcaattacggggtcattagttcatagcccatatatggagttccgcgttacataact
tacggtaaatggcccgcctggctgaccgcccaacgacccccgcccattgacgtcaataatgacgtatgttcccatagt
aacgccaatagggactttccattgacgtcaatgggtggagtatttacggtaaactgcccacttggcagtacatcaagtg
tatcatatgccaagtacgccccctattgacgtcaatgacggtaaatggcccgcctggcattatgcccagtacatgacctt
atgggactttcctacttggcagtacatctacgtattagtcatcgctattaccatggtgatgcggttttggcagtacatcaatg
ggcgtggatagcggtttgactcacggggatttccaagtctccaccccattgacgtcaatgggagtttgttttggcaccaa
aatcaacgggactttccaaaatgtcgtaacaactccgccccattgacgcaaatgggcggtaggcgtgtacggtggga
ggtctatataagcagagctctccctatcagtgatagagatctccctatcagtgatagagatcgtcgacgagctcgtttag
tgaaccgtcagatcgcctggagacgccatccacgctgttttgacctccatagaagacaccgggaccgatccagcctc
cggactctagcgtttaaacttaagcttggtaccgagctcggatctgccaccATGTGGTGGCGCCTGTGGT
GGCTGCTGCTGCTGCTGCTGCTGCTGTGGCCCATGGTGTGGGCCGCAGCAGA
GAGTAAACGGAATAAGCCAGGGAAGGCAACAGGTAAAGGAAAACCTGTCAATA
ATAAGTGGTTAAATAATGCAGGTAAAGACTTAGGTTCTCCTGTTCCAGATCGTAT
AGCTAATAAACTACGTGATAAGGAGTTTAAAAGTTTCGATGATTTTCGTAAGAAA
TTCTGGGAAGAAGTGTCAAAAGATCCTGAGTTAAGTAAACAATTTAGTCGAAAC
AATAATGATCGAATGAAGGTTGGAAAAGCGCCCAAGACTAGAACCCAGGATGTT
TCAGGGAAGGCAACTTCATTCGCACTTCATCATGAGAAGCCGATCAGCCAAAAT
GGTGGTGTCTATGATATGGATAACATCAGCGTGGTAACACCTAAACGTGCTATT
GATATTCACCGAGGTAAAAGCGGAGGTGGCTCATCAGGATCCGGTAGAGTCCA
ACCAACAGAATCTATTGTTAGATTTCCTAATATTACAAACTTGTGCCCTTTTGGT
GAAGTTTTTAACGCCACCAGATTTGCATCTGTTTATGCTTGGAACAGGAAGAGA
ATCAGCAACTGTGTTGCTGATTATTCTGTCCTATATAATTCCGCATCATTTTCCA
CTTTTAAGTGTTATGGAGTGTCTCCTACTAAATTAAATGATCTCTGCTTTACTAAT
GTCTATGCAGATTCATTTGTAATTAGAGGTGATGAAGTCAGACAAATCGCTCCA
GGGCAAACTGGAAAGATTGCTGATTATAATTATAAATTACCAGATGATTTTACAG
GCTGCGTTATAGCTTGGAATTCTAACAATCTTGATTCTAAGGTTGGTGGTAATTA
TAATTACCTGTATAGATTGTTTAGGAAGTCTAATCTCAAACCTTTTGAGAGAGAT
ATTTCAACTGAAATCTATCAGGCCGGTAGCACACCTTGTAATGGTGTTGAAGGT
TTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCA
ACTGTTTGTGGACCTAAAAAGTCTACTAATTTGGTTAAAAACAAATGTGTCAATT
TCCATCATCATCACCACCACtgagcggccgcttacccatacgatgtgccagattacgcctaagtcgagtc
tagagggcccgtttaaacccgctgatcagcctcgactgtgccttctagttgccagccatctgttgtttgcccctcccccgtg
ccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctg
gggatgcggtgggctctatggcttctgaggcggaaagaaccagctggggctctagggggtatccccacgcgccctgt
agcggcgcattaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgggggctccctttagggtt
ccgatttagtgctttacggcacctcgaccccaaaaaacttgattagggtgatggttcacgtacctagaagttcctattccg
aagttcctattctctagaaagtataggaacttccttggccaaaaagcctgaactcaccgcgacgtctgtcgagaagtttc
tgatcgaaaagttcgacagcgtctccgacctgatgcagctctcggagggcgaagaatctcgtgctttcagcttcgatgt
aggagggcgtggatatgtcctgcgggtaaatagctgcgccgatggtttctacaaagatcgttatgtttatcggcactttgc
atcggccgcgctcccgattccggaagtgcttgacattggggagttcagcgagagcctgacctattgcatctcccgccgt
gcacagggtgtcacgttgcaagacctgcctgaaaccgaactgcccgctgttctgcagccggtcgcggaggccatgg
atgcgatcgctgcggccgatcttagccagacgagcgggttcggcccattcggaccgcaaggaatcggtcaatacac
tacatggcgtgatttcatatgcgcgattgctgatccccatgtgtatcactggcaaactgtgatggacgacaccgtcagtg
cgtccgtcgcgcaggctctcgatgagctgatgctttgggccgaggactgccccgaagtccggcacctcgtgcacgcg
gatttcggctccaacaatgtcctgacggacaatggccgcataacagcggtcattgactggagcgaggcgatgttcgg
ggattcccaatacgaggtcgccaacatcttcttctggaggccgtggttggcttgtatggagcagcagacgcgctacttc
gagcggaggcatccggagcttgcaggatcgccgcggctccgggcgtatatgctccgcattggtcttgaccaactctat
cagagcttggttgacggcaatttcgatgatgcagcttgggcgcagggtcgatgcgacgcaatcgtccgatccggagc
cgggactgtcgggcgtacacaaatcgcccgcagaagcgcggccgtctggaccgatggctgtgtagaagtactcgc
cgatagtggaaaccgacgccccagcactcgtccgagggcaaaggaatagcacgtactacgagatttcgattccac
cgccgccttctatgaaaggttgggcttcggaatcgttttccgggacgccggctggatgatcctccagcgcggggatctc
atgctggagttcttcgcccaccccaacttgtttattgcagcttataatggttacaaataaagcaatagcatcacaaatttca
caaataaagcatttttttcactgcattctagttgtggtttgtccaaactcatcaatgtatcttatcatgtctgtataccgtcgacc
tctagctagagcttggcgtaatcatggtcatagctgtttcctgtgtgaaattgttatccgctcacaattccacacaacatac
gagccggaagcataaagtgtaaagcctggggtgcctaatgagtgagctaactcacattaattgcgttgcgctcactgc
ccgctttccagtcgggaaacctgtcgtgccagctgcattaatgaatcggccaacgcgcggggagaggcggtttgcgt
attgggcgctcttccgcttcctcgctcactgactcgctgcgctcggtcgttcggctgcggcgagcggtatcagctcactca
aaggcggtaatacggttatccacagaatcaggggataacgcaggaaagaacatgtgagcaaaaggccagcaaa
aggccaggaaccgtaaaaaggccgcgttgctggcgtttttccataggctccgcccccctgacgagcatcacaaaaat
cgacgctcaagtcagaggtggcgaaacccgacaggactataaagataccaggcgtttccccctggaagctccctcg
tgcgctctcctgttccgaccctgccgcttaccggatacctgtccgcctttctcccttcgggaagcgtggcgctttctcatag
ctcacgctgtaggtatctcagttcggtgtaggtcgttcgctccaagctgggctgtgtgcacgaaccccccgttcagcccg
accgctgcgccttatccggtaactatcgtcttgagtccaacccggtaagacacgacttatcgccactggcagcagcca
ctggtaacaggattagcagagcgaggtatgtaggcggtgctacagagttcttgaagtggtggcctaactacggctaca
ctagaagaacagtatttggtatctgcgctctgctgaagccagttaccttcggaaaaagagttggtagctcttgatccggc
aaacaaaccaccgctggtagcggtggtttttttgtttgcaagcagcagattacgcgcagaaaaaaaggatctcaaga
agatcctttgatcttttctacggggtctgacgctcagtggaacgaaaactcacgttaagggattttggtcatgagattatca
aaaaggatcttcacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcctgactccccgtc
gtgtagataactacgatacgggagggcttaccatctggccccagtgctgcaatgataccgcgagacccacgctcacc
ggctccagatttatcagcaataaaccagccagccggaagggccgagcgcagaagtggtcctgcaactttatccgcc
tccatccagtctattaattgttgccgggaagctagagtaagtagttcgccagttaatagtttgcgcaacgttgttgccattg
ctacaggcatcgtggtgtcacgctcgtcgtttggtatggcttcattcagctccggttcccaacgatcaaggcgagttacat
gatcccccatgttgtgcaaaaaagcggttagctccttcggtcctccgatcgttgtcagaagtaagttggccgcagtgttat
cactcatggttatggcagcactgcataattctcttactgtcatgccatccgtaagatgcttttctgtgactggtgagtactca
accaagtcattctgagaatagtgtatgcggcgaccgagttgctcttgcccggcgtcaatacgggataataccgcgcca
catagcagaactttaaaagtgctcatcattggaaaacgttcttcggggcgaaaactctcaaggatcttaccgctgttga
gatccagttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactcttcctttttc
aatattattgaagcatttatcagggttattgtctcatgagcggatacatatttgaatgtatttagaaaaataaacaaatagg
ggttccgcgcacatttccccgaaaagtgccacctgacgtc
SEQ ID NO: 10
>DU67793_pcDNA5D FRT TO SP C7 S-RBD His GFP 41574 (7031 bp)
gacggatcgggagatctcccgatcccctatggtgcactctcagtacaatctgctctgatgccgcatagtta
agccagtatctgctccctgcttgtgtgttggaggtcgctgagtagtgcgcgagcaaaatttaagctacaac
aaggcaaggcttgaccgacaattgcatgaagaatctgcttagggttaggcgttttgcgctgcttcgcgatg
tacgggccagatatacgcgttgacattgattattgactagttattaatagtaatcaattacggggtcatta
gttcatagcccatatatggagttccgcgttacataacttacggtaaatggcccgcctggctgaccgcccaa
cgacccccgcccattgacgtcaataatgacgtatgttcccatagtaacgccaatagggactttccattgac
gtcaatgggtggagtatttacggtaaactgcccacttggcagtacatcaagtgtatcatatgccaagtacg
ccccctattgacgtcaatgacggtaaatggcccgcctggcattatgcccagtacatgaccttatgggactt
tcctacttggcagtacatctacgtattagtcatcgctattaccatggtgatgcggttttggcagtacatca
atgggcgtggatagcggtttgactcacggggatttccaagtctccaccccattgacgtcaatgggagtttg
ttttggcaccaaaatcaacgggactttccaaaatgtcgtaacaactccgccccattgacgcaaatgggcgg
taggcgtgtacggtgggaggtctatataagcagagctctccctatcagtgatagagatctccctatcagtg
atagagatcgtcgacgagctcgtttagtgaaccgtcagatcgcctggagacgccatccacgctgttttgac
ctccatagaagacaccgggaccgatccagcctccggactctagcgtttaaacttaagcttggtaccgagct
cggatctgccaccATGTGGTGGCGCCTGTGGTGGCTGCTGCTGCTGCTGCTGCTGCTGTGGCCCATGGTGT
GGGCCGCAGCAGAGAGTAAACGGAATAAGCCAGGGAAGGCAACAGGTAAAGGAAAACCTGTCAATAATAAG
TGGTTAAATAATGCAGGTAAAGACTTAGGTTCTCCTGTTCCAGATCGTATAGCTAATAAACTACGTGATAA
GGAGTTTAAAAGTTTCGATGATTTTCGTAAGAAATTCTGGGAAGAAGTGTCAAAAGATCCTGAGTTAAGTA
AACAATTTAGTCGAAACAATAATGATCGAATGAAGGTTGGAAAAGCGCCCAAGACTAGAACCCAGGATGTT
TCAGGGAAGGCAACTTCATTCGCACTTCATCATGAGAAGCCGATCAGCCAAAATGGTGGTGTCTATGATAT
GGATAACATCAGCGTGGTAACACCTAAACGTGCTATTGATATTCACCGAGGTAAAAGCGGAGGTGGCTCAT
CAGGATCCGGTAGAGTCCAACCAACAGAATCTATTGTTAGATTTCCTAATATTACAAACTTGTGCCCTTTT
GGTGAAGTTTTTAACGCCACCAGATTTGCATCTGTTTATGCTTGGAACAGGAAGAGAATCAGCAACTGTGT
TGCTGATTATTCTGTCCTATATAATTCCGCATCATTTTCCACTTTTAAGTGTTATGGAGTGTCTCCTACTA
AATTAAATGATCTCTGCTTTACTAATGTCTATGCAGATTCATTTGTAATTAGAGGTGATGAAGTCAGACAA
ATCGCTCCAGGGCAAACTGGAAAGATTGCTGATTATAATTATAAATTACCAGATGATTTTACAGGCTGCGT
TATAGCTTGGAATTCTAACAATCTTGATTCTAAGGTTGGTGGTAATTATAATTACCTGTATAGATTGTTTA
GGAAGTCTAATCTCAAACCTTTTGAGAGAGATATTTCAACTGAAATCTATCAGGCCGGTAGCACACCTTGT
AATGGTGTTGAAGGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACTGTTTGTGGACCTA
AAAAGTCTACTAATTTGGTTAAAAACAAATGTGTCAATTTCGGAGGTCATCATCATCACCACCACGGAGGT
GGCTCATCAGGATCAGGTgcggccgctatggtgagcaagggcgaggagctgttcaccggggtggtgcccat
cctggtcgagctggacggcgacgtaaacggccacaagttcagcgtgtccggcgagggcgagggcgatgcca
cctacggcaagctgaccctgaagttcatctgcaccaccggcaagctgcccgtgccctggcccaccctcgtg
accaccctgacctacggcgtgcagtgcttcagccgctaccccgaccacatgaagcagcacgacttcttcaa
gtccgccatgcccgaaggctacgtccaggagcgcaccatcttcttcaaggacgacggcaactacaagaccc
gcgccgaggtgaagttcgagggcgacaccctggtgaaccgcatcgagctgaagggcatcgacttcaaggag
gacggcaacatcctggggcacaagctggagtacaactacaacagccacaacgtctatatcatggccgacaa
gcagaagaacggcatcaaggtgaacttcaagatccgccacaacatcgaggacggcagcgtgcagctcgccg
accactaccagcagaacacccccatcggcgacggccccgtgctgctgcccgacaaccactacctgagcacc
cagtccgccctgagcaaagaccccaacgagaagcgcgatcacatggtcctgctggagttcgtgaccgccgc
cgggatcactctcggcatggacgagctgtacaagtccggactctaagggcccgtttaaacccgctgatcag
cctcgactgtgccttctagttgccagccatctgttgtttgcccctcccccgtgccttccttgaccctggaa
ggtgccactcccactgtcctttcctaataaaatgaggaaattgcatcgcattgtctgagtaggtgtcattc
tattctggggggtggggtggggcaggacagcaagggggaggattgggaagacaatagcaggcatgctgggg
atgcggtgggctctatggcttctgaggcggaaagaaccagctggggctctagggggtatccccacgcgccc
tgtagcggcgcattaagcgcggcgggtgtggtggttacgcgcagcgtgaccgctacacttgccagcgccct
agcgcccgctcctttcgctttcttcccttcctttctcgccacgttcgccggctttccccgtcaagctctaa
atcgggggctccctttagggttccgatttagtgctttacggcacctcgaccccaaaaaacttgattagggt
gatggttcacgtacctagaagttcctattccgaagttcctattctctagaaagtataggaacttccttggc
caaaaagcctgaactcaccgcgacgtctgtcgagaagtttctgatcgaaaagttcgacagcgtctccgacc
tgatgcagctctcggagggcgaagaatctcgtgctttcagcttcgatgtaggagggcgtggatatgtcctg
cgggtaaatagctgcgccgatggtttctacaaagatcgttatgtttatcggcactttgcatcggccgcgct
cccgattccggaagtgcttgacattggggagttcagcgagagcctgacctattgcatctcccgccgtgcac
agggtgtcacgttgcaagacctgcctgaaaccgaactgcccgctgttctgcagccggtcgcggaggccatg
gatgcgatcgctgcggccgatcttagccagacgagcgggttcggcccattcggaccgcaaggaatcggtca
atacactacatggcgtgatttcatatgcgcgattgctgatccccatgtgtatcactggcaaactgtgatgg
acgacaccgtcagtgcgtccgtcgcgcaggctctcgatgagctgatgctttgggccgaggactgccccgaa
gtccggcacctcgtgcacgcggatttcggctccaacaatgtcctgacggacaatggccgcataacagcggt
cattgactggagcgaggcgatgttcggggattcccaatacgaggtcgccaacatcttcttctggaggccgt
ggttggcttgtatggagcagcagacgcgctacttcgagcggaggcatccggagcttgcaggatcgccgcgg
ctccgggcgtatatgctccgcattggtcttgaccaactctatcagagcttggttgacggcaatttcgatga
tgcagcttgggcgcagggtcgatgcgacgcaatcgtccgatccggagccgggactgtcgggcgtacacaaa
tcgcccgcagaagcgcggccgtctggaccgatggctgtgtagaagtactcgccgatagtggaaaccgacgc
cccagcactcgtccgagggcaaaggaatagcacgtactacgagatttcgattccaccgccgccttctatga
aaggttgggcttcggaatcgttttccgggacgccggctggatgatcctccagcgcggggatctcatgctgg
agttcttcgcccaccccaacttgtttattgcagcttataatggttacaaataaagcaatagcatcacaaat
ttcacaaataaagcatttttttcactgcattctagttgtggtttgtccaaactcatcaatgtatcttatca
tgtctgtataccgtcgacctctagctagagcttggcgtaatcatggtcatagctgtttcctgtgtgaaatt
gttatccgctcacaattccacacaacatacgagccggaagcataaagtgtaaagcctggggtgcctaatga
gtgagctaactcacattaattgcgttgcgctcactgcccgctttccagtcgggaaacctgtcgtgccagct
gcattaatgaatcggccaacgcgcggggagaggcggtttgcgtattgggcgctcttccgcttcctcgctca
ctgactcgctgcgctcggtcgttcggctgcggcgagcggtatcagctcactcaaaggcggtaatacggtta
tccacagaatcaggggataacgcaggaaagaacatgtgagcaaaaggccagcaaaaggccaggaaccgtaa
aaaggccgcgttgctggcgtttttccataggctccgcccccctgacgagcatcacaaaaatcgacgctcaa
gtcagaggtggcgaaacccgacaggactataaagataccaggcgtttccccctggaagctccctcgtgcgc
tctcctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacgaac
cccccgttcagcccgaccgctgcgccttatccggtaactatcgtcttgagtccaacccggtaagacacgac
ttatcgccactggcagcagccactggtaacaggattagcagagcgaggtatgtaggcggtgctacagagtt
cttgaagtggtggcctaactacggctacactagaagaacagtatttggtatctgcgctctgctgaagccag
ttaccttcggaaaaagagttggtagctcttgatccggcaaacaaaccaccgctggtagcggtggttttttt
gtttgcaagcagcagattacgcgcagaaaaaaaggatctcaagaagatcctttgatcttttctacggggtc
tgacgctcagtggaacgaaaactcacgttaagggattttggtcatgagattatcaaaaaggatcttcacct
agatccttttaaattaaaaatgaagttttaaatcaatctaaagtatatatgagtaaacttggtctgacagt
taccaatgcttaatcagtgaggcacctatctcagcgatctgtctatttcgttcatccatagttgcctgact
ccccgtcgtgtagataactacgatacgggagggcttaccatctggccccagtgctgcaatgataccgcgag
acccacgctcaccggctccagatttatcagcaataaaccagccagccggaagggccgagcgcagaagtggt
cctgcaactttatccgcctccatccagtctattaattgttgccgggaagctagagtaagtagttcgccagt
taatagtttgcgcaacgttgttgccattgctacaggcatcgtggtgtcacgctcgtcgtttggtatggctt
cattcagctccggttcccaacgatcaaggcgagttacatgatcccccatgttgtgcaaaaaagcggttagc
tccttcggtcctccgatcgttgtcagaagtaagttggccgcagtgttatcactcatggttatggcagcact
gcataattctcttactgtcatgccatccgtaagatgcttttctgtgactggtgagtactcaaccaagtcat
tctgagaatagtgtatgcggcgaccgagttgctcttgcccggcgtcaatacgggataataccgcgccacat
agcagaactttaaaagtgctcatcattggaaaacgttcttcggggcgaaaactctcaaggatcttaccgct
gttgagatccagttcgatgtaacccactcgtgcacccaactgatcttcagcatcttttactttcaccagcg
tttctgggtgagcaaaaacaggaaggcaaaatgccgcaaaaaagggaataagggcgacacggaaatgttga
atactcatactcttcctttttcaatattattgaagcatttatcagggttattgtctcatgagcggatacat
atttgaatgtatttagaaaaataaacaaataggggttccgcgcacatttccccgaaaagtgccacctgacg
tc
SEQ ID NO: 11
>DU67817 pcDNA5D FRT TO SP C7 S-RBD N-ntd His (6752 bp)
gacggatcgggagatctcccgatcccctatggtgcactctcagtacaatctgctctgatgccgcatagtta
agccagtatctgctccctgcttgtgtgttggaggtcgctgagtagtgcgcgagcaaaatttaagctacaac
aaggcaaggcttgaccgacaattgcatgaagaatctgcttagggttaggcgttttgcgctgcttcgcgatg
tacgggccagatatacgcgttgacattgattattgactagttattaatagtaatcaattacggggtcatta
gttcatagcccatatatggagttccgcgttacataacttacggtaaatggcccgcctggctgaccgcccaa
cgacccccgcccattgacgtcaataatgacgtatgttcccatagtaacgccaatagggactttccattgac
gtcaatgggtggagtatttacggtaaactgcccacttggcagtacatcaagtgtatcatatgccaagtacg
ccccctattgacgtcaatgacggtaaatggcccgcctggcattatgcccagtacatgaccttatgggactt
tcctacttggcagtacatctacgtattagtcatcgctattaccatggtgatgcggttttggcagtacatca
atgggcgtggatagcggtttgactcacggggatttccaagtctccaccccattgacgtcaatgggagtttg
ttttggcaccaaaatcaacgggactttccaaaatgtcgtaacaactccgccccattgacgcaaatgggcgg
taggcgtgtacggtgggaggtctatataagcagagctctccctatcagtgatagagatctccctatcagtg
atagagatcgtcgacgagctcgtttagtgaaccgtcagatcgcctggagacgccatccacgctgttttgac
ctccatagaagacaccgggaccgatccagcctccggactctagcgtttaaacttaagcttggtaccgagct
cggatctgccaccATGTGGTGGCGCCTGTGGTGGCTGCTGCTGCTGCTGCTGCTGCTGTGGCCCATGGTGT
GGGCCGCAGCAGAGAGTAAACGGAATAAGCCAGGGAAGGCAACAGGTAAAGGAAAACCTGTCAATAATAAG
TGGTTAAATAATGCAGGTAAAGACTTAGGTTCTCCTGTTCCAGATCGTATAGCTAATAAACTACGTGATAA
GGAGTTTAAAAGTTTCGATGATTTTCGTAAGAAATTCTGGGAAGAAGTGTCAAAAGATCCTGAGTTAAGTA
AACAATTTAGTCGAAACAATAATGATCGAATGAAGGTTGGAAAAGCGCCCAAGACTAGAACCCAGGATGTT
TCAGGGAAGGCAACTTCATTCGCACTTCATCATGAGAAGCCGATCAGCCAAAATGGTGGTGTCTATGATAT
GGATAACATCAGCGTGGTAACACCTAAACGTGCTATTGATATTCACCGAGGTAAAAGCGGAGGTGGCTCAT
CAGGATCCGGTAGAGTCCAACCAACAGAATCTATTGTTAGATTTCCTAATATTACAAACTTGTGCCCTTTT
GGTGAAGTTTTTAACGCCACCAGATTTGCATCTGTTTATGCTTGGAACAGGAAGAGAATCAGCAACTGTGT
TGCTGATTATTCTGTCCTATATAATTCCGCATCATTTTCCACTTTTAAGTGTTATGGAGTGTCTCCTACTA
AATTAAATGATCTCTGCTTTACTAATGTCTATGCAGATTCATTTGTAATTAGAGGTGATGAAGTCAGACAA
ATCGCTCCAGGGCAAACTGGAAAGATTGCTGATTATAATTATAAATTACCAGATGATTTTACAGGCTGCGT
TATAGCTTGGAATTCTAACAATCTTGATTCTAAGGTTGGTGGTAATTATAATTACCTGTATAGATTGTTTA
GGAAGTCTAATCTCAAACCTTTTGAGAGAGATATTTCAACTGAAATCTATCAGGCCGGTAGCACACCTTGT
AATGGTGTTGAAGGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACTGTTTGTGGACCTA
AAAAGTCTACTAATTTGGTTAAAAACAAATGTGTCAATTTCGGAGGTGGCTCATCAGGATCAGGTGGTTTA
CCCAATAATACTGCGTCTTGGTTCACCGCTCTCACTCAACATGGCAAGGAAGACCTTAAATTCCCTCGAGG
ACAAGGCGTTCCAATTAACACCAATAGCAGTCCAGATGACCAAATTGGCTACTACCGAAGAGCTACCAGAC
GAATTCGTGGTGGTGACGGTAAAATGAAAgacCTCAGTCCAAGATGGTATTTCTACTACCTAGGAACTGGG
CCAGAAGCTGGACTTCCCTATGGTGCTAACAAAGACGGCATCATATGGGTTGCAACTGAGGGAGCCTTGAA
TACACCAAAAGATCACATTGGCACCCGCAATCCTGCTAACAATGCTGCAATCGTGCTACAACTTCCTCAAG
GAACAACATTGCCAAAAGGCTTCTACGCAGAAGGGAGCAGAGGCGGCAGTCATCATCATCACCACCACtga
gcggccgcttacccatacgatgtgccagattacgcctaagtcgagtctagagggcccgtttaaacccgctg
atcagcctcgactgtgccttctagttgccagccatctgttgtttgcccctcccccgtgccttccttgaccc
tggaaggtgccactcccactgtcctttcctaataaaatgaggaaattgcatcgcattgtctgagtaggtgt
cattctattctggggggtggggtggggcaggacagcaagggggaggattgggaagacaatagcaggcatgc
tggggatgcggtgggctctatggcttctgaggcggaaagaaccagctggggctctagggggtatccccacg
cgccctgtagcggcgcattaagcgcggcgggtgtggtggttacgcgcagcgtgaccgctacacttgccagc
gccctagcgcccgctcctttcgctttcttcccttcctttctcgccacgttcgccggctttccccgtcaagc
tctaaatcgggggctccctttagggttccgatttagtgctttacggcacctcgaccccaaaaaacttgatt
agggtgatggttcacgtacctagaagttcctattccgaagttcctattctctagaaagtataggaacttcc
ttggccaaaaagcctgaactcaccgcgacgtctgtcgagaagtttctgatcgaaaagttcgacagcgtctc
cgacctgatgcagctctcggagggcgaagaatctcgtgctttcagcttcgatgtaggagggcgtggatatg
tcctgcgggtaaatagctgcgccgatggtttctacaaagatcgttatgtttatcggcactttgcatcggcc
gcgctcccgattccggaagtgcttgacattggggagttcagcgagagcctgacctattgcatctcccgccg
tgcacagggtgtcacgttgcaagacctgcctgaaaccgaactgcccgctgttctgcagccggtcgcggagg
ccatggatgcgatcgctgcggccgatcttagccagacgagcgggttcggcccattcggaccgcaaggaatc
ggtcaatacactacatggcgtgatttcatatgcgcgattgctgatccccatgtgtatcactggcaaactgt
gatggacgacaccgtcagtgcgtccgtcgcgcaggctctcgatgagctgatgctttgggccgaggactgcc
ccgaagtccggcacctcgtgcacgcggatttcggctccaacaatgtcctgacggacaatggccgcataaca
gcggtcattgactggagcgaggcgatgttcggggattcccaatacgaggtcgccaacatcttcttctggag
gccgtggttggcttgtatggagcagcagacgcgctacttcgagcggaggcatccggagcttgcaggatcgc
cgcggctccgggcgtatatgctccgcattggtcttgaccaactctatcagagcttggttgacggcaatttc
gatgatgcagcttgggcgcagggtcgatgcgacgcaatcgtccgatccggagccgggactgtcgggcgtac
acaaatcgcccgcagaagcgcggccgtctggaccgatggctgtgtagaagtactcgccgatagtggaaacc
gacgccccagcactcgtccgagggcaaaggaatagcacgtactacgagatttcgattccaccgccgccttc
tatgaaaggttgggcttcggaatcgttttccgggacgccggctggatgatcctccagcgcggggatctcat
gctggagttcttcgcccaccccaacttgtttattgcagcttataatggttacaaataaagcaatagcatca
caaatttcacaaataaagcatttttttcactgcattctagttgtggtttgtccaaactcatcaatgtatct
tatcatgtctgtataccgtcgacctctagctagagcttggcgtaatcatggtcatagctgtttcctgtgtg
aaattgttatccgctcacaattccacacaacatacgagccggaagcataaagtgtaaagcctggggtgcct
aatgagtgagctaactcacattaattgcgttgcgctcactgcccgctttccagtcgggaaacctgtcgtgc
cagctgcattaatgaatcggccaacgcgcggggagaggcggtttgcgtattgggcgctcttccgcttcctc
gctcactgactcgctgcgctcggtcgttcggctgcggcgagcggtatcagctcactcaaaggcggtaatac
ggttatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcgacg
ctcaagtcagaggtggcgaaacccgacaggactataaagataccaggcgtttccccctggaagctccctcg
tgcgctctcctgttccgaccctgccgcttaccggatacctgtccgcctttctcccttcgggaagcgtggcg
ctttctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaagac
acgacttatcgccactggcagcagccactggtaacaggattagcagagcgaggtatgtaggcggtgctaca
gagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtt
tttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatctt
cacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcc
tgactccccgtcgtgtagataactacgatacgggagggcttaccatctggccccagtgctgcaatgatacc
gcgagacccacgctcaccggctccagatttatcagcaataaaccagccagccggaagggccgagcgcagaa
gtggtcctgcaactttatccgcctccatccagtctattaattgttgccgggaagctagagtaagtagttcg
ccagttaatagtttgcgcaacgttgttgccattgctacaggcatcgtggtgtcacgctcgtcgtttggtat
ggcttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatggca
gcactgcataattctcttactgtcatgccatccgtaagatgcttttctgtgactggtgagtactcaaccaa
gtcattctgagaatagtgtatgcggcgaccgagttgctcttgcccggcgtcaatacgggataataccgcgc
cacatagcagaactttaaaagtgctcatcattggaaaacgttcttcggggcgaaaactctcaaggatctta
ccgctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacggaaat
gttgaatactcatactcttcctttttcaatattattgaagcatttatcagggttattgtctcatgagcgga
tacatatttgaatgtatttagaaaaataaacaaataggggttccgcgcacatttccccgaaaagtgccacc
tgacgtc
Seq ID No: 47
>DU70680. pETDuet-1 HBc-Im7 F→L 6His-tet-inducible ColE7-linker-
TEV_site_hIL17A; 7634 bp
GGGAATTGTGAGCGGATAACAATTCCCCTCTAGAAATAATTTTGTTTAACTTTAAGAAGGAGATATACCat
ggacattgacccgtataaagaatttggagcttctgtggagttactctcttttttgccttctgacttctttc
cttctattcgagatctcctcgacaccgcctcagctctgtatcgggaggccttagagtctccggaacattgt
tcacctcaccatacagcactcaggcaagctattctgtgttggggtgagttgatgaatttggccacctgggt
gggaagtaatttgggaggaggtggaagcggaggaggtggatctgaactgaaaaatagtattagtgattaca
cagaggctgagtttgttcaacttcttaaggaaattgaaaaagagaatgttgctgcaactgatgatgtgtta
gatgtgttactcgaacacctagtaaaaattactgagcatccagatggaacggatctgatttattatcctag
tgataatagagacgatagccccgaagggattgtcaaggaaattaaagaatggcgagctgctaacggtaagc
caggatttaaacagggcggaggaggtggaagcggaggaggtggatctccagcatccagggaattagtagtc
agctatgttaatgttaatatgggcctaaaaatcagacaactactgtggtttcacatttcctgtcttacttt
tggaagagaaactgttcttgaatatttggtgtcttttggagtgtggattcgcactcctcctgcttacagac
caccaaatgcccctatcttatcaacacttccggaaactactgttgttCATCACCATCATCACCACtaataa
GCGGCCGCTTAACCTAGGCTGCTGCCACCGCTGAGCAATAACTAGCATAACCCCTTGGGGCCTCTAAACGG
GTCTTGAGGGGTTTTTTGTTGTACACGGCCGCATAATCGgttgacactctatcattgatagagttatttta
ccactccctatcagtgatagagaaAagtgaaatgaatagttcgacaaaaatctagataacgagggcaaCAT
ATGGAGAGTAAACGGAATAAGCCAGGGAAGGCAACAGGTAAAGGAAAACCTGTCAATAATAAGTGGTTAAA
TAATGCAGGTAAAGACTTAGGTTCTCCTGTTCCAGATCGTATAGCTAATAAACTACGTGATAAGGAGTTTA
AAAGTTTCGATGATTTTCGTAAGAAATTCTGGGAAGAAGTGTCAAAAGATCCTGAGTTAAGTAAACAATTT
AGTCGAAACAATAATGATCGAATGAAGGTTGGAAAAGCGCCCAAGACTAGAACCCAGGATGTTTCAGGGAA
GGCAACTTCATTCGCACTTCATCATGAGAAGCCGATCAGCCAAAATGGTGGTGTCTATGATATGGATAACA
TCAGCGTGGTAACACCTAAACGTGCTATTGATATTCACCGAGGTAAAAGCGGAGGTGGCTCATCAGGATCt
GGTGAAAACCTGTATTTTCAGGGatccGGAGGTGGCTCAggaatcacaatcccacgaaatccaggatgccc
aaattctgaggacaagaacttcccccggactgtgatggtcaacctgaacatccataaccggaataccaata
ccaatcccaaaaggtcctcagattactacaaccgatccacctcaccttggaatctccaccgcaatgaggac
cctgagagatatccctctgtgatctgggaggcaaagtgccgccacttgggctgcatcaacgctgatgggaa
cgtggactaccacatgaactctgtccccatccagcaagagatcctggtcctgcgcagggagcctccacact
gccccaactccttccggctggagaagatactggtgtccgtgggctgcacctgtgtcaccccgattgtccac
catgtggcctaataaGGTACCCTCGAGTCTGGTAAAGAAACCGCTGCTGCGAAATTTGAACGCCAGCACAT
GGACTCGTCTACTAGCGCAGCTTAATTAACCTAGGCTGCTGCCACCGCTGAGCAATAACTAGCATAACCCC
TTGGGGCCTCTAAACGGGTCTTGAGGGGTTTTTTGCTGAAAGGAGGAACTATATCCGGATTGGCGAATGGG
ACGCGCCCTGTAGCGGCGCATTAAGCGCGGCGGGTGTGGTGGTTACGCGCAGCGTGACCGCTACACTTGCC
AGCGCCCTAGCGCCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGACCCCAAAAAACTTG
ATTAGGGTGATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCGGTCTATTCTTTTGA
TTTATAAGGGATTTTGCCGATTTCGGCCTATTGGTTAAAAAATGAGCTGATTTAACAAAAATTTAACGCGA
ATTTTAACAAAATATTAACGTTTACAATTTCTGGCGGCACGATGGCATGAGATTATCAAAAAGGATCTTCA
CCTAGATCCTTTTAAATTAAAAATGAAGTTTTAAATCAATCTAAAGTATATATGAGTAAACTTGGTCTTta
GGACCCACTTTCACATTTAAGTTGTTTTTCTAATCCGCAgATGATCAATTCAAGGCCGAATAAGAAGGCTG
GCTCTGCACCTTGGTGATCAAATAATTCGATAGCTTGTCGTAATAATGGCGGCATACTATCAGTAGTAGGT
GTTTCCCTTTCTTCTTTAGCGACTTGATGCTCTTGATCTTCCAATACGCAACCTAAAGTAAAATGCCCCAC
AGCGCTGAGTGCATATAATGCATTCTCTAGTGAAAAACCTTGTTGGCATAAAAAGGCTAATTGATTTTCGA
GAGTTTCATACTGTTTTTCTGTAGGCCGTGTACCTAAATGTACTTTTGCTCCATCGCGATGACTTAGTAAA
GCACATCTAAAACTTTTAGCGTTATTACGTAAAAAATCTTGCCAGCTTTCCCCTTCTAAAGGGCAAAAGTG
AGTATGGTGCCTATCTAACATCTCAATGGCTAAGGCGTCGAGCAAAGCCCGCTTATTTTTTACATGCCAAT
ACAATGTAGGCTGCTCTACACCTAGCTTCTGGGCGAGTTTACGGGTTGTTAAACCTTCGATTCCGACCTCA
TTAAGCAGCTCTAATGCGCTGTTAATCACTTTACTTTTATCTAATCTAGACATcattaattccTACCAATG
CTTAATCAGTGAGGCACCTATCTCAGCGATCTGTCTATTTCGTTCATCCATAGTTGCCTGACTCCCCGTCG
TGTAGATAACTACGATACGGGAGGGCTTACCATCTGGCCCCAGTGCTGCAATGATACCGCGAGACCCACGC
TCACCGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCATTCTGAGAA
TAGTGTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGAGAT
CCAGTTCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATACTCAT
ACTCTTCCTTTTTCAATCATGATTGAAGCATTTATCAGGGTTATTGTCTCATGAGCGGATACATATTTGAA
TGTATTTAGAAAAATAAACAAATAGGTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCAACTCTTTTTCCGAAGGT
AACTGGCTTCAGCAGAGCGCAGATACCAAATACTGTCCTTCTAGTGTAGCCGTAGTTAGGCCACCACTTCA
AGAACTCTGTAGCACCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACGACCGAGCGCAGCGAGTC
AGTGAGCGAGGAAGCGGAAGAGCGCCTGATGCGGTATTTTCTCCTTACGCATCTGTGCGGTATTTCACACC
GCATATATGGTGCACTCTCAGTACAATCTGCTCTGATGCCGCATAGTTAAGCCAGTATACACTCCGCTATC
GCTACGTGACTGGGTCATGGCTGCGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCG
TCATCACCGAAACGCGCGAGGCAGCTGCGGTAAAGCTCATCAGCGTGGTCGTGAAGCGATTCACAGATGTC
TGCCTGTTCATCCGCGTCCAGCTCGTTGAGTTTCTCCAGAAGCGTTAATGTCTGGCTTCTGATAAAGCGGG
CCATGTTAAGGGCGGTTTTTTCCTGTTTGGTCACTGATGCCTCCGTGTAAGGGGGATTTCTGTTCATGGGG
GTAATGATACCGATGAAACGAGAGAGGATGCTCACGATACGGGTTACTGATGATGAACATGCCCGGTTACT
GGAACGTTGTGAGGGTAAACAACTGGCGGTATGGATGCGGCGGGACCAGAGAAAAATCACTCAGGGTCAAT
GCCAGCGCTTCGTTAATACAGATGTAGGTGTTCCACAGGGTAGCCAGCAGCATCCTGCGATGCAGATCCGG
AACATAATGGTGCAGGGCGCTGACTTCCGCGTTTCCAGACTTTACGAAACACGGAAACCGAAGACCATTCA
TGTTGTTGCTCAGGTCGCAGACGTTTTGCAGCAGCAGTCGCTTCACGTTCGCTCGCGTATCGGTGATTCAT
TCTGCTAACCAGTAAGGCAACCCCGCCAGCCTAGCCGGGTCCTCAACGACAGGAGCACGATCATGCTAGTC
ATGCCCCGCGCCCACCGGAAGGAGCTGACTGGGTTGAAGGCTCTCAAGGGCATCGGTCGAGATCCCGGTGC
CTAATGAGTGAGCTAACTTACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCCAGGGTGGTTTT
TCTTTTCACCAGTGAGACGGGCAACAGCTGATTGCCCTTCACCGCCTGGCCCTGAGAGAGTTGCAGCAAGC
GGTCCACGCTGGTTTGCCCCAGCAGGCGAAAATCCTGTTTGATGGTGGTTAACGGCGGGATATAACATGAG
CTGTCTTCGGTATCGTCGTATCCCACTACCGAGATGTCCGCACCAACGCGCAGCCCGGACTCGGTAATGGC
GCGCATTGCGCCCAGCGCCATCTGATCGTTGGCAACCAGCATCGCAGTGGGAACGATGCCCTCATTCAGCA
TTTGCATGGTTTGTTGAAAACCGGACATGGCACTCCAGTCGCCTTCCCGTTCCGCTATCGGCTGAATTTGA
TTGCGAGTGAGATATTTATGCCAGCCAGCCAGACGCAGACGCGCCGAGACAGAACTTAATGGGCCCGCTAA
CAGCGCGATTTGCTGGTGACCCAATGCGACCAGATGCTCCACGCCCAGTCGCGTACCGTCTTCATGGGAGA
AAATAATACTGTTGATGGGTGTCTGGTCAGAGACATCAAGAAATAACGCCGGAACATTAGTGCAGGCAGCT
TCCACAGCAATGGCATCCTGGTCATCCAGCGGATAGTTAATGATCAGCCCACTGACGCGTTGCGCGAGAAG
ATTGTGCACCGCCGCTTTACAGGCTTCGACGCCGCTTCGTTCTACCATCGACACCACCACGCTGGCACCCA
GTTGATCGGCGCGAGATTTAATCGCCGCGACAATTTGCGACGGCGCGTGCAGGGCCAGACTGGAGGTGGCA
ACGCCAATCAGCAACGACTGTTTGCCCGCCAGTIGTTGTGCCACGCGGTTGGGAATGTAATTCAGCTCCGC
CATCGCCGCTTCCACTTTTTCCCGCGTTTTCGCAGAAACGTGGCTGGCCTGGTTCACCACGCGGGAAACGG
TCTGATAAGAGACACCGGCATACTCTGCGACATCGTATAACGTTACTGGTTTCACATTCACCACCCTGAAT
TGACTCTCTTCCGGGCGCTATCATGCCATACCGCGAAAGGTTTTGCGCCATTCGATGGTGTCCGGGATCTC
GACGCTCTCCCTTATGCGACTCCTGCATTAGGAAGCAGCCCAGTAGTAGGTTGAGGCCGTTGAGCACCGCC
GCCGCAAGGAATGGTGCATGCAAGGAGATGGCGCCCAACAGTCCCCCGGCCACGGGGCCTGCCACCATACC
CACGCCGAAACAAGCGCTCATGAGCCCGAAGTGGCGAGCCCGATCTTCCCCATCGGTGATGTCGGCGATAT
AGGCGCCAGCAACCGCACCTGTGGCGCCGGTGATGCCGGCCACGATGCGTCCGGCGTAGAGGATCGAGATC
GATCTCGATCCCGCGAAATTAATACGACTCACTATAG
Seq ID No: 48
>DU70612. pETDuet-1 HBc-Im7 F→L 6His-tet-inducible ColE7-linker-
TEV_site_mIL33; 7700 bp
GGGGAATTGTGAGCGGATAACAATTCCCCTCTAGAAATAATTTTGTTTAACTTTAAGAAGGAGATATACCa
tggacattgacccgtataaagaatttggagcttctgtggagttactctcttttttgccttctgacttcttt
ccttctattcgagatctcctcgacaccgcctcagctctgtatcgggaggccttagagtctccggaacattg
ttcacctcaccatacagcactcaggcaagctattctgtgttggggtgagttgatgaatttggccacctggg
tgggaagtaatttgggaggaggtggaagcggaggaggtggatctgaactgaaaaatagtattagtgattac
acagaggctgagtttgttcaacttcttaaggaaattgaaaaagagaatgttgctgcaactgatgatgtgtt
agatgtgttactcgaacacctagtaaaaattactgagcatccagatggaacggatctgatttattatccta
gtgataatagagacgatagccccgaagggattgtcaaggaaattaaagaatggcgagctgctaacggtaag
ccaggatttaaacagggcggaggaggtggaagcggaggaggtggatctccagcatccagggaattagtagt
cagctatgttaatgttaatatgggcctaaaaatcagacaactactgtggtttcacatttcctgtcttactt
ttggaagagaaactgttcttgaatatttggtgtcttttggagtgtggattcgcactcctcctgcttacaga
ccaccaaatgcccctatcttatcaacacttccggaaactactgttgttCATCACCATCATCACCACtaata
aGCGGCCGCTTAACCTAGGCTGCTGCCACCGCTGAGCAATAACTAGCATAACCCCTTGGGGCCTCTAAACG
GGTCTTGAGGGGTTTTTTGTTGTACACGGCCGCATAATCGgttgacactctatcattgatagagttatttt
accactccctatcagtgatagagaaAagtgaaatgaatagttcgacaaaaatctagataacgagggcaaCA
TATGGAGAGTAAACGGAATAAGCCAGGGAAGGCAACAGGTAAAGGAAAACCTGTCAATAATAAGTGGTTAA
ATAATGCAGGTAAAGACTTAGGTTCTCCTGTTCCAGATCGTATAGCTAATAAACTACGTGATAAGGAGTTT
AAAAGTTTCGATGATTTTCGTAAGAAATTCTGGGAAGAAGTGTCAAAAGATCCTGAGTTAAGTAAACAATT
TAGTCGAAACAATAATGATCGAATGAAGGTTGGAAAAGCGCCCAAGACTAGAACCCAGGATGTTTCAGGGA
AGGCAACTTCATTCGCACTTCATCATGAGAAGCCGATCAGCCAAAATGGTGGTGTCTATGATATGGATAAC
ATCAGCGTGGTAACACCTAAACGTGCTATTGATATTCACCGAGGTAAAAGCGGAGGTGGCTCATCAGGATC
tGGTGAAAACCTGTATTTTCAGGGatccAgcattcagggcaccagcctgctgacccagagcccggcgagcc
tgagcacctataacgatcagagcgtgagctttgtgctggaaaacggctgctatgtgattaacgtggataaa
agcggcaaagatcaggaacaggatcaggtgctgctgcgctattataaaagcccgtgcccggcgagccagag
cggcgatggcgtggatggcaagaaactgatggtgaacatgagcccgattaaagataccgatatttggctgc
atgcgaacgataaagattatagcgtggaactgcagcgcggcgatgtgagcccgccagaacaggcgttcttt
gtgctgcataagaaaagcagcgattttgtgagctttgaatgcaaaaacctgccgggcacctatattggcgt
gaaagataaccagctggcgctggtggaagagaaagatgaaagctgcaacaacattatgtttaaactgagca
aaatttaataaGGTACCCTCGAGTCTGGTAAAGAAACCGCTGCTGCGAAATTTGAACGCCAGCACATGGAC
TCGTCTACTAGCGCAGCTTAATTAACCTAGGCTGCTGCCACCGCTGAGCAATAACTAGCATAACCCCTTGG
GGCCTCTAAACGGGTCTTGAGGGGTTTTTTGCTGAAAGGAGGAACTATATCCGGATTGGCGAATGGGACGC
GCCCTGTAGCGGCGCATTAAGCGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCAAAAAACTTGATTA
GGGTGATGGTTCACGTAGTGGGCCATCGCCCTGATAGACGGTTTTTCGCCCTTTGACGTTGGAGTCCACGT
TCTTTAATAGTGGACTCTTGTTCCAAACTGGAACAACACTCAACCCTATCTCGGTCTATTCTTTTGATTTA
TAAGGGATTTTGCCGATTTCGGCCTATTGGTTAAAAAATGAGCTGATTTAACAAAAATTTAACGCGAATTT
TAACAAAATATTAACGTTTACAATTTCTGGCGGCACGATGGCATGAGATTATCAAAAAGGATCTTCACCTA
GATCCTTTTAAATTAAAAATGAAGTTTTAAATCAATCTAAAGTATATATGAGTAAACTTGGTCTTtaGGAC
CCACTTTCACATTTAAGTTGTTTTTCTAATCCGCAgATGATCAATTCAAGGCCGAATAAGAAGGCTGGCTC
TGCACCTTGGTGATCAAATAATTCGATAGCTTGTCGTAATAATGGCGGCATACTATCAGTAGTAGGTGTTT
CCCTTTCTTCTTTAGCGACTTGATGCTCTTGATCTTCCAATACGCAACCTAAAGTAAAATGCCCCACAGCG
CTGAGTGCATATAATGCATTCTCTAGTGAAAAACCTTGTTGGCATAAAAAGGCTAATTGATTTTCGAGAGT
TTCATACTGTTTTTCTGTAGGCCGTGTACCTAAATGTACTTTTGCTCCATCGCGATGACTTAGTAAAGCAC
ATCTAAAACTTTTAGCGTTATTACGTAAAAAATCTTGCCAGCTTTCCCCTTCTAAAGGGCAAAAGTGAGTA
TGGTGCCTATCTAACATCTCAATGGCTAAGGCGTCGAGCAAAGCCCGCTTATTTTTTACATGCCAATACAA
TGTAGGCTGCTCTACACCTAGCTTCTGGGCGAGTTTACGGGTTGTTAAACCTTCGATTCCGACCTCATTAA
GCAGCTCTAATGCGCTGTTAATCACTTTACTTTTATCTAATCTAGACATcattaattccTACCAATGCTTA
ATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTGAG
CAAAAACAGGAAGGCAAAATGCCGCAAAAAAGGGAATAAGGGCGACACGGAAATGTTGAATACTCATACTC
TTCCTTTTTCAATCATGATTGAAGCATTTATCAGGGTTATTGTCTCATGAGCGGATACATATTTGAATGTA
TTTAGAAAAATAAACAAATAGGTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCTTTTTCCGAAGGTAACT
GGCTTCAGCAGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCTGTGCGGTATTTCACACCGCAT
ATATGGTGCACTCTCAGTACAATCTGCTCTGATGCCGCATAGTTAAGCCAGTATACACTCCGCTATCGCTA
CGTGACTGGGTCATGGCTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAGAGGTTTTCACCGTCAT
CACCGAAACGCGCGAGGCAGCTGCGGTAAAGCTCATCAGCGTGGTCGTGAAGCGATTCACAGATGTCTGCC
TGTTCATCCGCGTCCAGCTCGTTGAGTTTCTCCAGAAGCGTTAATGTCTGGCTTCTGATAAAGCGGGCCAT
GTTAAGGGCGGTTTTTTCCTGTTTGGTCACTGATGCCTCCGTGTAAGGGGGATTTCTGTTCATGGGGGTAA
TGATACCGATGAAACGAGAGAGGATGCTCACGATACGGGTTACTGATGATGAACATGCCCGGTTACTGGAA
CGTTGTGAGGGTAAACAACTGGCGGTATGGATGCGGCGGGACCAGAGAAAAATCACTCAGGGTCAATGCCA
GCGCTTCGTTAATACAGATGTAGGTGTTCCACAGGGTAGCCAGCAGCATCCTGCGATGCAGATCCGGAACA
TAATGGTGCAGGGCGCTGACTTCCGCGTTTCCAGACTTTACGAAACACGGAAACCGAAGACCATTCATGTT
GTTGCTCAGGTCGCAGACGTTTTGCAGCAGCAGTCGCTTCACGTTCGCTCGCGTATCGGTGATTCATTCTG
CTAACCAGTAAGGCAACCCCGCCAGCCTAGCCGGGTCCTCAACGACAGGAGCACGATCATGCTAGTCATGC
CCCGCGCCCACCGGAAGGAGCTGACTGGGTTGAAGGCTCTCAAGGGCATCGGTCGAGATCCCGGTGCCTAA
TGAGTGAGCTAACTTACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCCAGGGTGGTTTTTCTT
TTCACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGCTGT
CTTCGGTATCGTCGTATCCCACTACCGAGATGTCCGCACCAACGCGCAGCCCGGACTCGGTAATGGCGCGC
ATTGCGCCCAGCGCCATCTGATCGTTGGCAACCAGCATCGCAGTGGGAACGATGCCCTCATTCAGCATTTG
CATGGTTTGTTGAAAACCGGACATGGCACTCCAGTCGCCTTCCCGTTCCGCTATCGGCTGAATTTGATTGC
GAGTGAGATATTTATGCCAGCCAGCCAGACGCAGACGCGCCGAGACAGAACTTAATGGGCCCGCTAACAGC
GCGATTTGCTGGTGACCCAATGCGACCAGATGCTCCACGCCCAGTCGCGTACCGTCTTCATGGGAGAAAAT
AATACTGTTGATGGGTGTCTGGTCAGAGACATCAAGAAATAACGCCGGAACATTAGTGCAGGCAGCTTCCA
CAGCAATGGCATCCTGGTCATCCAGCGGATAGTTAATGATCAGCCCACTGACGCGTTGCGCGAGAAGATTG
TGCACCGCCGCTTTACAGGCTTCGACGCCGCTTCGTTCTACCATCGACACCACCACGCTGGCACCCAGTTG
ATCGGCGCGAGATTTAATCGCCGCGACAATTTGCGACGGCGCGTGCAGGGCCAGACTGGAGGTGGCAACGC
CAATCAGCAACGACTGTTTGCCCGCCAGTTGTTGTGCCACGCGGTTGGGAATGTAATTCAGCTCCGCCATC
GCCGCTTCCACTTTTTCCCGCGTTTTCGCAGAAACGTGGCTGGCCTGGTTCACCACGCGGGAAACGGTCTG
ATAAGAGACACCGGCATACTCTGCGACATCGTATAACGTTACTGGTTTCACATTCACCACCCTGAATTGAC
TCTCTTCCGGGCGCTATCATGCCATACCGCGAAAGGTTTTGCGCCATTCGATGGTGTCCGGGATCTCGACG
CTCTCCCTTATGCGACTCCTGCATTAGGAAGCAGCCCAGTAGTAGGTTGAGGCCGTTGAGCACCGCCGCCG
CAAGGAATGGTGCATGCAAGGAGATGGCGCCCAACAGTCCCCCGGCCACGGGGCCTGCCACCATACCCACG
CCGAAACAAGCGCTCATGAGCCCGAAGTGGCGAGCCCGATCTTCCCCATCGGTGATGTCGGCGATATAGGC
GCCAGCAACCGCACCTGTGGCGCCGGTGATGCCGGCCACGATGCGTCCGGCGTAGAGGATCGAGATCGATC
TCGATCCCGCGAAATTAATACGACTCACTATA

TABLE 2
Amino acid sequences of the component protein parts used in the VLPs of
the invention

| | | SEQ | | |
|---|---|---|---|---|
| | | ID | | |
| Name | Description | NO: | Plasmid | Sequence |
| HBclm7 | HBc-capsid-in- | 12 | DU67866 | MDIDPYKEFGASVELLSFLPSDFFPSIRDLLDTASALYRE |
| | frame-fused- | | | ALESPEHCSPHHTALRQAILCWGELMNLATWVGSNL |
| | with lm7 | | | GGGGSGGGGSELKNSISDYTEAEFVQLLKEIEKENVAA |
| | including linkers | | | TDDVLDVLLEHFVKITEHPDGTDLIYYPSDNRDDSPEGI |
| | | | | VKEIKEWRAANGKPGFKQGGGGGSGGGGSPASRELV |
| | | | | VSYVNVNMGLKIRQLLWFHISCLTFGRETVLEYLVSFG |
| | | | | VWIRTPPAYRPPNAPILSTLPETTVV |
| HBclm7 | HBc-capsid-in- | 13 | DU67867 | MDIDPYKEFGASVELLSFLPSDFFPSIRDLLDTASALYRE |
| hh | frame-fused- | | | ALESPEHCSPHHTALRQAILCWGELMNLATWVGSNL |
| | with lm7-with- | | | GGGGSGGGGSELKNSISDYTEAEFVQLLKEIEKENVAA |
| | terminal- | | | TDDVLDVLLEHFVKITEHPDGTDLIYYPSDNRDDSPEGI |
| | HexaHis | | | VKEIKEWRAANGKPGFKQGGGGGSGGGGSPASRELV |
| | including linkers | | | VSYVNVNMGLKIRQLLWFHISCLTFGRETVLEYLVSFG |
| | | | | VWIRTPPAYRPPNAPILSTLPETTVVHHHHHH |
| HBclm7- | HBc-capsid- | 14 | DU70080 | MDIDPYKEFGASVELLSFLPSDFFPSIRDLLDTASALYRE |
| F/L | with-c-terminal- | | | ALESPEHCSPHHTALRQAILCWGELMNLATWVGSNL |
| | hexaHis-in- | | | GGGGSGGGGSELKNSISDYTEAEFVQLLKEIEKENVAA |
| | frame-fused- | | | TDDVLDVLLEHLVKITEHPDGTDLIYYPSDNRDDSPEGI |
| | with lm7 with | | | VKEIKEWRAANGKPGFKQGGGGGSGGGGSPASRELV |
| | F97L mutation | | | VSYVNVNMGLKIRQLLWFHISCLTFGRETVLEYLVSFG |
| | including linkers | | | VWIRTPPAYRPPNAPILSTLPETTVVHHHHHH |
| HBcBsA | HBc-capsid- | 15 | DU-Bn | MDIDPYKEFGASVELLSFLPSDFFPSIRDLLDTASALYRE |
| AE | with-c-terminal- | | | ALESPEHCSPHHTALRQAILCWGELMNLATWVGSNL |
| | hexaHis in- | | | GGGGSGGGGSKKAVINGEQIRSISDLHQTLKKELALPE |
| | frame-fused- | | | YYGENLDALWDALTGWVEYPLVLEWRQFEQSKQLTE |
| | with Barstar | | | NGAESVLQVFREAKAEGADITIELSGGGGGSGGGGSP |
| | (including C40A, | | | ASRELVVSYVNVNMGLKIRQLLWFHISCLTFGRETVLE |
| | C82A; 187E | | | YLVSFGVWIRTPPAYRPPNAPILSTLPETTVVHHHHHH |
| | mutations) and | | | |
| | including linkers | | | |
| C7-IL13 | ColE7-catalytic- | 16 | DU70076 | ESKRNKPGKATGKGKPVNNKWLNNAGKDLGSPVPD |
| | domain-fused- | | | RIANKLRDKEFKSFDDFRKKFWEEVSKDPELSKQFSRN |
| | to-hIL13 | | | NNDRMKVGKAPKTRTQDVSGKATSFALHHEKPISQN |
| | including linker | | | GGVYDMDNISVVTPKRAIDIHRGKSGGGSSGSGGPVP |
| | | | | PSTALRELIEELVNITQNQKAPLCNGSMVWSINLTAG |
| | | | | MYCAALESLINVSGCSAIEKTQRMLSGFCPHKVSAGQ |
| | | | | FSSLHVRDTKIEVAQFVKDLLLHLKKLFREGQFN |
| C7-IL13- | ColE7-catalytic- | 17 | DU70080 | ESKRNKPGKATGKGKPVNNKWLNNAGKDLGSPVPD |
| TEV | domain-fused- | | | RIANKLRDKEFKSFDDFRKKFWEEVSKDPELSKQFSRN |
| | to-hIL13 | | | NNDRMKVGKAPKTRTQDVSGKATSFALHHEKPISQN |
| | including linker | | | GGVYDMDNISVVTPKRAIDIHRGKSGGGSSGSGENLY |
| | including TEV | | | FQGGPVPPSTALRELIEELVNITQNQKAPLCNGSMVW |
| | protease site | | | SINLTAGMYCAALESLINVSGCSAIEKTQRMLSGFCPH |
| | | | | KVSAGQFSSLHVRDTKIEVAQFVKDLLLHLKKLFREGQ |
| | | | | FN |
| C7-IL33 | ColE7-catalytic- | 18 | | ESKRNKPGKATGKGKPVNNKWLNNAGKDLGSPVPD |
| | domain-fused- | | | RIANKLRDKEFKSFDDFRKKFWEEVSKDPELSKQFSRN |
| | to-hIL33 | | | NNDRMKVGKAPKTRTQDVSGKATSFALHHEKPISQN |
| | including linker | | | GGVYDMDNISVVTPKRAIDIHRGKSGGGSSGSGSSIQ |
| | | | | GTSLLTQSPASLSTYNDQSVSFVLENGCYVINVDKSGK |
| | | | | DQEQDQVLLRYYKSPCPASQSGDGVDGKKLMVNMS |
| | | | | PIKDTDIWLHANDKDYSVELQRGDVSPPEQAFFVLHK |
| | | | | KSSDFVSFECKNLPGTYIGVKDNQLALVEEKDESCNNI |
| | | | | MFKLSKI |
| C7-IL33- | ColE7-catalytic- | 19 | DU70126 | ESKRNKPGKATGKGKPVNNKWLNNAGKDLGSPVPD |
| TEV | domain-fused- | | | RIANKLRDKEFKSFDDFRKKFWEEVSKDPELSKQFSRN |
| | to-hIL33 | | | NNDRMKVGKAPKTRTQDVSGKATSFALHHEKPISQN |
| | including linker | | | GGVYDMDNISVVTPKRAIDIHRGKSGGGSSGSGENLY |
| | including TEV | | | FQGSSIQGTSLLTQSPASLSTYNDQSVSFVLENGCYVIN |
| | protease site | | | VDKSGKDQEQDQVLLRYYKSPCPASQSGDGVDGKKL |
| | | | | MVNMSPIKDTDIWLHANDKDYSVELQRGDVSPPEQA |
| | | | | FFVLHKKSSDFVSFECKNLPGTYIGVKDNQLALVEEKDE |
| | | | | SCNNIMFKLSKI |
| C7-PG | ColE7-catalytic- | 20 | DU65750 | TYKLILNGKTLKGETTTEAVDAATAEKVFKQYANDNGV |
| | domain-C- | | | DGEWTYDDATKTFTVTEKPEVIDASELTPAVTTYKLVI |
| | terminally- | | | NGKTLKGETTTEAVDAATAEKVFKQYANDNGVDGE |
| | fused-to- | | | WTYDDATKTFTVTEKPEVIDASELTPAVTTYKLVINGKT |
| | Protein-G | | | LKGETTTKAVDAETAEKAFKQYANDNGVDGVWTYDD |
| | | | | ATKTFTVTEGGGSSGSGGGGSSGSGENLYFQGESKRN |
| | | | | KPGKATGKGKPVNNKWLNNAGKDLGSPVPDRIANKL |
| | | | | RDKEFKSFDDFRKKFWEEVSKDPELSKQFSRNNNDR |
| | | | | MKVGKAPKTRTQDVSGKATSFALHHEKPISQNGGVY |
| | | | | DMDNISVVTPKRAIDIHRGKS |
| Bn-IL13 | Barnase-with- | 21 | DU-Bn | AQVINTFDGVADYLQTYHKLPDNYITKSEAQALGWVA |
| | catalytically- | | | SKGNLADVAPGKSIGGDIFSNREGKLPGKSGRTWRW |
| | inactivating | | | ADINYTSGFRNSDRILYSSDWLIYKTTDHYQTFTKIRGG |
| | mutation | | | GSSGSGENLYFQGGPVPPSTALRELIEELVNITQNQKA |
| | E73W-fused-to- | | | PLCNGSMVWSINLTAGMYCAALESLINVSGCSAIEKT |
| | IL13 | | | QRMLSGFCPHKVSAGQFSSLHVRDTKIEVAQFVKDLLL |
| | | | | HLKKLFREGQFN |
| ColE7- | ColicinE7- | 46 | DU70680 | MESKRNKPGKATGKGKPVNNKWLNNAGKDLGS |
| IL17 | fused-to- | | | PVPDRIANKLRDKEFKSFDDFRKKFWEEVSKDPEL |
| | human-IL17A- | | | SKQFSRNNNDRMKVGKAPKTRTQDVSGKATSFA |
| | with_linker- | | | LHHEKPISQNGGVYDMDNISVVTPKRAIDIHRGK |
| | and-TEV- | | | SGGGSSGSGENLYFQGITIPRNPGCPNSEDKNFPR |
| | cleavage-site | | | TVMVNLNIHNRNTNTNPKRSSDYYNRSTSPWNL |
| | | | | HRNEDPERYPSVIWEAKCRHLGCINADGNVDYH |
| | | | | MNSVPIQQEILVLRREPPHCPNSFRLEKILVSVGCT |
| | | | | CVTPIVHHVA |

Linkers:
(SEQ ID NO: 33)
GGGGSGGGGS
(SEQ ID NO: 34)
GGGGGSGGGGS
(SEQ ID NO: 35)
SGGGSSGSG
Barstar:
(SEQ ID NO: 36)
KKAVINGEQIRSISDLHQTLKKELALPEYYGENLDALWDALTGWVEYPL
VLEWRQFEQSKQLTENGAESVLQVFREAKAEGADITIELS
lm7:
(SEQ ID NO: 37)
ELKNSISDYTEAEFVQLLKEIEKENVAATDDVLDVLLEHFVKITEHPDG
TDLIYYPSDNRDDSPEGIVKEIKEWRAANGKPGFKQ
Barnase:
(SEQ ID NO: 38)
AQVINTFDGVADYLQTYHKLPDNYITKSEAQALGWVASKGNLADVAPGK
SIGGDIFSNREGKLPGKSGRTWRWADINYTSGFRNSDRILYSSDWLIYK
TTDHYQTFTKIR.
ColE7:
(SEQ ID NO: 39)
ESKRNKPGKATGKGKPVNNKWLNNAGKDLGSPVPDRIANKLRDKEFKSF
DDFRKKFWEEVSKDPELSKQFSRNNNDRMKVGKAPKTRTQDVSGKATSF
ALHHEKPISQNGGVYDMDNISVVTPKRAIDIHRGKS
HBc:
(SEQ ID NO: 40)
MDIDPYKEFGASVELLSFLPSDFFPSIRDLLDTASALYREALESPEHCS
PHHTALRQAILCWGELMNLATWVGSNL[X]PASRELVVSYVNVNMGLKI
RQLLWFHISCLTFGRETVLEYLVSFGVWIRTPPAYRPPNAPILSTLPET
TVV

TABLE 3
Nucleic acid sequences encoding the parts used in the VLPs of the invention

| | | SEQ | | |
|---|---|---|---|---|
| | | ID | | |
| Name | Description | NO: | Plasmid | sequence |
| HBclm7 | HBc-capsid-in- | 22 | DU67866 | atggacattgacccgtataaagaatttggagcttctgtggagttactct |
| | frame-fused-with | | | cttttttgccttctgacttctttccttctattcgagatctcctcgacaccgc |
| | lm7 including | | | ctcagctctgtatcgggaggccttagagtctccggaacattgttcacct |
| | linkers | | | caccatacagcactcaggcaagctattctgtgttggggtgagttgatga |
| | | | | atttggccacctgggtgggaagtaatttgggaggaggtggaagcgga |
| | | | | ggaggtggatctgaactgaaaaatagtattagtgattacacagaggct |
| | | | | gagtttgttcaacttcttaaggaaattgaaaaagagaatgttgctgca |
| | | | | actgatgatgtgttagatgtgttactcgaacactttgtaaaaattactg |
| | | | | agcatccagatggaacggatctgatttattatcctagtgataatagag |
| | | | | acgatagccccgaagggattgtcaaggaaattaaagaatggcgagct |
| | | | | gctaacggtaagccaggatttaaacagggcggaggaggtggaagcg |
| | | | | gaggaggtggatctccagcatccagggaattagtagtcagctatgtta |
| | | | | atgttaatatgggcctaaaaatcagacaactactgtggtttcacatttc |
| | | | | ctgtcttacttttggaagagaaactgttcttgaatatttggtgtcttttgg |
| | | | | agtgtggattcgcactcctcctgcttacagaccaccaaatgcccctatc |
| | | | | ttatcaacacttccggaaactactgttgtt |
| HBclm7hh | HBc-capsid-in- | 23 | DU67867 | atggacattgacccgtataaagaatttggagcttctgtggagttactct |
| | frame-fused-with | | | cttttttgccttctgacttctttccttctattcgagatctcctcgacaccgc |
| | lm7-with- | | | ctcagctctgtatcgggaggccttagagtctccggaacattgttcacct |
| | terminal-HexaHis | | | caccatacagcactcaggcaagctattctgtgttggggtgagttgatga |
| | including linkers | | | atttggccacctgggtgggaagtaatttgggaggaggtggaagcgga |
| | | | | ggaggtggatctgaactgaaaaatagtattagtgattacacagaggct |
| | | | | gagtttgttcaacttcttaaggaaattgaaaaagagaatgttgctgca |
| | | | | actgatgatgtgttagatgtgttactcgaacactttgtaaaaattactg |
| | | | | agcatccagatggaacggatctgatttattatcctagtgataatagag |
| | | | | acgatagccccgaagggattgtcaaggaaattaaagaatggcgagct |
| | | | | gctaacggtaagccaggatttaaacagggcggaggaggtggaagcg |
| | | | | gaggaggtggatctccagcatccagggaattagtagtcagctatgtta |
| | | | | atgttaatatgggcctaaaaatcagacaactactgtggtttcacatttc |
| | | | | ctgtcttacttttggaagagaaactgttcttgaatatttggtgtcttttgg |
| | | | | agtgtggattcgcactcctcctgcttacagaccaccaaatgcccctatc |
| | | | | ttatcaacacttccggaaactactgttgttCATCACCATCATCAC |
| | | | | CAC |
| HBclm7- | HBc-capsid-with- | 24 | DU70080, | atggacattgacccgtataaagaatttggagcttctgtggagttactct |
| F/L | c-terminal- | | DU67923 | cttttttgccttctgacttctttccttctattcgagatctcctcgacaccgc |
| | hexaHis-in-frame- | | | ctcagctctgtatcgggaggccttagagtctccggaacattgttcacct |
| | fused-with Im7 | | | caccatacagcactcaggcaagctattctgtgttggggtgagttgatga |
| | with F97L | | | atttggccacctgggtgggaagtaatttgggaggaggtggaagcgga |
| | mutation | | | ggaggtggatctgaactgaaaaatagtattagtgattacacagaggct |
| | including linkers | | | gagtttgttcaacttcttaaggaaattgaaaaagagaatgttgctgca |
| | | | | actgatgatgtgttagatgtgttactcgaacacctagtaaaaattactg |
| | | | | agcatccagatggaacggatctgatttattatcctagtgataatagag |
| | | | | acgatagccccgaagggattgtcaaggaaattaaagaatggcgagct |
| | | | | gctaacggtaagccaggatttaaacagggcggaggaggtggaagcg |
| | | | | gaggaggtggatctccagcatccagggaattagtagtcagctatgtta |
| | | | | atgttaatatgggcctaaaaatcagacaactactgtggtttcacatttc |
| | | | | ctgtcttacttttggaagagaaactgttcttgaatatttggtgtcttttgg |
| | | | | agtgtggattcgcactcctcctgcttacagaccaccaaatgcccctatc |
| | | | | ttatcaacacttccggaaactactgttgttCATCACCATCATCAC |
| | | | | CAC |
| HBcBs | HBc-capsid-with- | 25 | DU-Bn | atggacattgacccgtataaagaatttggagcttctgtggagttactct |
| AAE | c-terminal- | | | cttttttgccttctgacttctttccttctattcgagatctcctcgacaccgc |
| | hexaHis in-frame- | | | ctcagctctgtatcgggaggccttagagtctccggaacattgttcacct |
| | fused-with | | | caccatacagcactcaggcaagctattctgtgttggggtgagttgatga |
| | Barstar (including | | | atttggccacctgggtgggaagtaatttgggaggaggtggaagcgga |
| | C40A, C82A; I87E | | | ggaggtggatctaaaaaagcagtcattaacggggaacaaatcagaa |
| | mutations) and | | | gtatcagcgacctccaccagacattgaaaaaggagcttgcccttccgg |
| | including linkers | | | aatactacggtgaaaacctggacgctttatgggatgcgctgaccggat |
| | | | | gggtggagtacccgctcgttttggaatggaggcagtttgaacaaagca |
| | | | | agcagctgactgaaaatggcgccgagagtgtgcttcaggttttccgtg |
| | | | | aagcgaaagcggaaggcgcggacatcaccatcgaactttctggcgga |
| | | | | ggaggtggaagcggaggaggtggatctccagcatccagggaattagt |
| | | | | agtcagctatgttaatgttaatatgggcctaaaaatcagacaactact |
| | | | | gtggtttcacatttcctgtcttacttttggaagagaaactgttcttgaata |
| | | | | tttggtgtcttttggagtgtggattcgcactcctcctgcttacagaccac |
| | | | | caaatgcccctatcttatcaacacttccggaaactactgttgttCATC |
| | | | | ACCATCATCACCAC |
| C7- | ColE7-catalytic- | 26 | | TGGAGAGTAAACGGAATAAGCCAGGGAAGGCAACA |
| IL13 | domain-fused-to- | | | GGTAAAGGAAAACCTGTCAATAATAAGTGGTTAAATA |
| | hIL13 including | | | ATGCAGGTAAAGACTTAGGTTCTCCTGTTCCAGATCG |
| | linker | | | TATAGCTAATAAACTACGTGATAAGGAGTTTAAAAGT |
| | | | | TTCGATGATTTTCGTAAGAAATTCTGGGAAGAAGTGT |
| | | | | CAAAAGATCCTGAGTTAAGTAAACAATTTAGTCGAAA |
| | | | | CAATAATGATCGAATGAAGGTTGGAAAAGCGCCCAA |
| | | | | GACTAGAACCCAGGATGTTTCAGGGAAGGCAACTTC |
| | | | | ATTCGCACTTCATCATGAGAAGCCGATCAGCCAAAAT |
| | | | | GGTGGTGTCTATGATATGGATAACATCAGCGTGGTAA |
| | | | | CACCTAAACGTGCTATTGATATTCACCGAGGTAAAAG |
| | | | | CGGAGGTGGCTCATCAGGATCtGGTGGCCCGGTGCCT |
| | | | | CCAAGCACTGCCCTGCGTGAGTTAATCGAAGAATTAG |
| | | | | TCAACATTACCCAAAACCAGAAGGCACCGCTGTGCAA |
| | | | | CGGTTCAATGGTCTGGAGCATCAATCTGACAGCCGGT |
| | | | | ATGTATTGCGCGGCGCTTGAAAGCCTGATTAACGTGA |
| | | | | GTGGTTGTTCTGCCATCGAAAAAACTCAACGTATGTT |
| | | | | GTCCGGCTTTTGCCCACACAAGGTATCTGCCGGTCAA |
| | | | | TTCAGTTCATTGCACGTTCGTGATACAAAAATTGAGG |
| | | | | TAGCCCAGTTCGTGAAGGACTTACTGTTACATTTGAA |
| | | | | AAAGTTATTCCGTGAAGGGCAATTTAAC |
| C7- | ColE7-catalytic- | 27 | DU70080 | ATGGAGAGTAAACGGAATAAGCCAGGGAAGGCAAC |
| IL13- | domain-fused-to- | | | AGGTAAAGGAAAACCTGTCAATAATAAGTGGTTAAAT |
| TEV | hIL13 including | | | AATGCAGGTAAAGACTTAGGTTCTCCTGTTCCAGATC |
| | linker including | | | GTATAGCTAATAAACTACGTGATAAGGAGTTTAAAAG |
| | TEV protease site | | | TTTCGATGATTTTCGTAAGAAATTCTGGGAAGAAGTG |
| | | | | TCAAAAGATCCTGAGTTAAGTAAACAATTTAGTCGAA |
| | | | | ACAATAATGATCGAATGAAGGTTGGAAAAGCGCCCA |
| | | | | AGACTAGAACCCAGGATGTTTCAGGGAAGGCAACTT |
| | | | | CATTCGCACTTCATCATGAGAAGCCGATCAGCCAAAA |
| | | | | TGGTGGTGTCTATGATATGGATAACATCAGCGTGGTA |
| | | | | ACACCTAAACGTGCTATTGATATTCACCGAGGTAAAA |
| | | | | GCGGAGGTGGCTCATCAGGATCtGGTGAAAACCTGTA |
| | | | | TTTTCAGGGatccGGCCCGGTGCCTCCAAGCACTGCCC |
| | | | | TGCGTGAGTTAATCGAAGAATTAGTCAACATTACCCA |
| | | | | AAACCAGAAGGCACCGCTGTGCAACGGTTCAATGGT |
| | | | | CTGGAGCATCAATCTGACAGCCGGTATGTATTGCGCG |
| | | | | GCGCTTGAAAGCCTGATTAACGTGAGTGGTTGTTCTG |
| | | | | CCATCGAAAAAACTCAACGTATGTTGTCCGGCTTTTGC |
| | | | | CCACACAAGGTATCTGCCGGTCAATTCAGTTCATTGC |
| | | | | ACGTTCGTGATACAAAAATTGAGGTAGCCCAGTTCGT |
| | | | | GAAGGACTTACTGTTACATTTGAAAAAGTTATTCCGT |
| | | | | GAAGGGCAATTTAAC |
| C7- | ColE7-catalytic- | 28 | | TGGAGAGTAAACGGAATAAGCCAGGGAAGGCAACA |
| IL33 | domain-fused-to- | | | GGTAAAGGAAAACCTGTCAATAATAAGTGGTTAAATA |
| | hIL33 including | | | ATGCAGGTAAAGACTTAGGTTCTCCTGTTCCAGATCG |
| | linker | | | TATAGCTAATAAACTACGTGATAAGGAGTTTAAAAGT |
| | | | | TTCGATGATTTTCGTAAGAAATTCTGGGAAGAAGTGT |
| | | | | CAAAAGATCCTGAGTTAAGTAAACAATTTAGTCGAAA |
| | | | | CAATAATGATCGAATGAAGGTTGGAAAAGCGCCCAA |
| | | | | GACTAGAACCCAGGATGTTTCAGGGAAGGCAACTTC |
| | | | | ATTCGCACTTCATCATGAGAAGCCGATCAGCCAAAAT |
| | | | | GGTGGTGTCTATGATATGGATAACATCAGCGTG29GT |
| | | | | AACACCTAAACGTGCTATTGATATTCACCGAGGTAAA |
| | | | | AGCGGAGGTGGCTCATCAGGATCtGGTGAAAACCTGT |
| | | | | ATTTTCAGGGatccGGCCCGGTGCCTCCAAGCACTGCC |
| | | | | CTGCGTGAGTTAATCGAAGAATTAGTCAACATTACCC |
| | | | | AAAACCAGAAGGCACCGCTGTGCAACGGTTCAATGG |
| | | | | TCTGGAGCATCAATCTGACAGCCGGTATGTATTGCGC |
| | | | | GGCGCTTGAAAGCCTGATTAACGTGAGTGGTTGTTCT |
| | | | | GCCATCGAAAAAACTCAACGTATGTTGTCCGGCTTTT |
| | | | | GCCCACACAAGGTATCTGCCGGTCAATTCAGTTCATT |
| | | | | GCACGTTCGTGATACAAAAATTGAGGTAGCCCAGTTC |
| | | | | GTGAAGGACTTACTGTTACATTTGAAAAAGTTATTCC |
| | | | | GTGAAGGGCAATTTAAC |
| C7- | ColE7-catalytic- | 29 | DU70126 | ATGGAGAGTAAACGGAATAAGCCAGGGAAGGCAAC |
| IL33- | domain-fused-to- | | | AGGTAAAGGAAAACCTGTCAATAATAAGTGGTTAAAT |
| TEV | hIL33 including | | | AATGCAGGTAAAGACTTAGGTTCTCCTGTTCCAGATC |
| | linker including | | | GTATAGCTAATAAACTACGTGATAAGGAGTTTAAAAG |
| | TEV protease site | | | TTTCGATGATTTTCGTAAGAAATTCTGGGAAGAAGTG |
| | | | | TCAAAAGATCCTGAGTTAAGTAAACAATTTAGTCGAA |
| | | | | ACAATAATGATCGAATGAAGGTTGGAAAAGCGCCCA |
| | | | | AGACTAGAACCCAGGATGTTTCAGGGAAGGCAACTT |
| | | | | CATTCGCACTTCATCATGAGAAGCCGATCAGCCAAAA |
| | | | | TGGTGGTGTCTATGATATGGATAACATCAGCGTGGTA |
| | | | | ACACCTAAACGTGCTATTGATATTCACCGAGGTAAAA |
| | | | | GCGGAGGTGGCTCATCAGGATCtGGTGAAAACCTGTA |
| | | | | TTTTCAGGGatccAgcattcagggcaccagcctgctgacccagag |
| | | | | cccggcgagcctgagcacctataacgatcagagcgtgagctttgtgct |
| | | | | ggaaaacggctgctatgtgattaacgtggataaaagcggcaaagatc |
| | | | | aggaacaggatcaggtgctgctgcgctattataaaagcccgtgcccgg |
| | | | | cgagccagagcggcgatggcgtggatggcaagaaactgatggtgaa |
| | | | | catgagcccgattaaagataccgatatttggctgcatgcgaacgataa |
| | | | | agattatagcgtggaactgcagcgcggcgatgtgagcccgccagaac |
| | | | | aggcgttctttgtgctgcataagaaaagcagcgattttgtgagctttga |
| | | | | atgcaaaaacctgccgggcacctatattggcgtgaaagataaccagc |
| | | | | tggcgctggtggaagagaaagatgaaagctgcaacaacattatgttt |
| | | | | aaactgagcaaaatt |
| C7-PG | ColE7-catalytic- | 30 | DU65750 | atgACCTATAAACTGATTCTGAACGGCAAAACGCTGAA |
| | domain-C- | | | AGGTGAAACCACCACCGAAGCAGTGGATGCCGCAAC |
| | terminally-fused- | | | CGCCGAAAAAGTTTTCAAACAGTACGCCAACGATAAT |
| | to-Protein-G | | | GGCGTTGATGGTGAATGGACCTACGATGATGCAACG |
| | | | | AAAACCTTCACGGTGACCGAAAAACCGGAAGTTATTG |
| | | | | ATGCCAGCGAACTGACGCCGGCAGTGACGACCTACA |
| | | | | AACTGGTTATCAATGGCAAAACCCTGAAAGGTGAAAC |
| | | | | GACCACCGAAGCCGTGGATGCAGCCACCGCAGAAAA |
| | | | | AGTGTTTAAACAGTACGCGAACGATAATGGCGTGGA |
| | | | | CGGCGAATGGACGTATGATGATGCCACCAAAACGTTT |
| | | | | ACCGTTACGGAAAAACCGGAAGTGATCGATGCATCT |
| | | | | GAACTGACCCCGGCGGTGACCACGTATAAACTGGTTA |
| | | | | TCAACGGTAAAACGCTGAAAGGCGAAACCACGACCA |
| | | | | AAGCCGTGGATGCAGAAACCGCGGAAAAAGCCTTTA |
| | | | | AACAGTATGCAAACGATAATGGCGTGGATGGTGTTT |
| | | | | GGACCTACGATGATGCCACGAAAACCTTCACGGTTAC |
| | | | | CGAAggcggtgggagtagcggaagtgggggtggtggatccagtGG |
| | | | | tTCTGGgGAAAAtCTGTACTTCCAGGGTGAGAGTAAAC |
| | | | | GGAATAAGCCAGGGAAGGCAACAGGTAAAGGAAAA |
| | | | | CCTGTCAATAATAAGTGGTTAAATAATGCAGGTAAAG |
| | | | | ACTTAGGTTCTCCTGTTCCAGATCGTATAGCTAATAAA |
| | | | | CTACGTGATAAGGAGTTTAAAAGTTTCGATGATTTTC |
| | | | | GTAAGAAATTCTGGGAAGAAGTGTCAAAAGATCCTG |
| | | | | AGTTAAGTAAACAATTTAGTCGAAACAATAATGATCG |
| | | | | AATGAAGGTTGGAAAAGCGCCCAAGACTAGAACCCA |
| | | | | GGATGTTTCAGGGAAGGCAACTTCATTCGCACTTCAT |
| | | | | CATGAGAAGCCGATCAGCCAAAATGGTGGTGTCTAT |
| | | | | GATATGGATAACATCAGCGTGGTAACACCTAAACGTG |
| | | | | CTATTGATATTCACCGAGGTAAAAGC |
| T7- | T7-mutant- | 31 | T7- | TAATACGACTCACTATAGG |
| mutant | promoter | | mutant | |
| Bn- | Barnase-with- | 32 | DU-Bn | ATGgcacaggttatcaacacgtttgacggggttgcggattatcttcag |
| IL13 | catalytically- | | | acatatcataagctacctgataattacattacaaaatcagaagcacaa |
| | inactivating | | | gccctcggctgggtggcatcaaaagggaaccttgcagacgtcgctccg |
| | mutation E73W- | | | gggaaaagcatcggcggagacatcttctcaaacagggaaggcaaac |
| | fused-to-IL13 | | | tcccgggcaaaagcggacgaacatggcgtTGGgcggatattaacta |
| | | | | tacatcaggcttcagaaattcagaccggattctttactcaagcgactgg |
| | | | | ctgatttacaaaacaacggaccattatcagacctttacaaaaatcaga |
| | | | | GGAGGTGGCTCATCAGGATCtGGTGAAAACCTGTATT |
| | | | | TTCAGGGatccGGCCCGGTGCCTCCAAGCACTGCCCTG |
| | | | | CGTGAGTTAATCGAAGAATTAGTCAACATTACCCAAA |
| | | | | ACCAGAAGGCACCGCTGTGCAACGGTTCAATGGTCTG |
| | | | | GAGCATCAATCTGACAGCCGGTATGTATTGCGCGGCG |
| | | | | CTTGAAAGCCTGATTAACGTGAGTGGTTGTTCTGCCA |
| | | | | TCGAAAAAACTCAACGTATGTTGTCCGGCTTTTGCCCA |
| | | | | CACAAGGTATCTGCCGGTCAATTCAGTTCATTGCACG |
| | | | | TTCGTGATACAAAAATTGAGGTAGCCCAGTTCGTGAA |
| | | | | GGACTTACTGTTACATTTGAAAAAGTTATTCCGTGAA |
| | | | | GGGCAATTTAACtaataaGGTACCCTCGAGTCTGGTAA |
| | | | | AGAAACCGCTGCTGCGAAATTTGAACGCCAGCACATG |
| | | | | GACTCG |
| C7-RBD | ColE7 fused N- | 41 | | GAGAGTAAACGGAATAAGCCAGGGAAGGCAACAGG |
| | terminally to | | | TAAAGGAAAACCTGTCAATAATAAGTGGTTAAATAAT |
| | SARS2-Spike | | | GCAGGTAAAGACTTAGGTTCTCCTGTTCCAGATCGTA |
| | Receptor binding | | | TAGCTAATAAACTACGTGATAAGGAGTTTAAAAGTTT |
| | domain | | | CGATGATTTTCGTAAGAAATTCTGGGAAGAAGTGTCA |
| | | | | AAAGATCCTGAGTTAAGTAAACAATTTAGTCGAAACA |
| | | | | ATAATGATCGAATGAAGGTTGGAAAAGCGCCCAAGA |
| | | | | CTAGAACCCAGGATGTTTCAGGGAAGGCAACTTCATT |
| | | | | CGCACTTCATCATGAGAAGCCGATCAGCCAAAATGGT |
| | | | | GGTGTCTATGATATGGATAACATCAGCGTGGTAACAC |
| | | | | CTAAACGTGCTATTGATATTCACCGAGGTAAAAGCGG |
| | | | | AGGTGGCTCATCAGGATCCGGTAGAGTCCAACCAAC |
| | | | | AGAATCTATTGTTAGATTTCCTAATATTACAAACTTGT |
| | | | | GCCCTTTTGGTGAAGTTTTTAACGCCACCAGATTTGCA |
| | | | | TCTGTTTATGCTTGGAACAGGAAGAGAATCAGCAACT |
| | | | | GTGTTGCTGATTATTCTGTCCTATATAATTCCGCATCA |
| | | | | TTTTCCACTTTTAAGTGTTATGGAGTGTCTCCTACTAA |
| | | | | ATTAAATGATCTCTGCTTTACTAATGTCTATGCAGATT |
| | | | | CATTTGTAATTAGAGGTGATGAAGTCAGACAAATCGC |
| | | | | TCCAGGGCAAACTGGAAAGATTGCTGATTATAATTAT |
| | | | | AAATTACCAGATGATTTTACAGGCTGCGTTATAGCTT |
| | | | | GGAATTCTAACAATCTTGATTCTAAGGTTGGTGGTAA |
| | | | | TTATAATTACCTGTATAGATTGTTTAGGAAGTCTAATC |
| | | | | TCAAACCTTTTGAGAGAGATATTTCAACTGAAATCTAT |
| | | | | CAGGCCGGTAGCACACCTTGTAATGGTGTTGAAGGTT |
| | | | | TTAATTGTTACTTTCCTTTACAATCATATGGTTTCCAAC |
| | | | | CCACTAATGGTGTTGGTTACCAACCATACAGAGTAGT |
| | | | | AGTACTTTCTTTTGAACTTCTACATGCACCAGCAACTG |
| | | | | TTTGTGGACCTAAAAAGTCTACTAATTTGGTTAAAAAC |
| | | | | AAATGTGTCAATTT |
| C7- | ColE7-N-terminally | 42 | | GAGAGTAAACGGAATAAGCCAGGGAAGGCAACAGG |
| RBD- | fused to SARS | | | TAAAGGAAAACCTGTCAATAATAAGTGGTTAAATAAT |
| nucleo | Spike-Receptor- | | | GCAGGTAAAGACTTAGGTTCTCCTGTTCCAGATCGTA |
| capsid | binding-domain in | | | TAGCTAATAAACTACGTGATAAGGAGTTTAAAAGTTT |
| | tandem with non- | | | CGATGATTTTCGTAAGAAATTCTGGGAAGAAGTGTCA |
| | RNA-binding C- | | | AAAGATCCTGAGTTAAGTAAACAATTTAGTCGAAACA |
| | terminal fragment | | | ATAATGATCGAATGAAGGTTGGAAAAGCGCCCAAGA |
| | of SARS2- | | | CTAGAACCCAGGATGTTTCAGGGAAGGCAACTTCATT |
| | nucleocapsid | | | CGCACTTCATCATGAGAAGCCGATCAGCCAAAATGGT |
| | protein | | | GGTGTCTATGATATGGATAACATCAGCGTGGTAACAC |
| | | | | CTAAACGTGCTATTGATATTCACCGAGGTAAAAGCGG |
| | | | | AGGTGGCTCATCAGGATCCGGTAGAGTCCAACCAAC |
| | | | | AGAATCTATTGTTAGATTTCCTAATATTACAAACTTGT |
| | | | | GCCCTTTTGGTGAAGTTTTTAACGCCACCAGATTTGCA |
| | | | | TCTGTTTATGCTTGGAACAGGAAGAGAATCAGCAACT |
| | | | | GTGTTGCTGATTATTCTGTCCTATATAATTCCGCATCA |
| | | | | TTTTCCACTTTTAAGTGTTATGGAGTGTCTCCTACTAA |
| | | | | ATTAAATGATCTCTGCTTTACTAATGTCTATGCAGATT |
| | | | | CATTTGTAATTAGAGGTGATGAAGTCAGACAAATCGC |
| | | | | TCCAGGGCAAACTGGAAAGATTGCTGATTATAATTAT |
| | | | | AAATTACCAGATGATTTTACAGGCTGCGTTATAGCTT |
| | | | | GGAATTCTAACAATCTTGATTCTAAGGTTGGTGGTAA |
| | | | | TTATAATTACCTGTATAGATTGTTTAGGAAGTCTAATC |
| | | | | TCAAACCTTTTGAGAGAGATATTTCAACTGAAATCTAT |
| | | | | CAGGCCGGTAGCACACCTTGTAATGGTGTTGAAGGTT |
| | | | | TTAATTGTTACTTTCCTTTACAATCATATGGTTTCCAAC |
| | | | | CCACTAATGGTGTTGGTTACCAACCATACAGAGTAGT |
| | | | | AGTACTTTCTTTTGAACTTCTACATGCACCAGCAACTG |
| | | | | TTTGTGGACCTAAAAAGTCTACTAATTTGGTTAAAAAC |
| | | | | AAATGTGTCAATTTCGGAGGTGGCTCATCAGGATCAG |
| | | | | GTGGTTTACCCAATAATACTGCGTCTTGGTTCACCGCT |
| | | | | CTCACTCAACATGGCAAGGAAGACCTTAAATTCCCTC |
| | | | | GAGGACAAGGCGTTCCAATTAACACCAATAGCAGTCC |
| | | | | AGATGACCAAATTGGCTACTACCGAAGAGCTACCAGA |
| | | | | CGAATTCGTGGTGGTGACGGTAAAATGAAAgacCTCA |
| | | | | GTCCAAGATGGTATTTCTACTACCTAGGAACTGGGCC |
| | | | | AGAAGCTGGACTTCCCTATGGTGCTAACAAAGACGGC |
| | | | | ATCATATGGGTTGCAACTGAGGGAGCCTTGAATACAC |
| | | | | CAAAAGATCACATTGGCACCCGCAATCCTGCTAACAA |
| | | | | TGCTGCAATCGTGCTACAACTTCCTCAAGGAACAACA |
| | | | | TTGCCAAAAGGCTTCTACGCAGAAGGGAGCAGAGGC |
| | | | | GGCAGT |
| C7- | ColE7-N-terminally | 43 | | GAGAGTAAACGGAATAAGCCAGGGAAGGCAACAGG |
| RBD- | fused to Spike- | | | TAAAGGAAAACCTGTCAATAATAAGTGGTTAAATAAT |
| GFP | Receptor-binding- | | | GCAGGTAAAGACTTAGGTTCTCCTGTTCCAGATCGTA |
| | domain with c- | | | TAGCTAATAAACTACGTGATAAGGAGTTTAAAAGTTT |
| | terminal eGFP | | | CGATGATTTTCGTAAGAAATTCTGGGAAGAAGTGTCA |
| | | | | AAAGATCCTGAGTTAAGTAAACAATTTAGTCGAAACA |
| | | | | ATAATGATCGAATGAAGGTTGGAAAAGCGCCCAAGA |
| | | | | CTAGAACCCAGGATGTTTCAGGGAAGGCAACTTCATT |
| | | | | CGCACTTCATCATGAGAAGCCGATCAGCCAAAATGGT |
| | | | | GGTGTCTATGATATGGATAACATCAGCGTGGTAACAC |
| | | | | CTAAACGTGCTATTGATATTCACCGAGGTAAAAGCGG |
| | | | | AGGTGGCTCATCAGGATCCGGTAGAGTCCAACCAAC |
| | | | | AGAATCTATTGTTAGATTTCCTAATATTACAAACTTGT |
| | | | | GCCCTTTTGGTGAAGTTTTTAACGCCACCAGATTTGCA |
| | | | | TCTGTTTATGCTTGGAACAGGAAGAGAATCAGCAACT |
| | | | | GTGTTGCTGATTATTCTGTCCTATATAATTCCGCATCA |
| | | | | TTTTCCACTTTTAAGTGTTATGGAGTGTCTCCTACTAA |
| | | | | ATTAAATGATCTCTGCTTTACTAATGTCTATGCAGATT |
| | | | | CATTTGTAATTAGAGGTGATGAAGTCAGACAAATCGC |
| | | | | TCCAGGGCAAACTGGAAAGATTGCTGATTATAATTAT |
| | | | | AAATTACCAGATGATTTTACAGGCTGCGTTATAGCTT |
| | | | | GGAATTCTAACAATCTTGATTCTAAGGTTGGTGGTAA |
| | | | | TTATAATTACCTGTATAGATTGTTTAGGAAGTCTAATC |
| | | | | TCAAACCTTTTGAGAGAGATATTTCAACTGAAATCTAT |
| | | | | CAGGCCGGTAGCACACCTTGTAATGGTGTTGAAGGTT |
| | | | | TTAATTGTTACTTTCCTTTACAATCATATGGTTTCCAAC |
| | | | | CCACTAATGGTGTTGGTTACCAACCATACAGAGTAGT |
| | | | | AGTACTTTCTTTTGAACTTCTACATGCACCAGCAACTG |
| | | | | TTTGTGGACCTAAAAAGTCTACTAATTTGGTTAAAAAC |
| | | | | AAATGTGTCAATTTCGGAGGTCATCATCATCACCACC |
| | | | | ACGGAGGTGGCTCATCAGGATCAGGTgcggccgctatggt |
| | | | | gagcaagggcgaggagctgttcaccggggtggtgcccatcctggtcg |
| | | | | agctggacggcgacgtaaacggccacaagttcagcgtgtccggcgag |
| | | | | ggcgagggcgatgccacctacggcaagctgaccctgaagttcatctgc |
| | | | | accaccggcaagctgcccgtgccctggcccaccctcgtgaccaccctg |
| | | | | acctacggcgtgcagtgcttcagccgctaccccgaccacatgaagcag |
| | | | | cacgacttcttcaagtccgccatgcccgaaggctacgtccaggagcgc |
| | | | | accatcttcttcaaggacgacggcaactacaagacccgcgccgaggt |
| | | | | gaagttcgagggcgacaccctggtgaaccgcatcgagctgaagggca |
| | | | | tcgacttcaaggaggacggcaacatcctggggcacaagctggagtac |
| | | | | aactacaacagccacaacgtctatatcatggccgacaagcagaagaa |
| | | | | cggcatcaaggtgaacttcaagatccgccacaacatcgaggacggca |
| | | | | gcgtgcagctcgccgaccactaccagcagaacacccccatcggcgac |
| | | | | ggccccgtgctgctgcccgacaaccactacctgagcacccagtccgcc |
| | | | | ctgagcaaagaccccaacgagaagcgcgatcacatggtcctgctgga |
| | | | | gttcgtgaccgccgccgggatcactctcggcatggacgagctgtacaa |
| | | | | g |
| C7-IL17 | ColE7-N-terminally | 45 | | ATGGAGAGTAAACGGAATAAGCCAGGGAAGGC |
| | fused to human | | | AACAGGTAAAGGAAAACCTGTCAATAATAAGTG |
| | IL17a | | | GTTAAATAATGCAGGTAAAGACTTAGGTTCTCCT |
| | | | | GTTCCAGATCGTATAGCTAATAAACTACGTGATA |
| | | | | AGGAGTTTAAAAGTTTCGATGATTTTCGTAAGAA |
| | | | | ATTCTGGGAAGAAGTGTCAAAAGATCCTGAGTTA |
| | | | | AGTAAACAATTTAGTCGAAACAATAATGATCGAA |
| | | | | TGAAGGTTGGAAAAGCGCCCAAGACTAGAACCC |
| | | | | AGGATGTTTCAGGGAAGGCAACTTCATTCGCACT |
| | | | | TCATCATGAGAAGCCGATCAGCCAAAATGGTGG |
| | | | | TGTCTATGATATGGATAACATCAGCGTGGTAACA |
| | | | | CCTAAACGTGCTATTGATATTCACCGAGGTAAAA |
| | | | | GCGGAGGTGGCTCATCAGGATCtGGTGAAAACC |
| | | | | TGTATTTTCAGGGatccGGAGGTGGCTCAggaatca |
| | | | | caatcccacgaaatccaggatgcccaaattctgaggacaagaa |
| | | | | cttcccccggactgtgatggtcaacctgaacatccataaccgga |
| | | | | ataccaataccaatcccaaaaggtcctcagattactacaaccga |
| | | | | tccacctcaccttggaatctccaccgcaatgaggaccctgagag |
| | | | | atatccctctgtgatctgggaggcaaagtgccgccacttgggctg |
| | | | | catcaacgctgatgggaacgtggactaccacatgaactctgtcc |
| | | | | ccatccagcaagagatcctggtcctgcgcagggagcctccacac |
| | | | | tgccccaactccttccggctggagaagatactggtgtccgtggg |
| | | | | ctgcacctgtgtcaccccgattgtccaccatgtggcctaataa |

T7 promoter parent sequence:
gcataat (SEQ ID NO: 44)

1. A virus-like particle (VLP) comprising:
One or more hepatitis B capsid proteins
One or more pairs of binding proteins, each pair of binding proteins comprising a first binding protein and a second binding protein, wherein the pair of binding proteins comprises a bacterial toxin and its inhibitor
One or more functional molecules
wherein each hepatitis B capsid protein is attached to a first binding protein, wherein each functional molecule is attached to a second binding protein, and wherein the first and second binding proteins are bound to each other.

2. A VLP according to claim 1, wherein the first binding protein is the bacterial toxin inhibitor, and the second binding protein is the bacterial toxin.

3. A VLP according to claim 1 or 2, wherein each functional molecule is indirectly attached to a second binding protein via a third binding protein, preferably the third binding protein is protein G.

4. A VLP according to claim 1, 2 or 3, wherein the first binding protein comprises a chemical modification, optionally wherein the chemical modification is attached to a second functional molecule.

5. A VLP according to any preceding claim, wherein each functional molecule may be the same or different, preferably wherein the functional molecule is selected from: a protein or non-protein antigen or an epitope thereof, an antigen binding protein, or a flourescent molecule.

6. A VLP according to claim 3, wherein each functional molecule is an antigen binding protein, preferably wherein the antigen binding protein is selected from an antibody or binding fragment thereof, an antibody mimetic, and an aptamer, preferably wherein the antigen binding protein is an antibody, preferably an antibody which is capable of binding an antigen of interest.

7. A virus-like particle (VLP) comprising:
One or more viral capsid proteins
One or more first binding proteins comprising a chemical modification, wherein each first binding protein is a bacterial toxin inhibitor;
One or more functional molecules
wherein each viral capsid protein is attached to a first binding protein, and wherein each chemical modification is attached to a functional molecule.

8. A VLP according to claim 7, wherein the viral capsid protein is a Hepatitis B capsid protein.

9. A VLP according to claim 7 or 8, further comprising a second binding protein, wherein the second binding protein is bound to the first binding protein, optionally wherein the second binding protein is attached to a second functional molecule.

10. A VLP according to claim 9, wherein the second binding protein is a bacterial toxin, preferably a bacterial toxin which binds to the bacterial toxin inhibitor.

11. A VLP according to claim 9 or 10, wherein each second functional molecule is indirectly attached to a second binding protein via a third binding protein, preferably the third binding protein is protein G.

12. A VLP according to any of claims 7-11, wherein each functional molecule may be the same or different, preferably wherein the functional molecule is selected from: a protein or non-protein antigen or an epitope thereof, an antigen binding protein, or a flourescent molecule.

13. A VLP according to claim 11, wherein the each functional molecule is an antigen binding protein, preferably wherein the antigen binding protein is selected from an antibody or binding fragment thereof, an antibody mimetic, and an aptamer, preferably wherein the antigen binding protein is an antibody, preferably an antibody which is capable of binding an antigen of interest.

14. A VLP according to any preceding claim, wherein the bacterial toxin inhibitor is selected from Im7, Im8, Im9, Im2, and Barstar.

15. A VLP according to any of claim 1-6, or 10-14, wherein the bacterial toxin is a bacterial nuclease, preferably selected from: ColE7, ColE8, ColE9, ColE2, and Barnase.

16. A VLP according to any of claim 1-6 or 10-15, wherein the bacterial toxin and its inhibitor are Barnase and Barstar, preferably wherein the Barstar amino acid sequence comprises one or more of the following substitutions: C40A, C82A, and I87E, and/or wherein the amino acid sequence of Barnase comprises the following substitution: E73W.

17. A VLP according to any of claim 1-6 or 10-15, wherein the bacterial toxin and its inhibitor are ColE7 and Im7, preferably wherein the amino acid sequence of Im7 comprises the following substitution: F41L, and/or wherein the amino acid sequence of ColE7 comprises one or more of the following substitutions: Arg538Ala, Glu542Ala, and His569Ala.

18. A VLP according to any of claim 1-6, or 8-17, wherein the first binding protein is inserted into the major immunodominant region of the hepatitis B capsid protein, preferably the first binding protein is inserted between amino acid residues 76 and 80 of the major immunodominant region of the hepatitis B capsid protein.

19. A VLP according to any of claim 1-6, or 8-18, wherein the hepatitis B capsid protein comprises an amino acid sequence with the following amino acid deletions: E77 and D78.

20. A VLP according to any of claims 4-19, wherein the chemical modification is an alkane having an amine group, preferably a 1-10 carbon alkane having an amine group, preferably wherein the chemical modification is selected from DEAE or octylamine.

21. A VLP according to claim 5 or 12, wherein the non-protein antigen is selected from a: sugar, lipid, carbohydrate, or small molecule chemical.

22. A VLP according to claim 5 or 12, wherein the protein antigen is derived from an infectious agent, preferably wherein the protein antigen is derived from a virus, bacterium, fungus, protozoan, archaeon, preferably wherein the protein antigen is derived from a virus, preferably wherein the virus is selected from: Adeno-associated virus, Chikungunya virus, Crimean-Congo hemorrhagic fever virus, Dengue virus, Ebolavirus, Echovirus, Encephalomyocarditis virus, Epstein-Barr virus, Hantaan virus, Hepatitis A virus, Hepatitis B virus, Hepatitis C virus, Hepatitis D virus, Hepatitis E virus, Human adenovirus, Human astrovirus, Human coronavirus, Human cytomegalovirus, Human enterovirus, Human herpesvirus, Human immunodeficiency virus, Human papillomavirus, Human parainfluenza, Human respiratory syncytial virus, Human rhinovirus, Human torovirus, Influenza A virus, Influenza B virus, Influenza C virus, Japanese encephalitis virus, Polyomavirus, Kunjin virus, Lassa virus, Measles virus, Molluscum contagiosum virus, Mumps virus, Nipah virus, Poliovirus, Rabies virus, Rotavirus A, Rotavirus B, Rotavirus C, Rubella virus, Sagiyama virus, Salivirus A, Sapporo virus, Sindbis virus, Toscana virus, Uukuniemi virus, Varicella-zoster virus, Venezuelan equine encephalitis virus, Vesicular stomatitis virus, Western equine encephalitis virus, West Nile virus, Yellow fever virus, Zika virus, preferably wherein the protein antigen is derived from a coronavirus, preferably from SARS-CoV-2, preferably the protein antigen is the whole or part of a spike protein derived from SARS-CoV-2, or the whole or part of a nucleocapsid protein derived from SARS-CoV-2.

23. A VLP according to claim 5 or 12, wherein the protein antigen is derived from a non-infectious agent, preferably wherein the protein antigen is derived from an inflammatory molecule, a molecule causing degenerative changes in nervous, cartilage or bone tissue, or a molecule causing worsening of a neoplastic disease, preferably wherein the protein antigen is an inflammatory molecule selected from a chemokine, cytokine, or protease, preferably wherein the protein antigen is selected from: IL1, IL2, Il3, Il4, IL5, Il6, Il7, IL8, IL9, IL10, IL11, IL12, IL13, IL17, IL33, TNFα, TNFβ, IFNα, IFNβ, IFNγ, G-CSF, GM-CSF, M-CSF, erythropoietin, and TGFβ, preferably wherein the protein antigen is the whole or part of IL13, IL17 or IL33.

24. A VLP according to claim 5 or 12, wherein the flourescent molecule is selected from: GFP, EBFP, EBFP2, Azurite, GFPuv, T-saphhire, Cerulean, CFP, mCFP, mTurquoise2, CyPet, mKeima-red, tagCFP, AmCyan1, mTFP1, midoriishi cyan, turboGFP, tagGFP, emerald, azami green, ZsGreen1, YFP, tagYFP, EYFP, topaz, venus, mCtrine, YPet, turboYFP, ZsYellow1, Kusabira Orange, mOrange, allophycocyanin, mkO, RFP, turboRFP, tdTomato, tagRFP, dsRed, mStrawberry, turboFP602, asRed2, J-red, R-phycoerythrin, B-phycoerythrin, mCherry, HcRed, Katusha, P3, peridin chlorophyll, mKate, turboFP635, mPlum, mRaspberry, preferably wherein the flourescent molecule is GFP.

25. A capsid fusion protein comprising a viral capsid protein fused to a binding protein, wherein the binding protein is a bacterial toxin inhibitor.

26. A functional fusion protein comprising a functional molecule fused to a binding protein wherein the binding protein is a bacterial toxin.

27. A functional fusion protein comprising a first binding protein fused to a further binding protein, wherein the first binding protein is a bacterial toxin, and the further binding protein is able to bind to functional molecules that are antigen binding proteins.

28. One or more nucleic acids encoding the capsid fusion protein according to claim 25 or the functional fusion protein according to claim 26 or 27.

29. One or more vectors comprising the one or more nucleic acids of claim 28.

30. A host cell comprising the one or more nucleic acids of claim 28 or the one or more vectors of claim 29.

31. A host cell comprising one or more vectors, the one or more vectors comprising a first nucleic acid encoding a hepatitis B capsid protein attached to a first binding protein; and a second nucleic acid encoding a functional molecule attached to a second binding protein; wherein the first and second binding proteins are capable of binding to each other.

32. A host cell comprising one or more vectors, the one or more vectors comprising a first nucleic acid encoding a viral capsid protein attached to a first binding protein; and a second nucleic acid encoding a functional molecule; wherein the functional molecule is capable of binding to the first binding protein via a chemical modification of the first binding protein.

33. A process of producing a virus-like particle (VLP) in a single host cell comprising:
(a) Providing a host cell comprising one or more vectors, wherein the one or more vectors comprise:
(i) a first nucleic acid encoding a viral capsid protein attached to a first binding protein; and
(ii) (a) a second nucleic acid encoding a functional molecule optionally attached to a second binding protein; and/or
(b) a third nucleic acid encoding a second binding protein attached to a third binding protein;
(b) Culturing the host cell under conditions to express the proteins from the first, second and/or third nucleic acids respectively;
(c) Forming virus-like particles from the proteins.

34. A process of producing a virus-like particle (VLP), comprising;
(a) Providing a first host cell comprising one or more vectors, wherein the one or more vectors comprise:
(i) a first nucleic acid encoding a hepatitis B capsid protein attached to a first binding protein, wherein the first binding protein is a bacterial toxin inhibitor;
(b) Providing a one or more further host cell(s) comprising one or more vectors, wherein the one or more vectors comprise:
a second nucleic acid encoding a functional molecule optionally attached to a second binding protein, wherein the second binding protein is a bacterial toxin; and/or
(ii) a third nucleic acid encoding a second binding protein attached to a third binding protein, wherein the second binding protein is a bacterial toxin;
(c) Culturing the host cells under conditions to express the proteins from the first, second and/or third nucleic acids respectively;
(d) Recovering the proteins;
(e) Mixing the proteins to form virus-like particles.

35. An immunogenic composition comprising the virus-like particle of any of claims 1-25.

36. A virus-like particle (VLP) of any of claims 1-24, or an immunogenic composition of claim 35 for use as a medicament.

37. A virus-like particle (VLP) of any of claims 1-24, or an immunogenic composition of claim 35 for use in the prevention and/or treatment of infectious diseases, cardiovascular diseases, cancer, inflammatory diseases, autoimmune diseases, neurological disease, rheumatological degenerative disease, or addiction.

38. Use of the virus-like particle (VLP) of any of claims 1-24 in research, or in the diagnosis of a disease.

39. A method of diagnosing a disease in a subject comprising:
(a) Providing a virus like particle according to any of claims 1-24, wherein the functional molecule is an antibody directed towards an antigen derived from a disease causing agent;
(b) Mixing the virus like particle with a suitable sample from the subject;
(c) Detecting whether the virus like particle precipitates;
(d) Diagnosing the presence of a disease if the VLP precipitates.
